SEQUENCE LISTING (142 sequences)

SEQ ID NO: 1 - protein, 108 aa, Mus sp.
QIVLTQSPAIMSASLGERVTMTCTASSSVSSSYLHWYQKKPGSSPKLWVYSTSNLASGVPVRFSGSGSGTSYSLTISSMEAEDAATYYCHQHHRSPVTFGSGTKLEMK

SEQ ID NO: 2 - protein, 107 aa, Mus sp.
DIQMTQSPASQSASLGESVTFTCLASQTIGTWLAWYQQRPGKSPQLLIYAATSLADGVPSRFSGSGSGTQFSFNIRSLQAEDFASYYCQQVYTTPLTFGGGTKLEIK

SEQ ID NO: 3 - protein, 107 aa, Mus sp.
DIQMTQSPASQSASLGESVTFTCLASQTIGTWLGWYQQKPGKSPQLLIYRSTTLADGVPSRFSGSGSGTKFSFKISSLQAADFASYYCQQLYSAPYTFGGGTKLEIR

SEQ ID NO: 4 - protein, 112 aa, Mus sp.
DVLLTQTPLSLPVSLGDQASISCRSSQNIVHSNGNTYLQWYLQKPGQSPKLLIYKVSNRFSGVPDRFSGSGSGTDFTLKISRVEAEDLGVYYCFQGSHVPFTFGAGTKLELK

SEQ ID NO: 5 - protein, 107 aa, synthetic construct
DIQMTQTTSSLSASLGDRVTISCRASQDIYKYLNWYQQKPDGTLKLLIYYTSGLHSGVPSRFSGSGSGTDFSLTISNLEPEDIATYFCQQDSKFPWTFGGDTKLEIK

SEQ ID NO: 6 - protein, 108 aa, Mus sp.
QIVLTQSPAIMSASLGERVTMTCTASSSVSSSYFHWYQQKPGSSPKLWIYRTSNLASGVPGRFSGSGSGTSYSLTISSMEAEDAATYYCHQFHRSPLTFGAGTKLELK

SEQ ID NO: 7 - protein, 107 aa, Mus sp.
DIVMTQSQKFLSTSVGVRVSVTCKASQDVGTNVLWYQQKIGQSPKPLIYSASYRHSGVPDRFTGSGSGTDFTLIISNVQSEDLAEYFCQQYSRYPLTFGPGTKLELK

SEQ ID NO: 8 - protein, 107 aa, Mus sp.
DIVMTQSQKFLSTSVGVRVSVTCKASQDVGTNVLWYQQKIGQSPKALIYSASYRHSGVPDRFTGSGSGTDFTLIITNVQSEDLAEYFCQQYSRYPLTFGPGTKLELK

SEQ ID NO: 9 - protein, 107 aa, Mus sp.
DIVMTQSQKFMSATVGGRVNITCKASQNVGRAVAWYQQKPGQSPKLLTHSASNRYTGVPDRFTGSGSGTDFTLTITNMQSEDLADYFCQQYSSYPLTFGAGTKLDLK

SEQ ID NO: 10 - protein, 107 aa, Mus sp.
DIQMTQSPASQSASLGESVTFSCLASQTIGTWLGWYQQKPGKSPQLLIYRATSLADGVPSRFSGSGSGTNFSFKISSLQAEDLASYYCQQLYSGPYTFGGGTKLEIR

SEQ ID NO: 11 - protein, 119 aa, Mus sp.
QVQLQQSGTELLKPGASVKLSCKASGNTVTSYWMHWVKQRPGQGLEWIGEILPSTGRTNYNENFKGKAMLTVDKSSSTAYMQLSSLASEDSAVYYCTIVYFGNPWFAYWGQGTLVTVSA

SEQ ID NO: 12 - protein, 125 aa, Mus sp.
EVQLQQSGPELVKPGASVKLSCKASGYTFTDNYMNWVRQSHGKSLEWIGRVNPSNGDTKYNQNFKGKATLTVDKSLSTAYMQLNGLTSEDSAVYYCGRTKNFYSSYSYDDAMDYWGQGTSVTVSS

SEQ ID NO: 13 - protein, 124 aa, Mus sp.
EVQLQQSGAEFVRPGASVKFSCTASGFNIKDDYIHWVRQRPEQGLEWVGRIDPANGNTKYAPKFQDKATITADTSSNTAYLQLSSLTSEDTAVYYCAKSFPNNYYSYDDAFAYWGQGTLVTVSA

SEQ ID NO: 14 - protein, 118 aa, Mus sp.
QVQLKESGPVLVAPSQSLSITCTVSGFSLTKFGVHWIRQTPGKGLEWLGVIWAGGPTNYNSALMSRLTISKDISQSQVFLRIDSLQTDDTAMYYCAKQIYYSTLVDYWGQGTSVTVSS

SEQ ID NO: 15 - protein, 120 aa, Mus sp.
QVQLKESGPGLVAPSQSLFITCTVSGFSLSSYEINWVRQVPGKGLEWLGVIWTGITTNYNSALISRLSISKDNSKSLVFLKMNSLQTDDTAIYYCARGTGTGFYYAMDYWGQGTSVTVSS

SEQ ID NO: 16 - protein, 119 aa, Mus sp.
QVQLQQPGADFVRPGASMRLSCKASGYSFTSSWIHWVKQRPGQGLEWIGEINPGNVRTNYNENFRNKATLTVDKSSTTAYMQLRSLTSADSAVYYCTVVFYGEPYFPYWGQGTLVTVSA

SEQ ID NO: 17 - protein, 119 aa, Mus sp.
QVQLKESGPGLVAPSQSLSITCTVSGFSLTNYAVHWVRQFPGKGLEWLGVIWSDGSTDFNAPFKSRLSINKDNSKSQVFFKMNSLQIDDTAIYYCARKGGYSGSWFAYWGQGTLVTVSA

SEQ ID NO: 18 - protein, 119 aa, Mus sp.
QVQLKESGPGLVAPSQSLSITCTVSGFSLTNYAVHWVRQFPGKGLEWLGVIWSDGSTDYNAPFKSRLSINKDNSKSQVFFKMNSLQTDDTAIYYCARKGGYSGSWFAYWGQGTLVTVSA

SEQ ID NO: 19 - protein, 117 aa, Mus sp.
QVQLKESGPVLVAPSQSLSITCTVSGFSLTNYGVHWVRQPPGKGLEWLGVIWPVGSTNYNSALMSRLSIHKDNSKSQVFLRMNSLQTDDTAIYYCAKMDWDDFFDYWGQGTTLTVSS

SEQ ID NO: 20 - protein, 124 aa, Mus sp.
EVQLQQSGAELVRPGASVRLSCTASGFNIKDDYIHWVRQRPKQGLEWLGRIDPANGNTKYDPRFQDKATITADTSSNTAYLHLSSLTSEDTAVYYCAKSFPDNYYSYDDAFAYWGQGTLVTVSA

SEQ ID NO: 21 - protein, 12 aa, Mus sp.
TASSSVSSSYLH

SEQ ID NO: 22 - protein, 11 aa, Mus sp.
LASQTIGTWLA

SEQ ID NO: 23 - protein, 11 aa, Mus sp.
LASQTIGTWLG

SEQ ID NO: 24 - protein, 16 aa, Mus sp.
RSSQNIVHSNGNTYLQ

SEQ ID NO: 25 - protein, 11 aa, Mus sp.
RASQDIYKYLN

SEQ ID NO: 26 - protein, 12 aa, Mus sp.
TASSSVSSSYFH

SEQ ID NO: 27 - protein, 11 aa, Mus sp.
KASQDVGTNVL

SEQ ID NO: 28 - protein, 11 aa, Mus sp.
KASQNVGRAVA

SEQ ID NO: 29 - protein, 11 aa, Mus sp.
LASQTIGTWLG

SEQ ID NO: 30 - protein, 7 aa, Mus sp.
STSNLAS

SEQ ID NO: 31 - protein, 7 aa, Mus sp.
AATSLAD

SEQ ID NO: 32 - protein, 7 aa, Mus sp.
RSTTLAD

SEQ ID NO: 33 - protein, 7 aa, Mus sp.
KVSNRFS

SEQ ID NO: 34 - protein, 7 aa, Mus sp.
YTSGLHS

SEQ ID NO: 35 - protein, 7 aa, Mus sp.
RTSNLAS

SEQ ID NO: 36 - protein, 7 aa, Mus sp.
SASYRHS

SEQ ID NO: 37 - protein, 7 aa, Mus sp.
SASNRYT

SEQ ID NO: 38 - protein, 7 aa, Mus sp.
RATSLAD

SEQ ID NO: 39 - protein, 9 aa, Mus sp.
HQHHRSPVT

SEQ ID NO: 40 - protein, 9 aa, synthetic construct
QQVYTTPLT

SEQ ID NO: 41 - protein, 9 aa, Mus sp.
QQLYSAPYT

SEQ ID NO: 42 - protein, 9 aa, Mus sp.
FQGSHVPFT

SEQ ID NO: 43 - protein, 9 aa, Mus sp.
QQDSKFPWT

SEQ ID NO: 44 - protein, 9 aa, Mus sp.
HQFHRSPLT

SEQ ID NO: 45 - protein, 9 aa, Mus sp.
QQYSRYPLT

SEQ ID NO: 46 - protein, 9 aa, Mus sp.
QQYSSYPLT

SEQ ID NO: 47 - protein, 9 aa, Mus sp.
QQLYSGPYT

SEQ ID NO: 48 - protein, 10 aa, Mus sp.
GNTVTSYWMH

SEQ ID NO: 49 - protein, 10 aa, Mus sp.
GYTFTDNYMN

SEQ ID NO: 50 - protein, 10 aa, Mus sp.
GFNIKDDYIH

SEQ ID NO: 51 - protein, 10 aa, Mus sp.
GFSLTKFGVH

SEQ ID NO: 52 - protein, 10 aa, Mus sp.
GFSLSSYEIN

SEQ ID NO: 53 - protein, 10 aa, Mus sp.
GYSFTSSWIH

SEQ ID NO: 54 - protein, 10 aa, Mus sp.
GFSLTNYAVH

SEQ ID NO: 55 - protein, 10 aa, Mus sp.
GFSLTNYGVH

SEQ ID NO: 56 - protein, 10 aa, Mus sp.
GFNIKDDYIH

SEQ ID NO: 57 - protein, 17 aa, Mus sp.
EILPSTGRTNYNENFKG

SEQ ID NO: 58 - protein, 17 aa, Mus sp.
RVNPSNGDTKYNQNFKG

SEQ ID NO: 59 - protein, 17 aa, Mus sp.
RIDPANGNTKYAPKFQD

SEQ ID NO: 60 - protein, 16 aa, Mus sp.
VIWAGGPTNYNSALMS

SEQ ID NO: 61 - protein, 16 aa, Mus sp.
VIWTGITTNYNSALIS

SEQ ID NO: 62 - protein, 15 aa, Mus sp.
EINPGNVRTNYNENF

SEQ ID NO: 63 - protein, 16 aa, Mus sp.
VIWSDGSTDFNAPFKS

SEQ ID NO: 64 - protein, 16 aa, Mus sp.
VIWSDGSTDYNAPFKS

SEQ ID NO: 65 - protein, 16 aa, Mus sp.
VIWPVGSTNYNSALMS

SEQ ID NO: 66 - protein, 17 aa, Mus sp.
RIDPANGNTKYDPRFQD

SEQ ID NO: 67 - protein, 10 aa, Mus sp.
VYFGNPWFAY

SEQ ID NO: 68 - protein, 16 aa, Mus sp.
TKNFYSSYSYDDAMDY

SEQ ID NO: 69 - protein, 15 aa, Mus sp.
SFPNNYYSYDDAFAY

SEQ ID NO: 70 - protein, 10 aa, Mus sp.
QIYYSTLVDY

SEQ ID NO: 71 - protein, 12 aa, Mus sp.
GTGTGFYYAMDY

SEQ ID NO: 72 - protein, 10 aa, Mus sp.
VFYGEPYFPY

SEQ ID NO: 73 - protein, 11 aa, Mus sp.
KGGYSGSWFAY

SEQ ID NO: 74 - protein, 9 aa, Mus sp.
MDWDDFFDY

SEQ ID NO: 75 - protein, 15 aa, Mus sp.
SFPDNYYSYDDAFAY

SEQ ID NO: 76 - protein, 108 aa, synthetic construct
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIK

SEQ ID NO: 77 - protein, 108 aa, synthetic construct
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIK

SEQ ID NO: 78 - protein, 108 aa, synthetic construct
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIK

SEQ ID NO: 79 - protein, 108 aa, synthetic construct
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIK

SEQ ID NO: 80 - protein, 108 aa, synthetic construct [sequence omitted]

SEQ ID NO: 81 - protein, 108 aa, synthetic construct
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIK

SEQ ID NO: 82 - protein, 108 aa, synthetic construct
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIK

SEQ ID NO: 83 - protein, 108 aa, synthetic construct
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIK

SEQ ID NO: 84 - protein, 107 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIK

SEQ ID NO: 85 - protein, 107 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIK

SEQ ID NO: 86 - protein, 107 aa, synthetic construct
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIK

SEQ ID NO: 87 - protein, 119 aa, synthetic construct
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSS

SEQ ID NO: 88 - protein, 119 aa, synthetic construct
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSS

SEQ ID NO: 89 - protein, 119 aa, synthetic construct [sequence omitted]

SEQ ID NO: 90 - protein, 119 aa, synthetic construct
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSS

SEQ ID NO: 91 - protein, 119 aa, synthetic construct
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSS

SEQ ID NO: 92 - protein, 119 aa, synthetic construct
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSS

SEQ ID NO: 93 - protein, 119 aa, synthetic construct
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSS

SEQ ID NO: 94 - protein, 119 aa, synthetic construct
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSS

SEQ ID NO: 95 - protein, 119 aa, synthetic construct
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSS

SEQ ID NO: 96 - protein, 119 aa, synthetic construct
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSS

SEQ ID NO: 97 - protein, 119 aa, synthetic construct
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSS

SEQ ID NO: 98 - protein, 119 aa, synthetic construct
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSS

SEQ ID NO: 99 - protein, 119 aa, synthetic construct
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSS

SEQ ID NO: 100 - protein, 119 aa, synthetic construct
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSS

SEQ ID NO: 101 - protein, 119 aa, synthetic construct
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSS

SEQ ID NO: 102 - protein, 7 aa, Mus sp.
RTSTLAS

SEQ ID NO: 103 - protein, 7 aa, Mus sp.
RTSILAS

SEQ ID NO: 104 - protein, 7 aa, Mus sp.
RTSRLAS

SEQ ID NO: 105 - protein, 7 aa, Mus sp.
RTSQLAS

SEQ ID NO: 106 - protein, 7 aa, Mus sp.
RTSKLAS

SEQ ID NO: 107 - protein, 10 aa, Mus sp.
GFSLTDYAVH

SEQ ID NO: 108 - protein, 15 aa, Mus sp.
EILPGVVRTNYNENF

SEQ ID NO: 109 - protein, 15 aa, Mus sp.
EINPGAVRTNYNENF

SEQ ID NO: 110 - protein, 15 aa, Mus sp.
EINPGLVRTNYNENF

SEQ ID NO: 111 - protein, 15 aa, Mus sp.
EINPGSVRTNYNENF

SEQ ID NO: 112 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 107 aa, synthetic construct [sequence omitted]

SEQ ID NO: 114 - protein, 215 aa, synthetic construct
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 215 aa, synthetic construct
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 215 aa, synthetic construct
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 215 aa, synthetic construct
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 215 aa, synthetic construct
QIVLTQSPGTLSLSPGERATMTCTASSSVSSSYFHWYQQKPGQAPRLWIYRTSRLASGVPDRFSGSGSGTDFTLTISRLEPEDAATYYCHQFHRSPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 215 aa, synthetic construct
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 215 aa, synthetic construct
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 215 aa, synthetic construct
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 214 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 214 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 214 aa, synthetic construct
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 449 aa, synthetic construct
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 449 aa, synthetic construct
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 449 aa, synthetic construct
QVQLVQSGAEVKKPGASVKVSCKASGYSFTSSWIHWVKQAPGQGLEWMGEINPGNVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 449 aa, synthetic construct
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 449 aa, synthetic construct
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 449 aa, synthetic construct
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 449 aa, synthetic construct
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 449 aa, synthetic construct
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 449 aa, synthetic construct
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 449 aa, synthetic construct
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 449 aa, synthetic construct
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 449 aa, synthetic construct
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 449 aa, synthetic construct
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 449 aa, synthetic construct
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 449 aa, synthetic construct
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 7 aa, Mus sp.
RTSHLAS

SEQ ID NO: 141 - protein, 5 aa, Mus sp.
SSWIH

SEQ ID NO: 142 - protein, 17 aa, Mus sp.
EINPGNVRTNYNENFRN

(19) United States
(12) Patent Application Publication
(10) Pub. No.: US 2023/0115617 A1
(43) Pub. Date: Apr. 13, 2023

(54) ANTI-IL-36R ANTIBODIES FOR TREATMENT OF PALMOPLANTAR PUSTULOSIS

(71) Applicant: Boehringer Ingelheim International GmbH, Ingelheim am Rhein (DE)

(72) Inventors: Patrick BAUM, Biberach an der Riss (DE); Janine LAMAR, Ingelheim am Rhein (DE); Steven John PADULA, Wiesbaden (DE); Christian THOMA, Biberach an der Riss (DE)

(21) Appl. No.: 18/054,303

(22) Filed: Nov. 10, 2022

Related U.S. Application Data
(63) Continuation of application No. 16/722,133, filed on Dec. 20, 2019, now abandoned.
(60) Provisional application No. 62/891,464, filed on Aug. 26, 2019.
(60) Provisional application No. 62/815,431, filed on Mar. 8, 2019.
(60) Provisional application No. 62/785,316, filed on Dec. 27, 2018.

(51) Int. Cl. C07K 16/28; A61P 17/00

(52) U.S. Cl. CPC C07K 16/2866; A61P 17/00; A61K 9/0019

(57) ABSTRACT

The present invention relates to the treatment of or alleviation of signs and symptoms of palmoplantar pustulosis (PPP) with anti-IL-36R antibodies in a patient.

### SEQUENCE LISTING

The instant application contains a Sequence Listing which has been submitted electronically in XML format and is hereby incorporated by reference in its entirety. Said XML copy, created on Jul. 27, 2022, is named 09-0686-US-5.XML and is 134 bytes in size.

### TECHNICAL FIELD OF THE INVENTION

The present invention relates to methods and compositions for treatment of palmoplantar pustulosis (PPP). More specifically, the invention relates to administration of an anti-interleukin-36 receptor (anti-IL-36R) antibody to a subject with PPP. Still more specifically, the invention relates to administration of a dosing regimen of an anti-IL-36R antibody to a subject with PPP.

### BACKGROUND

Palmoplantar pustulosis, also known as palmoplantar pustular psoriasis (PPP) is a disease with a high unmet medical need. PPP is a chronic disease and a form of pustular psoriasis (as is Generalized Pustular Psoriasis, GPP). Recent evidence suggests that PPP is a genetically distinct entity from chronic plaque psoriasis as the major genetic determinant PSORS1 for plaque psoriasis has not been found in the pustular forms of psoriasis (PPP and GPP) patients. Experimental and human genetic data imply that the IL36 pathway drives the pustular psoriasis diseases of PPP and GPP.

PPP may be considered a rare disease. PPP is characterized by the presence of sterile pustules on palms and/or soles. Despite the limited area of skin involvement in PPP, the disease is very debilitating with a large impact on quality of life including ability to work. PPP symptoms include pruritus, burning sensations, and pain. In severe cases, the skin affliction makes walking or other activities of daily living challenging if not impossible. No approved treatment is available for PPP further highlighting the high need for an effective treatment option.

Genetic human studies have established a link between IL36R signaling and PPP: The same hypomorphic missense mutation in IL36RN reported for GPP has also been observed in PPP, albeit to a lesser extent as compared to GPP.

Further genetic linkage between PPP and the IL36 pathway has been recently disclosed. For example, mutations in other genes linked to the IL36 pathway such as CARD14 and AP1S3 have been linked to the pathogenesis of all forms of pustular psoriasis including PPP. CARD14 is specifically and predominately expressed in keratinocytes in the skin. It acts downstream of the IL36 pathway and is a known activator of NF-kB signaling. Mutations in the coding sequence (c.11T>G and c.97C>T) in AP1S3 have been linked to the pathogenesis of all forms of pustular psoriasis including PPP. The gene encodes a subunit of the AP-1 complex. Functionally the occurrence of these rare mutations causes a destabilizing the AP-1 complex and could be linked to impaired Toll-like receptor 3 signaling and subsequent expression of the anti-inflammatory mediator IFN-β.

Currently there is no standard of care available for the treatment of PPP (i.e., no approved therapy). PPP is notoriously difficult to treat. Patients usually end up being treated with the currently available systemic treatment options including retinoids, PUVA, methotrexate, ciclosporine and topical corticosteroids. Unfortunately, these options are usually not effective in reducing duration and severity of PPP. Thus, there is high unmet medical need for PPP.

### SUMMARY OF THE INVENTION

The present invention addresses the above need by providing biotherapeutics, in particular antibodies, which bind to IL-36R and provide therapeutic or prophylactic therapy for acute and/or chronic PPP and the associated signs and symptoms such as PPP flares (including new appearance or worsening of pustules).

In one aspect, the present invention relates to a method of treating palmoplantar pustulosis (PPP) in a patient, said method including administering or having administered to the patient a therapeutically effective amount of an anti-IL-36R antibody.

In another aspect, the present invention relates to a method of treating moderate to severe PPP in a patient, including administering or having administered to the patient a therapeutically effective amount of an anti-IL-36R antibody.

In another aspect, the present invention relates to a method of treating chronic disease conditions associated with PPP (including periodic appearance or worsening of pustules) in a patient, including administering or having administered to the patient a therapeutically effective amount of an anti-IL-36R antibody.

In another aspect, the present invention relates to a method of reducing or alleviating signs and symptoms of an acute or chronic phase flare-up (including new appearance or worsening of pustules) of PPP in a patient, said method including administering or having administered to the patient a therapeutically effective amount of an anti-IL-36R antibody.

In another aspect, the present invention relates to a method of reducing the severity and duration of PPP flares (including new appearance or worsening of pustules), said method comprising including administering or having administered to the patient a therapeutically effective amount of an anti-IL-36R antibody.

In another aspect, the present invention relates to a method of treating a skin disorder associated with acute PPP (including new appearance or worsening of pustules), said method including administering or having administered to the patient a therapeutically effective amount of an anti-IL-36R antibody.

In another aspect, the present invention relates to a method of preventing the recurrence of PPP flares (including new appearance or worsening of pustules) in a patient treated with an anti-IL-36R antibody of the present invention.

In another aspect, the present invention relates to a method of achieving a PPP ASI50 at week 16 in a patient treated with an anti-IL-36R antibody.

In another aspect, the present invention relates to a method of achieving a complete resolution of PPP symptoms in a patient treated with an anti-IL-36R antibody; wherein the PPP symptoms comprise pustule, erythema, crust, or scaling and the complete resolution comprises a PPP PGA score of 0 (clear, e.g., on signs of PPP; no scaling or crusts or pustule remains) or 1 (almost clear, slight scaling and/or erythema and/or slight crusts; very few new (yellow) and/or old (brown) pustules).

In another aspect, the present invention relates to a method of treating PPP in a patient, including:

(a) obtaining a biological sample from said patient, wherein the biological sample is obtained from source including lesional skin or whole blood;

(b) performing or having performed sequencing assay or expression analysis of one or more of genes;

(c) administering to the patient an effective amount of an anti-IL-36R antibody based on the gene sequencing assay or expression analysis results. In an embodiment relating to this aspect, the one or more of genes is IL36RN, CARD14, AP1S3, HLA-C, C15orf48, CCL20, CXCR2, IGHA1, IL17A, IL17F, IL36A, IL36B, IL36RN, LCN2, MIR155HG, S100A12, S100A7, S100A8, VNN1, CXCR2, IL36G, IL36RN, PI3, S100A12 and/or VNN3 in lesional skin or whole blood of the patient. For example, if the expression of the gene is above or below a threshold level, the treatment with an anti-IL-36R antibody occurs, otherwise not.

In one embodiment related to any of the aspects and embodiments described herein, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

In one embodiment related to any of the aspects and embodiments described herein, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In one embodiment related to any of the aspects and embodiments described herein, the anti-IL-36R antibody includes:

I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

In one embodiment related to any of the aspects and embodiments described herein, the anti-IL-36R antibody includes:

(i) a light chain variable region comprising the amino acid sequence of SEQ ID NO: 77; and a heavy chain variable region comprising the amino acid sequence of SEQ ID NO: 87; or

(ii) a light chain variable region comprising the amino acid sequence of SEQ ID NO: 77; and a heavy chain variable region comprising the amino acid sequence of SEQ ID NO: 88; or

(iii) a light chain variable region comprising the amino acid sequence of SEQ ID NO: 77; and a heavy chain variable region comprising the amino acid sequence of SEQ ID NO: 89; or

(iv) a light chain variable region comprising the amino acid sequence of SEQ ID NO: 80; and a heavy chain variable region comprising the amino acid sequence of SEQ ID NO: 87; or

(v) a light chain variable region comprising the amino acid sequence of SEQ ID NO: 80; and a heavy chain variable region comprising the amino acid sequence of SEQ ID NO: 88; or

(vi) a light chain variable region comprising the amino acid sequence of SEQ ID NO: 80; and a heavy chain variable region comprising the amino acid sequence of SEQ ID NO: 89; or

(vii) a light chain variable region comprising the amino acid sequence of SEQ ID NO: 85; and a heavy chain variable region comprising the amino acid sequence of SEQ ID NO: 100; or

(viii) a light chain variable region comprising the amino acid sequence of SEQ ID NO: 85; and a heavy chain variable region comprising the amino acid sequence of SEQ ID NO:101; or

(ix) a light chain variable region comprising the amino acid sequence of SEQ ID NO: 86; and a heavy chain variable region comprising the amino acid sequence of SEQ ID NO: 100; or

(x) a light chain variable region comprising the amino acid sequence of SEQ ID NO: 86; and a heavy chain variable region comprising the amino acid sequence of SEQ ID NO:101.

In one embodiment related to any of the aspects and embodiments described herein, the anti-IL-36R antibody includes:

i. a light chain comprising the amino acid sequence of SEQ ID NO: 115; and a heavy chain comprising the amino acid sequence of SEQ ID NO: 125; or

ii. a light chain comprising the amino acid sequence of SEQ ID NO: 115; and a heavy chain comprising the amino acid sequence of SEQ ID NO: 126; or

iii. a light chain comprising the amino acid sequence of SEQ ID NO: 115; and a heavy chain comprising the amino acid sequence of SEQ ID NO: 127; or

iv. a light chain comprising the amino acid sequence of SEQ ID NO: 118; and a heavy chain comprising the amino acid sequence of SEQ ID NO: 125; or

v. a light chain comprising the amino acid sequence of SEQ ID NO: 118; and a heavy chain comprising the amino acid sequence of SEQ ID NO: 126; or

vi. a light chain comprising the amino acid sequence of SEQ ID NO: 118; and a heavy chain comprising the amino acid sequence of SEQ ID NO: 127; or

vii. a light chain comprising the amino acid sequence of SEQ ID NO: 123; and a heavy chain comprising the amino acid sequence of SEQ ID NO: 138; or

viii. a light chain comprising the amino acid sequence of SEQ ID NO: 123; and a heavy chain comprising the amino acid sequence of SEQ ID NO: 139; or

ix. a light chain comprising the amino acid sequence of SEQ ID NO: 124; and a heavy chain comprising the amino acid sequence of SEQ ID NO: 138.

In an embodiment relating to any of the aspects and embodiments described herein, the anti-IL-36R antibody is administered subcutaneously or intravenously or by both routes simultaneously or sequentially and in any order. In a related embodiment, the subcutaneous administration comprises administration of 300 mg or 600 mg dose of the anti-IL-36R antibody. In a related embodiment, the intravenous administration comprises administering 300 mg, 600 mg, 900 mg or 1200 mg dose of the anti-IL-36R antibody. In a related embodiment, the subcutaneous administration is conducted at qw (once every week), q2w (once every 2 weeks), q4w (once every 4 weeks), q6w (once every 6 weeks) or q8w (once every 8 weeks) interval, or a combination thereof. In a related embodiment, the intravenous administration is conducted at q4w (once every 4 weeks), q8w (once every 8 weeks) or q12w (once every 12 weeks) interval, or a combination thereof.

In another embodiment relating to any of the aspects and embodiments described herein, the anti-IL-36R antibody is administered subcutaneously or intravenously or by both routes simultaneously or sequentially and in any order. In a related embodiment, the subcutaneous administration comprises an initial dose. In a related embodiment, the subcutaneous administration further comprises a subsequent dose. In a related embodiment, the administration of the anti-IL-36R antibody includes an initial dose and a subsequent dose. In a related embodiment, the initial dose is administered intravenously or subcutaneously. In a related embodiment, the subsequent dose is administered subcutaneously. In a related embodiment, the initial dose is 150 mg, 300 mg or 600 mg. In a related embodiment, the initial dose of 150 mg or 300 mg is administered per day (in consecutive days) for two weeks. In a related embodiment, the initial dose of 600 mg is administered once per week for two weeks including weeks 0 and 1; weeks 0 and 2; weeks 0 and 3; or weeks 0 and 4. In a related embodiment, the initial dose of 600 mg is administered once per week for three weeks including weeks 0, 1 and 2; weeks 0, 1 and 3; weeks 0, 1 and 4; weeks 0, 2 and 3; weeks 0, 2 and 4; or weeks 0, 3 and 4. In a related embodiment, the initial dose of 600 mg is administered once per week for four weeks including weeks 0, 1, 2 and 3; weeks 0, 1, 2 and 4; weeks 0, 1, 3 and 4; or weeks 0, 2, 3 and 4. In a related embodiment, the initial dose of 600 mg is administered twice per week for 2 weeks. In a related embodiment, the initial dose of 600 mg is administered twice per week for 3 weeks. In a related embodiment, the initial dose of 600 mg is administered twice per week for 4 weeks. In a related embodiment, the initial dose is 3000 mg (administered in 600 mg doses at, for example, day 1, week 1, week 2, week 3 and week 4). In a related embodiment, the initial dose is 1500 mg (administered in 300 mg doses at, for example, day 1, week 1, week 2, week 3 and week 4). In a related embodiment, the initial dose is 900 mg or 1200 mg administered IV (intravenously) or SC (subcutaneously) at q4w, q8w or q12w. In a related embodiment, the subsequent dose is 300 mg or 600 mg administered SC. In a related embodiment, the subsequent dose administration begins two to four weeks after the initial dose administration ends. In a related embodiment, the subsequent dose of 300 mg or 600 mg is administered q2w (once every 2 weeks), q4w (once every 4 weeks), q6w (once every 6 weeks) or q8w (once every 8 weeks). In a related embodiment, the subsequent dose is 600 mg administered q4w. In a related embodiment, the subsequent dose is 300 mg administered q4w. In a related embodiment, the subsequent dose is 300 mg administered q4w for eight weeks and q8w thereafter.

In one embodiment, the anti-IL-36R antibody administration at any of the dose regimens described herein results in one or more of the following endpoints:
(a) Palmoplantar Pustular Psoriasis Area and Severity Index 50 (PPP ASI50) at week 16 (e.g., achieving PPP ASI50 at week 16);
(b) reduction in the number of patients with drug-related Adverse Events (AEs) (e.g., achieving a reduced number of patients with AEs compared to placebo);
(c) PPP Physicians Global Assessment (PPP PGA) score of 0 or 1=clear/almost clear at week 16 (e.g., achieving a PPP PGA score of 0 or 1 at week 16);
(d) PPP ASI75 at week 16 (e.g., achieving PPP ASI75 at week 16);
(e) Percent change from baseline in the PPP ASI at week 16 (e.g., achieving a positive or an improved percent change from baseline in the PPP ASI at week 16);
(f) change from baseline in Pain Visual Analog Scale (VAS) score collected at Week 16 and all other visits (e.g., achieving an improved change in Pain VAS score for pain on palm and/or soles (PPP Pain VAS) and/or one for muscular and joint pain as compared to placebo over time);
(g) Clinical Improvement assessed via Dermatology Life Quality Index (DLQI) at week 16 and all other visits collected compared to baseline (e.g., achieving an improved or positive DLQI at week 16 compared to baseline);
(h) PPP ASI50 at all other visits collected (e.g., achieving PPP ASI50 over time);
(i) Modified (precise) PPP ASI scores at week 16 and all other visits collected (e.g., achieving an improved PPP ASI at week 16 and over time);
(j) Treatment success defined as achieving a clinical response of 0 or 1=clear/almost clear via PPP Physicians Global Assessment (PPP PGA) at all other visits collected (e.g., achieving a PPP PGA of 0 or 1 over time);
(k) PPP ASI75 at all other visits collected (e.g., achieving PPP ASI75 over time);
(l) Percent change from baseline in the PPP ASI at all other visits collected (e.g., achieving a positive or an improved percent change from baseline in the PPP ASI over time);
(m) Time (days) to achieving PPP ASI50 (e.g., achieving a PPP ASI50 at a shorter time compared to placebo);
(n) Time (days) to loss of PPP ASI50 (e.g., achieving a longer time to loss of PPP ASI50 as compared to placebo);
(o) Change in plaque psoriasis BSA involvement at week 16 in patients with concurrent plaque psoriasis at baseline (e.g., achieving an improved or a positive change in plaque psoriasis BSA at week 16 in patients with concurrent plaque psoriasis at baseline);
(p) superior efficacy over guselkumab (e.g., achieving 5% or more superior efficacy over guselkumab over time); or
(q) at least about 40% superiority to placebo in achieving PPP ASI50 at week 16 (e.g., achieving about 40% or more improvement in PPP ASI50 over placebo at week 16).

In one embodiment, the anti-IL-36R antibody administration of any of the dose regimens described herein to a subject suffering from PPP or its related signs and symptoms results in one or more of the following outcomes:
(a) the subject achieves a 50% reduction in PPP ASI (PPP ASI50) at week 16; or
(b) the subject experience a reduction in the number of drug-related Adverse Events (AEs) as compared to other treatments (e.g., guselkumab); or
(c) the subject experiences an improvement in his or her pustule severity (as compared to baseline) at week 16; or
(d) the anti-IL-36R antibody treatment shows a superior efficacy over guselkumab at week 16; or
(e) the subject achieves a PPP Physicians Global Assessment (PPP PGA) score of 0 or 1 (clear/almost clear) at week 16; or
(f) the subject achieves a Psoriasis Area and Severity Index for PPP (PPP ASI) 75 at week 16; or
(g) the subject experiences an improvement from baseline in the PPP ASI at week 16; or
(h) the subject achieves an improved change from baseline in Pain Visual Analog Scale (VAS) score at week 16; or
(i) the subject achieves a clinical improvement from baseline as assessed via Dermatology Life Quality Index (DLQI) at week 16; or
(j) the subject achieves a PPP ASI50 at visit 1, 2, 3, 4, 5, 6, 7, 8, 9, 10 or all other visits; or
(k) the subject achieves a reduction in PPP ASI scores at week 16 and all other visits; or
(l) the subject achieves PPP Physicians Global Assessment (PPP PGA) score of 0 or 1 (clear/almost clear) at visit 1, 2, 3, 4, 5, 6, 7, 8, 9, 10 or all other visits;
(m) the subject achieves a PPP ASI75 at visit 1, 2, 3, 4, 5, 6, 7, 8, 9, 10 or all other visits after treatment with the anti-IL-36R antibody;
(n) the subject experiences a percent change from baseline in the PPP ASI at visit 1, 2, 3, 4, 5, 6, 7, 8, 9, 10 or all other visits; or
(o) the subject experiences a lesser time to achieving PPP ASI50 as compared to other treatments (e.g., guselkumab); or
(p) the subject experiences a longer time to loss of PPP ASI50 as compared to other treatments (e.g., guselkumab);
(q) the subject experiences an improved change in plaque psoriasis BSA involvement at week 16 in subjects with concurrent plaque psoriasis at baseline; or
(r) the subject experiences a superiority over placebo in achieving PPP ASI50 at week 12; or
(s) the subject achieves a change in PPP ASI from baseline at week 16; or
(t) the subject achieves a positive or improved change in Pain VAS score from baseline at week 12; or
(u) the subject achieves a positive or improved PPP SI change from baseline at week 12; or
(v) the subject achieves a positive or improved PPP ASI change from baseline at week 52; or
(w) the subject achieves a reduction in occurrence of Treatment Emergent Adverse Events (TEAEs) from baseline overtime or at week 16; or
(x) the subject achieves a positive or improved change in pustule count from baseline over time; or
(y) the subject achieves a positive or improved change in pustular severity from baseline over time; or
(z) the subject achieves a PPP PGA clear/almost clear as compared to baseline or placebo over time; or
(aa) the subject achieves a PPP PGA pustule clear/almost clear as compared to baseline or placebo over time; or
(bb) the subject achieves a positive change from baseline in total score of PPQLI

(Palmoplantar Quality of Life Instrument), DLQI (Dermatology Life Quality Index), PSS (Psoriasis Symptom Scale), and BASDAI (Bath Ankylosing Spondylitis Disease Activity Index) over time; or
(cc) the subject achieves a PPP ASI50 over time; or
(dd) the subject achieves a PPP ASI75 over time; or
(ee) the subject achieves a positive or improved percent change from baseline in the PPP ASI over time; or
(ff) the subject achieves a positive or improved PPSI change as compared to baseline over time; or
(gg) the subject achieves a positive or improved change in Pain VAS score for pain on palm and/or soles (PPP Pain VAS) and/or one for muscular and joint pain as compared to baseline or placebo over time; or
(hh) the subject achieves a shorter time to PPP ASI75 as compared to baseline or placebo over time; or
(ii) the subject achieves a shorter time to PPP ASI50 as compared to baseline or placebo over time; or
(jj) the subject achieves a longer time to loss of PPP ASI75 as compared to baseline or placebo over time; or
(kk) the subject achieves a longer time to loss of PPP ASI50 as compared to baseline or placebo over time; or
(ll) the subject achieves a positive or improved change in PASI as compared to baseline or placebo over time; or
(mm) the subject achieves a positive or improved change in sPGA as compared to baseline or placebo over time; or
(nn) the subject achieves a positive or improved percent change in TPSS as compared with baseline or placebo over time; or
(oo) the subject achieves a positive or improved pharmacokinetic as compared to baseline or placebo over time; or
(pp) the subject achieves an improved gene expression change for the genes disclosed herein as an indication that the treatment is efficacious as compared with baseline or placebo over time; or
(qq) the subject achieves a PPP PGA of 0 or 1 at a reduced time as compared with baseline or placebo over time.

In one embodiment, the present invention relates to a method of preventing the recurrence of PPP flares (including new appearance or worsening of pustules), said method(s) including administering or having administered to the PPP patient a therapeutically effective amount of an anti-IL-36R antibody of the present invention subcutaneously or intravenously or by both routes according to any of the dose regimens listed in Tables 1-4.

In one embodiment, the present invention relates to a method of achieving a PPP Physicians Global Assessment (PPP PGA) score of 0 or 1=clear/almost clear at week 16, said method(s) including administering or having administered to the PPP patient a therapeutically effective amount of an anti-IL-36R antibody of the present invention subcutaneously or intravenously or by both routes according to any of the dose regimens listed in Tables 1-4.

In one embodiment, the present invention relates to a method of achieving a PPP Physicians Global Assessment (PPP PGA) score of 0 or 1=clear/almost clear at week 16, said method(s) including administering or having administered to the PPP patient a therapeutically effective amount of an anti-IL-36R antibody of the present invention subcutaneously or intravenously or by both routes according to any of the dose regimens listed in Tables 1-4.

In an embodiment relating to any of the aspects and embodiments described herein, the anti-IL-36R antibody or an antigen binding fragment thereof (disclosed herein) is present in a stable pharmaceutical formulation (as described in co-pending U.S. provisional application No. 62/815,405, filed Mar. 8, 2019, the entire content of which is hereby incorporated herein by reference in its entirety) for administration to a subject according to any one of the aspects of the present invention.

In one embodiment, the method of treatment according to any of the aspects described herein, includes administering to the subject a therapeutic amount of a stable pharmaceutical formulation comprising from about 20 mg/mL to about 150 mg/mL of an anti-IL-36R antibody (disclosed herein), about 20 mM to about 80 mM of a pharmaceutically acceptable buffer (e.g., acetate buffer), about 100 mM to about 250 mM of a pharmaceutically acceptable tonicifying agent (e.g., sucrose), about 0 mM to about 80 mM of a pharmaceutically acceptable stabilizing agent (e.g., arginine) or a pharmaceutically acceptable salt thereof, about 0 to about 150 mM of a pharmaceutically acceptable salt (e.g., sodium chloride), and a pharmaceutically acceptable surfactant (e.g., polysorbate 20) in an amount about 0 g/L to about 1.5 g/L, wherein the palmoplantar pustulosis (PPP) in the subject is treated, prevented or ameliorated, wherein the moderate to severe PPP in the subject treated, wherein the signs and symptoms of an acute phase flare-up (including new appearance or worsening of pustules) of PPP in the subject is reduced or alleviated, wherein the severity and duration of PPP flares in the subject is reduced, wherein the skin disorder associated with acute PPP (including new appearance or worsening of pustules) in the subject is treated, wherein the recurrence of PPP flares in the subject is reduced or prevented, wherein the PPP ASI 50 at week 16 in the subject is achieved, wherein the complete resolution of PPP symptoms in the subject is achieved, or wherein any of the endpoints listed above are achieved. In a related embodiment, the stable pharmaceutical formulation is an aqueous pharmaceutical formulation. In a related embodiment, the pH of the aqueous pharmaceutical formulation is about 5 to about 7. In a related embodiment, the pharmaceutical formulation is for an intravenous administration to the subject. In a related embodiment, the pharmaceutical formulation is for a subcutaneous or an intravenous administration to the subject. In a related embodiment, the pharmaceutical formulation for an intravenous administration comprises an anti-IL-36R antibody in an amount of about 60 mg/mL. In a related embodiment, the pharmaceutical formulation for a subcutaneous or an intravenous administration comprises an anti-IL-36R antibody in an amount of about 150 mg/mL. In a related embodiment, the pharmaceutical formulation for an intravenous administration comprises an anti-IL-36R antibody in an amount of about 20 mg/mL.

Additional features and advantages of the present invention will be set forth in the description below, and in part will be apparent from the description, or may be learned by practice of the subject technology. It is to be understood that both the foregoing general description and the following detailed description are exemplary and explanatory and are intended to provide further explanation of the present invention as claimed.

### BRIEF DESCRIPTION OF THE DRAWINGS

The accompanying drawings, which are included to provide further understanding of the present invention and are incorporated in and constitute a part of this specification, illustrate aspects of the subject technology and together with the description serve to explain the principles of the present invention.

FIG. 1 shows the study design in Example 1.

FIG. 2 shows the study design in Example 2.

FIG. 3 shows the study disposition described in Example 1. Notations in the figure are as follows: *Last treatment administered at Visit X (Week 12). †From end of treatment until Visit 13 (end of trial).

FIG. 4 shows lesional biomarker analysis comparing gene expression levels for patients (n=23) with a PPP ASI above/below the median at baseline.

FIG. 5 shows the scatter plot for PPP ASI percent change from baseline at Week 16 vs PPP ASI percent change from baseline at screening.

FIG. 6A shows the mean percent change from baseline in PPP ASI score over time in patients with improvement in the PPP ASI score from screening to baseline (screening ≥1.2× baseline).

FIG. 6B shows the mean percent change from baseline in PPP ASI score over time in patients with no improvement in the PPP ASI score from screening to baseline (screening <1.2× baseline).

FIG. 7 shows the mean PPP ASI scores at week 16 in the overall population and groups for baseline PPP ASI score median and baseline PPP ASI score>median.

FIG. 8A shows the mean percent change from baseline in PPP ASI over time in patients with baseline PPP ASI score>median (16.7).

FIG. 8B shows the mean percent change from baseline in pustule severity (Part of PPP ASI Score) over time in patients with baseline PPP ASI score>median (16.7).

FIG. 9A shows boxplot of mRNA fold change per gene by baseline PPP ASI worst affected area (<=median, >median) at baseline for gene: C15orf48.

FIG. 9B shows boxplot of mRNA fold change per gene by baseline PPP ASI worst affected area (<=median, >median) at baseline for gene: CCL20.

FIG. 9C shows boxplot of mRNA fold change per gene by baseline PPP ASI worst affected area (<=median, >median) at baseline for gene: CXCR2.

FIG. 9D shows boxplot of mRNA fold change per gene by baseline PPP ASI worst affected area (<=median, >median) at baseline for gene: IGHA1.

FIG. 9E shows boxplot of mRNA fold change per gene by baseline PPP ASI worst affected area (<=median, >median) at baseline for gene: IL17A.

FIG. 9F shows boxplot of mRNA fold change per gene by baseline PPP ASI worst affected area (<=median, >median) at baseline for gene: IL17F.

FIG. 9G shows boxplot of mRNA fold change per gene by baseline PPP ASI worst affected area (<=median, >median) at baseline for gene: IL36A.

FIG. 9H shows boxplot of mRNA fold change per gene by baseline PPP ASI worst affected area (<=median, >median) at baseline for gene: IL36B.

FIG. 9I shows boxplot of mRNA fold change per gene by baseline PPP ASI worst affected area (<=median, >median) at baseline for gene: IL36RN.

FIG. 9J shows boxplot of mRNA fold change per gene by baseline PPP ASI worst affected area (<=median, >median) at baseline for gene: LCN2.

FIG. 9K shows boxplot of mRNA fold change per gene by baseline PPP ASI worst affected area (<=median, >median) at baseline for gene: MIR155HG.

FIG. 9L shows boxplot of mRNA fold change per gene by baseline PPP ASI worst affected area (<=median, >median) at baseline for gene: S100A12.

FIG. 9M shows boxplot of mRNA fold change per gene by baseline PPP ASI worst affected area (<=median, >median) at baseline for gene: S100A7.

FIG. 9N shows boxplot of mRNA fold change per gene by baseline PPP ASI worst affected area (<=median, >median) at baseline for gene: S100AB.

FIG. 9O shows boxplot of mRNA fold change per gene by baseline PPP ASI worst affected area (<=median, >median) at baseline for gene: VNN1.

FIG. 10A shows boxplot of mRNA fold change per gene by baseline PPP ASI (<=median, >median) at baseline for gene: CXCR2.

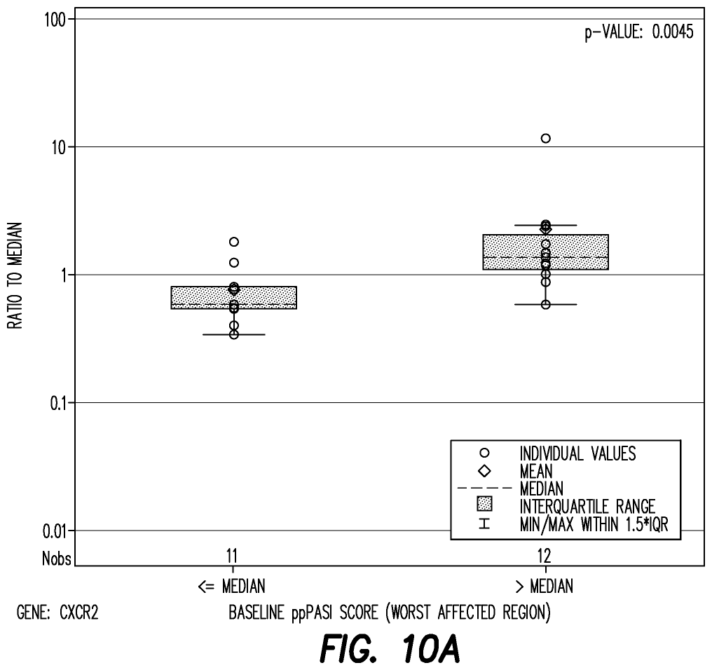

FIG. 10B shows boxplot of mRNA fold change per gene by baseline PPP ASI (<=median, >median) at baseline for gene: IL36G.

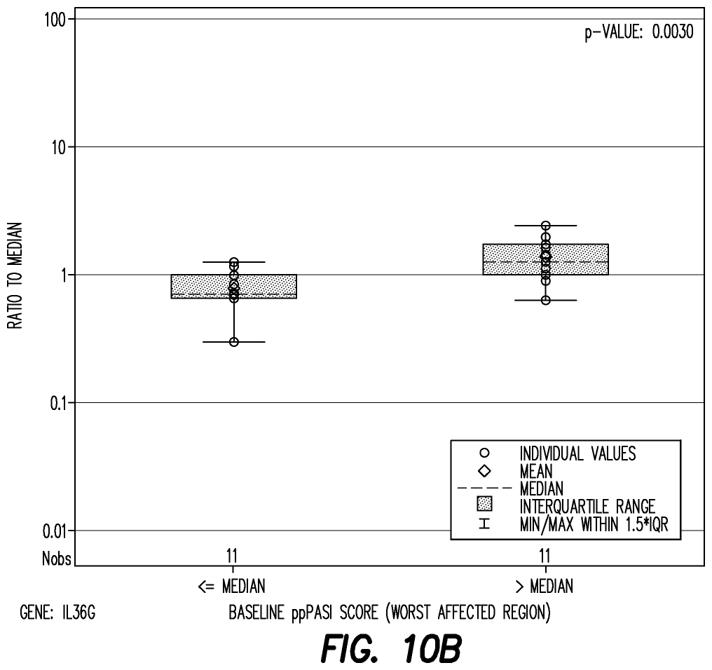

FIG. 10C shows boxplot of mRNA fold change per gene by baseline PPP ASI (<=median, >median) at baseline for gene: IL36RN.

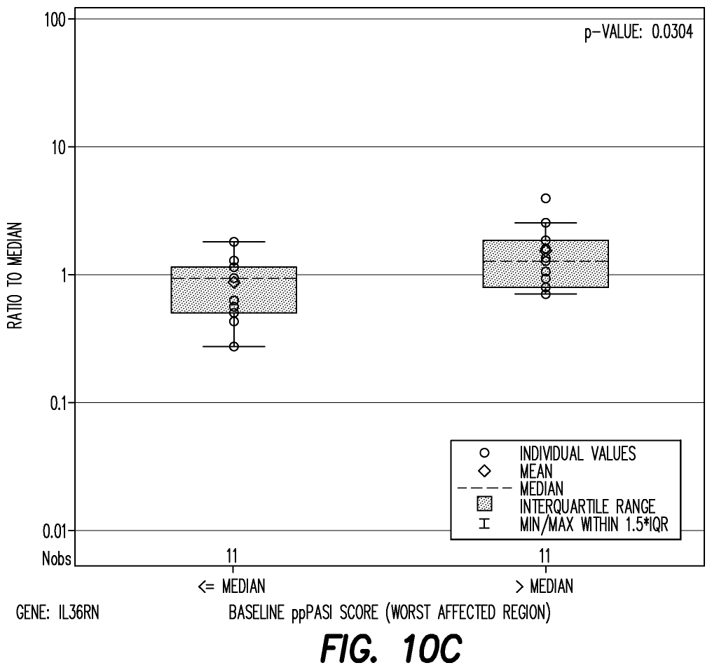

FIG. 10D shows boxplot of mRNA fold change per gene by baseline PPP ASI (<=median, >median) at baseline for gene: PI3.

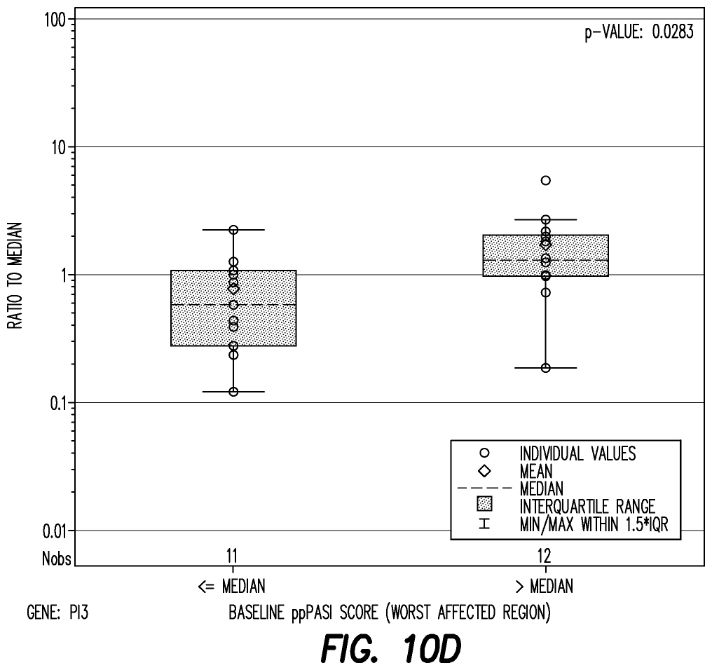

FIG. 10E shows boxplot of mRNA fold change per gene by baseline PPP ASI (<=median, >median) at baseline for gene: S100A12.

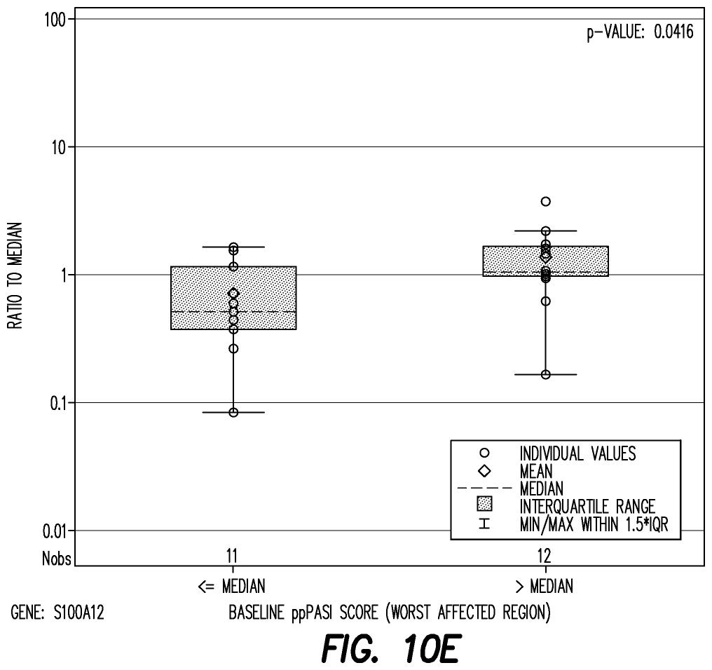

FIG. 10F shows boxplot of mRNA fold change per gene by baseline PPP ASI (<=median, >median) at baseline for gene: VNN3.

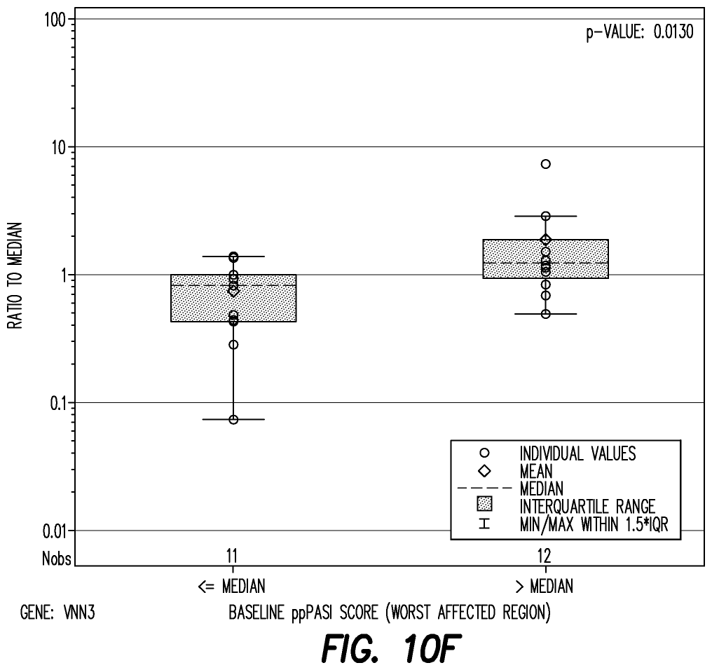

FIG. 11 shows the study design in Example 6; LD1=total loading dose of 3000 mg (loading dose of 600 mg at Visit 2 to 6, i.e., Day 1 (or Week 0), Week 1, 2, 3, and 4); LD2=total loading dose of 1500 mg (loading dose of 300 mg at Visit 2 to 6, i.e., Day 1, Week 1, 2, 3, and 4).

### DETAILED DESCRIPTION OF THE INVENTION

In the following detailed description, numerous specific details are set forth to provide a full understanding of the present invention. It will be apparent, however, to one ordinarily skilled in the art that the subject technology may be practiced without some of these specific details. In other instances, well-known structures and techniques have not been shown in detail so as not to obscure the present invention.

The invention therefore relates to compositions and methods for treating and/or prophylaxis of PPP and its signs and symptoms. More specifically, the invention relates to compositions and methods for treating and/or prophylaxis of moderate to severe PPP, acute PPP (including new appearance or worsening of pustules), chronic PPP, and/or PPP flares in a mammal with an anti-IL-36R antibody or an antigen-binding fragment thereof of the present invention. The compositions and methods include administering to the mammal a therapeutically effective amount of an anti-IL-36R antibody or an antigen-binding fragment thereof, wherein the anti-IL-36R antibody is administered based on the dose regimen disclosed herein. In an embodiment, the anti-IL-36R antibody is administered in one or more initial dose(s) administered subcutaneously and/or intravenously followed by one or more subsequent dose(s) administered subcutaneously and/or intravenously.

Without wishing to be bound by this theory it is believed that anti-IL-36R antibodies or antigen-binding fragments thereof bind to human anti-IL-36R and thus interfere with the binding of IL-36 agonists, and in doing so block at least partially the signaling cascade from the IL-36R to inflammatory mediators. The anti-IL36R antibodies of the present invention are disclosed in U.S. Pat. No. 9,023,995 or WO2013/074569, the entire content of each of which is incorporated herein by reference.

There is currently no drug specifically approved for the treatment of PPP and it is notoriously difficult to treat. Patients usually end up being treated with the currently available systemic treatment options including retinoids, PUVA, methotrexate, ciclosporine and topical corticosteroids. Unfortunately, the current treatment options are not effective in reducing duration and severity of PPP. Thus, there is high unmet medical need for PPP.

Based on the limitations described above, current therapeutic options are not suitable for life-long treatment and do not provide sustained responses in most patients. Therefore, there is a high need to develop (i) a highly effective treatment with rapid onset of action for patients with PPP; and (ii) to develop an effective treatment of chronic PPP, which reliably prevents the occurrence of flares (including new appearance or worsening of pustules) and is safe and tolerable for lifelong treatment.

Genetic and functional linkage studies have demonstrated linkage between the IL36 pathway and PPP.

IL36R is a cell surface receptor involved in inflammatory responses in skin and gut. It is a novel member of the IL1R family that forms a heterodimeric complex with the IL1R accessory protein. The heterodimeric IL36R system with stimulating (IL36α, IL36β, IL36γ) and inhibitory ligands (IL36Ra) shares a number of structural and functional similarities to other members of the IL1/IL1R family, such as IL1, IL18 and IL33 (R17-3602). All IL1 family members (IL1α, IL1β, IL18, IL36α, IL36β, IL36γ, and IL38) signal through a unique, cognate receptor protein which, upon ligand binding, recruits the common IL1 RacP subunit and activates NFkB and MAP kinase pathways in receptor-positive cell types. In human skin tissues, IL36R is expressed in keratinocytes, dermal fibroblasts and infiltrating myeloid cells. IL36R activation in skin tissue drives the production of inflammatory mediators (e.g. CCL20, MIP-1β, TNF-α, IL12, IL17, IL23, TGF-β) and modulates the expression of tissue remodeling genes (e.g. MMPs, TGF-β). Therefore, the link between GPP and mutations in the IL36RN is somewhat analogous to the well-established neonatal onset of sterile multifocal osteomyelitis, periostitis, and pustulosis caused by absence of interleukin-1-receptor antagonist. In this case, absence of the receptor antagonist allows unopposed action of interleukin-1, resulting in life-threatening systemic inflammation with skin and bone involvement. These clinical features responded to empirical treatment with the recombinant interleukin-1-receptor antagonist anakinra.

### I. Definitions

A phrase such as “an aspect” does not imply that such aspect is essential to the present invention or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure.

The term “about” shall generally mean an acceptable degree of error or variation for the quantity measured given the nature or precision of the measurements. Typical, exemplary degrees of error or variation are within 5% or within 3% or within 1% of a given value or range of values. For example, the expression of “about 100” includes 105 and 95 or 103 and 97 or 101 and 99, and all values in between (e.g., 95.1, 95.2, etc. for range of 95-105; or 97.1, 97.2, etc. for the range of 97-103; 99.1, 99.2, etc. for the range of 99-101). Numerical quantities given herein are approximates unless stated otherwise, meaning that the term “about” can be inferred when not expressly stated.

As used herein, the term “pharmaceutical formulation” or “formulation” refers to the process but also the product of a process in which an active drug or agent is combined with chemical substances to produce a final medicinal or drug product, the final formulation therefore refers to medicinal products such as liquids, powders or compositions. Therefore, in one embodiment, a pharmaceutical formulation is a pharmaceutical composition. A “pharmaceutical composition” refers in this context to a liquid or powder preparation which is in such form as to permit the biological activity of the active ingredient(s) to be unequivocally effective, and which contains no additional components which are significantly toxic to the subjects to which the composition would be administered. Such compositions are sterile. A “powder” refers to a freeze-dried or lyophilized or a spray-dried pharmaceutical composition for parenteral use. The powder is reconstituted or dissolved typically in water. Lyophilisation is a low temperature dehydration process which involves freezing the product, lowering pressure, then removing the ice by sublimation. Freeze drying results in a high quality product because of the low temperature used in processing. For a well-developed lyophilized formulation, the shape and appearance of the product is maintained over time and the quality of the rehydrated product is excellent. Spray drying is another method of producing a dry powder from a liquid or slurry by rapidly drying with a hot gas and with the goal of achieving a consistent particle size distribution.

The terms “initial dose,” “subsequent doses,” refer to the temporal sequence of administration of the IL-36R antagonist. Thus, the “initial dose” is the dose which is administered at the beginning of the treatment regimen (also referred to as the “baseline dose”); the “subsequent doses” are the doses which are administered after the initial dose. The initial, subsequent doses may all contain the same amount of anti-IL-36R antibody or an antigen binding fragment thereof, but generally may differ from one another in terms of the amount of the antibody administered or the frequency of administration. In certain embodiments, however, the amount of the anti-IL-36R antibody contained in the initial, subsequent doses varies from one another during the course of treatment. In certain embodiments, the one or more initial doses each comprise a first amount of the antibody or antigen-binding fragment thereof and the one or more subsequent doses each comprise a second amount of the antibody or antigen-binding fragment thereof. In some embodiments, the first amount of antibody or fragment thereof is 1.5×, 2×, 2.5×, 3×, 3.5×, 4×, or 5× the second or subsequent amount of the antibody or antigen-binding fragment thereof. In certain embodiments, one or more (e.g., 1, 2, 3, 4, or 5 or more) initial doses are administered at the beginning of the treatment regimen as “loading doses” or “leading doses” followed by subsequent doses that are administered on a less frequent basis (e.g., “maintenance doses”). For example, an anti-IL-36R antibody may be administered to a subject with PPP at one or more initial doses (or loading doses or leading doses) of about 150 mg, about 300 mg, about 600 mg, about 900 mg, or about 1200 mg followed by one or more subsequent doses (or maintenance doses) of about 300 mg or 600 mg. In one embodiment, the one or more initial doses and the one or more subsequent doses each include 300 mg or 600 mg dose of the anti-IL-36R antibody.

As used herein “buffer” refers to a buffered solution that resists changes in pH by the action of its acid-base conjugate components. The “pH” herein refers to the acidity or basicity of the composition at room temperature. Standard methods to measure the pH of a composition are known to the skilled in the art. Typically, measuring pH consists of calibrating the instrument, placing the electrodes in a well-mixed sample, and then reading the pH directly from the pH meter. The exemplary buffers of the present invention include acetate, citrate, histidine, succinate, phosphate and Tris.

As used herein, the term “tonicifying agent” or “tonicity agent” or “tonicifyer” refers to substances providing an osmotic pressure equivalent to that of serum in the body including salts (e.g. sodium chloride, potassium chloride, magnesium chloride) or sugars (e.g. sucrose, trehalose, sorbitol, magnesium sulfate (MgSO₄), glycerol, mannitol or dextrose). In addition, sugars present in the solution act as a cryoprotectant for the protein which allows the drug substance to be frozen without damage. This permits shipment in the frozen form and long-term storage of the drug substance prior to the filling of drug product. The exemplary tonicifying agents of the present invention include sodium chloride, potassium chloride, magnesium chloride (salts) and/or sucrose, trehalose, sorbitol, magnesium sulfate (MgSO₄), glycerol, mannitol or dextrose (sugars).

As used herein, the term “stabilizer” or “stabilizing agent” refers to substances contributing to the stability of the active ingredient in a pharmaceutical formulation. The exemplary stabilizing agents of the present invention include arginine, histidine, glycine, cysteine, proline, methionine, lysine, or pharmaceutically acceptable salts thereof.

As used herein, the term “surfactant” refers to substances which tend to reduce the surface tension of a liquid in which they are dissolved. The exemplary surfactants of the present invention include poloxamer 188, polysorbate 20, polysorbate 40, polysorbate 60 or polysorbate 80.

The terms, “antibody”, “anti-IL-36R antibody”, “humanized anti-IL-36R antibody”, “humanized anti-IL-36R epitope antibody”, and “variant humanized anti-IL-36R epitope antibody” specifically encompass monoclonal antibodies (including full length monoclonal antibodies), polyclonal antibodies, multispecific antibodies (e.g., bispecific antibodies), antibodies with minor modifications such as N- and/or C-terminal truncation, and antibody fragments such as variable domains and other portions of antibodies that exhibit a desired biological activity, e.g., IL-36R binding.

The term “monoclonal antibody” (mAb) refers to an antibody that is highly specific, being directed against a single antigenic determinant, an “epitope”. Therefore, the modifier “monoclonal” is indicative of antibodies directed to the identical epitope and is not to be construed as requiring production of the antibody by any particular method. It should be understood that monoclonal antibodies can be made by any technique or methodology known in the art; including e.g., the hybridoma method (Kohler et al., 1975, Nature 256:495), or recombinant DNA methods known in the art (see, e.g., U.S. Pat. No. 4,816,567), or methods of isolation of monoclonal recombinantly produced using phage antibody libraries, using techniques described in Clackson et al., 1991, Nature 352: 624-628, and Marks et al., 1991, J. Mol. Biol. 222: 581-597.

The term “monomer” refers to a homogenous form of an antibody. For example, for a full-length antibody, monomer means a monomeric antibody having two identical heavy chains and two identical light chains.

Chimeric antibodies consist of the heavy and light chain variable regions of an antibody from one species (e.g., a non-human mammal such as a mouse) and the heavy and light chain constant regions of another species (e.g., human) antibody and can be obtained by linking the DNA sequences encoding the variable regions of the antibody from the first species (e.g., mouse) to the DNA sequences for the constant regions of the antibody from the second (e.g. human) species and transforming a host with an expression vector containing the linked sequences to allow it to produce a chimeric antibody. Alternatively, the chimeric antibody also could be one in which one or more regions or domains of the heavy and/or light chain is identical with, homologous to, or a variant of the corresponding sequence in a monoclonal antibody from another immunoglobulin class or isotype, or from a consensus or germline sequence. Chimeric antibodies can include fragments of such antibodies, provided that the antibody fragment exhibits the desired biological activity of its parent antibody, for example binding to the same epitope (see, e.g., U.S. Pat. No. 4,816,567; and Morrison et al., 1984, Proc. Natl. Acad. Sci. USA 81: 6851-6855).

The terms, “antibody fragment”, “anti-IL-36R antibody fragment”, “anti-IL-36R epitope antibody fragment”, “humanized anti-IL-36R antibody fragment”, “humanized anti-IL-36R epitope antibody fragment”, “variant humanized anti-IL-36R epitope antibody fragment” refer to a portion of a full length anti-IL-36R antibody, in which a variable region or a functional capability is retained, for example, specific IL-36R epitope binding. Examples of antibody fragments include, but are not limited to, a Fab, Fab′, F(ab′)₂, Fd, Fv, scFv and scFv-Fc fragment, a diabody, a linear antibody, a single-chain antibody, a minibody, a diabody formed from antibody fragments, and multispecific antibodies formed from antibody fragments.

The term “intravenous administration” refers to introduction of an agent into the vein of an animal or human patient over a period of time which may be a few seconds to greater than approximately 15 minutes. For intravenous infusion, the administration period is generally between approximately 30 to 90 minutes.

The term “intravenous bolus” or “intravenous push” refers to drug administration into a vein of an animal or human such that the body receives the drug in approximately 15 minutes or less, generally 5 minutes or less.

The term “subcutaneous administration” refers to introduction of an agent under the skin of an animal or human patient, preferable within a pocket between the skin and underlying tissue, by relatively slow, sustained delivery from a drug receptacle. Pinching or drawing the skin up and away from underlying tissue may create the pocket.

The term “subcutaneous infusion” refers to introduction of a drug under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human patient, where bolus drug delivery is less than approximately 15 minutes; in another aspect, less than 5 minutes, and in still another aspect, less than 60 seconds. In yet even another aspect, administration is within a pocket between the skin and underlying tissue, where the pocket may be created by pinching or drawing the skin up and away from underlying tissue.

The term “mammal” for purposes of treatment refers to any animal classified as a mammal, including humans, domesticated and farm animals, and zoo, sports, or pet animals, such as dogs, horses, cats, cows, and the like. Preferably, the mammal is human.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby preventing or removing one or more signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the humanized anti-IL-36R antibody composition, the result should be considered an effective treatment of the underlying disorder regardless of whether all the symptoms of the disorder are alleviated or not.

The term “therapeutically effective amount” is used to refer to an amount of an active agent that relieves or ameliorates one or more of the symptoms of the disorder being treated. In another aspect, the therapeutically effective amount refers to a target serum concentration that has been shown to be effective in, for example, slowing disease progression. Efficacy can be measured in conventional ways, depending on the condition to be treated.

The term “prophylactically effective amount” is used to refer to an amount effective, at dosages and for periods of time necessary, to achieve the desired prophylactic result. Typically, a prophylactic dose is used in subjects prior to the onset of a PPP flare and/or prior to the onset of symptoms of PPP such as to prevent or inhibit the occurrence of acute flares. In an embodiment, a subcutaneous dose as contemplated herein is a prophylactic dose that is used in a patient with acute PPP (including new appearance or worsening of pustules), after the initial or induction dose, to prevent a possible recurrence of the PPP flares in the patient.

The term “package insert” is used to refer to instructions customarily included in commercial packages of therapeutic products, that contain information about the indications, usage, administration, contraindications and/or warnings concerning the use of such therapeutic products.

### II. Antibodies

The anti-IL36R antibodies of the present invention are disclosed in U.S. Pat. No. 9,023,995 or WO2013/074569, the entire content of each of which is incorporated herein by reference.

In one aspect, described and disclosed herein are anti-IL-36R antibodies, in particular humanized anti-IL-36R antibodies, and compositions and articles of manufacture comprising one or more anti-IL-36R antibody, in particular one or more humanized anti-IL-36R antibody of the present invention. Also described are binding agents that include an antigen-binding fragment of an anti-IL-36 antibody, in particular a humanized anti-IL-36R antibody.

Mode of Action

An anti-IL-36R antibody of the present invention is a humanized antagonistic monoclonal IgG1 antibody that blocks human IL36R signaling. Binding of an anti-IL-36R antibody of the present invention to IL36R is anticipated to prevent the subsequent activation of IL36R by cognate ligands (IL36 α, β and γ) and downstream activation of pro-inflammatory and pro-fibrotic pathways with the aim to reduce epithelial cell/fibroblast/immune cell-mediated inflammation and interrupt the inflammatory response that drives pathogenic cytokine production in palmoplantar pustular psoriasis (PPP). As provided herein, an anti-IL-36R antibody of the present invention has been tested and proved to be effective in treating patients with PPP, a severe inflammatory skin disease driven by uncontrolled IL36 activity.

IL-36R is also known as IL-1RL2 and IL-1 Rrp2. It has been reported that agonistic IL-36 ligands (α, β, or γ) initiate the signaling cascade by engaging the IL-36 receptor which then forms a heterodimer with the IL-1 receptor accessory protein (IL-1RAcP). IL-36 antagonist ligands (IL-36RA/IL1F5, IL-38/ILF10) inhibit the signaling cascade.

Variable regions and CDRs of representative antibodies of the present invention are disclosed below:

### Anti-IL-36R Mouse Antibody Sequences

Variable regions and CDRs of representative mouse lead antibodies of the present invention (mouse leads) are shown below:

Light Chain Variable Region (VK) Amino Acid Sequences
>33D10B12vK Protein (antibody 33D10)
QIVLTQSPAIMSASLGERVTMTCTASSSVSSSYLHWYQKKPGSSPKLWVYSTSNLASGVPVRF
SGSGSGTSYSLTISSMEAEDAATYYCHQHHRSPVTFGSGTKLEMK (SEQ ID NO: 1)
>172C8B12 vK protein (antibody 172C8)
DIQMTQSPASQSASLGESVTFTCLASQTIGTWLAWYQQRPGKSPQLLIYAATSLADGVPSRFS
GSGSGTQFSFNIRSLQAEDFASYYCQQVYTTPLTFGGGTKLEIK (SEQ ID NO: 2)
>67E7E8 vK protein (antibody 67E7)
DIQMTQSPASQSASLGESVTFTCLASQTIGTWLGWYQQKPGKSPQLLIYRSTTLADGVPSRFS
GSGSGTKFSFKISSLQAADFASYYCQQLYSAPYTFGGGTKLEIR (SEQ ID NO: 3)
>78C8D1 vK Protein (antibody 78C8)
DVLLTQTPLSLPVSLGDQASISCRSSQNIVHSNGNTYLQWYLQKPGQSPKLLIYKVSNRFSGVP
DRFSGSGSGTDFTLKISRVEAEDLGVYYCFQGSHVPFTFGAGTKLELK (SEQ ID NO: 4)
>81A1D1 vK Protein (antibody 81A1)
DIQMTQTTSSLSASLGDRVTISCRASQDIYKYLNWYQQKPDGTLKLLIYYTSGLHSGVPSRFSG
SGSGTDFSLTISNLEPEDIATYFCQQDSKFPWTFGGDTKLEIK (SEQ ID NO: 5)
>81B4E11 vK Protein (antibody 81B4)
QIVLTQSPAIMSASLGERVTMTCTASSSVSSSYFHWYQQKPGSSPKLWIYRTSNLASGVPGRF
SGSGSGTSYSLTISSMEAEDAATYYCHQFHRSPLTFGAGTKLELK (SEQ ID NO: 6)
>73C5C10 vK protein (antibody 73C5)
DIVMTQSQKFLSTSVGVRVSVTCKASQDVGTNVLWYQQKIGQSPKPLIYSASYRHSGVPDRFT
GSGSGTDFTLIISNVQSEDLAEYFCQQYSRYPLTFGPGTKLELK (SEQ ID NO: 7)
>73F6F8 vK protein (antibody 73F6)
DIVMTQSQKFLSTSVGVRVSVTCKASQDVGTNVLWYQQKIGQSPKALIYSASYRHSGVPDRFT
GSGSGTDFTLIITNVQSEDLAEYFCQQYSRYPLTFGPGTKLELK (SEQ ID NO: 8)
>76E10E8 vK protein (antibody 76E10)
DIVMTQSQKFMSATVGGRVNITCKASQNVGRAVAWYQQKPGQSPKLLTHSASNRYTGVPDRF
TGSGSGTDFTLTITNMQSEDLADYFCQQYSSYPLTFGAGTKLDLK (SEQ ID NO: 9)
>89A12B8 vK protein (antibody 89A12)
DIQMTQSPASQSASLGESVTFSCLASQTIGTWLGWYQQKPGKSPQLLIYRATSLADGVPSRFS
GSGSGTNFSFKISSLQAEDLASYYCQQLYSGPYTFGGGTKLEIR (SEQ ID NO: 10)
Heavy Chain Variable Region (VH) Amino Acid Sequences
>33D10B12vH Protein (antibody 33D10)
QVQLQQSGTELLKPGASVKLSCKASGNTVTSYWMHWVKQRPGQGLEWIGEILPSTGRTNYNE
NFKGKAMLTVDKSSSTAYMQLSSLASEDSAVYYCTIVYFGNPWFAYWGQGTLVTVSA (SEQ
ID NO: 11)
>172C8B12 vH protein (antibody 172C8)
EVQLQQSGPELVKPGASVKLSCKASGYTFTDNYMNWVRQSHGKSLEWIGRVNPSNGDTKYN
QNFKGKATLTVDKSLSTAYMQLNGLTSEDSAVYYCGRTKNFYSSYSYDDAMDYWGQGTSVTV
SS (SEQ ID NO: 12)
>67E7E8 vH protein (antibody 67E7)
EVQLQQSGAEFVRPGASVKFSCTASGFNIKDDYIHWVRQRPEQGLEWVGRIDPANGNTKYAP
KFQDKATITADTSSNTAYLQLSSLTSEDTAVYYCAKSFPNNYYSYDDAFAYWGQGTLVTVSA
(SEQ ID NO: 13)
>78C8D1 vH Protein (antibody 78C8)
QVQLKESGPVLVAPSQSLSITCTVSGFSLTKFGVHWIRQTPGKGLEWLGVIWAGGPTNYNSAL
MSRLTISKDISQSQVFLRIDSLQTDDTAMYYCAKQIYYSTLVDYWGQGTSVTVSS (SEQ ID NO:
14)
>81A1D1 vH Protein (antibody 81A1)
QVQLKESGPGLVAPSQSLFITCTVSGFSLSSYEINWVRQVPGKGLEWLGVIWTGITTNYNSALI
SRLSISKDNSKSLVFLKMNSLQTDDTAIYYCARGTGTGFYYAMDYWGQGTSVTVSS (SEQ ID
NO: 15)
>81B4E11 vH Protein (antibody 81B4)
QVQLQQPGADFVRPGASMRLSCKASGYSFTSSWIHWVKQRPGQGLEWIGEINPGNVRTNYN
ENFRNKATLTVDKSSTTAYMQLRSLTSADSAVYYCTVVFYGEPYFPYWGQGTLVTVSA (SEQ
ID NO: 16)
>73C5C10 vH Protein (antibody 73C5)
QVQLKESGPGLVAPSQSLSITCTVSGFSLTNYAVHWVRQFPGKGLEWLGVIWSDGSTDFNAP
FKSRLSINKDNSKSQVFFKMNSLQIDDTAIYYCARKGGYSGSWFAYWGQGTLVTVSA (SEQ ID
NO: 17)
>73F6F8 vH protein (antibody 73F6)
QVQLKESGPGLVAPSQSLSITCTVSGFSLTNYAVHWVRQFPGKGLEWLGVIWSDGSTDYNAP
FKSRLSINKDNSKSQVFFKMNSLQTDDTAIYYCARKGGYSGSWFAYWGQGTLVTVSA (SEQ
ID NO: 18)
>76E10E8 vH protein (antibody 76E10)
QVQLKESGPVLVAPSQSLSITCTVSGFSLTNYGVHWVRQPPGKGLEWLGVIWPVGSTNYNSA
LMSRLSIHKDNSKSQVFLRMNSLQTDDTAIYYCAKMDWDDFFDYWGQGTTLTVSS(SEQ ID
NO: 19)
>89A12B8 vH Protein (antibody 89A12)
EVQLQQSGAELVRPGASVRLSCTASGFNIKDDYIHWVRQRPKQGLEWLGRIDPANGNTKYDP
RFQDKATITADTSSNTAYLHLSSLTSEDTAVYYCAKSFPDNYYSYDDAFAYWGQGTLVTVSA
(SEQ ID NO: 20)
Light chain CDR-1 (L-CDR1) Amino Acid Sequences
>33D10G1 L-CDR1
TASSSVSSSYLH (SEQ ID NO: 21)
>172C8B12 L-CDR1
LASQTIGTWLA (SEQ ID NO: 22)
>67E7E8 L-CDR1
LASQTIGTWLG (SEQ ID NO: 23)
>78C8D1 L-CDR1
RSSQNIVHSNGNTYLQ (SEQ ID NO: 24)
>81A1D1 L-CDR1
RASQDIYKYLN (SEQ ID NO: 25)
>81B4E11 L-CDR1
TASSSVSSSYFH (SEQ ID NO: 26)
>73C5C10 L-CDR1
KASQDVGTNVL (SEQ ID NO: 27)
>73F6F8 L-CDR1
KASQDVGTNVL (SEQ ID NO: 27)
>76E10E8 L-CDR1
KASQNVGRAVA (SEQ ID NO: 28)
>89A12B8 L-CDR1
LASQTIGTWLG (SEQ ID NO: 29)
Light chain CDR-2 (L-CDR2) Amino Acid Sequences
>33D10B12 L-CDR2
STSNLAS (SEQ ID NO: 30)
>172C8B12 L-CDR2
AATSLAD (SEQ ID NO: 31)
>67E7E8 L-CDR2
RSTTLAD (SEQ ID NO: 32)
>78C8D1 L-CDR2
KVSNRFS (SEQ ID NO: 33)
>81A1D1 L-CDR2
YTSGLHS (SEQ ID NO: 34)
>81B4E11 L-CDR2
RTSNLAS (SEQ ID NO: 35)
>73C5C10 L-CDR2
SASYRHS (SEQ ID NO: 36)
>73F6F8 L-CDR2
SASYRHS (SEQ ID NO: 36)
>76E10E8 L-CDR2
SASNRYT (SEQ ID NO: 37)
>89A12B8 L-CDR2
RATSLAD (SEQ ID NO: 38)
Light chain CDR-3 (L-CDR3) Amino Acid Sequences
>33D10B12 L-CDR3
HQHHRSPVT (SEQ ID NO: 39)
>172C8B12 L-CDR3
QQVYTTPLT (SEQ ID NO: 40)
>67E7E8 L-CDR3
QQLYSAPYT (SEQ ID NO: 41)
>78C8D1 L-CDR3
FQGSHVPFT (SEQ ID NO: 42)
>81A1D1 L-CDR3
QQDSKFPWT (SEQ ID NO: 43)
>81B4E11 L-CDR3
HQFHRSPLT (SEQ ID NO: 44)
>73C5C10 L-CDR3
QQYSRYPLT (SEQ ID NO: 45)
>73F6F8 L-CDR3
QQYSRYPLT (SEQ ID NO: 45)
>76E10E8 L-CDR3
QQYSSYPLT (SEQ ID NO: 46)
>89A12B8 L-CDR3
QQLYSGPYT (SEQ ID NO: 47)
Heavy chain CDR-1 (H-CDR1) Amino Acid Sequences
>33D10B12 H-CDR1
GNTVTSYWMH (SEQ ID NO: 48)
>172C8B12 H-CDR1
GYTFTDNYMN (SEQ ID NO: 49)
>67E7E8 H-CDR1
GFNIKDDYIH (SEQ ID NO: 50)
>78C8D1 H-CDR1
GFSLTKFGVH (SEQ ID NO: 51)
>81A1D1 H-CDR1
GFSLSSYEIN (SEQ ID NO: 52)
>81B4E11 H-CDR1
GYSFTSSWIH (SEQ ID NO: 53)
>73C5C10 H-CDR1
GFSLTNYAVH (SEQ ID NO: 54)
>73F6F8 H-CDR1
GFSLTNYAVH (SEQ ID NO: 54)
>76E10E8 H-CDR1
GFSLTNYGVH (SEQ ID NO: 55)
>89A12B8 H-CDR1
GFNIKDDYIH (SEQ ID NO: 56)
Heavy chain CDR-2 (H-CDR2) Amino Acid Sequences
>33D10B12 H-CDR2
EILPSTGRTNYNENFKG (SEQ ID NO: 57)
>172C8B12 H-CDR2
RVNPSNGDTKYNQNFKG (SEQ ID NO: 58)
>67E7E8 H-CDR2
RIDPANGNTKYAPKFQD (SEQ ID NO: 59)
>78C8D1 H-CDR2
VIWAGGPTNYNSALMS (SEQ ID NO: 60)
>81A1D1 H-CDR2
VIWTGITTNYNSALIS (SEQ ID NO: 61)
>81B4E11 H-CDR2
EINPGNVRTNYNENF (SEQ ID NO: 62)
>73C5C10 H-CDR2
VIWSDGSTDFNAPFKS (SEQ ID NO: 63)
>73F6F8 H-CDR2
VIWSDGSTDYNAPFKS (SEQ ID NO: 64)
>76E10E8 H-CDR2
VIWPVGSTNYNSALMS (SEQ ID NO: 65)
>89A12B8 H-CDR2
RIDPANGNTKYDPRFQD (SEQ ID NO: 66)
Heavy chain CDR-3 (H-CDR3) Amino Acid Sequences
>33D10B12 H-CDR3
VYFGNPWFAY (SEQ ID NO: 67)
>172C8B12 H-CDR3
TKNFYSSYSYDDAMDY (SEQ ID NO: 68)
>67E7E8 H-CDR3
SFPNNYYSYDDAFAY (SEQ ID NO: 69)
>78C8D1 H-CDR3
QIYYSTLVDY (SEQ ID NO: 70)
>81A1D1 H-CDR3
GTGTGFYYAMDY (SEQ ID NO: 71)
>81B4E11 H-CDR3
VFYGEPYFPY (SEQ ID NO: 72)
>73C5C10 H-CDR3
KGGYSGSWFAY (SEQ ID NO: 73)
>73F6F8 H-CDR3
KGGYSGSWFAY (SEQ ID NO: 73)
>76E10E8 H-CDR3
MDWDDFFDY (SEQ ID NO: 74)
>89A12B8 H-CDR3
SFPDNYYSYDDAFAY (SEQ ID NO: 75)

### Anti-IL-36R Mouse CDR Sequences

A summary of the CDR sequences of the lead mouse antibodies is shown below:

| Antibody | H-CDR Sequences | L-CDR Sequences |
|---|---|---|
| 33D10 | GNTVTSYWMH (H-CDR1) | TASSSVSSSYLH |
| | SEQ ID No: 48 | (L-CDR1) |
| | EILPSTGRTNYNENFKG | SEQ ID No: 21 |
| | (H-CDR2) | STSNLAS (L-CDR2) |
| | SEQ ID No: 57 | SEQ ID No: 30 |
| | VYFGNPWFAY (H-CDR3) | HQHHRSPVT (L-CDR3) |
| | SEQ ID No: 67 | SEQ ID No: 39 |
| 172C8 | GYTFTDNYMN (H-CDR1) | LASQTIGTWLA (L-CDR1) |
| | SEQ ID No: 49 | SEQ ID No: 22 |
| | RVNPSNGDTKYNQNFKG | AATSLAD (L-CDR2) |
| | (H-CDR2) | SEQ ID No: 31 |
| | SEQ ID No: 58 | QQVYTTPLT (L-CDR3) |
| | TKNFYSSYSYDDAMDY | SEQ ID No: 40 |
| | (H-CDR3) | |
| | SEQ ID No: 68 | |
| 67E7 | GFNIKDDYIH (H-CDR1) | LASQTIGTWLG (L-CDR1) |
| | SEQ ID No: 50 | SEQ ID No: 23 |
| | RIDPANGNTKYAPKFQD | RSTTLAD (L-CDR2) |
| | (H-CDR2) | SEQ ID No: 32 |
| | SEQ ID No: 59 | QQLYSAPYT (L-CDR3) |
| | SFPNNYYSYDDAFAY | SEQ ID No: 41 |
| | (H-CDR3) | |
| | SEQ ID No: 69 | |
| 78C8 | GFSLTKFGVH (H-CDR1) | RSSQNIVHSNGNTYLQ |
| | SEQ ID No: 51 | (L-CDR1) |
| | VIWAGGPTNYNSALMS | SEQ ID No: 24 |
| | (H-CDR2) | KVSNRFS (L-CDR2) |
| | SEQ ID No: 60 | SEQ ID No: 33 |
| | QIYYSTLVDY (H-CDR3) | FQGSHVPFT (L-CDR3) |
| | SEQ ID No: 70 | SEQ ID No: 42 |
| 81A1 | GFSLSSYEIN (H-CDR1) | RASQDIYKYLN (L-CDR1) |
| | SEQ ID No: 52 | SEQ ID No: 25 |
| | VIWTGITTNYNSALIS | YTSGLHS (L-CDR2) |
| | (H-CDR2) | SEQ ID No: 34 |
| | SEQ ID No: 61 | QQDSKFPWT (L-CDR3) |
| | GTGTGFYYAMDY | SEQ ID No: 43 |
| | (H-CDR3) | |
| | SEQ ID No: 71 | |
| 81B4 | GYSFTSSWIH (H-CDR1) | TASSSVSSSYFH |
| | SEQ ID No: 53 | (L-CDR1) |
| | EINPGNVRTNYNENF | SEQ ID No: 26 |
| | (H-CDR2) | RTSNLAS (L-CDR2) |
| | SEQ ID No: 62 | SEQ ID No: 35 |
| | VFYGEPYFPY (H-CDR3) | HQFHRSPLT (L-CDR3) |
| | SEQ ID No: 72 | SEQ ID No: 44 |
| 73C5 | GFSLTNYAVH (H-CDR1) | KASQDVGTNVL (L-CDR1) |
| | SEQ ID No: 54 | SEQ ID No: 27 |
| | VIWSDGSTDFNAPFKS | SASYRHS (L-CDR2) |
| | (H-CDR2) | SEQ ID No: 36 |
| | SEQ ID No: 63 | QQYSRYPLT (L-CDR3) |
| | KGGYSGSWFAY (H- | SEQ ID No: 45 |
| | CDR3) SEQ ID No: 73 | |
| 73F6 | GFSLTNYAVH (H-CDR1) | KASQDVGTNVL (L-CDR1) |
| | SEQ ID No: 54 | SEQ ID No:27 |
| | VIWSDGSTDYNAPFKS | SASYRHS (L-CDR2) |
| | (H-CDR2) | SEQ ID No: 36 |
| | SEQ ID No: 64 | QQYSRYPLT (L-CDR3) |
| | KGGYSGSWFAY | SEQ ID No: 45 |
| | (H-CDR3) | |
| | SEQ ID No: 73 | |
| 76E10 | GFSLTNYGVH (H-CDR1) | KASQNVGRAVA (L-CDR1) |
| | SEQ ID No: 55 | SEQ ID No: 28 |
| | VIWPVGSTNYNSALMS | SASNRYT (L-CDR2) |
| | (H-CDR2) | SEQ ID No: 37 |
| | SEQ ID No: 65 | QQYSSYPLT (L-CDR3) |
| | MDWDDFFDY (H-CDR3) | SEQ ID No: 46 |
| | SEQ ID No: 74 | |
| 89A12 | GFNIKDDYIH (H-CDR1) | LASQTIGTWLG (L-CDR1) |
| | SEQ ID No: 56 | SEQ ID No: 29 |
| | RIDPANGNTKYDPRFQD | RATSLAD (L-CDR2) |
| | (H-CDR2) | SEQ ID No: 38 |
| | SEQ ID No: 66 | QQLYSGPYT (L-CDR3) |
| | SFPDNYYSYDDAFAY | SEQ ID No: 47 |
| | (H-CDR3) | |
| | SEQ ID No: 75 | |

### Anti-IL-36R Humanized Antibody Sequences

Human framework sequences were selected for the mouse leads based on the framework homology, CDR structure, conserved canonical residues, conserved interface packing residues and other parameters to produce humanized variable regions (see Example 5).

Representative humanized variable regions derived from antibodies 81B4 and 73C5 are shown below.

Light Chain Variable Region (VK) Amino Acid
Sequences
>81B4vK32_3 vK protein
(SEQ ID NO: 76)
EIVLTQSPGTLSLSPGERATMSCTASSSVSSSYFHWYQQKPGQAPRLL
IYRTSTLASGIPDRFSGSGSGTDFTLTISRLEPEDAATYYCHQFHRSP
LTFGQGTKLEIK
>81B4vK32_105 vK protein
(SEQ ID NO: 77)
EIVLTQSPGTLSLSPGERATMSCTASSSVSSSYFHWYQQKPGQAPRLL
IYRTSILASGVPDRFSGSGSGTDFTLTISRLEPEDFATYYCHQFHRSP
LTFGQGTKLEIK
>81B4vK32_116 vK protein
(SEQ ID NO: 78)
EIVLTQSPGTLSLSPGERATMSCTASSSVSSSYFHWYQQKPGQAPRLW
IYRTSRLASGVPDRFSGSGSGTDFTLTISRLEPEDAATYYCHQFHRSP
LTFGQGTKLEIK
>81B4vK32_127 vK protein
(SEQ ID NO: 79)
EIVLTQSPGTLSLSPGERATMTCTASSSVSSSYFHWYQQKPGQAPRLL
IYRTSRLASGVPDRFSGSGSGTDFTLTISRLEPEDFAVYYCHQFHRSP
LTFGQGTKLEIK
>81B4vK32_138 vK protein
(SEQ ID NO: 80)
QIVLTQSPGTLSLSPGERATMTCTASSSVSSSYFHWYQQKPGQAPRLW
IYRTSRLASGVPDRFSGSGSGTDFTLTISRLEPEDAATYYCHQFHRSP
LTFGAGTKLEIK
>81B4vK32_140 vK protein
(SEQ ID NO: 81)
QIVLTQSPGTLSLSPGERVTMSCTASSSVSSSYFHWYQQKPGQAPRLL
IYRTSQLASGIPDRFSGSGSGTDFTLTISRLEPEDAATYYCHQFHRSP
LTFGQGTKLEIK
>81B4vK32_141 vK protein
(SEQ ID NO: 82)
QIVLTQSPGTLSLSPGERATMTCTASSSVSSSYFHWYQQKPGQAPRLL
IYRTSKLASGVPDRFSGSGSGTDFTLTISRLEPEDFATYYCHQFHRSP
LTFGQGTKLEIK
>81B4vK32_147 vK protein
(SEQ ID NO: 83)
EIVLTQSPGTLSLSPGERATMSCTASSSVSSSYFHWYQQKPGQAPRLL
IYRTSHLASGIPGRFSGSGSGTDFTLTISRLEPEDAAVYYCHQFHRSP
LTFGQGTKLEIK
>73C5vK39_2 vK protein
(SEQ ID NO: 84)
EIVMTQSPATLSVSPGVRATLSCKASQDVGTNVLWYQQKPGQAPRPLI
YSASYRHSGIPDRFSGSGSGTEFTLTISSLQSEDFAEYFCQQYSRYPL
TFGQGTKLEIK
>73C5vK39_7 vK protein
(SEQ ID NO: 85)
EIVMTQSPATLSVSPGVRATLSCKASQDVGTNVLWYQQKPGQAPRPLI
YSASYRHSGIPDRFSGSGSGTEFTLTISSLQSEDFAVYYCQQYSRYPL
TFGQGTKLEIK
>73C5vK39_15 vK protein
(SEQ ID NO: 86)
EIVMTQSPATLSVSPGVRATLSCKASQDVGTNVLWYQQKPGQAPRPLI
YSASYRHSGIPARFSGSGSGTEFTLTISSLQSEDFAEYYCQQYSRYPL
TFGQGTKLEIK
Heavy Chain Variable Region (VH) Amino Acid
Sequences
>81B4vH33_49 vH Protein
(SEQ ID NO: 87)
QVQLVQSGAEVKKPGASVKVSCKASGYSFTSSWIHWVRQAPGQGLEWI
GEINPGNVRTNYNENFRNKATMTVDTSISTAYMELSRLRSDDTAVYYC
AVVFYGEPYFPYWGQGTLVTVSS
>81B4vH33_85T vH Protein
(SEQ ID NO: 88)
QVQLVQSGAEVKKPGASVKVSCKASGYSFTSSWIHWVRQRPGQGLEWI
GEINPGNVRTNYNENFRNRVTMTVDTSISTAYMELSRLRSDDTAVYYC
TVVFYGEPYFPYWGQGTLVTVSS
>81B4vH33_90 vH Protein
(SEQ ID NO: 89)
QVQLVQSGAEVKKPGASVKVSCKASGYSFTSSWIHWVKQAPGQGLEWM
GEINPGNVRTNYNENFRNKVTMTVDTSISTAYMELSRLRSDDTAVYYC
TVVFYGEPYFPYWGQGTLVTVSS
>81B4vH33_93 vH Protein
(SEQ ID NO: 90)
QVQLVQSGAEVKKPGASVKVSCKASGYSFTSSWIHWVRQRPGQGLEWM
GEINPGNVRTNYNENFRNRATLTRDTSISTAYMELSRLRSDDTAVYYC
AVVFYGEPYFPYWGQGTLVTVSS
>81B4vH50_22 vH Protein
(SEQ ID NO: 91)
QVQLVQSGAEVKKPGASVKVSCKASGYSFTSSWIHWVRQRPGQGLEWM
GEILPGVVRTNYNENFRNKVTMTVDTSISTAYMELSRLRSDDTAVYYC
TVVFYGEPYFPYWGQGTLVTVSS
>81B4vH50_30 vH Protein
(SEQ ID NO: 92)
QVQLVQSGAEVKKPGASVKVSCKASGYSFTSSWIHWVRQRPGQGLEWI
GEINPGAVRTNYNENFRNRVTMTVDTSISTAYMELSRLRSDDTAVYYC
TVVFYGEPYFPYWGQGTLVTVSS
>81B4vH51_13 vH Protein
(SEQ ID NO: 93)
QVQLVQSGAEVKKPGASVKVSCKASGYSFTSSWIHWVRQAPGQGLEWI
GEINPGLVRTNYNENFRNKVTMTVDTSISTAYMELSRLRSDDTAVYYC
AVVFYGEPYFPYWGQGTLVTVSS
>81B4vH51_15 vH Protein
(SEQ ID NO: 94)
QVQLVQSGAEVKKPGASVKVSCKASGYSFTSSWIHWVRQAPGQGLEWI
GEINPGAVRTNYNENFRNKVTMTVDTSISTAYMELSRLRSDDTAVYYC
AVVFYGEPYFPYWGQGTLVTVSS
>81B4vH52_83 vH Protein
(SEQ ID NO: 95)
QVQLVQSGAEVKKPGASVKVSCKASGYSFTSSWIHWVRQAPGQGLEWI
GEINPGSVRTNYNENFRNKATMTVDTSISTAYMELSRLRSDDTAVYYC
AVVFYGEPYFPYWGQGTLVTVSS
>73C5vH46_4 vH Protein
(SEQ ID NO: 96)
QVQLQESGPGLVKPSETLSITCTVSGFSLTDYAVHWIRQPPGKGLEWI
GVIWSDGSTDYNAPFKSRVTINKDTSKSQVSFKMSSVQAADTAVYYCA
RKGGYSGSWFAYWGQGTLVTVSS
>73C5vH46_19 vH Protein
(SEQ ID NO: 97)
QVQLQESGPGLVKPSETLSITCTVSGFSLTDYAVHWIRQPPGKGLEWI
GVIWSDGSTDYNAPFKSRVTISKDTSKNQVSLKMNSLTTDDTAVYYCA
RKGGYSGSWFAYWGQGTLVTVSS
>73C5vH46_40 vH Protein
(SEQ ID NO: 98)
QVQLQESGPGLVKPSETLSITCTVSGFSLTDYAVHWIRQPPGKGLEWI
GVIWSDGSTDYNAPFKSRVTISKDNSKSQVSLKMNSVTVADTAVYYCA
RKGGYSGSWFAYWGQGTLVTVSS
>73C5vH47_65 vH Protein
(SEQ ID NO: 99)
QVQLQESGPGLVKPSETLSITCTVSGFSLTDYAVHWVRQPPGKGLEWI
GVIWSDGSTDYNAPFKSRVTISKDTSKNQVSFKLSSVTVDDTAVYYCA
RKGGYSGSWFAYWGQGTLVTVSS
>73C5vH47_77 vH Protein
(SEQ ID NO: 100)
QVQLQESGPGLVAPSETLSLTCTVSGFSLTDYAVHWIRQFPGKGLEWI
GVIWSDGSTDFNAPFKSRVTISKDTSKNQVSFKLSSVTTDDTAVYYCA
RKGGYSGSWFAYWGQGTLVTVSS
>73C5vH58_91 vH Protein
(SEQ ID NO: 101)
QVQLQESGPGLVKPSETLSITCTVSGFSLTDYAVHWIRQPPGKGLEWI
GVIWSDGSTDYNAPFKSRVTISKDNSKSQVSFKMSSVTADDTAVYYCA
RKGGYSGSWFAYWGQGTLVTVSS

The CDR sequences from the humanized variable regions derived from antibodies 81B4 and 73C5 shown above are depicted below.

L-CDR1 Amino Acid Sequences
>81B4vK32_3 L-CDR1
(SEQ ID NO: 26)
TASSSVSSSYFH
>81B4vK32_105 L-CDR1
(SEQ ID NO: 26)
TASSSVSSSYFH
>81B4vK32_116 L-CDR1
(SEQ ID NO: 26)
TASSSVSSSYFH
>81B4vK32_127 L-CDR1
(SEQ ID NO: 26)
TASSSVSSSYFH
>81B4vK32_138L-CDR1
(SEQ ID NO: 26)
TASSSVSSSYFH
>81B4vK32_140 L-CDR1
(SEQ ID NO: 26)
TASSSVSSSYFH
>81B4vK32_141 L-CDR1
(SEQ ID NO: 26)
TASSSVSSSYFH
>81B4vK32_147 L-CDR1
(SEQ ID NO: 26)
TASSSVSSSYFH
>73C5vK39_2 L-CDR1
(SEQ ID NO: 27)
KASQDVGTNVL
>73C5vK39_7 L-CDR1
(SEQ ID NO: 27)
KASQDVGTNVL
>73C5vK39_15 L-CDR1
(SEQ ID NO: 27)
KASQDVGTNVL
L-CDR2 Amino Acid Sequences
>81B4vK32_3 L-CDR2
(SEQ ID 102)
RTSTLAS
>81B4vK32_105 L-CDR2
(SEQ ID 103)
RTSILAS
>81B4vK32_116 L-CDR2
(SEQ ID 104)
RTSRLAS
>81B4vK32_127 L-CDR2
(SEQ ID 104)
RTSRLAS
>81B4vK32_138 L-CDR2
(SEQ ID 104)
RTSRLAS
>81B4vK32_140 L-CDR2
(SEQ ID 105)
RTSQLAS
>81B4vK32_141 L-CDR2
(SEQ ID 106)
RTSKLAS
>81B4vK32_147 L-CDR2
(SEQ ID 140)
RTSHLAS
>73C5vK39_2 L-CDR2
(SEQ ID NO: 36)
SASYRHS
>73C5vK39_7 L-CDR2
(SEQ ID NO: 36)
SASYRHS
>73C5vK39_15 L-CDR2
(SEQ ID NO: 36)
SASYRHS
L-CDR3 Amino Acid Sequences
>81B4vK32_3 L-CDR3
(SEQ ID NO: 44)
HQFHRSPLT
>81B4vK32_105 L-CDR3
(SEQ ID NO: 44)
HQFHRSPLT
>81B4vK32_116 L-CDR3
(SEQ ID NO: 44)
HQFHRSPLT
>81B4vK32_127 L-CDR3
(SEQ ID NO: 44)
HQFHRSPLT
>81B4vK32_138L-CDR3
(SEQ ID NO: 44)
HQFHRSPLT
>81B4vK32_140 L-CDR3
(SEQ ID NO: 44)
HQFHRSPLT
>81B4vK32_141 L-CDR3
(SEQ ID NO: 44)
HQFHRSPLT
>81B4vK32_147L-CDR3
(SEQ ID NO: 44)
HQFHRSPLT
>73C5vK39_2 L-CDR3
(SEQ ID NO: 45)
QQYSRYPLT
>73C5vK39_7 L-CDR3
(SEQ ID NO: 45)
QQYSRYPLT
>73C5vK39_15 L-CDR3
(SEQ ID NO: 45)
QQYSRYPLT
H-CDR1 Amino Acid Sequences
>81B4vH33_49 H-CDR1
(SEQ ID NO: 53)
GYSFTSSWIH
>81B4vH33_85TH-CDR1
(SEQ ID NO: 53)
GYSFTSSWIH
>81B4vH33_90 H-CDR1
(SEQ ID NO: 53)
GYSFTSSWIH
>81B4vH33_93 H-CDR1
(SEQ ID NO: 53)
GYSFTSSWIH
>81B4vH50_22H-CDR1
(SEQ ID NO: 53)
GYSFTSSWIH
>81B4vH50_30 H-CDR1
(SEQ ID NO: 53)
GYSFTSSWIH
>81B4vH51_13 H-CDR1
(SEQ ID NO: 53)
GYSFTSSWIH
>81B4vH51_15 H-CDR1
(SEQ ID NO: 53)
GYSFTSSWIH
>81B4vH52_83 H-CDR1
(SEQ ID NO: 53)
GYSFTSSWIH
>73C5vH46_4 H-CDR1
(SEQ ID NO: 107)
GFSLTDYAVH
>73C5vH46_19 H-CDR1
(SEQ ID NO: 107)
GFSLTDYAVH
>73C5vH46_40 H-CDR1
(SEQ ID NO: 107)
GFSLTDYAVH
>73C5vH47_65 H-CDR1
(SEQ ID NO: 107)
GFSLTDYAVH
>73C5vH47_77 H-CDR1
(SEQ ID NO: 107)
GFSLTDYAVH
>73C5vH58_91 H-CDR1
(SEQ ID NO: 107)
GFSLTDYAVH
H-CDR1
(SEQ ID NO: 141)
SSWIH
H-CDR2 Amino Acid Sequences
>81B4vH33_49 H-CDR2
(SEQ ID NO: 62)
EINPGNVRTNYNENF
>81B4vH33_85T H-CDR2
(SEQ ID NO: 62)
EINPGNVRTNYNENF
>81B4vH33_90 H-CDR2
(SEQ ID NO: 62)
EINPGNVRTNYNENF
>81B4vH33_93 H-CDR2
(SEQ ID NO: 62)
EINPGNVRTNYNENF
>81B4vH50_22 H-CDR2
(SEQ ID NO: 108)
EILPGVVRTNYNENF
>81B4vH50_30 H-CDR2
(SEQ ID NO: 109)
EINPGAVRTNYNENF
>81B4vH51_13H-CDR2
(SEQ ID NO: 110)
EINPGLVRTNYNENF
>81B4vH51_15H-CDR2
(SEQ ID NO: 109)
EINPGAVRTNYNENF
>81B4vH52_83 H-CDR2
(SEQ ID NO: 111)
EINPGSVRTNYNENF
>73C5vH46_4 H-CDR2
(SEQ ID NO: 64)
VIWSDGSTDYNAPFKS
>73C5vH46_19H-CDR2
(SEQ ID NO: 64)
VIWSDGSTDYNAPFKS
>73C5vH46_40 H-CDR2
(SEQ ID NO: 64)
VIWSDGSTDYNAPFKS
>73C5vH47_65 H-CDR2
(SEQ ID NO: 64)
VIWSDGSTDYNAPFKS
>73C5vH47_77 H-CDR2
(SEQ ID NO: 63)
VIWSDGSTDFNAPFKS
>73C5vH58_91 H-CDR2
(SEQ ID NO: 64)
VIWSDGSTDYNAPFKS
H-CDR2
(SEQ ID NO: 142)
EINPGNVRTNYNENFRN
H-CDR3 Amino Acid Sequences
>81B4vH33_49 H-CDR3
(SEQ ID NO: 72)
VFYGEPYFPY
>81B4vH33_85T H-CDR3
(SEQ ID NO: 72)
VFYGEPYFPY
>81B4vH33_90 H-CDR3
(SEQ ID NO: 72)
VFYGEPYFPY
>81B4vH33_93 H-CDR3
(SEQ ID NO: 72)
VFYGEPYFPY
>81B4vH50_22 H-CDR3
(SEQ ID NO: 72)
VFYGEPYFPY
>81B4vH50_30 H-CDR3
(SEQ ID NO: 72)
VFYGEPYFPY
>81B4vH51_13 H-CDR3
(SEQ ID NO: 72)
VFYGEPYFPY
>81B4vH51_15 H-CDR3
(SEQ ID NO: 72)
VFYGEPYFPY
>81B4vH52_83 H-CDR3
(SEQ ID NO: 72)
VFYGEPYFPY
>73C5vH46_4 H-CDR3
(SEQ ID NO: 73)
KGGYSGSWFAY
>73C5vH46_19 H-CDR3
(SEQ ID NO: 73)
KGGYSGSWFAY
>73C5vH46_40 H-CDR3
(SEQ ID NO: 73)
KGGYSGSWFAY
>73C5vH47_65 H-CDR3
(SEQ ID NO: 73)
KGGYSGSWFAY
>73C5vH47_77 H-CDR3
(SEQ ID NO: 73)
KGGYSGSWFAY
>73C5vH58_91 H-CDR3
(SEQ ID NO: 73)
KGGYSGSWFAY

In one aspect, a variable region of the present invention is linked to a constant region. For example, a variable region of the present invention is linked to a constant region shown below to form a heavy chain or a light chain of an antibody.

Heavy Chain Constant region linked downstream of
a humanized variable heavy region:
(SEQ ID NO: 112)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
RVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREE
MTKNQVSLTCLVKGFYPSDIAVEWESNGQPENNYKTTPPVLDSDGSFF
LYSKLTVDKSRWQQGNVFSCSVMHEALHNHYTQKSLSLSPGK
Light Chain Constant region linked downstream of
a humanized variable light region:
(SEQ ID NO: 113)
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSS
PVTKSFNRGEC

Representative light chain and heavy chain sequences of the present invention are shown below (humanized variable regions derived from antibodies 81B4 and 73C5 linked to constant regions).

Light Chain Amino Acid Sequences
>81B4vK32_3 Light Chain
(SEQ ID NO: 114)
EIVLTQSPGTLSLSPGERATMSCTASSSVSSSYFHWYQQKPGQAPRLLIYRTSTLASGIPDRFSGSGSGTD
FTLTISRLEPEDAATYYCHQFHRSPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFNRG
EC
>81B4vK32_105 Light Chain
(SEQ ID NO: 115)
EIVLTQSPGTLSLSPGERATMSCTASSSVSSSYFHWYQQKPGQAPRLLIYRTSILASGVPDRFSGSGSGTD
FTLTISRLEPEDFATYYCHQFHRSPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFNRG
EC
>81B4vK32_116 Light Chain
(SEQ ID NO: 116)
EIVLTQSPGTLSLSPGERATMSCTASSSVSSSYFHWYQQKPGQAPRLWIYRTSRLASGVPDRFSGSGSGTD
FTLTISRLEPEDAATYYCHQFHRSPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFNRG
EC
>81B4vK32_127 Light Chain
(SEQ ID NO: 117)
EIVLTQSPGTLSLSPGERATMTCTASSSVSSSYFHWYQQKPGQAPRLLIYRTSRLASGVPDRFSGSGSGTD
FTLTISRLEPEDFAVYYCHQFHRSPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFNRG
EC
>81B4vK32_138 Light Chain
(SEQ ID NO: 118)
QIVLTQSPGTLSLSPGERATMTCTASSSVSSSYFHWYQQKPGQAPRLWIYRTSRLASGVPDRFSGSGSGTD
FTLTISRLEPEDAATYYCHQFHRSPLTFGAGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFNRG
EC
>81B4vK32_140 Light Chain
(SEQ ID NO: 119)
QIVLTQSPGTLSLSPGERVTMSCTASSSVSSSYFHWYQQKPGQAPRLLIYRTSQLASGIPDRFSGSGSGTD
FTLTISRLEPEDAATYYCHQFHRSPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFNRG
EC
>81B4vK32_141 Light Chain
(SEQ ID NO: 120)
QIVLTQSPGTLSLSPGERATMTCTASSSVSSSYFHWYQQKPGQAPRLLIYRTSKLASGVPDRFSGSGSGTD
FTLTISRLEPEDFATYYCHQFHRSPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFNRG
EC
>81B4vK32_147 Light Chain
(SEQ ID NO: 121)
EIVLTQSPGTLSLSPGERATMSCTASSSVSSSYFHWYQQKPGQAPRLLIYRTSHLASGIPGRFSGSGSGTD
FTLTISRLEPEDAAVYYCHQFHRSPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFNRG
EC
>73C5vK39_2 Light Chain
(SEQ ID NO: 122)
EIVMTQSPATLSVSPGVRATLSCKASQDVGTNVLWYQQKPGQAPRPLIYSASYRHSGIPDRFSGSGSGTEF
TLTISSLQSEDFAEYFCQQYSRYPLTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGE
C
>73C5vK39_7 Light Chain
(SEQ ID NO: 123)
EIVMTQSPATLSVSPGVRATLSCKASQDVGTNVLWYQQKPGQAPRPLIYSASYRHSGIPDRFSGSGSGTEF
TLTISSLQSEDFAVYYCQQYSRYPLTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGE
C
>73C5vK39_15 Light Chain
(SEQ ID NO: 124)
EIVMTQSPATLSVSPGVRATLSCKASQDVGTNVLWYQQKPGQAPRPLIYSASYRHSGIPARFSGSGSGTEF
TLTISSLQSEDFAEYYCQQYSRYPLTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGE
C
Heavy Chain Amino Acid Sequences
>81B4vH33_49 Heavy Chain
(SEQ ID NO: 125)
QVQLVQSGAEVKKPGASVKVSCKASGYSFTSSWIHWVRQAPGQGLEWIGEINPGNVRTNYNENFRNKATMT
VDTSISTAYMELSRLRSDDTAVYYCAVVFYGEPYFPYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>81B4vH33_85T Heavy Chain
(SEQ ID NO: 126)
QVQLVQSGAEVKKPGASVKVSCKASGYSFTSSWIHWVRQRPGQGLEWIGEINPGNVRTNYNENFRNRVTMT
VDTSISTAYMELSRLRSDDTAVYYCTVVFYGEPYFPYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>81B4vH33_90 Heavy Chain
(SEQ ID NO: 127)
QVQLVQSGAEVKKPGASVKVSCKASGYSFTSSWIHWVKQAPGQGLEWMGEINPGNVRTNYNENFRNKVTMT
VDTSISTAYMELSRLRSDDTAVYYCTVVFYGEPYFPYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>81B4vH33_93 Heavy Chain
(SEQ ID NO: 128)
QVQLVQSGAEVKKPGASVKVSCKASGYSFTSSWIHWVRQRPGQGLEWMGEINPGNVRTNYNENFRNRATLT
RDTSISTAYMELSRLRSDDTAVYYCAVVFYGEPYFPYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>81B4vH50_22 Heavy Chain
(SEQ ID NO: 129)
QVQLVQSGAEVKKPGASVKVSCKASGYSFTSSWIHWVRQRPGQGLEWMGEILPGVVRTNYNENFRNKVTMT
VDTSISTAYMELSRLRSDDTAVYYCTVVFYGEPYFPYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>81B4vH50_30 Heavy Chain
(SEQ ID NO: 130)
QVQLVQSGAEVKKPGASVKVSCKASGYSFTSSWIHWVRQRPGQGLEWIGEINPGAVRTNYNENFRNRVTMT
VDTSISTAYMELSRLRSDDTAVYYCTVVFYGEPYFPYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>81B4vH51_13 Heavy Chain
(SEQ ID NO: 131)
QVQLVQSGAEVKKPGASVKVSCKASGYSFTSSWIHWVRQAPGQGLEWIGEINPGLVRTNYNENFRNKVTMT
VDTSISTAYMELSRLRSDDTAVYYCAVVFYGEPYFPYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>81B4vH51_15 Heavy Chain
(SEQ ID NO: 132)
QVQLVQSGAEVKKPGASVKVSCKASGYSFTSSWIHWVRQAPGQGLEWIGEINPGAVRTNYNENFRNKVTMT
VDTSISTAYMELSRLRSDDTAVYYCAVVFYGEPYFPYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>81B4vH52_83 Heavy Chain
(SEQ ID NO: 133)
QVQLVQSGAEVKKPGASVKVSCKASGYSFTSSWIHWVRQAPGQGLEWIGEINPGSVRTNYNENFRNKATMT
VDTSISTAYMELSRLRSDDTAVYYCAVVFYGEPYFPYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>73C5vH46_4 Heavy Chain
(SEQ ID NO: 134)
QVQLQESGPGLVKPSETLSITCTVSGFSLTDYAVHWIRQPPGKGLEWIGVIWSDGSTDYNAPFKSRVTINK
DTSKSQVSFKMSSVQAADTAVYYCARKGGYSGSWFAYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>73C5vH46_19 Heavy Chain
(SEQ ID NO: 135)
QVQLQESGPGLVKPSETLSITCTVSGFSLTDYAVHWIRQPPGKGLEWIGVIWSDGSTDYNAPFKSRVTISK
DTSKNQVSLKMNSLTTDDTAVYYCARKGGYSGSWFAYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>73C5vH46_40 Heavy Chain
(SEQ ID NO: 136)
QVQLQESGPGLVKPSETLSITCTVSGFSLTDYAVHWIRQPPGKGLEWIGVIWSDGSTDYNAPFKSRVTISK
DNSKSQVSLKMNSVTVADTAVYYCARKGGYSGSWFAYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>73C5vH47_65 Heavy Chain
(SEQ ID NO: 137)
QVQLQESGPGLVKPSETLSITCTVSGFSLTDYAVHWVRQPPGKGLEWIGVIWSDGSTDYNAPFKSRVTISK
DTSKNQVSFKLSSVTVDDTAVYYCARKGGYSGSWFAYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>73C5vH47_77 Heavy Chain
(SEQ ID NO: 138)
QVQLQESGPGLVAPSETLSLTCTVSGFSLTDYAVHWIRQFPGKGLEWIGVIWSDGSTDFNAPFKSRVTISK
DTSKNQVSFKLSSVTTDDTAVYYCARKGGYSGSWFAYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
>73C5vH58_91 Heavy Chain
(SEQ ID NO: 139)
QVQLQESGPGLVKPSETLSITCTVSGFSLTDYAVHWIRQPPGKGLEWIGVIWSDGSTDYNAPFKSRVTISK
DNSKSQVSFKMSSVTADDTAVYYCARKGGYSGSWFAYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK

The CDRs listed above are defined using the Chothia numbering system (Al-Lazikani et al., (1997) JMB 273, 927-948).

In one aspect, an antibody of the present invention comprises 3 light chain CDRs and 3 heavy chain CDRs, for example as set forth above.

In one aspect, an antibody of the present invention comprises a light chain and a heavy chain variable region as set forth above. In one aspect, a light chain variable region of the invention is fused to a light chain constant region, for example a kappa or lambda constant region. In one aspect, a heavy chain variable region of the invention is fused to a heavy chain constant region, for example IgA, IgD, IgE, IgG or IgM, in particular, IgG₁, IgG₂, IgG₃ or IgG₄.

The present invention provides an anti-IL-36R antibody comprising a light chain comprising the amino acid sequence of SEQ ID NO: 115; and a heavy chain comprising the amino acid sequence of SEQ ID NO: 125 (Antibody B1).

The present invention provides an anti-IL-36R antibody comprising a light chain comprising the amino acid sequence of SEQ ID NO: 115; and a heavy chain comprising the amino acid sequence of SEQ ID NO: 126 (Antibody B2).

The present invention provides an anti-IL-36R antibody comprising a light chain comprising the amino acid sequence of SEQ ID NO: 115; and a heavy chain comprising the amino acid sequence of SEQ ID NO: 127 (Antibody B3).

The present invention provides an anti-IL-36R antibody comprising a light chain comprising the amino acid sequence of SEQ ID NO: 118; and a heavy chain comprising the amino acid sequence of SEQ ID NO: 125 (Antibody B4).

The present invention provides an anti-IL-36R antibody comprising a light chain comprising the amino acid sequence of SEQ ID NO: 118; and a heavy chain comprising the amino acid sequence of SEQ ID NO: 126 (Antibody B5).

The present invention provides an anti-IL-36R antibody comprising a light chain comprising the amino acid sequence of SEQ ID NO: 118; and a heavy chain comprising the amino acid sequence of SEQ ID NO: 127 Antibody B6).

The present invention provides an anti-IL-36R antibody comprising a light chain comprising the amino acid sequence of SEQ ID NO: 123; and a heavy chain comprising the amino acid sequence of SEQ ID NO: 138 (Antibody C3).

The present invention provides an anti-IL-36R antibody comprising a light chain comprising the amino acid sequence of SEQ ID NO: 123; and a heavy chain comprising the amino acid sequence of SEQ ID NO: 139 (Antibody C2).

The present invention provides an anti-IL-36R antibody comprising a light chain comprising the amino acid sequence of SEQ ID NO: 124; and a heavy chain comprising the amino acid sequence of SEQ ID NO: 138 (Antibody C1)

Representative antibodies of the present invention are shown below.

TABLEB

| Antibody | Light Chain Sequences | Heavy Chain Sequences |
|---|---|---|
| B1 | EIVLTQSPGTLSLSPGERATMSCTA | QVQLVQSGAEVKKPGASVKVSCKASGYSFTSS |
| | SSSVSSSYFHWYQQKPGQAPRLLIY | WIHWVRQAPGQGLEWIGEINPGNVRTNYNENF |
| | RTSILASGVPDRFSGSGSGTDFTLT | RNKATMTVDTSISTAYMELSRLRSDDTAVYYC |
| | ISRLEPEDFATYYCHQFHRSPLTFG | AVVFYGEPYFPYWGQGTLVTVSSASTKGPSVF |
| | QGTKLEIKRTVAAPSVFIFPPSDEQ | PLAPSSKSTSGGTAALGCLVKDYFPEPVTVSW |
| | LKSGTASVVCLLNNFYPREAKVQWK | NSGALTSGVHTFPAVLQSSGLYSLSSVVTVPS |
| | VDNALQSGNSQESVTEQDSKDSTYS | SSLGTQTYICNVNHKPSNTKVDKRVEPKSCDK |
| | LSSTLTLSKADYEKHKVYACEVTHQ | THTCPPCPAPEAAGGPSVFLFPPKPKDTLMIS |
| | GLSSPVTKSFNRGEC | RTPEVTCVVVDVSHEDPEVKFNWYVDGVEVHN |
| | (SEQ ID NO: 115) | AKTKPREEQYNSTYRVVSVLTVLHQDWLNGKE |
| | | YKCKVSNKALPAPIEKTISKAKGQPREPQVYT |
| | | LPPSREEMTKNQVSLTCLVKGFYPSDIAVEWE |
| | | SNGQPENNYKTTPPVLDSDGSFFLYSKLTVDK |
| | | SRWQQGNVFSCSVMHEALHNHYTQKSLSLSPG |
| | | K (SEQ ID NO: 125) |
| B2 | EIVLTQSPGTLSLSPGERATMSCTA | QVQLVQSGAEVKKPGASVKVSCKASGYSFTSS |
| | SSSVSSSYFHWYQQKPGQAPRLLIY | WIHWVRQRPGQGLEWIGEINPGNVRTNYNENF |
| | RTSILASGVPDRFSGSGSGTDFTLT | RNRVTMTVDTSISTAYMELSRLRSDDTAVYYC |
| | ISRLEPEDFATYYCHQFHRSPLTFG | TVVFYGEPYFPYWGQGTLVTVSSASTKGPSVF |
| | QGTKLEIKRTVAAPSVFIFPPSDEQ | PLAPSSKSTSGGTAALGCLVKDYFPEPVTVSW |
| | LKSGTASVVCLLNNFYPREAKVQWK | NSGALTSGVHTFPAVLQSSGLYSLSSVVTVPS |
| | VDNALQSGNSQESVTEQDSKDSTYS | SSLGTQTYICNVNHKPSNTKVDKRVEPKSCDK |
| | LSSTLTLSKADYEKHKVYACEVTHQ | THTCPPCPAPEAAGGPSVFLFPPKPKDTLMIS |
| | GLSSPVTKSFNRGEC | RTPEVTCVVVDVSHEDPEVKFNWYVDGVEVHN |
| | (SEQ ID NO: 115) | AKTKPREEQYNSTYRVVSVLTVLHQDWLNGKE |
| | | YKCKVSNKALPAPIEKTISKAKGQPREPQVYT |
| | | LPPSREEMTKNQVSLTCLVKGFYPSDIAVEWE |
| | | SNGQPENNYKTTPPVLDSDGSFFLYSKLTVDK |
| | | SRWQQGNVFSCSVMHEALHNHYTQKSLSLSPG |
| | | K (SEQ ID NO: 126) |
| B3 | EIVLTQSPGTLSLSPGERATMSCTA | QVQLVQSGAEVKKPGASVKVSCKASGYSFTSS |
| | SSSVSSSYFHWYQQKPGQAPRLLIY | WIHWVKQAPGQGLEWMGEINPGNVRTNYNENF |
| | RTSILASGVPDRFSGSGSGTDFTLT | RNKVTMTVDTSISTAYMELSRLRSDDTAVYYC |
| | ISRLEPEDFATYYCHQFHRSPLTFG | TVVFYGEPYFPYWGQGTLVTVSSASTKGPSVF |
| | QGTKLEIKRTVAAPSVFIFPPSDEQ | PLAPSSKSTSGGTAALGCLVKDYFPEPVTVSW |
| | LKSGTASVVCLLNNFYPREAKVQWK | NSGALTSGVHTFPAVLQSSGLYSLSSVVTVPS |
| | VDNALQSGNSQESVTEQDSKDSTYS | SSLGTQTYICNVNHKPSNTKVDKRVEPKSCDK |
| | LSSTLTLSKADYEKHKVYACEVTHQ | THTCPPCPAPEAAGGPSVFLFPPKPKDTLMIS |
| | GLSSPVTKSFNRGEC | RTPEVTCVVVDVSHEDPEVKFNWYVDGVEVHN |
| | (SEQ ID NO: 115) | AKTKPREEQYNSTYRVVSVLTVLHQDWLNGKE |
| | | YKCKVSNKALPAPIEKTISKAKGQPREPQVYT |
| | | LPPSREEMTKNQVSLTCLVKGFYPSDIAVEWE |
| | | SNGQPENNYKTTPPVLDSDGSFFLYSKLTVDK |
| | | SRWQQGNVFSCSVMHEALHNHYTQKSLSLSPG |
| | | K (SEQ ID NO: 127) |
| B4 | QIVLTQSPGTLSLSPGERATMTCTA | QVQLVQSGAEVKKPGASVKVSCKASGYSFTSS |
| | SSSVSSSYFHWYQQKPGQAPRLWIY | WIHWVRQAPGQGLEWIGEINPGNVRTNYNENF |
| | RTSRLASGVPDRFSGSGSGTDFTLT | RNKATMTVDTSISTAYMELSRLRSDDTAVYYC |
| | ISRLEPEDAATYYCHQFHRSPLTFG | AVVFYGEPYFPYWGQGTLVTVSSASTKGPSVF |
| | AGTKLEIKRTVAAPSVFIFPPSDEQ | PLAPSSKSTSGGTAALGCLVKDYFPEPVTVSW |
| | LKSGTASVVCLLNNFYPREAKVQWK | NSGALTSGVHTFPAVLQSSGLYSLSSVVTVPS |
| | VDNALQSGNSQESVTEQDSKDSTYS | SSLGTQTYICNVNHKPSNTKVDKRVEPKSCDK |
| | LSSTLTLSKADYEKHKVYACEVTHQ | THTCPPCPAPEAAGGPSVFLFPPKPKDTLMIS |
| | GLSSPVTKSFNRGEC | RTPEVTCVVVDVSHEDPEVKFNWYVDGVEVHN |
| | (SEQ ID NO: 118) | AKTKPREEQYNSTYRVVSVLTVLHQDWLNGKE |
| | | YKCKVSNKALPAPIEKTISKAKGQPREPQVYT |
| | | LPPSREEMTKNQVSLTCLVKGFYPSDIAVEWE |
| | | SNGQPENNYKTTPPVLDSDGSFFLYSKLTVDK |
| | | SRWQQGNVFSCSVMHEALHNHYTQKSLSLSPG |
| | | K (SEQ ID NO: 125) |
| B5 | QIVLTQSPGTLSLSPGERATMTCTA | QVQLVQSGAEVKKPGASVKVSCKASGYSFTSS |
| | SSSVSSSYFHWYQQKPGQAPRLWIY | WIHWVRQRPGQGLEWIGEINPGNVRTNYNENF |
| | RTSRLASGVPDRFSGSGSGTDFTLT | RNRVTMTVDTSISTAYMELSRLRSDDTAVYYC |
| | ISRLEPEDAATYYCHQFHRSPLTFG | TVVFYGEPYFPYWGQGTLVTVSSASTKGPSVF |
| | AGTKLEIKRTVAAPSVFIFPPSDEQ | PLAPSSKSTSGGTAALGCLVKDYFPEPVTVSW |
| | LKSGTASVVCLLNNFYPREAKVQWK | NSGALTSGVHTFPAVLQSSGLYSLSSVVTVPS |
| | VDNALQSGNSQESVTEQDSKDSTYS | SSLGTQTYICNVNHKPSNTKVDKRVEPKSCDK |
| | LSSTLTLSKADYEKHKVYACEVTHQ | THTCPPCPAPEAAGGPSVFLFPPKPKDTLMIS |
| | GLSSPVTKSFNRGEC | RTPEVTCVVVDVSHEDPEVKFNWYVDGVEVHN |
| | (SEQ ID NO: 118) | AKTKPREEQYNSTYRVVSVLTVLHQDWLNGKE |
| | | YKCKVSNKALPAPIEKTISKAKGQPREPQVYT |
| | | LPPSREEMTKNQVSLTCLVKGFYPSDIAVEWE |
| | | SNGQPENNYKTTPPVLDSDGSFFLYSKLTVDK |
| | | SRWQQGNVFSCSVMHEALHNHYTQKSLSLSPG |
| | | K (SEQ ID NO: 126) |
| B6 | QIVLTQSPGTLSLSPGERATMTCTA | QVQLVQSGAEVKKPGASVKVSCKASGYSFTSS |
| | SSSVSSSYFHWYQQKPGQAPRLWIY | WIHWVKQAPGQGLEWMGEINPGNVRTNYNENF |
| | RTSRLASGVPDRFSGSGSGTDFTLT | RNKVTMTVDTSISTAYMELSRLRSDDTAVYYC |
| | ISRLEPEDAATYYCHQFHRSPLTFG | TVVFYGEPYFPYWGQGTLVTVSSASTKGPSVF |
| | AGTKLEIKRTVAAPSVFIFPPSDEQ | PLAPSSKSTSGGTAALGCLVKDYFPEPVTVSW |
| | LKSGTASVVCLLNNFYPREAKVQWK | NSGALTSGVHTFPAVLQSSGLYSLSSVVTVPS |
| | VDNALQSGNSQESVTEQDSKDSTYS | SSLGTQTYICNVNHKPSNTKVDKRVEPKSCDK |
| | LSSTLTLSKADYEKHKVYACEVTHQ | THTCPPCPAPEAAGGPSVFLFPPKPKDTLMIS |
| | GLSSPVTKSFNRGEC | RTPEVTCVVVDVSHEDPEVKFNWYVDGVEVHN |
| | (SEQ ID NO: 118) | AKTKPREEQYNSTYRVVSVLTVLHQDWLNGKE |
| | | YKCKVSNKALPAPIEKTISKAKGQPREPQVYT |
| | | LPPSREEMTKNQVSLTCLVKGFYPSDIAVEWE |
| | | SNGQPENNYKTTPPVLDSDGSFFLYSKLTVDK |
| | | SRWQQGNVFSCSVMHEALHNHYTQKSLSLSPG |
| | | K (SEQ ID NO: 127) |

TABLEC

| Antibody | Light Chain Sequences | Heavy Chain Sequences |
|---|---|---|
| C1 | EIVMTQSPATLSVSPGVRATLSCKAS | QVQLQESGPGLVAPSETLSLTCTVSGFS |
| | QDVGTNVLWYQQKPGQAPRPLIYSAS | LTDYAVHWIRQFPGKGLEWIGVIWSDGS |
| | YRHSGIPARFSGSGSGTEFTLTISSL | TDFNAPFKSRVTISKDTSKNQVSFKLSS |
| | QSEDFAEYYCQQYSRYPLTFGQGTKL | VTTDDTAVYYCARKGGYSGSWFAYWGQG |
| | EIKRTVAAPSVFIFPPSDEQLKSGTA | TLVTVSSASTKGPSVFPLAPSSKSTSGG |
| | SVVCLLNNFYPREAKVQWKVDNALQS | TAALGCLVKDYFPEPVTVSWNSGALTSG |
| | GNSQESVTEQDSKDSTYSLSSTLTLS | VHTFPAVLQSSGLYSLSSVVTVPSSSLG |
| | KADYEKHKVYACEVTHQGLSSPVTKS | TQTYICNVNHKPSNTKVDKRVEPKSCDK |
| | FNRGEC (SEQ ID NO: 124) | THTCPPCPAPEAAGGPSVFLFPPKPKDT |
| | | LMISRTPEVTCVVVDVSHEDPEVKFNWY |
| | | VDGVEVHNAKTKPREEQYNSTYRVVSVL |
| | | TVLHQDWLNGKEYKCKVSNKALPAPIEK |
| | | TISKAKGQPREPQVYTLPPSREEMTKNQ |
| | | VSLTCLVKGFYPSDIAVEWESNGQPENN |
| | | YKTTPPVLDSDGSFFLYSKLTVDKSRWQ |
| | | QGNVFSCSVMHEALHNHYTQKSLSLSPG |
| | | K (SEQ ID NO: 138) |
| C2 | EIVMTQSPATLSVSPGVRATLSCKAS | QVQLQESGPGLVKPSETLSITCTVSGFS |
| | QDVGTNVLWYQQKPGQAPRPLIYSAS | LTDYAVHWIRQPPGKGLEWIGVIWSDGS |
| | YRHSGIPDRFSGSGSGTEFTLTISSL | TDYNAPFKSRVTISKDNSKSQVSFKMSS |
| | QSEDFAVYYCQQYSRYPLTFGQGTKL | VTADDTAVYYCARKGGYSGSWFAYWGQG |
| | EIKRTVAAPSVFIFPPSDEQLKSGTA | TLVTVSSASTKGPSVFPLAPSSKSTSGG |
| | SVVCLLNNFYPREAKVQWKVDNALQS | TAALGCLVKDYFPEPVTVSWNSGALTSG |
| | GNSQESVTEQDSKDSTYSLSSTLTLS | VHTFPAVLQSSGLYSLSSVVTVPSSSLG |
| | KADYEKHKVYACEVTHQGLSSPVTKS | TQTYICNVNHKPSNTKVDKRVEPKSCDK |
| | FNRGEC (SEQ ID NO: 123) | THTCPPCPAPEAAGGPSVFLFPPKPKDT |
| | | LMISRTPEVTCVVVDVSHEDPEVKFNWY |
| | | VDGVEVHNAKTKPREEQYNSTYRVVSVL |
| | | TVLHQDWLNGKEYKCKVSNKALPAPIEK |
| | | TISKAKGQPREPQVYTLPPSREEMTKNQ |
| | | VSLTCLVKGFYPSDIAVEWESNGQPENN |
| | | YKTTPPVLDSDGSFFLYSKLTVDKSRWQ |
| | | QGNVFSCSVMHEALHNHYTQKSLSLSPG |
| | | K (SEQ ID NO: 139) |
| C3 | EIVMTQSPATLSVSPGVRATLSCKAS | QVQLQESGPGLVAPSETLSLTCTVSGFS |
| | QDVGTNVLWYQQKPGQAPRPLIYSAS | LTDYAVHWIRQFPGKGLEWIGVIWSDGS |
| | YRHSGIPDRFSGSGSGTEFTLTISSL | TDFNAPFKSRVTISKDTSKNQVSFKLSS |
| | QSEDFAVYYCQQYSRYPLTFGQGTKL | VTTDDTAVYYCARKGGYSGSWFAYWGQG |
| | EIKRTVAAPSVFIFPPSDEQLKSGTA | TLVTVSSASTKGPSVFPLAPSSKSTSGG |
| | SVVCLLNNFYPREAKVQWKVDNALQS | TAALGCLVKDYFPEPVTVSWNSGALTSG |
| | GNSQESVTEQDSKDSTYSLSSTLTLS | VHTFPAVLQSSGLYSLSSVVTVPSSSLG |
| | KADYEKHKVYACEVTHQGLSSPVTKS | TQTYICNVNHKPSNTKVDKRVEPKSCDK |
| | FNRGEC (SEQ ID NO: 123) | THTCPPCPAPEAAGGPSVFLFPPKPKDT |
| | | LMISRTPEVTCVVVDVSHEDPEVKFNWY |
| | | VDGVEVHNAKTKPREEQYNSTYRVVSVL |
| | | TVLHQDWLNGKEYKCKVSNKALPAPIEK |
| | | TISKAKGQPREPQVYTLPPSREEMTKNQ |
| | | VSLTCLVKGFYPSDIAVEWESNGQPENN |
| | | YKTTPPVLDSDGSFFLYSKLTVDKSRWQ |
| | | QGNVFSCSVMHEALHNHYTQKSLSLSPG |
| | | K (SEQ ID NO: 138) |

In some aspects, the humanized antibody displays blocking activity, whereby it decreases the binding of IL-36 ligand to IL-36 receptor by at least 45%, by at least 50%, by at least 55%, by at least 60%, by at least 65%, by at least 70%, by at least 75%, by at least 80%, by at least 85%, by at least 90%, or by at least 95%. The ability of an antibody to block binding of IL-36 ligand to the IL-36 receptor can be measured using competitive binding assays known in the art. Alternatively, the blocking activity of an antibody can be measured by assessing the biological effects of IL-36, such as the production of IL-8, IL-6, and GM-CSF to determine if signaling mediated by the IL-36 receptor is inhibited.

In a further aspect, the present invention provides a humanized anti-IL-36R antibody having favorable biophysical properties. In one aspect, a humanized anti-IL-36R antibody of the present invention is present in at least 90% monomer form, or in at least 92% monomer form, or in at least 95% monomer form in a buffer. In a further aspect, a humanized anti-IL-36R antibody of the present invention remains in at least 90% monomer form, or in at least 92% monomer form, or in at least 95% monomer form in a buffer for one month or for four months.

In one aspect, a humanized antibody of the present invention is Antibody B1, Antibody B2, Antibody B3, Antibody B4, Antibody B5, Antibody B6, Antibody C1, Antibody C2, or Antibody C3. Accordingly, in one embodiment, a humanized antibody of the present invention comprises the light chain sequence of SEQ ID NO:115 and the heavy chain sequence of SEQ ID NO:125 (Antibody B1). In another embodiment, a humanized antibody of the present invention comprises the light chain sequence of SEQ ID NO:115 and the heavy chain sequence of SEQ ID NO:126 (Antibody B2). In another embodiment, a humanized antibody of the present invention comprises the light chain sequence of SEQ ID NO:115 and the heavy chain sequence of SEQ ID NO:127 (Antibody B3). In another embodiment, a humanized antibody of the present invention comprises the light chain sequence of SEQ ID NO:118 and the heavy chain sequence of SEQ ID NO:125 (Antibody B4). In another embodiment, a humanized antibody of the present invention comprises the light chain sequence of SEQ ID NO:118 and the heavy chain sequence of SEQ ID NO:126 (Antibody B5). In another embodiment, a humanized antibody of the present invention comprises the light chain sequence of SEQ ID NO:118 and the heavy chain sequence of SEQ ID NO:127 (Antibody B6). In another embodiment, a humanized antibody of the present invention comprises the light chain sequence of SEQ ID NO:124 and the heavy chain sequence of SEQ ID NO:138 (Antibody C1). In another embodiment, a humanized antibody of the present invention comprises the light chain sequence of SEQ ID NO:123 and the heavy chain sequence of SEQ ID NO:139 (Antibody C2). In another embodiment, a humanized antibody of the present invention comprises the light chain sequence of SEQ ID NO:123 and the heavy chain sequence of SEQ ID NO:138 (Antibody C3).

In a further embodiment, a humanized antibody of the present invention consists of the light chain sequence of SEQ ID NO:115 and the heavy chain sequence of SEQ ID NO:125 (Antibody B1). In another embodiment, a humanized antibody of the present invention consists of the light chain sequence of SEQ ID NO:115 and the heavy chain sequence of SEQ ID NO:126 (Antibody B2). In another embodiment, a humanized antibody of the present invention consists of the light chain sequence of SEQ ID NO:115 and the heavy chain sequence of SEQ ID NO:127 (Antibody B3). In another embodiment, a humanized antibody of the present invention consists of the light chain sequence of SEQ ID NO:118 and the heavy chain sequence of SEQ ID NO:125 (Antibody B4). In another embodiment, a humanized antibody of the present invention consists of the light chain sequence of SEQ ID NO:118 and the heavy chain sequence of SEQ ID NO:126 (Antibody B5). In another embodiment, a humanized antibody of the present invention consists of the light chain sequence of SEQ ID NO:118 and the heavy chain sequence of SEQ ID NO:127 (Antibody B6). In another embodiment, a humanized antibody of the present invention consists of the light chain sequence of SEQ ID NO:124 and the heavy chain sequence of SEQ ID NO:138 (Antibody C1). In another embodiment, a humanized antibody of the present invention consists of the light chain sequence of SEQ ID NO:123 and the heavy chain sequence of SEQ ID NO:139 (Antibody C2). In another embodiment, a humanized antibody of the present invention consists of the light chain sequence of SEQ ID NO:123 and the heavy chain sequence of SEQ ID NO:138 (Antibody C3).

In some embodiments, the humanized anti-IL-36R antibodies, including antigen-binding fragments thereof, such as heavy and light chain variable regions, comprise an amino acid sequence of the residues derived from Antibody B1, Antibody B2, Antibody B3, Antibody B4, Antibody B5, Antibody B6, Antibody C1, Antibody C2, or Antibody C3.

In a further embodiment, the present invention provides an anti-IL-36R antibody or antigen-binding fragment thereof that competitively binds to human anti-IL-36R with an antibody of the present invention, for example Antibody B1, Antibody B2, Antibody B3, Antibody B4, Antibody B5, Antibody B6, Antibody C1, Antibody C2 or Antibody C3 described herein. The ability of an antibody or antigen-binding fragment to competitively bind to IL-36R can be measured using competitive binding assays known in the art.

The humanized anti-IL-36R antibodies optionally include specific amino acid substitutions in the consensus or germline framework regions. The specific substitution of amino acid residues in these framework positions can improve various aspects of antibody performance including binding affinity and/or stability, over that demonstrated in humanized antibodies formed by “direct swap” of CDRs or HVLs into the human germline framework regions.

In some embodiments, the present invention describes other monoclonal antibodies with a light chain variable region having the amino acid sequence set forth in any one of SEQ ID NO:1-10. In some embodiments, the present invention describes other monoclonal antibodies with a heavy chain variable region having the amino acid sequence set forth in any one of SEQ ID NO:11-20. Placing such CDRs into FRs of the human consensus heavy and light chain variable domains will yield useful humanized antibodies of the present invention.

In particular, the present invention provides monoclonal antibodies with the combinations of light chain variable and heavy chain variable regions of SEQ ID NO:1/11, 2/12, 3/13, 4/14, 5/15, 6/16, 7/17, 8/18, 9/19, 10/20. Such variable regions can be combined with human constant regions.

In some embodiments, the present invention describes other humanized antibodies with light chain variable region sequences having the amino acid sequence set forth in any one of SEQ ID NO:76-86. In some embodiments, the present invention describes other humanized antibodies with heavy chain variable region sequences having the amino acid sequence set forth in any one of SEQ ID NO:87-101. In particular, the present invention provides monoclonal antibodies with the combinations of light chain variable and heavy chain variable regions of SEQ ID NO: 77/89, 80/88, 80/89, 77/87, 77/88, 80/87, 86/100, 85/101, 85/100. Such variable regions can be combined with human constant regions.

In a further embodiment, the present invention relates to an anti-IL-36R antibody or antigen-binding fragment thereof comprising a humanized light chain variable domain comprising the CDRs of SEQ ID NO:77 and framework regions having an amino acid sequence at least 90% identical, at least 93% identical or at least 95% identical to the amino acid sequence of the framework regions of the variable domain light chain amino acid sequence of SEQ ID NO:77 and a humanized heavy chain variable domain comprising the CDRs of SEQ ID NO:89 and framework regions having an amino acid sequence at least 90% identical, at least 93% identical or at least 95% identical to the amino acid sequence of the framework regions of the variable domain heavy chain amino acid sequence of SEQ ID NO:89. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:80 and framework regions having an amino acid sequence at least 90% identical, at least 93% identical or at least 95% identical to the amino acid sequence of the framework regions of the variable domain light chain amino acid sequence of SEQ ID NO:80 and a humanized heavy chain variable domain comprising the CDRs of SEQ ID NO:88 and framework regions having an amino acid sequence at least 90% identical, at least 93% identical or at least 95% identical to the amino acid sequence of the framework regions of the variable domain heavy chain amino acid sequence of SEQ ID NO:88. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:80 and framework regions having an amino acid sequence at least 90% identical, at least 93% identical or at least 95% identical to the amino acid sequence of the framework regions of the variable domain light chain amino acid sequence of SEQ ID NO:80 and a humanized heavy chain variable domain comprising the CDRs of SEQ ID NO:89 and framework regions having an amino acid sequence at least 90% identical, at least 93% identical or at least 95% identical to the amino acid sequence of the framework regions of the variable domain heavy chain amino acid sequence of SEQ ID NO:89. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:77 and framework regions having an amino acid sequence at least 90% identical, at least 93% identical or at least 95% identical to the amino acid sequence of the framework regions of the variable domain light chain amino acid sequence of SEQ ID NO:77 and a humanized heavy chain variable domain comprising the CDRs of SEQ ID NO:87 and framework regions having an amino acid sequence at least 90% identical, at least 93% identical or at least 95% identical to the amino acid sequence of the framework regions of the variable domain heavy chain amino acid sequence of SEQ ID NO:87. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:77 and framework regions having an amino acid sequence at least 90% identical, at least 93% identical or at least 95% identical to the amino acid sequence of the framework regions of the variable domain light chain amino acid sequence of SEQ ID NO:77 and a humanized heavy chain variable domain comprising the CDRs of SEQ ID NO:88 and framework regions having an amino acid sequence at least 90% identical, at least 93% identical or at least 95% identical to the amino acid sequence of the framework regions of the variable domain heavy chain amino acid sequence of SEQ ID NO:88. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:80 and framework regions having an amino acid sequence at least 90% identical, at least 93% identical or at least 95% identical to the amino acid sequence of the framework regions of the variable domain light chain amino acid sequence of SEQ ID NO:80 and a humanized heavy chain variable domain comprising the CDRs of SEQ ID NO:87 and framework regions having an amino acid sequence at least 90% identical, at least 93% identical or at least 95% identical to the amino acid sequence of the framework regions of the variable domain heavy chain amino acid sequence of SEQ ID NO:87. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:86 and framework regions having an amino acid sequence at least 90% identical, at least 93% identical or at least 95% identical to the amino acid sequence of the framework regions of the variable domain light chain amino acid sequence of SEQ ID NO:86 and a humanized heavy chain variable domain comprising the CDRs of SEQ ID NO:100 and framework regions having an amino acid sequence at least 90% identical, at least 93% identical or at least 95% identical to the amino acid sequence of the framework regions of the variable domain heavy chain amino acid sequence of SEQ ID NO:100. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:85 and framework regions having an amino acid sequence at least 90% identical, at least 93% identical or at least 95% identical to the amino acid sequence of the framework regions of the variable domain light chain amino acid sequence of SEQ ID NO:85 and a humanized heavy chain variable domain comprising the CDRs of SEQ ID NO:101 and framework regions having an amino acid sequence at least 90% identical, at least 93% identical or at least 95% identical to the amino acid sequence of the framework regions of the variable domain heavy chain amino acid sequence of SEQ ID NO:101. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:85 and framework regions having an amino acid sequence at least 90% identical, at least 93% identical or at least 95% identical to the amino acid sequence of the framework regions of the variable domain light chain amino acid sequence of SEQ ID NO:85 and a humanized heavy chain variable domain comprising the CDRs of SEQ ID NO:100 and framework regions having an amino acid sequence at least 90% identical, at least 93% identical or at least 95% identical to the amino acid sequence of the framework regions of the variable domain heavy chain amino acid sequence of SEQ ID NO:100. In one embodiment, the anti-IL-36R antibody is a humanized monoclonal antibody.

In some specific embodiments, the humanized anti-IL-36R antibodies disclosed herein comprise at least a heavy or a light chain variable domain comprising the CDRs or HVLs of the murine monoclonal antibodies or humanized antibodies as disclosed herein and the FRs of the human germline heavy and light chain variable domains.

In one further aspect, the present invention provides an anti-IL-36R antibody or antigen-binding fragment thereof comprising a light chain CDR1 (L-CDR1) sequence of any one of SEQ ID NO:21-29; a light chain CDR2 (L-CDR2) sequence of any one of SEQ ID NO:30-38; a light chain CDR3 (L-CDR3) sequence of any one of SEQ ID NO:39-47; a heavy chain CDR1 (H-CDR1) sequence of any one of SEQ ID NO:48-56; a heavy chain CDR2 (H-CDR2) sequence of any one of SEQ ID NO:57-66; and a heavy chain CDR3 (H-CDR3) sequence of any one of SEQ ID NO:67-75. In one aspect, the anti-IL-36R antibody or antigen-binding fragment thereof comprises a light chain variable region comprising a L-CDR1 listed above, a L-CDR2 listed above and a L-CDR3 listed above, and a heavy chain variable region comprising a H-CDR1 listed above, a H-CDR2 listed above and a H-CDR3 listed above.

In a further aspect, the present invention provides an anti-IL-36R antibody or antigen-binding fragment thereof comprising:
a) a L-CDR1, a L-CDR2, a L-CDR3, a H-CDR1, a H-CDR2 and a H-CDR3 sequence of SEQ ID NO:21, 30, 39, 48, 57 and 67, respectively; or
b) a L-CDR1, a L-CDR2, a L-CDR3, a H-CDR1, a H-CDR2 and a H-CDR3 sequence of SEQ ID NO:22, 31, 40, 49, 58 and 68, respectively; or
c) a L-CDR1, a L-CDR2, a L-CDR3, a H-CDR1, a H-CDR2 and a H-CDR3 sequence of SEQ ID NO:23, 32, 41, 50, 59 and 69, respectively; or
d) a L-CDR1, a L-CDR2, a L-CDR3, a H-CDR1, a H-CDR2 and a H-CDR3 sequence of SEQ ID NO:24, 33, 42, 51, 60 and 70, respectively; or
e) a L-CDR1, a L-CDR2, a L-CDR3, a H-CDR1, a H-CDR2 and a H-CDR3 sequence of SEQ ID NO:25, 34, 43, 52, 61 and 71, respectively; or
f) a L-CDR1, a L-CDR2, a L-CDR3, a H-CDR1, a H-CDR2 and a H-CDR3 sequence of SEQ ID NO:26, 35, 44, 53, 62 and 72, respectively; or
g) a L-CDR1, a L-CDR2, a L-CDR3, a H-CDR1, a H-CDR2 and a H-CDR3 sequence of SEQ ID NO:27, 36, 45, 54, 63 and 73, respectively; or
h) a L-CDR1, a L-CDR2, a L-CDR3, a H-CDR1, a H-CDR2 and a H-CDR3 sequence of SEQ ID NO:27, 36, 45, 54, 64 and 74, respectively; or
i) a L-CDR1, a L-CDR2, a L-CDR3, a H-CDR1, a H-CDR2 and a H-CDR3 sequence of SEQ ID NO:27, 36, 45, 54, 64 and 73, respectively; or
j) a L-CDR1, a L-CDR2, a L-CDR3, a H-CDR1, a H-CDR2 and a H-CDR3 sequence of SEQ ID NO:28, 37, 46, 55, 65 and 74, respectively; or
k) a L-CDR1, a L-CDR2, a L-CDR3, a H-CDR1, a H-CDR2 and a H-CDR3 sequence of SEQ ID NO:29, 38, 47, 56, 66 and 75, respectively.

In a further aspect, the present invention provides an anti-IL-36R antibody or antigen-binding fragment thereof comprising:
a) a L-CDR1, a L-CDR2, a L-CDR3, a H-CDR1, a H-CDR2 and a H-CDR3 sequence of SEQ ID NO:26, 103, 44, 53, 62 and 72, respectively; or
b) a L-CDR1, a L-CDR2, a L-CDR3, a H-CDR1, a H-CDR2 and a H-CDR3 sequence of SEQ ID NO:26, 104, 44, 53, 62 and 72, respectively; or
c) a L-CDR1, a L-CDR2, a L-CDR3, a H-CDR1, a H-CDR2 and a H-CDR3 sequence of SEQ ID NO:26, 104, 44, 141, 142 and 72, respectively; or
d) a L-CDR1, a L-CDR2, a L-CDR3, a H-CDR1, a H-CDR2 and a H-CDR3 sequence of SEQ ID NO:27, 36, 45, 107, 63 and 73, respectively; or
e) a L-CDR1, a L-CDR2, a L-CDR3, a H-CDR1, a H-CDR2 and a H-CDR3 sequence of SEQ ID NO:27, 36, 45, 107, 64 or 73, respectively.

In one aspect, the anti-IL-36R antibody or antigen-binding fragment thereof comprises a light chain variable region comprising a L-CDR1, L-CDR2 and L-CDR3 combination listed above, and a heavy chain variable region comprising a H-CDR1, H-CDR2 and H-CDR3 combination listed above.

In specific embodiments, it is contemplated that chimeric antibodies with switched CDR regions (i.e., for example switching one or two CDRs of one of the mouse antibodies or humanized antibody derived therefrom with the analogous CDR from another mouse antibody or humanized antibody derived therefrom) between these exemplary immunoglobulins may yield useful antibodies.

In certain embodiments, the humanized anti-IL-36R antibody is an antibody fragment. Various antibody fragments have been generally discussed above and there are techniques that have been developed for the production of antibody fragments. Fragments can be derived via proteolytic digestion of intact antibodies (see, e.g., Morimoto et al., 1992, Journal of Biochemical and Biophysical Methods 24:107-117; and Brennan et al., 1985, Science 229:81). Alternatively, the fragments can be produced directly in recombinant host cells. For example, Fab′-SH fragments can be directly recovered from E. coli and chemically coupled to form F(ab′)₂ fragments (see, e.g., Carter et al., 1992, Bio/Technology 10:163-167). By another approach, F(ab′)₂ fragments can be isolated directly from recombinant host cell culture. Other techniques for the production of antibody fragments will be apparent to the skilled practitioner. Accordingly, in one aspect, the present invention provides antibody fragments comprising the CDRs described herein, in particular one of the combinations of L-CDR1, L-CDR2, L-CDR3, H-CDR1, H-CDR2 and H-CDR3 described herein. In a further aspect, the present invention provides antibody fragments comprising the variable regions described herein, for example one of the combinations of light chain variable regions and heavy chain variable regions described herein.

Certain embodiments include an F(ab′)₂ fragment of a humanized anti-IL-36R antibody comprise a light chain sequence of any of SEQ ID NO: 115 or 118 in combination with a heavy chain sequence of SEQ ID NO: 125, 126 or 127. Such embodiments can include an intact antibody comprising such an F(ab′)₂.

Certain embodiments include an F(ab′)₂ fragment of a humanized anti-IL-36R antibody comprise a light chain sequence of any of SEQ ID NO: 123 or 124 in combination with a heavy chain sequence of SEQ ID NO: 138 or 139. Such embodiments can include an intact antibody comprising such an F(ab′)₂.

In some embodiments, the antibody or antibody fragment includes a constant region that mediates effector function. The constant region can provide antibody-dependent cellular cytotoxicity (ADCC), antibody-dependent cellular phagocytosis (ADCP) and/or complement-dependent cytotoxicity (CDC) responses against an anti-IL-36R expressing target cell. The effector domain(s) can be, for example, an Fc region of an Ig molecule.

The effector domain of an antibody can be from any suitable vertebrate animal species and isotypes. The isotypes from different animal species differ in the abilities to mediate effector functions. For example, the ability of human immunoglobulin to mediate CDC and ADCC/ADCP is generally in the order of IgM≈IgG₁≈IgG₃>IgG₂>IgG₄ and IgG₁≈IgG₃>IgG₂/IgM/IgG₄, respectively. Murine immunoglobulins mediate CDC and ADCC/ADCP generally in the order of murine IgM≈IgG₃>>IgG_{2b}>IgG₂ₐ>>IgG₁ and IgG_{2b}>IgG₂ₐ>IgG₁>>IgG₃, respectively. In another example, murine IgG₂a mediates ADCC while both murine IgG₂a and IgM mediate CDC.

### III. Pharmaceutical Doses and Administration

Anti-IL-36R antibodies of the present invention are typically administered to a patient as a pharmaceutical composition in which the antagonist is admixed with a pharmaceutically acceptable carrier or excipient, see, e. g., Remington's Pharmaceutical Sciences and US. Pharmacopeia: National Formulary, Mack Publishing Company, Easton, Pa. (1984). The pharmaceutical composition may be formulated in any manner suitable for the intended route of administration. Examples of pharmaceutical formulations include lyophilized powders, slurries, aqueous solutions, suspensions and sustained release formulations (see, e. g., Hardman et al. (2001) Goodman and Gilman's The Pharmacological Basis of Therapeutics, McGraw-Hill, New York, N. Y.; Gennaro (2000) Remington: The Science and Practice of Pharmacy, Lippincott, Williams, and Wilkins, New York, N. Y.; Avis et al. (eds.) (1993) Pharmaceutical Dosage Forms: Parenteral Medications, Marcel Dekker, NY; Lieberman et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N. Y.). Suitable routes of administration include intravenous injection (including intraarterial injection) and subcutaneous injection.

In one aspect, the present invention relates to a method of treating palmoplantar pustulosis (PPP) in a patient, said method including administering or having administered to the patient a therapeutically effective amount of an anti-IL-36R antibody.

In another aspect, the present invention relates to a method of treating moderate to severe PPP in a patient, including administering or having administered to the patient a therapeutically effective amount of an anti-IL-36R antibody.

In another aspect, the present invention relates to a method of treating chronic disease conditions associated with PPP (including periodic appearance or worsening of pustules) in a patient, including administering or having administered to the patient a therapeutically effective amount of an anti-IL-36R antibody.

In another aspect, the present invention relates to a method of reducing or alleviating signs and symptoms of an acute or chronic phase flare-up (including new appearance or worsening of pustules) of PPP in a patient, said method including administering or having administered to the patient a therapeutically effective amount of an anti-IL-36R antibody.

In another aspect, the present invention relates to a method of reducing the severity and duration of PPP flares (including new appearance or worsening of pustules), said method comprising including administering or having administered to the patient a therapeutically effective amount of an anti-IL-36R antibody.

In another aspect, the present invention relates to a method of treating a skin disorder associated with acute PPP (including new appearance or worsening of pustules), said method including administering or having administered to the patient a therapeutically effective amount of an anti-IL-36R antibody.

In another aspect, the present invention relates to a method of preventing the recurrence of PPP flares (including new appearance or worsening of pustules) in a patient treated with an anti-IL-36R antibody of the present invention.

In another aspect, the present invention relates to a method of achieving a PPP ASI50 at week 16 in a patient treated with an anti-IL-36R antibody.

In another aspect, the present invention relates to a method of achieving a complete resolution of PPP symptoms in a patient treated with an anti-IL-36R antibody; wherein the PPP symptoms comprise pustule, erythema, crust, or scaling and the complete resolution comprises a PPP PGA score of 0 (clear, e.g., on signs of PPP; no scaling or crusts or pustule remains) or 1 (almost clear, slight scaling and/or erythema and/or slight crusts; very few new (yellow) and/or old (brown) pustules).

In another aspect, the present invention relates to a method of treating PPP in a patient, including:
(a) obtaining a biological sample from said patient, wherein the biological sample is obtained from source including lesional skin or whole blood;
(b) performing or having performed sequencing assay or expression analysis of one or more of genes;
(c) administering to the patient an effective amount of an anti-IL-36R antibody based on the gene sequencing assay or expression analysis results. In an embodiment relating to this aspect, the one or more of genes is IL36RN, CARD14, AP1S3, HLA-C, C15orf48, CCL20, CXCR2, IGHA1, IL17A, IL17F, IL36A, IL36B, IL36RN, LCN2, MIR155HG, S100A12, S100A7, S100A8, VNN1, CXCR2, IL36G, IL36RN, PI3, S100A12 and/or VNN3 in lesional skin or whole blood of the patient. For example, if the expression of the gene is above a threshold level, the treatment with an anti-IL-36R antibody occurs, otherwise not.

In one embodiment related to any of the aspects and embodiments described herein, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

In one embodiment related to any of the aspects and embodiments described herein, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In one embodiment related to any of the aspects and embodiments described herein, the anti-IL-36R antibody includes:

I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

IV. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

V. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

VI. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

VII. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

In one embodiment related to any of the aspects and embodiments described herein, the anti-IL-36R antibody includes:

(i) a light chain variable region comprising the amino acid sequence of SEQ ID NO: 77; and a heavy chain variable region comprising the amino acid sequence of SEQ ID NO: 87; or

(ii) a light chain variable region comprising the amino acid sequence of SEQ ID NO: 77; and a heavy chain variable region comprising the amino acid sequence of SEQ ID NO: 88; or

(iii) a light chain variable region comprising the amino acid sequence of SEQ ID NO: 77; and a heavy chain variable region comprising the amino acid sequence of SEQ ID NO: 89; or

(iv) a light chain variable region comprising the amino acid sequence of SEQ ID NO: 80; and a heavy chain variable region comprising the amino acid sequence of SEQ ID NO: 87; or

(v) a light chain variable region comprising the amino acid sequence of SEQ ID NO: 80; and a heavy chain variable region comprising the amino acid sequence of SEQ ID NO: 88; or

(vi) a light chain variable region comprising the amino acid sequence of SEQ ID NO: 80; and a heavy chain variable region comprising the amino acid sequence of SEQ ID NO: 89; or

(vii) a light chain variable region comprising the amino acid sequence of SEQ ID NO: 85; and a heavy chain variable region comprising the amino acid sequence of SEQ ID NO: 100; or

(viii) a light chain variable region comprising the amino acid sequence of SEQ ID NO: 85; and a heavy chain variable region comprising the amino acid sequence of SEQ ID NO:101; or

(ix) a light chain variable region comprising the amino acid sequence of SEQ ID NO: 86; and a heavy chain variable region comprising the amino acid sequence of SEQ ID NO: 100; or

(x) a light chain variable region comprising the amino acid sequence of SEQ ID NO: 86; and a heavy chain variable region comprising the amino acid sequence of SEQ ID NO:101.

In one embodiment related to any of the aspects and embodiments described herein, the anti-IL-36R antibody includes:

i. a light chain comprising the amino acid sequence of SEQ ID NO: 115; and a heavy chain comprising the amino acid sequence of SEQ ID NO: 125; or

ii. a light chain comprising the amino acid sequence of SEQ ID NO: 115; and a heavy chain comprising the amino acid sequence of SEQ ID NO: 126; or

iii. a light chain comprising the amino acid sequence of SEQ ID NO: 115; and a heavy chain comprising the amino acid sequence of SEQ ID NO: 127; or

iv. a light chain comprising the amino acid sequence of SEQ ID NO: 118; and a heavy chain comprising the amino acid sequence of SEQ ID NO: 125; or

v. a light chain comprising the amino acid sequence of SEQ ID NO: 118; and a heavy chain comprising the amino acid sequence of SEQ ID NO: 126; or

vi. a light chain comprising the amino acid sequence of SEQ ID NO: 118; and a heavy chain comprising the amino acid sequence of SEQ ID NO: 127; or

vii. a light chain comprising the amino acid sequence of SEQ ID NO: 123; and a heavy chain comprising the amino acid sequence of SEQ ID NO: 138; or

viii. a light chain comprising the amino acid sequence of SEQ ID NO: 123; and a heavy chain comprising the amino acid sequence of SEQ ID NO: 139; or

ix. a light chain comprising the amino acid sequence of SEQ ID NO: 124; and a heavy chain comprising the amino acid sequence of SEQ ID NO: 138.

In an embodiment relating to any of the aspects and embodiments described herein, the anti-IL-36R antibody is administered subcutaneously or intravenously or by both routes simultaneously or sequentially and in any order. In a related embodiment, the subcutaneous administration comprises administration of 300 mg or 600 mg dose of the anti-IL-36R antibody. In a related embodiment, the intravenous administration comprises administering 300 mg, 600 mg, 900 mg or 1200 mg dose of the anti-IL-36R antibody. In a related embodiment, the subcutaneous administration is conducted at qw (once every week), q2w (once every 2 weeks), q4w (once every 4 weeks), q6w (once every 6 weeks) or q8w (once every 8 weeks) interval, or a combination thereof. In a related embodiment, the intravenous administration is conducted at q4w (once every 4 weeks), q8w (once every 8 weeks) or q12w (once every 12 weeks) interval, or a combination thereof.

In another embodiment relating to any of the aspects and embodiments described herein, the anti-IL-36R antibody is administered subcutaneously or intravenously or by both routes simultaneously or sequentially and in any order. In a related embodiment, the subcutaneous administration comprises an initial dose. In a related embodiment, the subcutaneous administration further comprises a subsequent dose. In a related embodiment, the administration of the anti-IL-36R antibody includes an initial dose and a subsequent dose. In a related embodiment, the initial dose is administered intravenously or subcutaneously. In a related embodiment, the subsequent dose is administered subcutaneously. In a related embodiment, the initial dose is 150 mg, 300 mg or 600 mg. In a related embodiment, the initial dose of 150 mg or 300 mg is administered per day (in consecutive days) for two weeks. In a related embodiment, the initial dose of 600 mg is administered once per week for two weeks including weeks 0 and 1; weeks 0 and 2; weeks 0 and 3; or weeks 0 and 4. In a related embodiment, the initial dose of 600 mg is administered once per week for three weeks including weeks 0, 1 and 2; weeks 0, 1 and 3; weeks 0, 1 and 4; weeks 0, 2 and 3; weeks 0, 2 and 4; or weeks 0, 3 and 4. In a related embodiment, the initial dose of 600 mg is administered once per week for four weeks including weeks 0, 1, 2 and 3; weeks 0, 1, 2 and 4; weeks 0, 1, 3 and 4; or weeks 0, 2, 3 and 4. In a related embodiment, the initial dose of 600 mg is administered twice per week for 2 weeks. In a related embodiment, the initial dose of 600 mg is administered twice per week for 3 weeks. In a related embodiment, the initial dose of 600 mg is administered twice per week for 4 weeks. In a related embodiment, the initial dose is 3000 mg (administered in 600 mg doses at, for example, day 1, week 1, week 2, week 3 and week 4). In a related embodiment, the initial dose is 1500 mg (administered in 300 mg doses at, for example, day 1, week 1, week 2, week 3 and week 4). In a related embodiment, the initial dose is 900 mg or 1200 mg administered IV (intravenously) or SC (subcutaneously) at q4w, q8w or q12w. In a related embodiment, the subsequent dose is 300 mg or 600 mg administered SC. In a related embodiment, the subsequent dose administration begins two to four weeks after the initial dose administration ends. In a related embodiment, the subsequent dose of 300 mg or 600 mg is administered q2w (once every 2 weeks), q4w (once every 4 weeks), q6w (once every 6 weeks) or q8w (once every 8 weeks). In a related embodiment, the subsequent dose is 600 mg administered q4w. In a related embodiment, the subsequent dose is 300 mg administered q4w. In a related embodiment, the subsequent dose is 300 mg administered q4w for eight weeks and q8w thereafter.

In one embodiment, the present invention relates to a method of preventing the recurrence of PPP flares (including new appearance or worsening of pustules), said method(s) including administering or having administered to the PPP patient a therapeutically effective amount of an anti-IL-36R antibody of the present invention subcutaneously or intravenously or by both routes according to any of the dose regimens listed in Tables 1-4 below.

In one embodiment, the present invention relates to a method of achieving a PPP Physicians Global Assessment (PPP PGA) score of 0 or 1=clear/almost clear at week 16, said method(s) including administering or having administered to the PPP patient a therapeutically effective amount of an anti-IL-36R antibody of the present invention subcutaneously or intravenously or by both routes according to any of the dose regimens listed in Tables 1-4 below.

In one embodiment, the present invention relates to a method of achieving a PPP Physicians Global Assessment (PPP PGA) score of 0 or 1=clear/almost clear at week 16, said method(s) including administering or having administered to the PPP patient a therapeutically effective amount of an anti-IL-36R antibody of the present invention subcutaneously or intravenously or by both routes according to any of the dose regimens listed in Tables 1-4 below.

Representative examples of dose regimens according to the present invention are disclosed in Tables 1-4 below.

TABLE 1
Doses and Dose Regimens

| Treatment dose | Dose |
|---|---|
| (mg) | frequency |
| 300 (SC) | qw |
| 300 (SC) | q2w |
| 300 (SC) | q4w |
| 300 (SC) | q6w |
| 300 (SC) | q8w |
| 600 (SC) | qw |
| 600 (SC) | q2w |
| 600 (SC) | q4w |
| 600 (SC) | q6w |
| 600 (SC) | q8w |
| 900 (SC) | q2w |
| 900 (SC) | q4w |
| 900 (SC) | q8w |
| 900 (SC) | q12w |

TABLE 2
Doses and Dose Regimens

| Treatment | |
|---|---|
| dose (mg) | Dose frequency |
| 900 (IV) | q4w |
| 900 (IV) | q8w |
| 900 (IV) | q12w |
| 1200 (IV) | q4w |
| 1200 (IV) | q8w |
| 1200 (IV) | q12w |

TABLE 3
Doses and Dose Regimens

| Initial dose | | Subsequent dose | |
|---|---|---|---|
| (e.g., lead-in | | (e.g., | |
| or induction | | maintenance) | Frequency of |
| dose) (mg) | Frequency of Initial dose | dose (mg) | subsequent doses |
| 600 (SC) | 5 times, Day 1-Week 4 | 600 (SC) | q4w, from week 8 & on |
| 600 (SC) | 5 times, Day 1-Week 4 | 300 (SC) | q4w, from week 8 & on |
| 300 (SC) | 5 times, Day 1-Week 4 | 600 (SC) | q4w, from week 8 & on |
| 300 (SC) | 5 times, Day 1-Week 4 | 300 (SC) | q4w, from weeks 8-16 |
| | | 300 (SC) | q8w, from week 20 & on |
| 900 (IV) | 2-3 times, Day 1-Week 4 | 600 (IV) | q6-8w, from week 8 & on |
| 900 (IV) | 2-3 times, Day 1-Week 4 | 300 (IV) | q6-8w, from week 8 & on |
| 600 (IV) | 2-3 times, Day 1-Week 4 | 600 (IV) | q6-8w, from week 8 & on |
| 600 (IV) | 2-3 times, Day 1-Week 4 | 300 (IV) | q6-8w, from week 8 & on |
| 300 (IV) | 2-3 times, Day 1-Week 4 | 600 (IV) | q6-8w, from week 8 & on |
| 300 (IV) | 2-3 times, Day 1-Week 4 | 300 (IV) | q6-8w, from weeks 8-16 |
| | | 300 (IV) | q10-12w, from week 20 & on |
| 900 (IV) | 2-3 times, Day 1-Week 4 | 600 (SC) | q6-8w, from week 8 & on |
| 900 (IV) | 2-3 times, Day 1-Week 4 | 300 (SC) | q6-8w, from week 8 & on |
| 600 (IV) | 2-3 times, Day 1-Week 4 | 600 (SC) | q4w, from week 8 & on |
| 600 (IV) | 2-3 times, Day 1-Week 4 | 300 (SC) | q4w, from week 8 & on |
| 300 (IV) | 2-3 times, Day 1-Week 4 | 600 (SC) | q4w, from week 8 & on |
| 300 (IV) | 2-3 times, Day 1-Week 4 | 300 (SC) | q4w, from weeks 8-16 |
| | | 300 (SC) | q8w, from week 20 & on |

TABLE 4
Doses and Dose Regimens

| Initial dose | | Subsequent dose | |
|---|---|---|---|
| (e.g., lead-in | | (e.g., | |
| or induction | | maintenance) | Frequency of |
| dose) (mg) | Frequency of Initial dose | dose (mg) | subsequent doses |
| 150 (SC) | Per day for 2 weeks | 300 (SC) | q2w |
| 300 (SC) | Per day for 2 weeks | 300 (SC) | q2w |
| 600 (SC) | At weeks 0 and 1 | 300 (SC) | q2w |
| 600 (SC) | At weeks 0 and 2 | 300 (SC) | q2w |
| 600 (SC) | At weeks 0 and 3 | 300 (SC) | q2w |
| 600 (SC) | At weeks 0 and 4 | 300 (SC) | q2w |
| 600 (SC) | At weeks 0, 1 and 2 | 300 (SC) | q2w |
| 600 (SC) | At weeks 0, 1 and 3 | 300 (SC) | q2w |
| 600 (SC) | At weeks 0, 1 and 4 | 300 (SC) | q2w |
| 600 (SC) | At weeks 0, 2 and 3 | 300 (SC) | q2w |
| 600 (SC) | At weeks 0, 2 and 4 | 300 (SC) | q2w |
| 600 (SC) | At weeks 0, 3 and 4 | 300 (SC) | q2w |
| 600 (SC) | At weeks 0,1,2 and 3 | 300 (SC) | q2w |
| 600 (SC) | At weeks 0,1,2 and 4 | 300 (SC) | q2w |
| 600 (SC) | At weeks 0,1,3 and 4 | 300 (SC) | q2w |
| 600 (SC) | At weeks 0, 2, 3 and 4 | 300 (SC) | q2w |
| 600 (SC) | Twice per week for 2 weeks | 300 (SC) | q2w |
| 600 (SC) | Twice per week for 3 weeks | 300 (SC) | q2w |
| 600 (SC) | Twice per week for 4 weeks | 300 (SC) | q2w |
| 150 (SC) | Per day for 2 weeks | 300 (SC) | q4w |
| 300 (SC) | Per day for 2 weeks | 300 (SC) | q4w |
| 600 (SC) | At weeks 0 and 1 | 300 (SC) | q4w |
| 600 (SC) | At weeks 0 and 2 | 300 (SC) | q4w |
| 600 (SC) | At weeks 0 and 3 | 300 (SC) | q4w |
| 600 (SC) | At weeks 0 and 4 | 300 (SC) | q4w |
| 600 (SC) | At weeks 0, 1 and 2 | 300 (SC) | q4w |
| 600 (SC) | At weeks 0, 1 and 3 | 300 (SC) | q4w |
| 600 (SC) | At weeks 0, 1 and 4 | 300 (SC) | q4w |
| 600 (SC) | At weeks 0, 2 and 3 | 300 (SC) | q4w |
| 600 (SC) | At weeks 0, 2 and 4 | 300 (SC) | q4w |
| 600 (SC) | At weeks 0, 3 and 4 | 300 (SC) | q4w |
| 600 (SC) | At weeks 0,1,2 and 3 | 300 (SC) | q4w |
| 600 (SC) | At weeks 0,1,2 and 4 | 300 (SC) | q4w |
| 600 (SC) | At weeks 0,1,3 and 4 | 300 (SC) | q4w |
| 600 (SC) | At weeks 0, 2, 3 and 4 | 300 (SC) | q4w |
| 600 (SC) | Twice per week for 2 weeks | 300 (SC) | q4w |
| 600 (SC) | Twice per week for 3 weeks | 300 (SC) | q4w |
| 600 (SC) | Twice per week for 4 weeks | 300 (SC) | q4w |
| 150 (SC) | Per day for 2 weeks | 300 (SC) | q6w |
| 300 (SC) | Per day for 2 weeks | 300 (SC) | q6w |
| 600 (SC) | At weeks 0 and 1 | 300 (SC) | q6w |
| 600 (SC) | At weeks 0 and 2 | 300 (SC) | q6w |
| 600 (SC) | At weeks 0 and 3 | 300 (SC) | q6w |
| 600 (SC) | At weeks 0 and 4 | 300 (SC) | q6w |
| 600 (SC) | At weeks 0, 1 and 2 | 300 (SC) | q6w |
| 600 (SC) | At weeks 0, 1 and 3 | 300 (SC) | q6w |
| 600 (SC) | At weeks 0, 1 and 4 | 300 (SC) | q6w |
| 600 (SC) | At weeks 0, 2 and 3 | 300 (SC) | q6w |
| 600 (SC) | At weeks 0, 2 and 4 | 300 (SC) | q6w |
| 600 (SC) | At weeks 0, 3 and 4 | 300 (SC) | q6w |
| 600 (SC) | At weeks 0,1,2 and 3 | 300 (SC) | q6w |
| 600 (SC) | At weeks 0,1,2 and 4 | 300 (SC) | q6w |
| 600 (SC) | At weeks 0,1,3 and 4 | 300 (SC) | q6w |
| 600 (SC) | At weeks 0, 2, 3 and 4 | 300 (SC) | q6w |
| 600 (SC) | Twice per week for 2 weeks | 300 (SC) | q6w |
| 600 (SC) | Twice per week for 3 weeks | 300 (SC) | q6w |
| 600 (SC) | Twice per week for 4 weeks | 300 (SC) | q6w |
| 150 (SC) | Per day for 2 weeks | 300 (SC) | q8w |
| 300 (SC) | Per day for 2 weeks | 300 (SC) | q8w |
| 600 (SC) | At weeks 0 and 1 | 300 (SC) | q8w |
| 600 (SC) | At weeks 0 and 2 | 300 (SC) | q8w |
| 600 (SC) | At weeks 0 and 3 | 300 (SC) | q8w |
| 600 (SC) | At weeks 0 and 4 | 300 (SC) | q8w |
| 600 (SC) | At weeks 0, 1 and 2 | 300 (SC) | q8w |
| 600 (SC) | At weeks 0, 1 and 3 | 300 (SC) | q8w |
| 600 (SC) | At weeks 0, 1 and 4 | 300 (SC) | q8w |
| 600 (SC) | At weeks 0, 2 and 3 | 300 (SC) | q8w |
| 600 (SC) | At weeks 0, 2 and 4 | 300 (SC) | q8w |
| 600 (SC) | At weeks 0, 3 and 4 | 300 (SC) | q8w |
| 600 (SC) | At weeks 0,1,2 and 3 | 300 (SC) | q8w |
| 600 (SC) | At weeks 0,1,2 and 4 | 300 (SC) | q8w |
| 600 (SC) | At weeks 0,1,3 and 4 | 300 (SC) | q8w |
| 600 (SC) | At weeks 0, 2, 3 and 4 | 300 (SC) | q8w |
| 600 (SC) | Twice per week for 2 weeks | 300 (SC) | q8w |
| 600 (SC) | Twice per week for 3 weeks | 300 (SC) | q8w |
| 600 (SC) | Twice per week for 4 weeks | 300 (SC) | q8w |
| 150 (SC) | Per day for 2 weeks | 600 (SC) | q2w |
| 300 (SC) | Per day for 2 weeks | 600 (SC) | q2w |
| 600 (SC) | At weeks 0 and 1 | 600 (SC) | q2w |
| 600 (SC) | At weeks 0 and 2 | 600 (SC) | q2w |
| 600 (SC) | At weeks 0 and 3 | 600 (SC) | q2w |
| 600 (SC) | At weeks 0 and 4 | 600 (SC) | q2w |
| 600 (SC) | At weeks 0, 1 and 2 | 600 (SC) | q2w |
| 600 (SC) | At weeks 0, 1 and 3 | 600 (SC) | q2w |
| 600 (SC) | At weeks 0, 1 and 4 | 600 (SC) | q2w |
| 600 (SC) | At weeks 0, 2 and 3 | 600 (SC) | q2w |
| 600 (SC) | At weeks 0, 2 and 4 | 600 (SC) | q2w |
| 600 (SC) | At weeks 0, 3 and 4 | 600 (SC) | q2w |
| 600 (SC) | At weeks 0,1,2 and 3 | 600 (SC) | q2w |
| 600 (SC) | At weeks 0,1,2 and 4 | 600 (SC) | q2w |
| 600 (SC) | At weeks 0,1,3 and 4 | 600 (SC) | q2w |
| 600 (SC) | At weeks 0, 2, 3 and 4 | 600 (SC) | q2w |
| 600 (SC) | Twice per week for 2 weeks | 600 (SC) | q2w |
| 600 (SC) | Twice per week for 3 weeks | 600 (SC) | q2w |
| 600 (SC) | Twice per week for 4 weeks | 600 (SC) | q2w |
| 150 (SC) | Per day for 2 weeks | 600 (SC) | q4w |
| 300 (SC) | Per day for 2 weeks | 600 (SC) | q4w |
| 600 (SC) | At weeks 0 and 1 | 600 (SC) | q4w |
| 600 (SC) | At weeks 0 and 2 | 600 (SC) | q4w |
| 600 (SC) | At weeks 0 and 3 | 600 (SC) | q4w |
| 600 (SC) | At weeks 0 and 4 | 600 (SC) | q4w |
| 600 (SC) | At weeks 0, 1 and 2 | 600 (SC) | q4w |
| 600 (SC) | At weeks 0, 1 and 3 | 600 (SC) | q4w |
| 600 (SC) | At weeks 0, 1 and 4 | 600 (SC) | q4w |
| 600 (SC) | At weeks 0, 2 and 3 | 600 (SC) | q4w |
| 600 (SC) | At weeks 0, 2 and 4 | 600 (SC) | q4w |
| 600 (SC) | At weeks 0, 3 and 4 | 600 (SC) | q4w |
| 600 (SC) | At weeks 0,1,2 and 3 | 600 (SC) | q4w |
| 600 (SC) | At weeks 0,1,2 and 4 | 600 (SC) | q4w |
| 600 (SC) | At weeks 0,1,3 and 4 | 600 (SC) | q4w |
| 600 (SC) | At weeks 0, 2, 3 and 4 | 600 (SC) | q4w |
| 600 (SC) | Twice per week for 2 weeks | 600 (SC) | q4w |
| 600 (SC) | Twice per week for 3 weeks | 600 (SC) | q4w |
| 600 (SC) | Twice per week for 4 weeks | 600 (SC) | q4w |
| 150 (SC) | Per day for 2 weeks | 600 (SC) | q6w |
| 300 (SC) | Per day for 2 weeks | 600 (SC) | q6w |
| 600 (SC) | At weeks 0 and 1 | 600 (SC) | q6w |
| 600 (SC) | At weeks 0 and 2 | 600 (SC) | q6w |
| 600 (SC) | At weeks 0 and 3 | 600 (SC) | q6w |
| 600 (SC) | At weeks 0 and 4 | 600 (SC) | q6w |
| 600 (SC) | At weeks 0, 1 and 2 | 600 (SC) | q6w |
| 600 (SC) | At weeks 0, 1 and 3 | 600 (SC) | q6w |
| 600 (SC) | At weeks 0, 1 and 4 | 600 (SC) | q6w |
| 600 (SC) | At weeks 0, 2 and 3 | 600 (SC) | q6w |
| 600 (SC) | At weeks 0, 2 and 4 | 600 (SC) | q6w |
| 600 (SC) | At weeks 0, 3 and 4 | 600 (SC) | q6w |
| 600 (SC) | At weeks 0,1,2 and 3 | 600 (SC) | q6w |
| 600 (SC) | At weeks 0,1,2 and 4 | 600 (SC) | q6w |
| 600 (SC) | At weeks 0,1,3 and 4 | 600 (SC) | q6w |
| 600 (SC) | At weeks 0, 2, 3 and 4 | 600 (SC) | q6w |
| 600 (SC) | Twice per week for 2 weeks | 600 (SC) | q6w |
| 600 (SC) | Twice per week for 3 weeks | 600 (SC) | q6w |
| 600 (SC) | Twice per week for 4 weeks | 600 (SC) | q6w |
| 150 (SC) | Per day for 2 weeks | 600 (SC) | q8w |
| 300 (SC) | Per day for 2 weeks | 600 (SC) | q8w |
| 600 (SC) | At weeks 0 and 1 | 600 (SC) | q8w |
| 600 (SC) | At weeks 0 and 2 | 600 (SC) | q8w |
| 600 (SC) | At weeks 0 and 3 | 600 (SC) | q8w |
| 600 (SC) | At weeks 0 and 4 | 600 (SC) | q8w |
| 600 (SC) | At weeks 0, 1 and 2 | 600 (SC) | q8w |
| 600 (SC) | At weeks 0, 1 and 3 | 600 (SC) | q8w |
| 600 (SC) | At weeks 0, 1 and 4 | 600 (SC) | q8w |
| 600 (SC) | At weeks 0, 2 and 3 | 600 (SC) | q8w |
| 600 (SC) | At weeks 0, 2 and 4 | 600 (SC) | q8w |
| 600 (SC) | At weeks 0, 3 and 4 | 600 (SC) | q8w |
| 600 (SC) | At weeks 0,1,2 and 3 | 600 (SC) | q8w |
| 600 (SC) | At weeks 0,1,2 and 4 | 600 (SC) | q8w |
| 600 (SC) | At weeks 0,1,3 and 4 | 600 (SC) | q8w |
| 600 (SC) | At weeks 0, 2, 3 and 4 | 600 (SC) | q8w |
| 600 (SC) | Twice per week for 2 weeks | 600 (SC) | q8w |
| 600 (SC) | Twice per week for 3 weeks | 600 (SC) | q8w |
| 600 (SC) | Twice per week for 4 weeks | 600 (SC) | q8w |

SC: subcutanous or subcutanously
IV: intravenous or intravenously

In a related embodiment, the anti-IL-36R antibody administration at any of the dose regimens described herein to a subject suffering from PPP and the related signs and symptoms results in one or more of the following outcomes or endpoints:
(a) the subject achieves a 50% reduction in PPP ASI (PPP ASI50) at week 16; or
(b) the subject experience a reduction in the number of drug-related Adverse Events (AEs) as compared to other treatments (e.g., guselkumab); or
(c) the subject experiences an improvement in his or her pustule severity (as compared to baseline) at week 16; or
(d) the anti-IL-36R antibody treatment shows a superior efficacy over guselkumab at week 16; or
(e) the subject achieves a PPP Physicians Global Assessment (PPP PGA) score of 0 or 1 (clear/almost clear) at week 16; or
(f) the subject achieves a Psoriasis Area and Severity Index for PPP (PPP ASI) 75 at week 16; or
(g) the subject experiences an improvement from baseline in the PPP ASI at week 16; or
(h) the subject achieves an improved change from baseline in Pain Visual Analog Scale (VAS) score at week 16; or
(i) the subject achieves a clinical improvement from baseline as assessed via Dermatology Life Quality Index (DLQI) at week 16; or
(j) the subject achieves a PPP ASI50 at visit 1, 2, 3, 4, 5, 6, 7, 8, 9, 10 or all other visits; or
(k) the subject achieves a reduction in PPP ASI scores at week 16 and all other visits; or
(l) the subject achieves PPP Physicians Global Assessment (PPP PGA) score of 0 or 1 (clear/almost clear) at visit 1, 2, 3, 4, 5, 6, 7, 8, 9, 10 or all other visits;
(m) the subject achieves a PPP ASI75 at visit 1, 2, 3, 4, 5, 6, 7, 8, 9, 10 or all other visits after treatment with the anti-IL-36R antibody;
(n) the subject experiences a percent change from baseline in the PPP ASI at visit 1, 2, 3, 4, 5, 6, 7, 8, 9, 10 or all other visits; or
(o) the subject experiences a lesser time to achieving PPP ASI50 as compared to other treatments (e.g., guselkumab); or
(p) the subject experiences a longer time to loss of PPP ASI50 as compared to other treatments (e.g., guselkumab);
(q) the subject experiences an improved change in plaque psoriasis BSA involvement at week 16 in subjects with concurrent plaque psoriasis at baseline; or
(r) the subject experiences a superiority over placebo in achieving PPP ASI50 at week 12; or
(s) the subject achieves a change in PPP ASI from baseline at week 16; or
(t) the subject achieves a positive or improved change in Pain VAS score from baseline at week 12; or
(u) the subject achieves a positive or improved PPP SI change from baseline at week 12; or
(v) the subject achieves a positive or improved PPP ASI change from baseline at week 52; or
(w) the subject achieves a reduction in occurrence of Treatment Emergent Adverse Events (TEAEs) from baseline overtime or at week 16; or
(x) the subject achieves a positive or improved change in pustule count from baseline over time; or
(y) the subject achieves a positive or improved change in pustular severity from baseline over time; or
(z) the subject achieves a PPP PGA clear/almost clear as compared to baseline or placebo over time; or
(aa) the subject achieves a PPP PGA pustule clear/almost clear as compared to baseline or placebo over time; or
(bb) the subject achieves a positive change from baseline in total score of PPQLI (Palmoplantar Quality of Life Instrument), DLQI (Dermatology Life Quality Index), PSS (Psoriasis Symptom Scale), and BASDAI (Bath Ankylosing Spondylitis Disease Activity Index) over time; or
(cc) the subject achieves a PPP ASI50 over time; or
(dd) the subject achieves a PPP ASI75 over time; or
(ee) the subject achieves a positive or improved percent change from baseline in the PPP ASI over time; or
(ff) the subject achieves a positive or improved PPSI change as compared to baseline over time; or
(gg) the subject achieves a positive or improved change in Pain VAS score for pain on palm and/or soles (PPP Pain VAS) and/or one for muscular and joint pain as compared to baseline or placebo over time; or
(hh) the subject achieves a shorter time to PPP ASI75 as compared to baseline or placebo over time; or
(ii) the subject achieves a shorter time to PPP ASI50 as compared to baseline or placebo over time; or
(jj) the subject achieves a longer time to loss of PPP ASI75 as compared to baseline or placebo over time; or
(kk) the subject achieves a longer time to loss of PPP ASI50 as compared to baseline or placebo over time; or
(ll) the subject achieves a positive or improved change in PASI as compared to baseline or placebo over time; or
(mm) the subject achieves a positive or improved change in sPGA as compared to baseline or placebo over time; or
(nn) the subject achieves a positive or improved percent change in TPSS as compared with baseline or placebo over time; or
(oo) the subject achieves a positive or improved pharmacokinetic as compared to baseline or placebo over time; or
(pp) the subject achieves an improved gene expression change for the genes disclosed herein as an indication that the treatment is efficacious as compared with baseline or placebo over time; or
(qq) the subject achieves a PPP PGA of 0 or 1 at a reduced time as compared with baseline or placebo over time.

In an embodiment relating to any of the above aspects, during or after treatment with an anti-IL-36R antibody of the present invention, the mammal or the patient is evaluated for improved Clinical Remission as defined by: (a) Palmoplantar Pustular Psoriasis Area and Severity Index 50 (PPP ASI50) at week 16; (b) reduction in the number of patients with drug-related Adverse Events (AEs); (c) PPP Physicians Global Assessment (PPP PGA) score of 0 or 1=clear/almost clear at week 16; (d) PPP ASI75 at week 16; (e) Percent change from baseline in the PPP ASI at week 16; (f) change from baseline in Pain Visual Analog Scale (VAS) score at Week 16 and all other visits collected; (g) Clinical Improvement assessed via Dermatology Life Quality Index (DLQI) at week 16 and all other visits collected compared to baseline; (h) PPP ASI50 at all other visits collected; (i) Modified (precise) PPP ASI scores at week 16 and all other visits collected; (j) Treatment success defined as achieving a clinical response of 0 or 1=clear/almost clear via PPP Physicians Global Assessment (PPP PGA) at all other visits collected; (k) PPP ASI75 at all other visits collected; (l) Percent change from baseline in the PPP ASI at all other visits collected; (m) Time (days) to achieving PPP ASI50; (n) Time (days) to loss of PPP ASI50; (o) Change in plaque psoriasis BSA involvement at week 16 in patients with concurrent plaque psoriasis at baseline; (p) Adverse reactions (including drug-related AEs) or the end points listed above or recited in Examples 1, 2 and 6. In a related embodiment, proportion of patients with a response to the administration is higher or significantly higher as compared to patients on placebo for any of the end points recited.

In one embodiment, the present invention relates to a method of treating palmoplantar pustulosis (PPP), a method of treating moderate to severe PPP, a method of treating severe PPP, a method of reducing or alleviating signs and symptoms of an acute phase flare-up of PPP (including periodic appearance or worsening of pustules), a method of reducing the severity and duration of PPP flares (including periodic appearance or worsening of pustules), or a method of treating a skin disorder associated with acute or chronic PPP in a patient, said method(s) including administering or having administered to the patient a therapeutically effective amount of an anti-IL-36R antibody of the present invention subcutaneously or intravenously or by both routes simultaneously or sequentially and in any order. In a related embodiment, the subcutaneous administration comprises administration of 300 mg or 600 mg or 900 mg dose of the anti-IL-36R antibody. In a related embodiment, the intravenous administration comprises administering 300 mg, 600 mg, 900 mg or 1200 mg dose of the anti-IL-36R antibody. In a related embodiment, the subcutaneous administration is conducted at qw (once every week), q2w (once every 2 weeks), q4w (once every 4 weeks), q6w (once every 6 weeks) or q8w (once every 8 weeks) interval, or a combination thereof. In a related embodiment, the intravenous administration is conducted at q4w (once every 4 weeks), q8w (once every 8 weeks) or q12w (once every 12 weeks) interval, or a combination thereof.

In one embodiment, the present invention relates to a method of treating palmoplantar pustulosis (PPP), a method of treating moderate to severe PPP, a method of treating severe PPP, a method of reducing or alleviating signs and symptoms of an acute phase flare-up of PPP (including periodic appearance or worsening of pustules), a method of reducing the severity and duration of PPP flares (including periodic appearance or worsening of pustules), or a method of treating a skin disorder associated with acute or chronic PPP in a patient, said method(s) including administering or having administered to the patient a therapeutically effective amount of an anti-IL-36R antibody of the present invention subcutaneously or intravenously or by both routes simultaneously or sequentially and in any order. In a related embodiment, the subcutaneous administration comprises an initial dose. In a related embodiment, the subcutaneous administration further comprises a subsequent dose. In a related embodiment, the administration of the anti-IL-36R antibody includes an initial dose and a subsequent dose. In a related embodiment, the initial dose is administered intravenously or subcutaneously. In a related embodiment, the subsequent dose is administered subcutaneously. In a related embodiment, the initial dose is 150 mg, 300 mg or 600 mg or 900 mg. In a related embodiment, the initial dose of 150 mg or 300 mg is administered per day (in consecutive days) for two weeks. In a related embodiment, the initial dose of 600 mg is administered once per week for two weeks including weeks 0 and 1; weeks 0 and 2; weeks 0 and 3; or weeks 0 and 4. In a related embodiment, the initial dose of 600 mg or 900 mg is administered once per week for three weeks including weeks 0, 1 and 2; weeks 0, 1 and 3; weeks 0, 1 and 4; weeks 0, 2 and 3; weeks 0, 2 and 4; or weeks 0, 3 and 4. In a related embodiment, the initial dose of 600 mg or 900 mg is administered once per week for four weeks including weeks 0, 1, 2 and 3; weeks 0, 1, 2 and 4; weeks 0, 1, 3 and 4; or weeks 0, 2, 3 and 4. In a related embodiment, the initial dose of 600 mg or 900 mg is administered twice per week for 2 weeks. In a related embodiment, the initial dose of 600 mg or 900 mg is administered twice per week for 3 weeks. In a related embodiment, the initial dose of 600 mg or 900 mg is administered twice per week for 4 weeks. In a related embodiment, the initial dose is 3000 mg (administered in 600 mg doses at, for example, day 1, week 1, week 2, week 3 and week 4). In a related embodiment, the initial dose is 1500 mg (administered in 300 mg doses at, for example, day 1, week 1, week 2, week 3 and week 4). In a related embodiment, the initial dose is 900 mg or 1200 mg administered IV (intravenously) or SC (subcutaneously) at q4w, q8w or q12w. In a related embodiment, the subsequent dose is 300 mg or 600 mg administered SC. In a related embodiment, the subsequent dose administration begins two to four weeks after the initial dose administration ends. In a related embodiment, the subsequent dose of 300 mg or 600 mg is administered q2w (once every 2 weeks), q4w (once every 4 weeks), q6w (once every 6 weeks) or q8w (once every 8 weeks). In a related embodiment, the subsequent dose is 600 mg administered q4w. In a related embodiment, the subsequent dose is 300 mg administered q4w. In a related embodiment, the subsequent dose is 300 mg administered q4w for eight weeks and q8w thereafter.

In an embodiment relating to any of the above aspects, the anti-IL-36R antibody or an antigen binding fragment thereof (disclosed herein) is present in a stable pharmaceutical formulation (as described in co-pending U.S. provisional application No. 62/815,405, filed Mar. 8, 2019, the entire content of which is hereby incorporated herein by reference in its entirety) for administration to a subject according to any one of the aspects of the present invention.

In another embodiment, the formulation comprises a therapeutic amount of an anti-IL-36R antibody (disclosed herein) and
i) a pharmaceutically acceptable buffer; or
ii) a pharmaceutically acceptable tonicifying agent; or
iii) a pharmaceutically acceptable stabilizing agent; or
iv) a pharmaceutically acceptable salt; or
v) a pharmaceutically acceptable surfactant; or
vi) a pharmaceutically acceptable buffer and a pharmaceutically acceptable tonicifying agent; or
vii) a pharmaceutically acceptable buffer, a pharmaceutically acceptable tonicifying agent and a pharmaceutically acceptable stabilizing agent; or
viii) a pharmaceutically acceptable buffer, a pharmaceutically acceptable tonicifying agent, a pharmaceutically acceptable stabilizing agent and a pharmaceutically acceptable salt; or
ix) a pharmaceutically acceptable buffer, a pharmaceutically acceptable tonicifying agent, a pharmaceutically acceptable stabilizing agent, a pharmaceutically acceptable salt and a pharmaceutically acceptable surfactant;
   each in pharmaceutically acceptable quantities and at a pharmaceutically acceptable pH.

In another embodiment, the anti-IL-36R antibody or antigen binding fragment thereof is present in the formulation at a concentration of about 15 mg/mL, about 20 mg/mL, about 25 mg/mL, about 30 mg/mL, about 60 mg/mL, about 75 mg/mL, about 80 mg/mL, about 100 mg/mL or about 150 mg/mL. In another related embodiment, the pharmaceutically acceptable buffer is present in the formulation at a concentration within the range from about 20 mM to about 80 mM, or at a concentration of about 20 mM, about 25 mM, about 35 mM, about 40 mM, about 45 mM, about 50 mM, about 60 mM. In another related embodiment, the pharmaceutically acceptable tonicifying agent is present in the formulation at a concentration within the range from about 100 mM to about 250 mM, or at a concentration of about 100 mM, about 120 mM, about 150 mM, about 180 mM, about 200 mM. In another related embodiment, the pharmaceutically acceptable stabilizing agent is present in the formulation at a concentration within the range from about 0 mM to about 80 mM, or at a concentration of about 25 mM or about 50 mM. In another related embodiment, the pharmaceutically acceptable salt is present in the formulation at a concentration of within the range from about 0 to about 150 mM, or at a concentration of about 3 mM, 5 mM, 10 mM, 25 mM or 50 mM. In another related embodiment, the pharmaceutically acceptable surfactant is present in the formulation at a concentration within the range from about 0 g/L to about 1.5 g/L, or at a concentration of about 0.1 g/L, 0.2 g/L, 0.4 g/L, 0.5 g/L or 1 g/L. In an embodiment related to any of the aspects and embodiments described herein, the formulation is characterized by a pH within the range from about 5 to about 8. In another related embodiment, the pH is about 5, about 5.5, about 6, about 6.5, about 7, about 7.5 or about 8.

In another embodiment, the buffer comprises histidine, phosphate, succinate, citrate, acetate or TRIS; the tonicifying agent is one or more sugar and/or polyol including sucrose, trehalose, sorbitol, magnesium sulfate (MgSO4), glycerol, mannitol or dextrose; the stabilizer comprises an amino acid including arginine, histidine, glycine, cysteine, proline, methionine, lysine, aspartate, glutamate or pharmaceutically acceptable salts thereof; the salt comprises sodium chloride (NaCl), magnesium chloride (MgCl2), potassium chloride (KCl), lithium chloride (LiCl), calcium chloride (CaCl2)), boric acid salts or zinc chloride (ZnCl2); and the surfactant comprises poloxamer 188, polysorbate 20, polysorbate 40, polysorbate 60 or polysorbate 80.

In one embodiment, the method of treatment according to any of the aspects described herein, includes administering to the mammal or patient a therapeutic amount of a stable pharmaceutical formulation comprising from about 20 mg/mL to about 150 mg/mL of an anti-IL-36R antibody, about 20 mM to about 80 mM of a pharmaceutically acceptable buffer (e.g., acetate buffer), about 100 mM to about 250 mM of a pharmaceutically acceptable tonicifying agent (e.g., sucrose), about 0 mM to about 80 mM of a pharmaceutically acceptable stabilizing agent (e.g., arginine) or a pharmaceutically acceptable salt thereof, about 0 to about 150 mM of a pharmaceutically acceptable salt (e.g., sodium chloride), and a pharmaceutically acceptable surfactant (e.g., polysorbate 20) in an amount about 0 g/L to about 1.5 g/L, wherein the palmoplantar pustulosis (PPP) in the subject is treated, prevented or ameliorated, wherein the moderate to severe PPP in the subject treated, wherein the signs and symptoms of an acute phase flare-up of PPP in the subject is reduced or alleviated, wherein the severity and duration of PPP flares in the subject is reduced, wherein the skin disorder associated with acute PPP (including new appearance or worsening of pustules) in the subject is treated, wherein the recurrence of PPP flares in the subject is reduced or prevented, wherein the PPP ASI 50 at week 16 in the subject is achieved, wherein the complete resolution of PPP symptoms in the subject is achieved. In a related embodiment, the stable pharmaceutical formulation is an aqueous pharmaceutical formulation. In a related embodiment, the pH of the aqueous pharmaceutical formulation is about 5 to about 7. In a related embodiment, the pharmaceutical formulation is for an intravenous administration to the mammal or patient. In a related embodiment, the pharmaceutical formulation is for a subcutaneous administration to the mammal or patient. In a related embodiment, the pharmaceutical formulation for the intravenous administration comprises an anti-IL-36R antibody in an amount of about 60 mg/mL. In a related embodiment, the pharmaceutical formulation for a subcutaneous administration comprises an anti-IL-36R antibody in an amount of about 150 mg/mL. In a related embodiment, the anti-IL-36R antibody comprising: (i) a light chain including an amino acid sequence set forth as SEQ ID NO:118 and a heavy chain including an amino acid sequence set forth as SEQ ID NO:125; or (ii) a light chain including an amino acid sequence set forth as SEQ ID NO:118 and a heavy chain including an amino acid sequence set forth as SEQ ID NO:126; or (iii) a light chain including an amino acid sequence set forth as SEQ ID NO:118 and a heavy chain including an amino acid sequence set forth as SEQ ID NO:127. In a related embodiment, the anti-IL-36R antibody comprising: a light chain variable region comprising the amino acid sequence of SEQ ID NO: 77; and a heavy chain variable region comprising the amino acid sequence of SEQ ID NO: 87; or a light chain variable region comprising the amino acid sequence of SEQ ID NO: 77; and a heavy chain variable region comprising the amino acid sequence of SEQ ID NO: 88; or a light chain variable region comprising the amino acid sequence of SEQ ID NO: 77; and a heavy chain variable region comprising the amino acid sequence of SEQ ID NO: 89; or a light chain variable region comprising the amino acid sequence of SEQ ID NO: 80; and a heavy chain variable region comprising the amino acid sequence of SEQ ID NO: 87; or a light chain variable region comprising the amino acid sequence of SEQ ID NO: 80; and a heavy chain variable region comprising the amino acid sequence of SEQ ID NO: 88; or a light chain variable region comprising the amino acid sequence of SEQ ID NO: 80; and a heavy chain variable region comprising the amino acid sequence of SEQ ID NO: 89.

In one embodiment, the method of treatment according to any of the preceding aspects, comprises administering to the mammal or patient a therapeutic amount of a stable pharmaceutical formulation selected from the group consisting of:
I. formulation including about 20 mg/mL to about 150 mg/mL of the anti-IL-36R antibody, about 40 mM histidine, about 120 mM sucrose, about 50 mM L-Arginine, about 5 mM NaCl and about 1.0 g/L Polysorbate 20, with a pH of about 6.0;
II. formulation including about 20 mg/mL to about 150 mg/mL of the anti-IL-36R antibody, about 45 mM acetate, about 150 mM sucrose, about 25 mM L-Arginine, about 0.4 g/L Polysorbate 20, with a pH of about 5.5;
III. formulation including about 20 mg/mL to about 150 mg/mL of the anti-IL-36R antibody, about 45 mM acetate, about 180 mM sucrose, about 25 mM Glycine, about 0.4 g/L Polysorbate 80, with a pH of about 5.5;
IV. formulation including about 20 mg/mL to about 150 mg/mL of the anti-IL-36R antibody, about 25 mM citrate, about 150 mM trehalose, about 25 mM methionine, about 0.2 g/L Polysorbate 20, with a pH of about 6.0;
V. formulation including about 20 mg/mL to about 150 mg/mL of the anti-IL-36R antibody, about 25 mM histidine, about 180 mM sucrose, about 20 mM mannitol, about 0.2 g/L Polysorbate 20, with a pH of about 6.5;
VI. formulation including about 20 mg/mL to about 150 mg/mL of the anti-IL-36R antibody, about 25 mM citrate, about 200 mM sucrose, about 0.4 g/L Polysorbate 80, with a pH of about 6.5;
VII. formulation including about 20 mg/mL to about 150 mg/mL of the anti-IL-36R antibody, about 45 mM acetate, about 150 mM sucrose, about 25 mM L-Arginine, about 0.4 g/L Polysorbate 20, with a pH of about 5.5;
VIII. formulation including about 20 mg/mL to about 150 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0;
IX. formulation including about 20 mg/mL to about 150 mg/mL of the anti-IL-36R antibody, about 25 mM acetate, about 100 mM mannitol, about 50 mM NaCl, about 0.2 g/L Polysorbate 20, with a pH of about 5.5;
X. formulation including about 20 mg/mL to about 150 mg/mL of the anti-IL-36R antibody, about 20 mM succinate, about 220 mM sucrose, about 0.1 g/L Polysorbate 80, with a pH of about 6.0; and
XI. formulation including about 20 mg/mL to about 150 mg/mL of the anti-IL-36R antibody, about 25 mM citrate, about 0.4 g/L Polysorbate 20, with a pH of about 6.5,
   wherein the palmoplantar pustulosis (PPP) in the subject is treated, prevented or ameliorated, wherein the moderate to severe PPP in the subject treated, wherein the signs and symptoms of an acute phase flare-up of PPP in the subject is reduced or alleviated, wherein the severity and duration of PPP flares in the subject is reduced, wherein the skin disorder associated with acute PPP (including new appearance or worsening of pustules) in the subject is treated, wherein the recurrence of PPP flares in the subject is reduced or prevented, wherein the PPP ASI 50 at week 16 in the subject is achieved, wherein the complete resolution of PPP symptoms in the subject is achieved. In a related embodiment, the stable pharmaceutical formulation is an aqueous pharmaceutical formulation. In a related embodiment, the pharmaceutical formulation is for an intravenous administration to the mammal or patient. In a related embodiment, the pharmaceutical formulation is for a subcutaneous administration to the mammal or patient. In a related embodiment, the pharmaceutical formulation for an intravenous administration comprises an anti-IL-36R antibody in an amount of about 60 mg/mL. In a related embodiment, the pharmaceutical formulation for a subcutaneous administration comprises an anti-IL-36R antibody in an amount of about 150 mg/mL. In a related embodiment, the anti-IL-36R antibody comprising: (i) a light chain including an amino acid sequence set forth as SEQ ID NO:118 and a heavy chain including an amino acid sequence set forth as SEQ ID NO:125; or (ii) a light chain including an amino acid sequence set forth as SEQ ID NO:118 and a heavy chain including an amino acid sequence set forth as SEQ ID NO:126; or (iii) a light chain including an amino acid sequence set forth as SEQ ID NO:118 and a heavy chain including an amino acid sequence set forth as SEQ ID NO:127. In a related embodiment, the anti-IL-36R antibody comprising: a light chain variable region comprising the amino acid sequence of SEQ ID NO: 77; and a heavy chain variable region comprising the amino acid sequence of SEQ ID NO: 87; or a light chain variable region comprising the amino acid sequence of SEQ ID NO: 77; and a heavy chain variable region comprising the amino acid sequence of SEQ ID NO: 88; or a light chain variable region comprising the amino acid sequence of SEQ ID NO: 77; and a heavy chain variable region comprising the amino acid sequence of SEQ ID NO: 89; or a light chain variable region comprising the amino acid sequence of SEQ ID NO: 80; and a heavy chain variable region comprising the amino acid sequence of SEQ ID NO: 87; or a light chain variable region comprising the amino acid sequence of SEQ ID NO: 80; and a heavy chain variable region comprising the amino acid sequence of SEQ ID NO: 88; or a light chain variable region comprising the amino acid sequence of SEQ ID NO: 80; and a heavy chain variable region comprising the amino acid sequence of SEQ ID NO: 89.

In one embodiment, the method of treatment according to any of the preceding aspects, comprises administering to the mammal or patient a therapeutic amount of a stable pharmaceutical formulation selected from the group consisting of:
I. formulation including about 20 mg/mL of the anti-IL-36R antibody, about 40 mM histidine, about 120 mM sucrose, about 50 mM L-Arginine, about 5 mM NaCl and about 1.0 g/L Polysorbate 20, with a pH of about 6.0;
II. formulation including about 60 mg/mL of the anti-IL-36R antibody, about 45 mM acetate, about 150 mM sucrose, about 25 mM L-Arginine, about 0.4 g/L Polysorbate 20, with a pH of about 5.5;
III. formulation including about 20 mg/mL of the anti-IL-36R antibody, about 45 mM acetate, about 180 mM sucrose, about 25 mM Glycine, about 0.4 g/L Polysorbate 80, with a pH of about 5.5;
IV. formulation including about 150 mg/mL of the anti-IL-36R antibody, about 25 mM citrate, about 150 mM trehalose, about 25 mM methionine, about 0.2 g/L Polysorbate 20, with a pH of about 6.0;
V. formulation including about 150 mg/mL of the anti-IL-36R antibody, about 25 mM histidine, about 180 mM sucrose, about 20 mM mannitol, about 0.2 g/L Polysorbate 20, with a pH of about 6.5;
VI. formulation including about 20 mg/mL of the anti-IL-36R antibody, about 25 mM citrate, about 200 mM sucrose, about 0.4 g/L Polysorbate 80, with a pH of about 6.5;
VII. formulation including about 150 mg/mL of the anti-IL-36R antibody, about 45 mM acetate, about 150 mM sucrose, about 25 mM L-Arginine, about 0.4 g/L Polysorbate 20, with a pH of about 5.5;
VIII. formulation including about 15 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0;
IX. formulation including about 80 mg/mL of the anti-IL-36R antibody, about 25 mM acetate, about 100 mM mannitol, about 50 mM NaCl, about 0.2 g/L Polysorbate 20, with a pH of about 5.5;
X. formulation including about 100 mg/mL of the anti-IL-36R antibody, about 20 mM succinate, about 220 mM sucrose, about 0.1 g/L Polysorbate 80, with a pH of about 6.0; and
XI. formulation including about 60 mg/mL of the anti-IL-36R antibody, about 25 mM citrate, about 0.4 g/L Polysorbate 20, with a pH of about 6.5,
   wherein the palmoplantar pustulosis (PPP) in the subject is treated, prevented or ameliorated, wherein the moderate to severe PPP in the subject treated, wherein the signs and symptoms of an acute phase flare-up of PPP in the subject is reduced or alleviated, wherein the severity and duration of PPP flares in the subject is reduced, wherein the skin disorder associated with acute PPP (including new appearance or worsening of pustules) in the subject is treated, wherein the recurrence of PPP flares in the subject is reduced or prevented, wherein the PPP ASI 50 at week 16 in the subject is achieved, wherein the complete resolution of PPP symptoms in the subject is achieved. In a related embodiment, the stable pharmaceutical formulation is an aqueous pharmaceutical formulation. In a related embodiment, the pharmaceutical formulation is for an intravenous administration to the mammal or patient. In a related embodiment, the pharmaceutical formulation is for a subcutaneous administration to the mammal or patient. In a related embodiment, the pharmaceutical formulation for an intravenous administration comprises an anti-IL-36R antibody in an amount of about 60 mg/mL. In a related embodiment, the pharmaceutical formulation for a subcutaneous administration comprises an anti-IL-36R antibody in an amount of about 150 mg/mL. In a related embodiment, the anti-IL-36R antibody comprising: (i) a light chain including an amino acid sequence set forth as SEQ ID NO:118 and a heavy chain including an amino acid sequence set forth as SEQ ID NO:125; or (ii) a light chain including an amino acid sequence set forth as SEQ ID NO:118 and a heavy chain including an amino acid sequence set forth as SEQ ID NO:126; or (iii) a light chain including an amino acid sequence set forth as SEQ ID NO:118 and a heavy chain including an amino acid sequence set forth as SEQ ID NO:127. In a related embodiment, the anti-IL-36R antibody comprising: a light chain variable region comprising the amino acid sequence of SEQ ID NO: 77; and a heavy chain variable region comprising the amino acid sequence of SEQ ID NO: 87; or a light chain variable region comprising the amino acid sequence of SEQ ID NO: 77; and a heavy chain variable region comprising the amino acid sequence of SEQ ID NO: 88; or a light chain variable region comprising the amino acid sequence of SEQ ID NO: 77; and a heavy chain variable region comprising the amino acid sequence of SEQ ID NO: 89; or a light chain variable region comprising the amino acid sequence of SEQ ID NO: 80; and a heavy chain variable region comprising the amino acid sequence of SEQ ID NO: 87; or a light chain variable region comprising the amino acid sequence of SEQ ID NO: 80; and a heavy chain variable region comprising the amino acid sequence of SEQ ID NO: 88; or a light chain variable region comprising the amino acid sequence of SEQ ID NO: 80; and a heavy chain variable region comprising the amino acid sequence of SEQ ID NO: 89.

In one embodiment, the present invention relates to a method of treating a patient with severe or moderate to severe PPP, said method including administering or having administered to the patient a therapeutically effective amount of an anti-IL-36R antibody of the present invention subcutaneously or intravenously or by both routes simultaneously or sequentially and in any order. In a related embodiment, the subcutaneous administration comprises administration of 300 mg or 600 mg dose or 900 mg of the anti-IL-36R antibody. In a related embodiment, the intravenous administration comprises administering 300 mg, 600 mg, 900 mg or 1200 mg dose of the anti-IL-36R antibody. In a related embodiment, the subcutaneous administration is conducted at qw (once every week), q2w (once every 2 weeks), q4w (once every 4 weeks), q6w (once every 6 weeks) or q8w (once every 8 weeks) interval, or a combination thereof. In a related embodiment, the intravenous administration is conducted at q4w (once every 4 weeks), q8w (once every 8 weeks) or q12w (once every 12 weeks) interval, or a combination thereof.

In another embodiment relating to any of the aspects and embodiments described herein, the anti-IL-36R antibody is administered subcutaneously or intravenously or by both routes simultaneously or sequentially and in any order. In a related embodiment, the subcutaneous administration comprises an initial dose. In a related embodiment, the subcutaneous administration further comprises a subsequent dose. In a related embodiment, the administration of the anti-IL-36R antibody includes an initial dose and a subsequent dose. In a related embodiment, the initial dose is administered intravenously or subcutaneously. In a related embodiment, the subsequent dose is administered subcutaneously. In a related embodiment, the initial dose is 150 mg, 300 mg or 600 mg or 900 mg. In a related embodiment, the initial dose of 150 mg or 300 mg is administered per day (in consecutive days) for two weeks. In a related embodiment, the initial dose of 600 mg or 900 mg is administered once per week for two weeks including weeks 0 and 1; weeks 0 and 2; weeks 0 and 3; or weeks 0 and 4. In a related embodiment, the initial dose of 600 mg or 900 mg is administered once per week for three weeks including weeks 0, 1 and 2; weeks 0, 1 and 3; weeks 0, 1 and 4; weeks 0, 2 and 3; weeks 0, 2 and 4; or weeks 0, 3 and 4. In a related embodiment, the initial dose of 600 mg or 900 mg is administered once per week for four weeks including weeks 0, 1, 2 and 3; weeks 0, 1, 2 and 4; weeks 0, 1, 3 and 4; or weeks 0, 2, 3 and 4. In a related embodiment, the initial dose of 600 mg or 900 mg is administered twice per week for 2 weeks. In a related embodiment, the initial dose of 600 mg or 900 mg is administered twice per week for 3 weeks. In a related embodiment, the initial dose of 600 mg or 900 mg is administered twice per week for 4 weeks. In a related embodiment, the initial dose is 3000 mg (administered in 600 mg doses at, for example, day 1, week 1, week 2, week 3 and week 4). In a related embodiment, the initial dose is 1500 mg (administered in 300 mg doses at, for example, day 1, week 1, week 2, week 3 and week 4). In a related embodiment, the initial dose is 900 mg or 1200 mg administered IV (intravenously) or SC (subcutaneously) at q4w, q8w or q12w. In a related embodiment, the subsequent dose is 300 mg or 600 mg administered SC. In a related embodiment, the subsequent dose administration begins two to four weeks after the initial dose administration ends. In a related embodiment, the subsequent dose of 300 mg or 600 mg is administered q2w (once every 2 weeks), q4w (once every 4 weeks), q6w (once every 6 weeks) or q8w (once every 8 weeks). In a related embodiment, the subsequent dose is 600 mg administered q4w. In a related embodiment, the subsequent dose is 300 mg administered q4w. In a related embodiment, the subsequent dose is 300 mg administered q4w for eight weeks and q8w thereafter.

In a related embodiment, the method achieves a PPP Physicians Global Assessment (PPP PGA) score of 0 or 1=clear/almost clear at week 16 in the patient. In a related embodiment, the method achieves PPP ASI50 at week 16 in the patient. In a related embodiment, the method reduces the pustule severity in the patient. In a related embodiment, the method is superior over guselkumab in treating the patient. In a related embodiment, the method achieves PPP ASI75 at week 16 in the patient. In a related embodiment, the method is at least 40% superior to placebo in achievement of PPP ASI50 at week 16 in the patient.

In one embodiment, the present invention relates to a method of achieving a PPP Physicians Global Assessment (PPP PGA) score of 0 or 1=clear/almost clear at week 16 in a PPP patient, said method(s) including administering or having administered to the PPP patient a therapeutically effective amount of an anti-IL-36R antibody of the present invention subcutaneously or intravenously or intravenously or by both routes simultaneously or sequentially and in any order. In a related embodiment, the subcutaneous administration comprises an initial dose. In a related embodiment, the subcutaneous administration further comprises a subsequent dose. In a related embodiment, the administration of the anti-IL-36R antibody includes an initial dose and a subsequent dose. In a related embodiment, the initial dose is administered intravenously or subcutaneously. In a related embodiment, the subsequent dose is administered subcutaneously. In a related embodiment, the initial dose is 150 mg, 300 mg or 600 mg or 900 mg. In a related embodiment, the initial dose of 150 mg or 300 mg is administered per day (in consecutive days) for two weeks. In a related embodiment, the initial dose of 600 mg or 900 mg is administered once per week for two weeks including weeks 0 and 1; weeks 0 and 2; weeks 0 and 3; or weeks 0 and 4. In a related embodiment, the initial dose of 600 mg or 900 mg is administered once per week for three weeks including weeks 0, 1 and 2; weeks 0, 1 and 3; weeks 0, 1 and 4; weeks 0, 2 and 3; weeks 0, 2 and 4; or weeks 0, 3 and 4. In a related embodiment, the initial dose of 600 mg or 900 mg is administered once per week for four weeks including weeks 0, 1, 2 and 3; weeks 0, 1, 2 and 4; weeks 0, 1, 3 and 4; or weeks 0, 2, 3 and 4. In a related embodiment, the initial dose of 600 mg or 900 mg is administered twice per week for 2 weeks. In a related embodiment, the initial dose of 600 mg or 900 mg is administered twice per week for 3 weeks. In a related embodiment, the initial dose of 600 mg or 900 mg is administered twice per week for 4 weeks. In a related embodiment, the initial dose is 3000 mg (administered in 600 mg doses at, for example, day 1, week 1, week 2, week 3 and week 4). In a related embodiment, the initial dose is 1500 mg (administered in 300 mg doses at, for example, day 1, week 1, week 2, week 3 and week 4). In a related embodiment, the initial dose is 900 mg or 1200 mg administered IV (intravenously) or SC (subcutaneously) at q4w, q8w or q12w. In a related embodiment, the subsequent dose is 300 mg or 600 mg administered SC. In a related embodiment, the subsequent dose administration begins two to four weeks after the initial dose administration ends. In a related embodiment, the subsequent dose of 300 mg or 600 mg is administered q2w (once every 2 weeks), q4w (once every 4 weeks), q6w (once every 6 weeks) or q8w (once every 8 weeks). In a related embodiment, the subsequent dose is 600 mg administered q4w. In a related embodiment, the subsequent dose is 300 mg administered q4w. In a related embodiment, the subsequent dose is 300 mg administered q4w for eight weeks and q8w thereafter. In one embodiment related to any of the aspects and their embodiment(s) described herein, at least 10%, 20%, 30%, 40%, 50%, 60%, 70% or 80% of the patients remain in clinical remission as measured by a PPP PGA score of 0 or 1 at Week 16 24, 36, 48, 60 or 72 of the treatment.

In one embodiment related to any of the aspects and their embodiment(s) described herein, at least 10%, 20%, 30%, 40%, 50%, 60%, 70% or 80% of the patients remain in clinical remission as measured by a change in PPP ASI50 from baseline at Week 16, 24, 36, 48, 60 or 72 of the treatment.

In one embodiment related to any of the aspects and their embodiment(s) described herein, at least 10%, 20%, 30%, 40%, 50%, 60%, 70% or 80% of the patients remain in clinical remission as measured by a change in PPP ASI50, PPP PGA score of 0 or 1, PPP ASI75 16, 24, 36, 48, 60 or 72 of the treatment. In a related embodiment, proportion of patients with a response to the administration is statistically significantly higher as compared to patients on placebo for any of the end points recited.

In an embodiment related to any of the aspects or their embodiments described herein, at least 10%, 20%, 30%, 40%, 50%, 60%, 70% or 80% of the patients remain in clinical remission as measured by change from baseline in Pain Visual Analog Scale (VAS) score at Week 16, 24, 36, 48, 60 or 72 of the treatment. In an embodiment related to any of the aspects and embodiments described herein, at least 10%, 20%, 30%, 40%, 50%, 60%, 70% or 80% of the patients remain in clinical remission as measured by clinical Improvement assessed via Dermatology Life Quality Index (DLQI) at Week 16, 24, 36, 48, 60 or 72 of the treatment. In an embodiment related to any of the aspects and embodiments described herein, at least 10%, 20%, 30%, 40%, 50%, 60%, 70% or 80% of the patients remain in clinical remission as measured by time (days) to achieving PPP ASI50 or time (days) to loss of PPP ASI50 at Week 16, 24, 36, 48, 60 or 72 of the treatment. In a related embodiment, proportion of patients with a response to the administration is statistically significantly higher as compared to patients on placebo for any of the end points recited.

In an embodiment related to any of the aspects or their embodiments described herein, at least 10%, 20%, 30%, 40%, 50%, 60%, 70% or 80% of the patients remain in clinical remission as measured by a PPP ASI50 at Week 16, 24, 36, 48, 60 or 72 of the treatment. In a related embodiment, the improved effects are maintained at higher percentage with an anti-IL-36R antibody of the present invention than with placebo. In a related embodiment, at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the mammals or patients maintain improved effects at Week 16, 24, 36, 48, 60 or 72 of the treatment with an anti-IL-36R antibody of the present invention than with placebo.

In an embodiment related to any of the aspects or their embodiments described herein, at least 10%, 20%, 30%, 40%, 50%, 60%, 70% or 80% of the patients remain in clinical remission as measured by a change in PPP PGA score of 0 or 1 from baseline at Week 16, 24, 36, 48, 60 or 72 of the treatment. In a related embodiment, the improved effects are maintained at higher percentage with an anti-IL-36R antibody of the present invention than with placebo. In a related embodiment, at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the mammals or patients maintain improved effects at Week 16, 24, 36, 48, 60 or 72 of the treatment with an anti-IL-36R antibody of the present invention than with placebo.

In an embodiment related to any of the aspects or their embodiments described herein, at least 10%, 20%, 30%, 40%, 50%, 60%, 70% or 80% of the patients remain in clinical remission as measured by a reduction in the number of patients with drug-related Adverse Events (AEs) at Week 16, 24, 36, 48, 60 or 72 of the treatment. In a related embodiment, the improved effects are maintained at higher percentage with an anti-IL-36R antibody of the present invention than with placebo. In a related embodiment, at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the mammals or patients maintain improved effects at Week 16, 24, 36, 48, 60 or 72 of the treatment with an anti-IL-36R antibody of the present invention than with placebo.

In an embodiment relating to any of the above aspects, the anti-IL36R antibody is an anti-IL-36R antibody of the present invention. In one embodiment, the anti-IL36R antibody is disclosed in U.S. Pat. No. 9,023,995 or WO2013/074569. In an embodiment relating to any of the above aspects, the improved effects (including the remission or improved symptoms) last for 4, 5, 6, 7, 8, 9, 10, 11, 12, 13, 14, 15, 16, 17, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks following the administration of an anti-IL-36R antibody of the present invention at the dose regimens provided.

### Pharmaceutical Compositions and Administration Thereof

The antibodies of the present invention can be administered either alone or in combination with other agents. Examples of antibodies for use in such pharmaceutical compositions are those that comprise an antibody or antibody fragment having the light chain variable region amino acid sequence of any of SEQ ID NO: 1-10. Examples of antibodies for use in such pharmaceutical compositions are also those that comprise a humanized antibody or antibody fragment having the heavy chain variable region amino acid sequence of any of SEQ ID NO: 11-20.

Further examples of antibodies for use in such pharmaceutical compositions are also those that comprise a humanized antibody or antibody fragment having the light chain variable region amino acid sequence of any of SEQ ID NO:76-86. Preferred antibodies for use in such pharmaceutical compositions are also those that comprise a humanized antibody or antibody fragment having the heavy chain variable region amino acid sequence of any of SEQ ID NO:87-101.

Further examples of antibodies for use in such pharmaceutical compositions are also those that comprise a humanized antibody or antibody fragment having the light chain variable region and heavy chain variable region of any of SEQ ID NO: 77 and 89, SEQ ID NO: 80 and 88, SEQ ID NO: 80 and 89, SEQ ID NO: 77 and 87, SEQ ID NO: 77 and 88, SEQ ID NO: 80 and 87, SEQ ID NO: 86 and 100, SEQ ID NO: 85 and 101, or SEQ ID NO: 85 and 10.

Further examples of antibodies for use in such pharmaceutical compositions are also those that comprise a humanized antibody having the light chain region amino acid sequence of any of SEQ ID NO:115, 118, 123 or 124. Preferred antibodies for use in such pharmaceutical compositions are also those that comprise humanized antibody having the heavy chain variable region amino acid sequence of any of SEQ ID NO:125, 126, 127, 138 or 139.

Further examples of antibodies for use in such pharmaceutical compositions are also those that comprise Antibody B1, Antibody B2, Antibody B3, Antibody B4, Antibody B5, Antibody B6, Antibody C1, Antibody C2 or Antibody C3.

Various delivery systems are known and can be used to administer the IL-36R binding agent. Methods of introduction include but are not limited to intradermal, intramuscular, intraperitoneal, intravenous, subcutaneous, intranasal, epidural, and oral routes. The IL-36R binding agent can be administered, for example by infusion, bolus or injection, and can be administered together with other biologically active agents such as chemotherapeutic agents. Administration can be systemic or local. In preferred embodiments, the administration is by subcutaneous injection. Formulations for such injections may be prepared in for example prefilled syringes that may be administered once every other week.

In one aspect, the invention provides an article of manufacture comprising a subcutaneous administration device, which delivers to a patient a fixed dose of an antibody of the present invention. In some embodiments, the subcutaneous administration device is a pre-filled syringe, an autoinjector, or a large volume infusion device. For example, MyDose™ product from Roche, a single use infusion device that enables the subcutaneous administration of large quantities of liquid medication, may be used as the administration device. Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park Ill.), YPSOMATE™, YPSOMATE 2.25™, VAIROJECT™ (Ypsomed AG, Burgdorf, Switzerland) to name only a few. Additional information relating to example delivery devices that could be used with an antibody of the present invention may be found, for example, in CH705992A2, WO2009/040602, WO2016/169748, WO2016/179713.

In specific embodiments, the IL-36R binding agent composition is administered by injection, by means of a catheter, by means of a suppository, or by means of an implant, the implant being of a porous, non-porous, or gelatinous material, including a membrane, such as a sialastic membrane, or a fiber. Typically, when administering the composition, materials to which the anti-IL-36R antibody or agent does not absorb are used.

In other embodiments, the anti-IL-36R antibody or agent is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

An IL-36R binding agent (e.g., an anti-IL-36R antibody) can be administered as pharmaceutical compositions comprising a therapeutically effective amount of the binding agent and one or more pharmaceutically compatible ingredients.

In one embodiment, the anti-IL-36R antibody or an antigen binding fragment thereof (disclosed herein) is present in a pharmaceutical formulation (as described in co-pending U.S. provisional application No. 62/815,405, filed Mar. 8, 2019, the entire content of which is hereby incorporated herein by reference in its entirety) suitable for administration to a mammal or patient according to any one of the aspects described herein. Various examples to this embodiment are described as numbered clauses (1, 2, 3, etc.) below for convenience. These are provided as examples and do not limit the subject technology. It is noted that any of the dependent clauses may be combined in any combination, and placed into a respective independent clause, e.g., clause 1. The other clauses can be presented in a similar manner.
1. A pharmaceutical formulation including:
   a. An anti-IL-36R antibody or an antigen binding fragment thereof, as disclosed herein, present at a concentration within the range from about 0.5 mg/mL to about 220 mg/mL; and
   b. A pharmaceutically acceptable buffer present at a concentration within the range from about 20 mM to about 80 mM;
   wherein the formulation is characterized by a pH within the range from about 5 to about 8 when in aqueous form.
2. The formulation of clause 1, wherein the formulation is in liquid or powder form.
3. The formulation of clause 1, wherein the anti-IL-36R antibody is present at a concentration of within the range from about 10 mg/mL to about 200 mg/mL.
4. The formulation of clause 1, wherein the anti-IL-36R antibody is present at a concentration of about 20 mg/mL.
5. The formulation of clause 1, wherein the anti-IL-36R antibody is present at a concentration of about 60 mg/mL.
6. The formulation of clause 1, wherein the anti-IL-36R antibody is present at a concentration of about 150 mg/mL.
7. The formulation of clause 1, wherein the buffer comprises histidine, phosphate, succinate, citrate, acetate or TRIS.
8. The formulation of clause 1, wherein the buffer comprises citrate or acetate.
9. The formulation of clause 1, wherein the buffer comprises histidine.
10. The formulation of clause 1, wherein the buffer comprises acetate.
11. The formulation of clause 1, wherein the formulation further comprises a pharmaceutically acceptable tonicifying agent present at a concentration within the range from about 100 mM to about 250 mM.
12. The formulation of clause 11, wherein the tonicifying agent is one or more sugar and/or polyol.
13. The formulation of clause 11, wherein the tonicifying agent is one or more sugar and/or polyol including sucrose, trehalose, sorbitol, magnesium sulfate (MgSO₄), glycerol, mannitol or dextrose.
14. The formulation of clause 11, wherein the tonicifying agent comprises sucrose or trehalose.
15. The formulation of clause 11, wherein the tonicifying agent comprises sucrose.
16. The formulation of clause 11, wherein the tonicifying agent comprises trehalose.
17. The formulation of clause 1, wherein the formulation further comprises a pharmaceutically acceptable stabilizer present at a concentration within the range from about 0 mM to about 80 mM.
18. The formulation of clause 17, wherein the stabilizer comprises an amino acid including arginine, histidine, glycine, cysteine, proline, methionine, lysine, aspartate, glutamate or pharmaceutically acceptable salts thereof.
19. The formulation of clause 17, wherein the stabilizer comprises L-arginine or pharmaceutically acceptable salts thereof.
20. The formulation of clause 1, wherein the formulation further comprises a pharmaceutically acceptable salt present at a concentration of within the range from about 0 to about 150 mM.
21. The formulation of clause 20, wherein the salt comprises sodium chloride (NaCl), magnesium chloride (MgCl₂), potassium chloride (KCl), lithium chloride (LiCl), calcium chloride (CaCl₂)), boric acid salts or zinc chloride (ZnCl₂).
22. The formulation of clause 20, wherein the salt comprises sodium chloride (NaCl).
23. The formulation of clause 1, wherein the formulation further comprises a pharmaceutically acceptable surfactant present at a concentration within the range from about 0 g/L to about 1.5 g/L.
24. The formulation of clause 23, wherein the surfactant comprises poloxamer 188, polysorbate 20, polysorbate 40, polysorbate 60 or polysorbate 80.
25. The formulation of clause 23, wherein the surfactant comprises polysorbate 20, polysorbate 40, polysorbate 60 or polysorbate 80.
26. The formulation of clause 23, wherein the surfactant comprises polysorbate 20.
27. The formulation of clause 23, wherein the surfactant comprises polysorbate 80.
28. A pharmaceutical formulation including:
   a. an anti-IL-36R antibody or an antigen binding fragment thereof, as disclosed herein, present at a concentration within the range from about 10 mg/mL to about 200 mg/mL;
   b. an acetate and/or histidine buffer present at a concentration within the range from about 20 mM to about 80 mM;
   c. sucrose and-/-or trehalose present at a concentration within the range from about 100 mM to about 250 mM;
   d. L-arginine and-/-or pharmaceutically acceptable salts thereof present at a concentration within the range from about 0 mM to about 80 mM;
   e. sodium chloride (NaCl) present at a concentration of within the range from about 0 to about 150 mM; and
   f. polysorbate 20 and/or polysorbate 80 present at a concentration within the range from about 0 g/L to about 1.5 g/L;
   wherein the formulation is characterized by a pH within the range from about 5 to about 7 when in aqueous form.
29. A pharmaceutical formulation including:
   a. an anti-IL-36R antibody or an antigen binding fragment thereof, as disclosed herein, present at a concentration of about 20 mg/mL;
   b. an citrate buffer present at a concentration at a concentration of about 25 mM;
   c. sucrose and/or trehalose present at a concentration of about 200 mM;
   d. polysorbate 80 present at a concentration of about 0.4 g/L;
   wherein the formulation is characterized by a pH within the range from about 6 to about 7 when in aqueous form.
30. A pharmaceutical formulation including:
   a. an anti-IL-36R antibody or an antigen binding fragment thereof, as disclosed herein, present at a concentration of about 60 mg/mL;
   b. an acetate buffer present at a concentration at a concentration of about 45 mM;
   c. sucrose and/or trehalose present at a concentration of about 150 mM;
   d. L-arginine or pharmaceutically acceptable salts thereof present at a concentration of about 25 mM; and
   e. polysorbate 20 present at a concentration of about 0.4 g/L;
   wherein the formulation is characterized by a pH within the range from about 5 to about 6 when in aqueous form.
31. A pharmaceutical formulation including:
   a. an anti-IL-36R antibody or an antigen binding fragment thereof, as disclosed herein, present at a concentration of about 150 mg/mL;
   b. an acetate buffer present at a concentration at a concentration of about 45 mM;
   c. sucrose or trehalose present at a concentration of about 150 mM;
   d. L-arginine or pharmaceutically acceptable salts thereof present at a concentration of about 25 mM; and
   e. polysorbate 20 present at a concentration of about 0.4 g/L;
   wherein the formulation is characterized by a pH within the range from about 5 to about 6 when in aqueous form.
32. The pharmaceutical formulation of any one of clauses 1-31, wherein the formulation is characterized by an osmolality within the range from about 210 mOsmol/kg to about 390 mOsm/kg.
33. The pharmaceutical formulation of any one of clauses 1-32, wherein less than about 5% of the antibody is present in an aggregate form in the formulation.
34. The pharmaceutical formulation of any one of clauses 1-33, wherein the formulation is sterile.
35. The pharmaceutical formulation of any one of clauses 1-34, wherein the formulation is stable upon freezing and thawing.
36. The pharmaceutical formulation of any of clauses 1-35, wherein the formulation comprises water or is reconstituted with water.
37. The pharmaceutical formulation of any of clauses 1-36, wherein the formulation has a pH of between about 5 to about 6 in liquid form or when reconstituted with water.
38. The pharmaceutical formulation of any of clauses 1-37, wherein the formulation has a pH of about 6 in liquid or when reconstituted with water.
39. The pharmaceutical formulation of any of clauses 1-37, wherein the formulation has at least one feature selected from the group consisting of:
   (i) Increased shelf life
   (ii) better temperature stability,
   (iii) decreased formation of aggregates,
   (iv) better chemical stability,
   (v) decreased viscosity, and as compared to a reference formulation.
40. The pharmaceutical formulation of any of clauses 1-37, wherein the formulation having at least one feature selected from the group consisting of:
   (a) decreased percentage of aggregates as measured by High Performance Size Exclusion Chromatography (HP-SEC),
   (b) higher percentage of monomers as measured by HP-SEC,
   (c) higher percentage of main peak (less degradation of charge variants) measured by CEX,
   (d) lower percentage of subvisual particles such as ≥10 μm and ≥25 μm, and
   (e) lower turbidity value in Formazin Nephelometry Units (FNU), after storage at about 40° C. as compared to the reference formulation.
41. A pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
      iii. a light chain including an amino acid sequence set forth as SEQ ID NO:118 and a heavy chain including an amino acid sequence set forth as SEQ ID NO:127;
         wherein the formulation is selected from the group consisting of:
         I. formulation including about 20 mg/mL of the anti-IL-36R antibody, about 40 mM histidine, about 120 mM sucrose, about 50 mM L-Arginine, about 5 mM NaCl and about 1.0 g/L Polysorbate 20, with a pH of about 6.0;
         II. formulation including about 60 mg/mL of the anti-IL-36R antibody, about 45 mM acetate, about 150 mM sucrose, about 25 mM L-Arginine, about 0.4 g/L Polysorbate 20, with a pH of about 5.5;
         III. formulation including about 20 mg/mL of the anti-IL-36R antibody, about 45 mM acetate, about 180 mM sucrose, about 25 mM Glycine, about 0.4 g/L Polysorbate 80, with a pH of about 5.5;
         IV. formulation including about 150 mg/mL of the anti-IL-36R antibody, about 25 mM citrate, about 150 mM trehalose, about 25 mM methionine, about 0.2 g/L Polysorbate 20, with a pH of about 6.0;
         V. formulation including about 150 mg/mL of the anti-IL-36R antibody, about 25 mM histidine, about 180 mM sucrose, about 20 mM mannitol, about 0.2 g/L Polysorbate 20, with a pH of about 6.5;
         VI. formulation including about 20 mg/mL of the anti-IL-36R antibody, about 25 mM citrate, about 200 mM sucrose, about 0.4 g/L Polysorbate 80, with a pH of about 6.5;
         VII. formulation including about 150 mg/mL of the anti-IL-36R antibody, about 45 mM acetate, about 150 mM sucrose, about 25 mM L-Arginine, about 0.4 g/L Polysorbate 20, with a pH of about 5.5;
         VIII. formulation including about 15 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0;
         IX. formulation including about 80 mg/mL of the anti-IL-36R antibody, about 25 mM acetate, about 100 mM mannitol, about 50 mM NaCl, about 0.2 g/L Polysorbate 20, with a pH of about 5.5;
         X. formulation including about 100 mg/mL of the anti-IL-36R antibody, about 20 mM succinate, about 220 mM sucrose, about 0.1 g/L Polysorbate 80, with a pH of about 6.0; and
         XI. formulation including about 60 mg/mL of the anti-IL-36R antibody, about 25 mM citrate, about 0.4 g/L Polysorbate 20, with a pH of about 6.5.
42. A pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
      iii. a light chain including an amino acid sequence set forth as SEQ ID NO:118 and a heavy chain including an amino acid sequence set forth as SEQ ID NO:127;
         wherein the formulation includes about 20 mg/mL of the anti-IL-36R antibody, about 40 mM histidine, about 120 mM sucrose, about 50 mM L-Arginine, about 5 mM NaCl and about 1.0 g/L Polysorbate 20, with a pH of about 6.0.
43. A pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
      iii. a light chain including an amino acid sequence set forth as SEQ ID NO:118 and a heavy chain including an amino acid sequence set forth as SEQ ID NO:127;
         wherein the formulation includes about 60 mg/mL of the anti-IL-36R antibody, about 45 mM acetate, about 150 mM sucrose, about 25 mM L-Arginine, about 0.4 g/L Polysorbate 20, with a pH of about 5.5.
44. A pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
      iii. a light chain including an amino acid sequence set forth as SEQ ID NO:118 and a heavy chain including an amino acid sequence set forth as SEQ ID NO:127;
         wherein the formulation includes about 20 mg/mL of the anti-IL-36R antibody, about 45 mM acetate, about 180 mM sucrose, about 25 mM Glycine, about 0.4 g/L Polysorbate 80, with a pH of about 5.5.
45. A pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
      iii. a light chain including an amino acid sequence set forth as SEQ ID NO:118 and a heavy chain including an amino acid sequence set forth as SEQ ID NO:127;
         wherein the formulation includes about 150 mg/mL of the anti-IL-36R antibody, about 25 mM citrate, about 150 mM trehalose, about 25 mM methionine, about 0.2 g/L Polysorbate 20, with a pH of about 6.0.
46. A pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
      iii. a light chain including an amino acid sequence set forth as SEQ ID NO:118 and a heavy chain including an amino acid sequence set forth as SEQ ID NO:127;
         wherein the formulation includes about 150 mg/mL of the anti-IL-36R antibody, about 25 mM histidine, about 180 mM sucrose, about 20 mM mannitol, about 0.2 g/L Polysorbate 20, with a pH of about 6.5.
47. A pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
      iii. a light chain including an amino acid sequence set forth as SEQ ID NO:118 and a heavy chain including an amino acid sequence set forth as SEQ ID NO:127;
         wherein the formulation includes about 20 mg/mL of the anti-IL-36R antibody, about 25 mM citrate, about 200 mM sucrose, about 0.4 g/L Polysorbate 80, with a pH of about 6.5.
48. A pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
      iii. a light chain including an amino acid sequence set forth as SEQ ID NO:118 and a heavy chain including an amino acid sequence set forth as SEQ ID NO:127;
         wherein the formulation includes about 150 mg/mL of the anti-IL-36R antibody, about 45 mM acetate, about 150 mM sucrose, about 25 mM L-Arginine, about 0.4 g/L Polysorbate 20, with a pH of about 5.5.
49. A pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
      iii. a light chain including an amino acid sequence set forth as SEQ ID NO:118 and a heavy chain including an amino acid sequence set forth as SEQ ID NO:127;
         wherein the formulation includes about 15 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0.
50. A pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
      iii. a light chain including an amino acid sequence set forth as SEQ ID NO:118 and a heavy chain including an amino acid sequence set forth as SEQ ID NO:127;
         wherein the formulation includes about 80 mg/mL of the anti-IL-36R antibody, about 25 mM acetate, about 100 mM mannitol, about 50 mM NaCl, about 0.2 g/L Polysorbate 20, with a pH of about 5.5.
51. A pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
      iii. a light chain including an amino acid sequence set forth as SEQ ID NO:118 and a heavy chain including an amino acid sequence set forth as SEQ ID NO:127;
         wherein the formulation includes about 100 mg/mL of the anti-IL-36R antibody, about 20 mM succinate, about 220 mM sucrose, about 0.1 g/L Polysorbate 80, with a pH of about 6.0.
52. A pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
      iii. a light chain including an amino acid sequence set forth as SEQ ID NO:118 and a heavy chain including an amino acid sequence set forth as SEQ ID NO:127;
         wherein the formulation includes about 60 mg/mL of the anti-IL-36R antibody, about 25 mM citrate, about 0.4 g/L Polysorbate 20, with a pH of about 6.5.
53. A pharmaceutical formulation including:
   an anti-IL-36R antibody or antigen-binding fragment thereof, including:
   a light chain variable region comprising the amino acid sequence of SEQ ID NO: 77; and a heavy chain variable region comprising the amino acid sequence of SEQ ID NO: 87; or
   a light chain variable region comprising the amino acid sequence of SEQ ID NO: 77; and a heavy chain variable region comprising the amino acid sequence of SEQ ID NO: 88; or
   a light chain variable region comprising the amino acid sequence of SEQ ID NO: 77; and a heavy chain variable region comprising the amino acid sequence of SEQ ID NO: 89; or
   a light chain variable region comprising the amino acid sequence of SEQ ID NO: 80; and a heavy chain variable region comprising the amino acid sequence of SEQ ID NO: 87; or
   a light chain variable region comprising the amino acid sequence of SEQ ID NO: 80; and a heavy chain variable region comprising the amino acid sequence of SEQ ID NO: 88; or
   a light chain variable region comprising the amino acid sequence of SEQ ID NO: 80; and a heavy chain variable region comprising the amino acid sequence of SEQ ID NO: 89;
   wherein the formulation includes: about 20 mg/mL of the anti-IL-36R antibody, about 40 mM histidine, about 120 mM sucrose, about 50 mM L-Arginine, about 5 mM NaCl and about 1.0 g/L Polysorbate 20, with a pH of about 6.0.
54. A pharmaceutical formulation including:
   an anti-IL-36R antibody or antigen-binding fragment thereof, including:
   a light chain variable region comprising the amino acid sequence of SEQ ID NO: 77; and a heavy chain variable region comprising the amino acid sequence of SEQ ID NO: 87; or
   a light chain variable region comprising the amino acid sequence of SEQ ID NO: 77; and a heavy chain variable region comprising the amino acid sequence of SEQ ID NO: 88; or
   a light chain variable region comprising the amino acid sequence of SEQ ID NO: 77; and a heavy chain variable region comprising the amino acid sequence of SEQ ID NO: 89; or
   a light chain variable region comprising the amino acid sequence of SEQ ID NO: 80; and a heavy chain variable region comprising the amino acid sequence of SEQ ID NO: 87; or
   a light chain variable region comprising the amino acid sequence of SEQ ID NO: 80; and a heavy chain variable region comprising the amino acid sequence of SEQ ID NO: 88; or
   a light chain variable region comprising the amino acid sequence of SEQ ID NO: 80; and a heavy chain variable region comprising the amino acid sequence of SEQ ID NO: 89;
   wherein the formulation includes: about 60 mg/mL of the anti-IL-36R antibody, about 45 mM acetate, about 150 mM sucrose, about 25 mM L-Arginine, about 0.4 g/L Polysorbate 20, with a pH of about 5.5.
55. A pharmaceutical formulation including:
   an anti-IL-36R antibody or antigen-binding fragment thereof, including:
   a light chain variable region comprising the amino acid sequence of SEQ ID NO: 77; and a heavy chain variable region comprising the amino acid sequence of SEQ ID NO: 87; or
   a light chain variable region comprising the amino acid sequence of SEQ ID NO: 77; and a heavy chain variable region comprising the amino acid sequence of SEQ ID NO: 88; or
   a light chain variable region comprising the amino acid sequence of SEQ ID NO: 77; and a heavy chain variable region comprising the amino acid sequence of SEQ ID NO: 89; or
   a light chain variable region comprising the amino acid sequence of SEQ ID NO: 80; and a heavy chain variable region comprising the amino acid sequence of SEQ ID NO: 87; or
   a light chain variable region comprising the amino acid sequence of SEQ ID NO: 80; and a heavy chain variable region comprising the amino acid sequence of SEQ ID NO: 88; or
   a light chain variable region comprising the amino acid sequence of SEQ ID NO: 80; and a heavy chain variable region comprising the amino acid sequence of SEQ ID NO: 89;
   wherein the formulation includes: about 20 mg/mL of the anti-IL-36R antibody, about 45 mM acetate, about 180 mM sucrose, about 25 mM Glycine, about 0.4 g/L Polysorbate 80, with a pH of about 5.5.
56. A pharmaceutical formulation including:
   an anti-IL-36R antibody or antigen-binding fragment thereof, including:
   a light chain variable region comprising the amino acid sequence of SEQ ID NO: 77; and a heavy chain variable region comprising the amino acid sequence of SEQ ID NO: 87; or
   a light chain variable region comprising the amino acid sequence of SEQ ID NO: 77; and a heavy chain variable region comprising the amino acid sequence of SEQ ID NO: 88; or
   a light chain variable region comprising the amino acid sequence of SEQ ID NO: 77; and a heavy chain variable region comprising the amino acid sequence of SEQ ID NO: 89; or
   a light chain variable region comprising the amino acid sequence of SEQ ID NO: 80; and a heavy chain variable region comprising the amino acid sequence of SEQ ID NO: 87; or
   a light chain variable region comprising the amino acid sequence of SEQ ID NO: 80; and a heavy chain variable region comprising the amino acid sequence of SEQ ID NO: 88; or
   a light chain variable region comprising the amino acid sequence of SEQ ID NO: 80; and a heavy chain variable region comprising the amino acid sequence of SEQ ID NO: 89;
   wherein the formulation includes: about 150 mg/mL of the anti-IL-36R antibody, about 25 mM citrate, about 150 mM trehalose, about 25 mM methionine, about 0.2 g/L Polysorbate 20, with a pH of about 6.0.
57. A pharmaceutical formulation including:
   an anti-IL-36R antibody or antigen-binding fragment thereof, including:
   a light chain variable region comprising the amino acid sequence of SEQ ID NO: 77; and a heavy chain variable region comprising the amino acid sequence of SEQ ID NO: 87; or
   a light chain variable region comprising the amino acid sequence of SEQ ID NO: 77; and a heavy chain variable region comprising the amino acid sequence of SEQ ID NO: 88; or
   a light chain variable region comprising the amino acid sequence of SEQ ID NO: 77; and a heavy chain variable region comprising the amino acid sequence of SEQ ID NO: 89; or
   a light chain variable region comprising the amino acid sequence of SEQ ID NO: 80; and a heavy chain variable region comprising the amino acid sequence of SEQ ID NO: 87; or
   a light chain variable region comprising the amino acid sequence of SEQ ID NO: 80; and a heavy chain variable region comprising the amino acid sequence of SEQ ID NO: 88; or
   a light chain variable region comprising the amino acid sequence of SEQ ID NO: 80; and a heavy chain variable region comprising the amino acid sequence of SEQ ID NO: 89;
   wherein the formulation includes: about 150 mg/mL of the anti-IL-36R antibody, about 25 mM histidine, about 180 mM sucrose, about 20 mM mannitol, about 0.2 g/L Polysorbate 20, with a pH of about 6.5.
58. A pharmaceutical formulation including:
   an anti-IL-36R antibody or antigen-binding fragment thereof, including:
   a light chain variable region comprising the amino acid sequence of SEQ ID NO: 77; and a heavy chain variable region comprising the amino acid sequence of SEQ ID NO: 87; or
   a light chain variable region comprising the amino acid sequence of SEQ ID NO: 77; and a heavy chain variable region comprising the amino acid sequence of SEQ ID NO: 88; or
   a light chain variable region comprising the amino acid sequence of SEQ ID NO: 77; and a heavy chain variable region comprising the amino acid sequence of SEQ ID NO: 89; or
   a light chain variable region comprising the amino acid sequence of SEQ ID NO: 80; and a heavy chain variable region comprising the amino acid sequence of SEQ ID NO: 87; or
   a light chain variable region comprising the amino acid sequence of SEQ ID NO: 80; and a heavy chain variable region comprising the amino acid sequence of SEQ ID NO: 88; or
   a light chain variable region comprising the amino acid sequence of SEQ ID NO: 80; and a heavy chain variable region comprising the amino acid sequence of SEQ ID NO: 89;
   wherein the formulation includes: about 20 mg/mL of the anti-IL-36R antibody, about 25 mM citrate, about 200 mM sucrose, about 0.4 g/L Polysorbate 80, with a pH of about 6.5.
59. A pharmaceutical formulation including:
   an anti-IL-36R antibody or antigen-binding fragment thereof, including:
   a light chain variable region comprising the amino acid sequence of SEQ ID NO: 77; and a heavy chain variable region comprising the amino acid sequence of SEQ ID NO: 87; or
   a light chain variable region comprising the amino acid sequence of SEQ ID NO: 77; and a heavy chain variable region comprising the amino acid sequence of SEQ ID NO: 88; or
   a light chain variable region comprising the amino acid sequence of SEQ ID NO: 77; and a heavy chain variable region comprising the amino acid sequence of SEQ ID NO: 89; or
   a light chain variable region comprising the amino acid sequence of SEQ ID NO: 80; and a heavy chain variable region comprising the amino acid sequence of SEQ ID NO: 87; or
   a light chain variable region comprising the amino acid sequence of SEQ ID NO: 80; and a heavy chain variable region comprising the amino acid sequence of SEQ ID NO: 88; or
   a light chain variable region comprising the amino acid sequence of SEQ ID NO: 80; and a heavy chain variable region comprising the amino acid sequence of SEQ ID NO: 89;
   wherein the formulation includes: about 150 mg/mL of the anti-IL-36R antibody, about 45 mM acetate, about 150 mM sucrose, about 25 mM L-Arginine, about 0.4 g/L Polysorbate 20, with a pH of about 5.5.
60. A pharmaceutical formulation including:
   an anti-IL-36R antibody or antigen-binding fragment thereof, including:
   a light chain variable region comprising the amino acid sequence of SEQ ID NO: 77; and a heavy chain variable region comprising the amino acid sequence of SEQ ID NO: 87; or
   a light chain variable region comprising the amino acid sequence of SEQ ID NO: 77; and a heavy chain variable region comprising the amino acid sequence of SEQ ID NO: 88; or
   a light chain variable region comprising the amino acid sequence of SEQ ID NO: 77; and a heavy chain variable region comprising the amino acid sequence of SEQ ID NO: 89; or
   a light chain variable region comprising the amino acid sequence of SEQ ID NO: 80; and a heavy chain variable region comprising the amino acid sequence of SEQ ID NO: 87; or
   a light chain variable region comprising the amino acid sequence of SEQ ID NO: 80; and a heavy chain variable region comprising the amino acid sequence of SEQ ID NO: 88; or
   a light chain variable region comprising the amino acid sequence of SEQ ID NO: 80; and a heavy chain variable region comprising the amino acid sequence of SEQ ID NO: 89;
   wherein the formulation includes: about 15 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0.
61. A pharmaceutical formulation including:
   an anti-IL-36R antibody or antigen-binding fragment thereof, including:
   a light chain variable region comprising the amino acid sequence of SEQ ID NO: 77; and a heavy chain variable region comprising the amino acid sequence of SEQ ID NO: 87; or
   a light chain variable region comprising the amino acid sequence of SEQ ID NO: 77; and a heavy chain variable region comprising the amino acid sequence of SEQ ID NO: 88; or
   a light chain variable region comprising the amino acid sequence of SEQ ID NO: 77; and a heavy chain variable region comprising the amino acid sequence of SEQ ID NO: 89; or
   a light chain variable region comprising the amino acid sequence of SEQ ID NO: 80; and a heavy chain variable region comprising the amino acid sequence of SEQ ID NO: 87; or
   a light chain variable region comprising the amino acid sequence of SEQ ID NO: 80; and a heavy chain variable region comprising the amino acid sequence of SEQ ID NO: 88; or
   a light chain variable region comprising the amino acid sequence of SEQ ID NO: 80; and a heavy chain variable region comprising the amino acid sequence of SEQ ID NO: 89;
   wherein the formulation includes: about 80 mg/mL of the anti-IL-36R antibody, about 25 mM acetate, about 100 mM mannitol, about 50 mM NaCl, about 0.2 g/L Polysorbate 20, with a pH of about 5.5.
62. A pharmaceutical formulation including:
   an anti-IL-36R antibody or antigen-binding fragment thereof, including:
   a light chain variable region comprising the amino acid sequence of SEQ ID NO: 77; and a heavy chain variable region comprising the amino acid sequence of SEQ ID NO: 87; or
   a light chain variable region comprising the amino acid sequence of SEQ ID NO: 77; and a heavy chain variable region comprising the amino acid sequence of SEQ ID NO: 88; or
   a light chain variable region comprising the amino acid sequence of SEQ ID NO: 77; and a heavy chain variable region comprising the amino acid sequence of SEQ ID NO: 89; or
   a light chain variable region comprising the amino acid sequence of SEQ ID NO: 80; and a heavy chain variable region comprising the amino acid sequence of SEQ ID NO: 87; or
   a light chain variable region comprising the amino acid sequence of SEQ ID NO: 80; and a heavy chain variable region comprising the amino acid sequence of SEQ ID NO: 88; or
   a light chain variable region comprising the amino acid sequence of SEQ ID NO: 80; and a heavy chain variable region comprising the amino acid sequence of SEQ ID NO: 89;
   wherein the formulation includes: about 100 mg/mL of the anti-IL-36R antibody, about 20 mM succinate, about 220 mM sucrose, about 0.1 g/L Polysorbate 80, with a pH of about 6.0.
63. A pharmaceutical formulation including:
   an anti-IL-36R antibody or antigen-binding fragment thereof, including:
   a light chain variable region comprising the amino acid sequence of SEQ ID NO: 77; and a heavy chain variable region comprising the amino acid sequence of SEQ ID NO: 87; or
   a light chain variable region comprising the amino acid sequence of SEQ ID NO: 77; and a heavy chain variable region comprising the amino acid sequence of SEQ ID NO: 88; or
   a light chain variable region comprising the amino acid sequence of SEQ ID NO: 77; and a heavy chain variable region comprising the amino acid sequence of SEQ ID NO: 89; or
   a light chain variable region comprising the amino acid sequence of SEQ ID NO: 80; and a heavy chain variable region comprising the amino acid sequence of SEQ ID NO: 87; or
   a light chain variable region comprising the amino acid sequence of SEQ ID NO: 80; and a heavy chain variable region comprising the amino acid sequence of SEQ ID NO: 88; or
   a light chain variable region comprising the amino acid sequence of SEQ ID NO: 80; and a heavy chain variable region comprising the amino acid sequence of SEQ ID NO: 89;
   wherein the formulation includes: about 60 mg/mL of the anti-IL-36R antibody, about 25 mM citrate, about 0.4 g/L Polysorbate 20, with a pH of about 6.5.
64. A pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
      iii. a light chain including an amino acid sequence set forth as SEQ ID NO:118 and a heavy chain including an amino acid sequence set forth as SEQ ID NO:127;
         wherein the formulation is selected from the group consisting of:
         I. formulation including about 20 mg/mL to about 150 mg/mL of the anti-IL-36R antibody, about 40 mM histidine, about 120 mM sucrose, about 50 mM L-Arginine, about 5 mM NaCl and about 1.0 g/L Polysorbate 20, with a pH of about 6.0;
         II. formulation including about 20 mg/mL to about 150 mg/mL of the anti-IL-36R antibody, about 45 mM acetate, about 150 mM sucrose, about 25 mM L-Arginine, about 0.4 g/L Polysorbate 20, with a pH of about 5.5;
         III. formulation including about 20 mg/mL to about 150 mg/mL of the anti-IL-36R antibody, about 45 mM acetate, about 180 mM sucrose, about 25 mM Glycine, about 0.4 g/L Polysorbate 80, with a pH of about 5.5;
         IV. formulation including about 20 mg/mL to about 150 mg/mL of the anti-IL-36R antibody, about 25 mM citrate, about 150 mM trehalose, about 25 mM methionine, about 0.2 g/L Polysorbate 20, with a pH of about 6.0;
         V. formulation including about 20 mg/mL to about 150 mg/mL of the anti-IL-36R antibody, about 25 mM histidine, about 180 mM sucrose, about 20 mM mannitol, about 0.2 g/L Polysorbate 20, with a pH of about 6.5;
         VI. formulation including about 20 mg/mL to about 150 mg/mL of the anti-IL-36R antibody, about 25 mM citrate, about 200 mM sucrose, about 0.4 g/L Polysorbate 80, with a pH of about 6.5;
         VII. formulation including about 20 mg/mL to about 150 mg/mL of the anti-IL-36R antibody, about 45 mM acetate, about 150 mM sucrose, about 25 mM L-Arginine, about 0.4 g/L Polysorbate 20, with a pH of about 5.5;
         VIII. formulation including about 20 mg/mL to about 150 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0;
         IX. formulation including about 20 mg/mL to about 150 mg/mL of the anti-IL-36R antibody, about 25 mM acetate, about 100 mM mannitol, about 50 mM NaCl, about 0.2 g/L Polysorbate 20, with a pH of about 5.5;
         X. formulation including about 20 mg/mL to about 150 mg/mL of the anti-IL-36R antibody, about 20 mM succinate, about 220 mM sucrose, about 0.1 g/L Polysorbate 80, with a pH of about 6.0; and
         XI. formulation including about 20 mg/mL to about 150 mg/mL of the anti-IL-36R antibody, about 25 mM citrate, about 0.4 g/L Polysorbate 20, with a pH of about 6.5.
65. A pharmaceutical formulation including:
   an anti-IL-36R antibody or antigen-binding fragment thereof, including:
   a light chain variable region comprising the amino acid sequence of SEQ ID NO: 77; and a heavy chain variable region comprising the amino acid sequence of SEQ ID NO: 87; or
   a light chain variable region comprising the amino acid sequence of SEQ ID NO: 77; and a heavy chain variable region comprising the amino acid sequence of SEQ ID NO: 88; or
   a light chain variable region comprising the amino acid sequence of SEQ ID NO: 77; and a heavy chain variable region comprising the amino acid sequence of SEQ ID NO: 89; or
   a light chain variable region comprising the amino acid sequence of SEQ ID NO: 80; and a heavy chain variable region comprising the amino acid sequence of SEQ ID NO: 87; or
   a light chain variable region comprising the amino acid sequence of SEQ ID NO: 80; and a heavy chain variable region comprising the amino acid sequence of SEQ ID NO: 88; or
   a light chain variable region comprising the amino acid sequence of SEQ ID NO: 80; and a heavy chain variable region comprising the amino acid sequence of SEQ ID NO: 89;
      wherein the formulation is selected from the group consisting of:
      I. formulation including about 20 mg/mL to about 150 mg/mL of the anti-IL-36R antibody, about 40 mM histidine, about 120 mM sucrose, about 50 mM L-Arginine, about 5 mM NaCl and about 1.0 g/L Polysorbate 20, with a pH of about 6.0;
      II. formulation including about 20 mg/mL to about 150 mg/mL of the anti-IL-36R antibody, about 45 mM acetate, about 150 mM sucrose, about 25 mM L-Arginine, about 0.4 g/L Polysorbate 20, with a pH of about 5.5;
      III. formulation including about 20 mg/mL to about 150 mg/mL of the anti-IL-36R antibody, about 45 mM acetate, about 180 mM sucrose, about 25 mM Glycine, about 0.4 g/L Polysorbate 80, with a pH of about 5.5;
      IV. formulation including about 20 mg/mL to about 150 mg/mL of the anti-IL-36R antibody, about 25 mM citrate, about 150 mM trehalose, about 25 mM methionine, about 0.2 g/L Polysorbate 20, with a pH of about 6.0;
      V. formulation including about 20 mg/mL to about 150 mg/mL of the anti-IL-36R antibody, about 25 mM histidine, about 180 mM sucrose, about 20 mM mannitol, about 0.2 g/L Polysorbate 20, with a pH of about 6.5;
      VI. formulation including about 20 mg/mL to about 150 mg/mL of the anti-IL-36R antibody, about 25 mM citrate, about 200 mM sucrose, about 0.4 g/L Polysorbate 80, with a pH of about 6.5;
      VII. formulation including about 20 mg/mL to about 150 mg/mL of the anti-IL-36R antibody, about 45 mM acetate, about 150 mM sucrose, about 25 mM L-Arginine, about 0.4 g/L Polysorbate 20, with a pH of about 5.5;
      VIII. formulation including about 20 mg/mL to about 150 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0;
      IX. formulation including about 20 mg/mL to about 150 mg/mL of the anti-IL-36R antibody, about 25 mM acetate, about 100 mM mannitol, about 50 mM NaCl, about 0.2 g/L Polysorbate 20, with a pH of about 5.5;
      X. formulation including about 20 mg/mL to about 150 mg/mL of the anti-IL-36R antibody, about 20 mM succinate, about 220 mM sucrose, about 0.1 g/L Polysorbate 80, with a pH of about 6.0; and
      XI. formulation including about 20 mg/mL to about 150 mg/mL of the anti-IL-36R antibody, about 25 mM citrate, about 0.4 g/L Polysorbate 20, with a pH of about 6.5.
66. A pharmaceutical product including a vial or syringe including the pharmaceutical formulation according to any of the preceding clauses for use in any one of the aspects of the present invention.
67. The pharmaceutical product according to clause 66 further including a pre-assembled injection device.
68. The pharmaceutical product of clause 67 wherein the pre-assembled injection device is an autoinjector or a syringe with or without a needle safety device.
69. A pre-assembled injection device including a pharmaceutical formulation according to any of the preceding clauses for use in any one of the aspects of the present invention.
70. The pre-assembled injection device according to clause 69, wherein said device is an autoinjector or a syringe with or without a needle safety device.
71. The pre-assembled injection device according to clause 69, wherein said formulation is suitable for intravenous, subcutaneous or intramuscular administration.
72. The pre-assembled injection device according to clause 70, wherein the autoinjector or the syringe with or without needle safety device includes a pharmaceutical formulation including:
   an anti-IL-36R antibody or antigen-binding fragment thereof, including:
      i. a light chain including an amino acid sequence set forth as SEQ ID NO:118 and a heavy chain including an amino acid sequence set forth as SEQ ID NO:125; or
      ii. a light chain including an amino acid sequence set forth as SEQ ID NO:118 and a heavy chain including an amino acid sequence set forth as SEQ ID NO:126; or
         a light chain including an amino acid sequence set forth as SEQ ID NO:118 and a heavy chain including an amino acid sequence set forth as SEQ ID NO:127; wherein the formulation is selected from the group consisting of:
         I. formulation including about 20 mg/ml of the anti-IL-36R antibody, about 40 mM histidine, about 120 mM sucrose, about 50 mM L-Arginine, about 5 mM NaCl and about 1.0 g/L Polysorbate 20, with a pH of about 6.0;
         II. formulation including about 60 mg/mL of the anti-IL-36R antibody, about 45 mM acetate, about 150 mM sucrose, about 25 mM L-Arginine, about 0.4 g/L Polysorbate 20, with a pH of about 5.5;
         III. formulation including about 20 mg/mL of the anti-IL-36R antibody, about 45 mM acetate, about 180 mM sucrose, about 25 mM Glycine, about 0.4 g/L Polysorbate 80, with a pH of about 5.5;
         IV. formulation including about 150 mg/mL of the anti-IL-36R antibody, about 25 mM citrate, about 150 mM trehalose, about 25 mM methionine, about 0.2 g/L Polysorbate 20, with a pH of about 6.0;
         V. formulation including about 150 mg/mL of the anti-IL-36R antibody, about 25 mM histidine, about 180 mM sucrose, about 20 mM mannitol, about 0.2 g/L Polysorbate 20, with a pH of about 6.5;
         VI. formulation including about 20 mg/mL of the anti-IL-36R antibody, about 25 mM citrate, about 200 mM sucrose, about 0.4 g/L Polysorbate 80, with a pH of about 6.5;
         VII. formulation including about 150 mg/mL of the anti-IL-36R antibody, about 45 mM acetate, about 150 mM sucrose, about 25 mM L-Arginine, about 0.4 g/L Polysorbate 20, with a pH of about 5.5;
         VIII. formulation including about 15 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0;
         IX. formulation including about 80 mg/mL of the anti-IL-36R antibody, about 25 mM acetate, about 100 mM mannitol, about 50 mM NaCl, about 0.2 g/L Polysorbate 20, with a pH of about 5.5;
         X. formulation including about 100 mg/mL of the anti-IL-36R antibody, about 20 mM succinate, about 220 mM sucrose, about 0.1 g/L Polysorbate 80, with a pH of about 6.0; and
         XI. formulation including about 60 mg/mL of the anti-IL-36R antibody, about 25 mM citrate, about 0.4 g/L Polysorbate 20, with a pH of about 6.5.
73. The pre-assembled injection device according to clause 70, wherein the autoinjector or the syringe with a needle safety device includes:
   a. about 300 mg of the antibody in about 2 mL formulation volume; or
   b. about 225 mg of the antibody in about 1.5 mL formulation volume; or
   c. about 150 mg of the antibody in about 1 mL formulation volume; or
   d. about 75 mg of the antibody in about 0.5 mL formulation volume; or
   e. about 60 mg of the antibody in about 0.4 mL formulation volume.
74. The vial according to clause 66, wherein the vial includes:
   a. about 1200 mg of the antibody in about 20 mL formulation volume; or
   b. about 900 mg of the antibody in about 15 mL formulation volume; or
   c. about 600 mg of the antibody in about 10 mL formulation volume; or
   d. about 300 mg of the antibody in about 150 mL formulation volume; or
   e. about 1500 mg of the antibody in about 2.5 mL formulation volume.
75. A pharmaceutical product, including: a vial including about 100 mg to 1500 mg of an anti-IL-36R antibody in powder form; instructions for reconstitution of the anti-IL-36R antibody; and instructions for preparing the reconstituted antibody for infusion, wherein the anti-IL-36R antibody comprises a light chain including an amino acid sequence set forth as SEQ ID NO:118 and a heavy chain including an amino acid sequence set forth as any one of SEQ ID Nos:125, 126 or 127; and the reconstitution instructions require reconstitution with water for injection to an extractable volume from 1 to 50 mL.
76. A method of treating palmoplantar pustulosis (PPP) in a subject, the method comprising administering or having administered to the subject a therapeutically effective amount of a humanized anti-interleukin-36 receptor (anti-IL-36R) antibody,
   wherein the humanized anti-IL-36R antibody comprises a light chain variable region comprising the amino acid sequence of SEQ ID NO: 26 (L-CDR1), the amino acid sequence of SEQ ID NO: 102, 103, 104, 105 106 or 140 (L-CDR2), and the amino acid sequence of SEQ ID NO: 44 (L-CDR3); and a heavy chain variable region comprising the amino acid sequence of SEQ ID NO: 53 or SEQ ID NO: 141 (H-CDR1), the amino acid sequence of SEQ ID NO: 62, 108, 109, 110, 111 or 142 (H-CDR2), and the amino acid sequence of SEQ ID NO: 72 (H-CDR3).
77. The method of clause 76, wherein the humanized anti-IL-36R antibody comprises:
   I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3);
   II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3);
   III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3);
   IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3);
   V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3);
   VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
   VII. a) a light chain variable region comprising the amino acid sequence of SEQ ID NO: 26 (L-CDR1), the amino acid sequence of SEQ ID NO: 104 (L-CDR2), and the amino acid sequence of SEQ ID NO: 44 (L-CDR3); and b) a heavy chain variable region comprising the amino acid sequence of SEQ ID NO: 141 (H-CDR1), the amino acid sequence of SEQ ID NO: 142 (H-CDR2), and the amino acid sequence of SEQ ID NO: 72 (H-CDR3).
78. The method of clause 77, wherein the humanized anti-IL-36R antibody comprises:
   a) a light chain variable region comprising the amino acid sequence of SEQ ID NO: 26 (L-CDR1), the amino acid sequence of SEQ ID NO: 104 (L-CDR2), and the amino acid sequence of SEQ ID NO: 44 (L-CDR3); and b) a heavy chain variable region comprising the amino acid sequence of SEQ ID NO: 141 (H-CDR1), the amino acid sequence of SEQ ID NO: 142 (H-CDR2), and the amino acid sequence of SEQ ID NO: 72 (H-CDR3).
79. The method of clause 76, wherein the humanized anti-IL-36R antibody comprises:
   (i) a light chain variable region comprising the amino acid sequence of SEQ ID NO: 77; and a heavy chain variable region comprising the amino acid sequence of SEQ ID NO: 87; or
   (ii) a light chain variable region comprising the amino acid sequence of SEQ ID NO: 77; and a heavy chain variable region comprising the amino acid sequence of SEQ ID NO: 88; or
   (iii) a light chain variable region comprising the amino acid sequence of SEQ ID NO: 77; and a heavy chain variable region comprising the amino acid sequence of SEQ ID NO: 89; or
   (iv) a light chain variable region comprising the amino acid sequence of SEQ ID NO: 80; and a heavy chain variable region comprising the amino acid sequence of SEQ ID NO: 87; or
   (v) a light chain variable region comprising the amino acid sequence of SEQ ID NO: 80; and a heavy chain variable region comprising the amino acid sequence of SEQ ID NO: 88; or
   (vi) a light chain variable region comprising the amino acid sequence of SEQ ID NO: 80; and a heavy chain variable region comprising the amino acid sequence of SEQ ID NO: 89; or
   (vii) a light chain variable region comprising the amino acid sequence of SEQ ID NO: 85; and a heavy chain variable region comprising the amino acid sequence of SEQ ID NO: 100; or
   (viii) a light chain variable region comprising the amino acid sequence of SEQ ID NO: 85; and a heavy chain variable region comprising the amino acid sequence of SEQ ID NO: 101; or
   (ix) a light chain variable region comprising the amino acid sequence of SEQ ID NO: 86; and a heavy chain variable region comprising the amino acid sequence of SEQ ID NO: 100; or
   (x) a light chain variable region comprising the amino acid sequence of SEQ ID NO: 86; and a heavy chain variable region comprising the amino acid sequence of SEQ ID NO: 101.
80. The method of clause 76, wherein the humanized anti-IL-36R antibody comprises:
   a light chain variable region having at least 94% identity to SEQ ID NO: 80; and a heavy chain variable region having at least 97% sequence identity to SEQ ID NO: 89.
81. The method of clause 80, wherein the humanized anti-IL-36R antibody comprises: a light chain variable region comprising the amino acid sequence of SEQ ID NO: 80; and a heavy chain variable region comprising the amino acid sequence of SEQ ID NO: 89.
82. The method of clause 76, wherein the humanized anti-IL-36R antibody comprises:
   i. a light chain comprising the amino acid sequence of SEQ ID NO: 115; and a heavy chain comprising the amino acid sequence of SEQ ID NO: 125; or
   ii. a light chain comprising the amino acid sequence of SEQ ID NO: 115; and a heavy chain comprising the amino acid sequence of SEQ ID NO: 126; or
   iii. a light chain comprising the amino acid sequence of SEQ ID NO: 115; and a heavy chain comprising the amino acid sequence of SEQ ID NO: 127; or
   iv. a light chain comprising the amino acid sequence of SEQ ID NO: 118; and a heavy chain comprising the amino acid sequence of SEQ ID NO: 125; or
   v. a light chain comprising the amino acid sequence of SEQ ID NO: 118; and a heavy chain comprising the amino acid sequence of SEQ ID NO: 126; or
   vi. a light chain comprising the amino acid sequence of SEQ ID NO: 118; and a heavy chain comprising the amino acid sequence of SEQ ID NO: 127; or
   vii. a light chain comprising the amino acid sequence of SEQ ID NO: 123; and a heavy chain comprising the amino acid sequence of SEQ ID NO: 138; or
   viii. a light chain comprising the amino acid sequence of SEQ ID NO: 123; and a heavy chain comprising the amino acid sequence of SEQ ID NO: 139; or
   ix. a light chain comprising the amino acid sequence of SEQ ID NO: 124; and a heavy chain comprising the amino acid sequence of SEQ ID NO: 138.
83. The method of clause 82, wherein the humanized anti-IL-36R antibody comprises: a light chain comprising the amino acid sequence of SEQ ID NO: 118 and a heavy chain comprising the amino acid sequence of SEQ ID NO: 127.
84. The method of clause 76, wherein the humanized anti-IL-36R antibody is a full-length antibody.
85. The method of clause 76, wherein the humanized anti-IL-36R antibody is an antibody fragment.
86. The method of clause 85, wherein the antibody fragment is selected from the group consisting of: Fab, Fab′, F(ab′)2, Fd, Fv, scFv and scFv-Fc fragment, a single-chain antibody, a minibody, and a diabody.
87. The method of clause 76, wherein the humanized anti-IL-36R antibody binds human IL-36R at a Kd<0.1 nM.
88. The method of clause 76, wherein the PPP is classified as moderate to severe PPP.
89. The method of clause 76, wherein signs and/or symptoms of an acute phase flare-up of PPP in the subject are reduced and/or alleviated after administration of the humanized anti-IL-36R antibody.
90. The method of clause 76, wherein severity and/or duration of PPP flares in the subject are reduced after administration of the humanized anti-IL-36R antibody.
91. The method of clause 76, wherein the anti-IL-36R antibody is administered subcutaneously or intravenously or by both routes simultaneously or sequentially and in any order.
92. The method of clause 91, wherein the subcutaneous administration comprises administration of 300 mg or 600 mg or 900 mg dose of the anti-IL-36R antibody.
93. The method of clause 91, wherein the intravenous administration comprises administering 300 mg, 600 mg, 900 mg or 1200 mg dose of the anti-IL-36R antibody.
94. The method of clause 92, wherein the subcutaneous administration comprises administering qw (once every week), q2w (once every 2 weeks), q4w (once every 4 weeks), q6w (once every 6 weeks), q8w (once every 8 weeks), or a combination thereof.
95. The method of clause 93, wherein the intravenous administration comprises administering q4w (once every 4 weeks), q8w (once every 8 weeks) or q12w (once every 12 weeks) interval, or a combination thereof.
96. The method of clause 76, wherein the anti-IL-36R antibody is administered by an initial dose, wherein the initial dose comprises administering intravenously or subcutaneously.
97. The method of clause 96, wherein the administration of the anti-IL-36R antibody further comprises a subsequent dose administered intravenously or subcutaneously.
98. The method of clause 96, wherein the initial dose is 150 mg, 300 mg or 600 mg or 900 mg.
99. The method of clause 98, wherein the initial dose of 150 mg or 300 mg is administered per day (in consecutive days) for two weeks.
100. The method of clause 98, wherein the initial dose of 600 mg or 900 mg is administered once per week for two weeks up to week 4, comprising administering at weeks 0 and 1; weeks 0 and 2; weeks 0 and 3; or weeks 0 and 4.
101. The method of clause 98, wherein the initial dose of 600 mg or 900 mg is administered once per week for three weeks up to week 4, comprising administering at weeks 0, 1 and 2; weeks 0, 1 and 3; weeks 0, 1 and 4; weeks 0, 2 and 3; weeks 0, 2 and 4; or weeks 0, 3 and 4.
102. The method of clause 98, wherein the initial dose of 600 mg or 900 mg is administered once per week for four weeks up to week 4, comprising administering at weeks 0, 1, 2 and 3; weeks 0, 1, 2 and 4; weeks 0, 1, 3 and 4; or weeks 0, 2, 3 and 4.
103. The method of clause 98, wherein the initial dose of 600 mg or 900 mg is administered twice per week for 2 weeks, twice per week for 3 weeks, or twice per week for 4 weeks.
104. The method of clause 98, wherein the initial dose of 600 mg or 300 mg is administered five times at day 1, week 1, week 2, week 3 and week 4.
105. The method of clause 98, wherein the initial dose of 900 mg, 600 mg or 300 mg is administered two to three times from day 1 to week 4.
106. The method of clause 97, wherein the subsequent dose is 300 mg or 600 mg.
107. The method of clause 106, wherein the subsequent dose administration begins two to four weeks after the initial dose administration ends.
108. The method of clause 106, wherein the subsequent dose of 300 mg or 600 mg is administered once every 2 weeks, once every 4 weeks, once every 6 weeks, or once every 8 weeks.
109. The method of clause 106, wherein the subsequent dose is administered q4w (once every 4 weeks) or q6-8w (once every 6-8 weeks) from week 8 onward.
110. The method of clause 106, wherein the subsequent dose of 300 mg is administered q4w from weeks 8 to 16 and q8w from week 20 onward.
111. The method of clause 106, wherein the subsequent dose of 300 mg is administered q6-8w from weeks 8 to 16 and q10-12w from week 20 onward.
112. The method according to any of clauses 76-111, wherein the subject has a Palmoplantar Pustular Physicians Global Reference (PPP PGA) score of 0 or 1 after administration of the humanized anti-IL-36R antibody.
113. The method according to any of clauses 76-111, wherein the subject has a PPP PGA score of 0 or 1 at week 16, 24, 36, 48, 60 or 72 after administration of the humanized anti-IL-36R antibody.
114. The method according to any of clauses 76-111, wherein the subject has a change in PPP ASI50 from baseline at week 16, 24, 36, 48, 60 or 72 after administration of the humanized anti-IL-36R antibody.
115. The method according to any of clauses 76-111, wherein the subject has a ppPAS150 at about week 16 after administration of the humanized anti-IL-36R antibody.

116. The method according to any of clauses 76-111, wherein after administration of the humanized anti-IL-36R antibody at least one of the following outcomes is achieved:
(a) the subject achieves a 50% reduction in PPP ASI (PPP ASI50) at or after about week 4, week 6, week 8, week 12, week 16 or week 24; or
(b) the subject experience at about 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% reduction in the number of drug-related Adverse Events (AEs) as compared to other treatments (e.g., guselkumab);
(c) the subject experiences at least 7%, 8%, 9%, 10%, 11%, 12%, 13%, 14.%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more improvement in his or her pustule severity (as compared to baseline) at or after about week 4, week 6, week 8, week 12, week 16 or week 24; or
(d) the anti-IL-36R antibody treatment shows a superior efficacy over guselkumab by at least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 0.41%, 42%, 43%, 44%, 45%, 46%, 47%, 48%, 49%, 50%, about 60%, about 70%, about 80%, about 90%, about 100%, about 150%, about 200% or more at or after about week 4, week 6, week 8, week 12, week 16 or week 24 or over time; or
(e) the subject achieves a PPP Physicians Global Assessment (PPP PGA) score of 0 or 1 (clear/almost clear) at or after about week 4, week 6, week 8, week 12, week 16 or week 24; or
(f) the subject achieves a Psoriasis Area and Severity Index for PPP (PPP ASI) 75 at or after about week 4, week 6, week 8, week 12, week 16 or week 24; or
(g) the subject experiences 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, about 60%, about 70%, about 80%, about 90%, about 100%, about 150%, about 200% or more improvement from baseline in the PPP ASI at or after about week 4, week 6, week 8, week 12, week 16 or week 24; or
(h) the subject achieves an improved change of 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, about 60%, about 70%, about 80%, about 90%, about 100%, about 150%, about 200% or more from baseline in Pain Visual Analog Scale (VAS) score at or after about week 4, week 6, week 8, week 12, week 16 or week 24; or
(i) the subject achieves a clinical improvement of 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, about 60%, about 70%, about 80%, about 90%, about 100%, about 150%, about 200% or more from baseline as assessed via Dermatology Life Quality Index (DLQI) at or after about week 4, week 6, week 8, week 12, week 16 or week 24; or
(j) the subject achieves a PPP ASI50 at visit 1, 2, 3, 4, 5, 6, 7, 8, 9, 10 or all other visits; or
(k) the subject achieves 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, about 60%, about 70%, about 80%, about 90%, or about 100% reduction in PPP ASI scores at week 16 and all other visits; or
(l) the subject achieves PPP Physicians Global Assessment (PPP PGA) score of 0 or 1 (clear/almost clear) at visit 1, 2, 3, 4, 5, 6, 7, 8, 9, 10 or all other visits;
(m) the subject achieves a PPP ASI75 at visit 1, 2, 3, 4, 5, 6, 7, 8, 9, 10 or all other visits;
(n) the subject experiences 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, about 60%, about 70%, about 80%, about 90%, or about 100% percent change from baseline in the PPP ASI at visit 1, 2, 3, 4, 5, 6, 7, 8, 9, 10 or all other visits; or
(o) the subject experiences a lesser time (in days, e.g., about 5, about 10, about 15, about 20, about 25, about 30, about 40, about 50, about 60, about 70, about 80, about 90, about 100, about 120, about 140, about 160, about 180, about 200, about 250, about 300 or more days) to achieving PPP ASI50 as compared to other treatments (e.g., guselkumab); or
(p) the subject experiences a longer time (in days, e.g., about 5, about 10, about 15, about 20, about 25, about 30, about 40, about 50, about 60, about 70, about 80, about 90, about 100, about 120, about 140, about 160, about 180, about 200, about 250, about 300 or more days) to loss of PPP ASI50 as compared to other treatments (e.g., guselkumab);
(q) the subject experiences 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, about 60%, about 70%, about 80%, about 90%, or about 100% of improved change in plaque psoriasis BSA involvement at or after about week 4, week 6, week 8, week 12, week 16 or week 24 in subjects with concurrent plaque psoriasis at baseline; or
(r) the subject experiences at least about 10%, about 20%, about 30%, about 40%, about 50%, about 60%, about 70%, about 80%, about 90%, about 100%, about 150%, about 200%, or about 300% superiority over placebo in achieving PPP ASI50 at or after about week 4, week 6, week 8, week 12, week 16 or week 24; or
(s) the subject achieves about 5%, about 10%, about 15%, about 20%, about 30%, about 40%, about 50%, about 60%, about 70%, about 80%, about 90%, about 100% or more of change in PPP ASI from baseline at or after about week 4, week 6, week 8, week 12, week 16 or week 24; or
(t) the subject achieves about 5%, about 10%, about 15%, about 20%, about 30%, about 40%, about 50%, about 60%, about 70%, about 80%, about 90%, about 100% or more of positive or improved change in Pain VAS score from baseline at or after about week 4, week 6, week 8, week 12, week 16 or week 24; or
(u) the subject achieves about 5%, about 10%, about 15%, about 20%, about 30%, about 40%, about 50%, about 60%, about 70%, about 80%, about 90%, about 100% or more of positive or improved PPP SI change from baseline at or after about week 4, week 6, week 8, week 12, week 16 or week 24; or
(v) the subject achieves about 5%, about 10%, about 15%, about 20%, about 30%, about 40%, about 50%, about 60%, about 70%, about 80%, about 90%, about 100% or more of positive or improved PPP ASI change from baseline at week 52; or
(w) the subject achieves about 5%, about 10%, about 15%, about 20%, about 30%, about 40%, about 50%, about 60%, about 70%, about 80%, about 90%, about 100% or more of reduction in occurrence of Treatment Emergent Adverse Events (TEAEs) from baseline overtime or at or after about week 4, week 6, week 8, week 12, week 16 or week 24; or
(x) the subject achieves about 5%, about 10%, about 15%, about 20%, about 30%, about 40%, about 50%, about 60%, about 70%, about 80%, about 90%, about 100% or more of a positive or improved change in pustule count from baseline over time or at or after about week 4, week 6, week 8, week 12, week 16 or week 24; or
(y) the subject achieves about 5%, about 10%, about 15%, about 20%, about 30%, about 40%, about 50%, about 60%, about 70%, about 80%, about 90%, about 100% or more of a positive or improved change in pustular severity from baseline over time or at or after about week 4, week 6, week 8, week 12, week 16 or week 24; or
(z) the subject achieves a PPP PGA clear/almost clear as compared to baseline or placebo over time or at or after about week 4, week 6, week 8, week 12, week 16 or week 24; or
(aa) the subject achieves a PPP PGA pustule clear/almost clear as compared to baseline or placebo over time or at or after about week 4, week 6, week 8, week 12, week 16 or week 24; or
(bb) the subject achieves about 5%, about 10%, about 15%, about 20%, about 30%, about 40%, about 50%, about 60%, about 70%, about 80%, about 90%, about 100% or more of a positive change from baseline in total score of PPQLI (Palmoplantar Quality of Life Instrument), DLQI (Dermatology Life Quality Index), PSS (Psoriasis Symptom Scale), and BASDAI (Bath Ankylosing Spondylitis Disease Activity Index) over time or at or after about week 4, week 6, week 8, week 12, week 16 or week 24; or
(cc) the subject achieves a PPP ASI50 over time or at or after about week 4, week 6, week 8, week 12, week 16 or week 24 or week 52; or
(dd) the subject achieves a PPP ASI75 over time or at or after about week 4, week 6, week 8, week 12, week 16 or week 24 or week 52; or
(ee) the subject achieves about 5%, about 10%, about 15%, about 20%, about 30%, about 40%, about 50%, about 60%, about 70%, about 80%, about 90%, about 100% or more of a positive or improved percent change from baseline in the PPP ASI over time or at or after about week 4, week 6, week 8, week 12, week 16 or week 24 or week 52; or
(ff) the subject achieves about 5%, about 10%, about 15%, about 20%, about 30%, about 40%, about 50%, about 60%, about 70%, about 80%, about 90%, about 100% or more of a positive or improved PPSI change as compared to baseline over time or at or after about week 4, week 6, week 8, week 12, week 16 or week 24 or week 52; or
(gg) the subject achieves about 5%, about 10%, about 15%, about 20%, about 30%, about 40%, about 50%, about 60%, about 70%, about 80%, about 90%, about 100% or more of a positive or improved change in Pain VAS score for pain on palm and/or soles (PPP Pain VAS) and/or one for muscular and joint pain as compared to baseline or placebo over time or at or after about week 4, week 6, week 8, week 12, week 16 or week 24 or week 52; or
(hh) the subject achieves a shorter time to PPP ASI75 (in days, e.g., about 5, about 10, about 15, about 20, about 25, about 30, about 40, about 50, about 60, about 70, about 80, about 90, about 100, about 120, about 140, about 160, about 180, about 200, about 250, about 300 or more days; or in weeks, e.g., 4 weeks, 8 weeks, 12 weeks, 16 weeks, 20 weeks, 24 weeks, or more) as compared to baseline or placebo over time or at or after about week 4, week 6, week 8, week 12, week 16 or week 24 or week 52; or
(ii) the subject achieves a shorter time to PPP ASI50 (in days, e.g., about 5, about 10, about 15, about 20, about 25, about 30, about 40, about 50, about 60, about 70, about 80, about 90, about 100, about 120, about 140, about 160, about 180, about 200, about 250, about 300 or more days; or in weeks, e.g., 4 weeks, 8 weeks, 12 weeks, 16 weeks, 20 weeks, 24 weeks, or more) as compared to baseline or placebo over time or at or after about week 4, week 6, week 8, week 12, week 16 or week 24 or week 52; or
(jj) the subject achieves a longer time to loss of PPP ASI75 (in days, e.g., about 5, about 10, about 15, about 20, about 25, about 30, about 40, about 50, about 60, about 70, about 80, about 90, about 100, about 120, about 140, about 160, about 180, about 200, about 250, about 300 or more days; or in weeks, e.g., 4 weeks, 8 weeks, 12 weeks, 16 weeks, 20 weeks, 24 weeks, or more) as compared to baseline or placebo over time or at or after about week 4, week 6, week 8, week 12, week 16 or week 24 or week 52; or
(kk) the subject achieves a longer time to loss of PPP ASI50 (in days, e.g., about 5, about 10, about 15, about 20, about 25, about 30, about 40, about 50, about 60, about 70, about 80, about 90, about 100, about 120, about 140, about 160, about 180, about 200, about 250, about 300 or more days) as compared to baseline or placebo over time or at or after about week 4, week 6, week 8, week 12, week 16 or week 24 or week 52; or
(ll) the subject achieves about 5%, about 10%, about 15%, about 20%, about 30%, about 40%, about 50%, about 60%, about 70%, about 80%, about 90%, about 100% or more of a positive or improved change in PASI as compared to baseline or placebo over time or at or after about week 4, week 6, week 8, week 12, week 16 or week 24 or week 52; or
(mm) the subject achieves about 5%, about 10%, about 15%, about 20%, about 30%, about 40%, about 50%, about 60%, about 70%, about 80%, about 90%, about 100% or more of a positive or improved change in sPGA as compared to baseline or placebo over time or at or after about week 4, week 6, week 8, week 12, week 16 or week 24 or week 52; or
(nn) the subject achieves about 5%, about 10%, about 15%, about 20%, about 30%, about 40%, about 50%, about 60%, about 70%, about 80%, about 90%, about 100% or more of a positive or improved percent change in TPSS as compared with baseline or placebo over time or at or after about week 4, week 6, week 8, week 12, week 16 or week 24 or week 52; or
(oo) the subject achieves about 5%, about 10%, about 15%, about 20%, about 30%, about 40%, about 50%, about 60%, about 70%, about 80%, about 90%, about 100% or more of a positive or improved pharmacokinetic as compared to baseline or placebo over time or at or after about week 4, week 6, week 8, week 12, week 16 or week 24 or week 52; or
(pp) the subject achieves about 1.2 fold, about 1.5 fold, about 2 fold, about 2.5 fold, about 3 fold, about 3.5 fold, about 4 fold or more of an improved gene expression change for the genes disclosed herein as an indication that the treatment is efficacious as compared with baseline or placebo over time at or after about week 4, week 6, week 8, week 12, week 16 or week 24 or week 52; or
(qq) the subject achieves a PPP PGA of 0 or 1 at a reduced time as compared with baseline or placebo over time or at or after about week 4, week 6, week 8, week 12, week 16 or week 24 or week 52.
117. The method according to any of clauses 76-111, wherein the step of administering the humanized anti-IL-36R antibody to the subject comprises administering a formulation to the subject that comprises the humanized anti-IL-36R antibody at a concentration within the range from about 20 mg/mL to about 150 mg/mL, a buffer present at a concentration within the range from about 20 mM to about 80 mM, and a tonicifying agent present at a concentration within the range from about 100 mM to about 250 mM, wherein the formulation is characterized by a pH within the range from about 5 to about 8.

Further, the pharmaceutical composition can be provided as a pharmaceutical kit comprising (a) a container containing a IL-36R binding agent (e.g., an anti-IL-36R antibody) in lyophilized form and (b) a second container containing a pharmaceutically acceptable diluent (e.g., sterile water) for injection. The pharmaceutically acceptable diluent can be used for reconstitution or dilution of the lyophilized anti-IL-36R antibody or agent. Optionally associated with such container(s) can be a notice in the form prescribed by a governmental agency regulating the manufacture, use or sale of pharmaceuticals or biological products, which notice reflects approval by the agency of manufacture, use or sale for human administration.

Such combination therapy administration can have an additive or synergistic effect on disease parameters (e.g., severity of a symptom, the number of symptoms, or frequency of relapse).

With respect to therapeutic regimens for combinatorial administration, in a specific embodiment, an anti-IL-36R antibody or IL-36R binding agent is administered concurrently with a therapeutic agent. In another specific embodiment, the therapeutic agent is administered prior or subsequent to administration of the anti-IL-36R antibody or IL-36R binding agent, by at least an hour and up to several months, for example at least an hour, five hours, 12 hours, a day, a week, a month, or three months, prior or subsequent to administration of the anti-IL-36R antibody or IL-36R binding agent.

The invention is further described in the following examples, which are not intended to limit the scope of the invention.

### EXAMPLES

### Example 1: Multi-Center, Double-Blind, Randomised, Placebo-Controlled, Phase IIa Study to Investigate Efficacy, Safety, Tolerability, Pharmacokinetics and Pharmacogenomics of Multiple Intravenous Doses of an Anti-IL-36R Antibody of the Present Invention in Patients with Palmoplantar Pustulosis (PPP)

### Abbreviations

ADA Anti-Drug Antibody
ADCC Antibody-Dependent Cellular Cytotoxicity
AE Adverse Event
AESI Adverse Event of Special Interest
ALT Alanine Aminotransferase
AMP Auxiliary Medicinal Product
API Active Pharmaceutical Ingredient
AST Aspartate Aminotransferase
AUC Area under the Curve
BI Boehringer Ingelheim
BSA Body Surface Area
CDC Complement-Dependent Cytotoxicity
CI Confidence Interval
Cmax Maximum measured concentration of the analyte in plasma
CML Local Clinical Monitor
CRA Clinical Research Associate
CRF Case Report Form
CRO Contract Research Organisation
CTP Clinical Trial Protocol
CTR Clinical Trial Report
DEDP Drug Exposure During Pregnancy
DILI Drug Induced Liver Injury
DLQI Dermatology Life Quality Index
DMC Data Monitoring Committee
DNA Desoxyribo Nucleid Acid
ECG Electrocardiogram
EDTA Ethylendiaminetetraacetic Acid
e.g. Example given
ELISA Enzyme Linked Immunosorbent Assay
EOT End of Trial
EudraCT European Clinical Trials Database
FAS Full Analysis Set
FcRn Neonatal Fc receptor
FIH First-in-human
GCP Good Clinical Practice
GMP Good Manufacturing Practice
GPP Generalized Pustular Psoriasis
HIV Human Immunodeficiency Virus
HV Healthy Volunteer
IB Investigator's Brochure
IBD Inflammatory Bowel Disease
i.e. id est
IEC Independent Ethics Committee
IgG Immunglobulin G
IHC Immunohistochemistry
IL Interleukin
IMP Investigational Medicinal Product
IRB Institutional Review Board
IRT Interactive Response Technology
ISF Investigator Site File
ITE Indirect Target Engagement
i.v. Intravenous
kDA Kilodalton
kg Kilogram
LPDD Last Patient Drug Discontinuation
mAb Monoclonal antibody
MedDRA Medical Dictionary for Drug Regulatory Activities
mg Milligram
mm Millimeter
MMRM Mixed Model Repeated Measures
MoA Mode of action
MRD Multiple rising dose
NCE New chemical entity
NIMP Non-Investigational Medicinal Product
NRI No Response Imputation
OPU Operative Unit
PD Pharmacodynamics
PGA Physicians Global Assessment
PK Pharmacokinetics
PoCC Proof of Clinical Concept
PPP Palmoplantar Pustulosis
PPP PGA Palmoplantar Pustulosis Physicians Global Assessment
PPP ASI Palmoplantar Pustular Psoriasis Area and Severity Index
PROs Patient Reported Outcomes
PUVA Psoralen plus UV-A
RCTC Rheumatology Common Toxicity Criteria
RDC Remote Data Capture
REP Residual effect period, after the last dose of medication with measureable drug levels or pharmacodynamic effects still likely to be present
RNA Ribonucleid Acid
SAE Serious Adverse Event
SAP Statistical Analysis Plan
s.c. subcutaneous
SD Standard Deviation
SOP Standard Operating Procedures
SRD Single rising dose
SUSARs Suspected Unexpected Serious Adverse Reactions
TCM Trial Clinical Monitor
TMDD Target Mediated Drug Disposition
TNF Tumor necrosis factor
TSAP Trial Statistical Analysis Plan
VAS Visual Analog Scale
WBC White Blood Count
WFI Water For Injection
WOCBP Women of Childbearing Potential

### 2.1 Rationale for Performing the Trial

BI 655130 is in development for the treatment of Palmoplantar Pustulosis. The first trial to be conducted in PPP patients is a proof-of-concept, phase IIa trial. The rationale to perform this trial is based on the published human genetic linkage between the target disease PPP and the IL36 pathway targeted by an anti-IL-36R antibody of the present invention, the functional linkage between the IL36 pathway and PPP and the high unmet medical need in PPP.

There is currently no drug specifically approved for the treatment of PPP and it is notoriously difficult to treat. Patients usually end up being treated with the currently available systemic treatment options including retinoids, PUVA, methotrexate, ciclosporine and topical corticosteroids. Unfortunately, the current treatment options are not effective in reducing duration and severity of PPP. Thus, there is high unmet medical need for PPP.

Recently, a FIH trial has been completed (see Section 1.2) which explored safety, tolerability, pharmacokinetics (PK), and pharmacodynamics of an anti-IL-36R antibody of the present invention following i.v. infusions of single rising doses of 0.001 mg/kg up to 10 mg/kg body weight in a healthy male population. The anti-IL-36R antibody of the present invention was safe and well tolerated. All doses higher than 0.001 mg/kg are biologically active, based on the highly sensitive and specific ITE assay (corresponding to the minimum anticipated biological effect level).

A multiple-rising dose, randomized, single-blind, placebo-controlled, phase I study in healthy volunteers is ongoing testing multiple doses of an anti-IL-36R antibody of the present invention up to 10 mg/kg. The objective of this first PPP trial is to evaluate efficacy, safety, tolerability, PK and pharmacogenomics of multiple doses of two dose groups of an anti-IL-36R antibody of the present invention administered to patients with PPP (for rationale of dose selection see Section 4.1.2).

The results from this trial will enable the design of the further developmental program.

### 2.2 Trial Objectives

The primary objective of this trial is to investigate the safety and efficacy of an anti-IL-36R antibody of the present invention in patients with PPP following multiple intravenous administrations of either 900 mg or 300 mg compared to placebo.

Further objectives are the assessment of the pharmacokinetics of an anti-IL-36R antibody of the present invention after multiple dosing in patients with PPP as well as the exploration of pharmacogenomics and the evaluation of surrogate markers (see Section 5.5).

A description of the endpoints to be determined, and the observations along with specific information as how to collect the data for that information, is provided in Section 5.

### 3.1 Overall Trial Design and Plan

This is a randomised, double-blind, placebo-controlled, parallel-design study. This design is appropriate for providing proof-of-concept and assessing the efficacy and safety of an anti-IL-36R antibody of the present invention compared to placebo in patients with PPP.

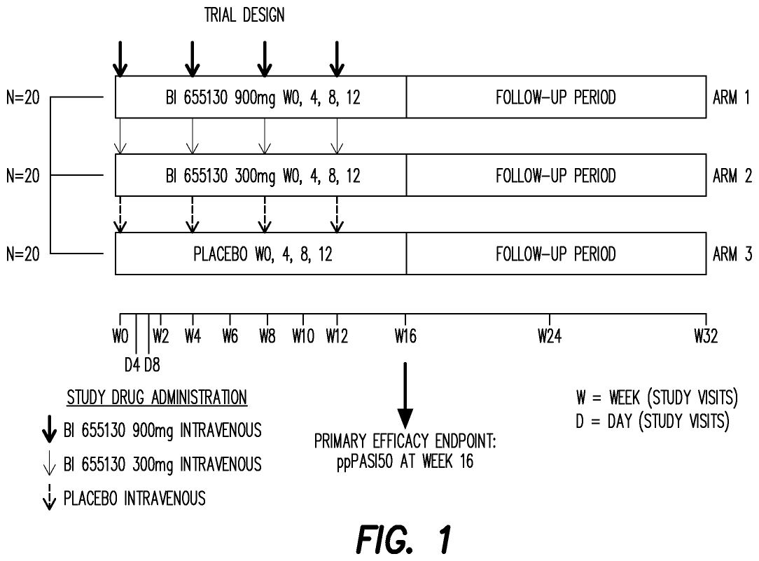

There will be two active dosing arms in this study along with a placebo control arm as shown in FIG. 1.

### 3.3 Selection of Trial Population

### 3.3.1 Main Diagnosis for Trial Entry

The study will be performed in adult patients diagnosed with Palmoplantar Pustulosis defined as presence of primary, persistent (>3 months duration), sterile, macroscopically visible pustules on the palms and/or soles, without or with plaque psoriasis.

A log of all patients enrolled into the trial (i.e. who have signed informed consent) will be maintained in the ISF at the investigational site irrespective of whether they have been treated with investigational drug or not.

Please refer to Section 8.3.1 (Source Documents) for the documentation requirements pertaining to the in- and exclusion criteria.

### 3.3.2 Inclusion Criteria

1. Signed and dated written informed consent in accordance with Good Clinical Practice (GCP) and local legislation prior to the start of any screening procedures.
2. Male or female patients, 18 to 65 years of age at screening.
3. Palmoplantar Pustulosis defined as presence of primary, persistent (>3 months duration), sterile, macroscopically visible pustules on the palms and/or soles, without or with plaque psoriasis on less than 10% of the body surface area.
4. Presence of active pustulation (yellow pustules) on palms and/or soles.
5. A minimum PPP ASI score of 12 and PPP PGA of at least moderate severity at baseline.
6. Women of childbearing potential (WOCBP)₁ and men able to father a child must use highly effective methods of birth control per ICH M3 (R2) that result in a low failure rate of less than 1% per year when used consistently and correctly. A list of contraception methods meeting these criteria is provided in the patient information.

3.3.3 Exclusion Criteria

1. Patients with associated plaque psoriasis ≥10% of the body surface area.
2. Women who are pregnant, nursing, or who plan to become pregnant while in the trial.
3. Severe, progressive, or uncontrolled renal, hepatic, haematological, endocrine, pulmonary, cardiac, neurologic, cerebral, or psychiatric disease, or signs and symptoms thereof.
4. Presence or known history of anti-TNF-induced PPP-like disease.
5. Patients with SAPHO (Synovitis-acne-pustulosis-hyperostosis-osteitis) syndrome.
6. Patient with a transplanted organ (with exception of a corneal transplant >12 weeks prior to screening) or who have ever received stem cell therapy (e.g., Prochymal).
7. Known history of lymphoproliferative disease, including lymphoma, or signs and symptoms suggestive of possible lymphoproliferative disease, such as lymphadenopathy and/or splenomegaly.
8. Any documented active or suspected malignancy or history of malignancy within 5 years prior to the screening visit, except appropriately treated basal or squamous cell carcinoma of the skin or in situ carcinoma of uterine cervix.
9. Patients who have previously undergone allergy immunotherapy for prevention of anaphylactic reactions.
10. Use of any restricted medication as specified in Table 4.2.2.1:1 or any drug considered likely to interfere with the safe conduct of the study, as assessed by the investigator.
11. Plans for administration of live vaccines during the study period or within 6 weeks prior to randomisation.
12. History of allergy/hypersensitivity to a systemically administered biologic agent or its excipients.
13. Active systemic infections during the last 2 weeks (exception: common cold) prior to randomisation, as assessed by the investigator.
14. Chronic or relevant acute infections including human immunodeficiency virus (HIV), viral hepatitis and (or) active or latent tuberculosis (patients with a positive QuantiFERON TB test are excluded. Patients with suspected false positive or undeterminable QuantiFERON TB result may be re-tested).
15. Major surgery performed within 12 weeks prior to randomisation or planned within 32 weeks after randomisation (e.g. hip replacement, aneurysm removal, stomach ligation), as assessed by the investigator.
16. Total white blood count (WBC)<3,000/μL, or platelets <100,000/μL or neutrophils <1,500/μL, or hemoglobin <8.5 g/dL at screening.
17. Aspartate aminotransferase (AST) or alanine aminotransferase (ALT)>2× the upper limit of normal, or total bilirubin >1.5× the upper limit of normal (patients with Gilbert's syndrome are not excluded) at screening.
18. Currently enrolled in another investigational device or drug study, or less than 30 days since ending another investigational device or drug study(s), or receiving other investigational treatment(s).
19. Chronic alcohol or drug abuse or any condition that, in the investigator's opinion, makes them an unreliable study subject or unlikely to complete the trial.
20. Previous randomisation in this trial.

### 4.0 Treatments

### 4.1 Investigational Treatments

The investigational product has been manufactured by BI Pharma GmbH & Co. KG. The anti-IL-36R antibody of the present invention is a heterodimer with a molecular weight of approximately 146 kDa. The anti-IL-36R antibody of the present invention as a drug product is formulated at a concentration of 20 mg/mL. Active Pharmaceutical Ingredient (API) in a buffer consisting of 25 mM sodium citrate, 200 mM sucrose, 0.04% w/v polysorbate 80 at pH 6 and water for injection (WFI). All excipients are of compendium quality (e.g. USP, Ph.Eur.).

### 4.1.1 Identity of the Investigational Medicinal Products

The characteristics of the test product are given below:

TABLE 4.1.1:1
Test product

| | |
|---|---|
| Substance: | BI 655130 |
| Pharmaceutical | IMP concentrate consisting of BI 655130 in a buffer of |
| formulation: | 25 mM sodium citrate, 200 mM sucrose, 0.04% w/v |
| | polysorbate 80 at pH 6 and water for injection. |
| Source: | BI Pharma GmbH & Co. KG, Germany |
| Unit strength: | 150 mg/7.5 mL |
| Posology: | 900 mg or 300 mg every 4 weeks at |
| | Day 1. 29, 57 and 85. |
| Route of Administration: | i.v. infusion |
| Duration of Use: | 12 weeks |

TABLE 41.1:2
Placebo

| | |
|---|---|
| Substance | Placebo |
| | A buffer of 25 mM sodium citrate, 200 mM sucrose. |
| Pharmaceutical | 0.04% w/v polysorbate 80 at pH 6 and water for |
| formulation | injection. |
| Source: | BI Pharma GmbH & Co. KG, Germany |
| Unit strength: | 0 mg/7. 5 mL |
| Posology | 0 mg every 4 weeks at day 1,29,57 and 85. |
| Route of Administration | iv. infusion |
| Duration of Use | 12 weeks |

### 4.1.2 Selection of Dose in the Trial

The doses of 300 mg and 900 mg for this trial were selected on the basis of data obtained in the completed SRD trial 1368.1 and the ongoing MRD trial 1368.2. In these trials the clinical safety and tolerability profile of the anti-IL-36R antibody of the present invention has been tested and found favourable (safe and well tolerated) in male healthy volunteers treated with i.v. single doses up to 20 mg/kg or multiple doses up to 10 mg/kg body weight once a week for up to 4 weeks. There were no dose limiting adverse events, in particular no signs of infusion reactions.

Under the assumption of an increasing exposure/response relationship, the highest dose schedule leading to an exposure that is safe and tolerable is expected to provide the best chance to show clinical efficacy and achieve a positive proof-of-clinical-concept. To maximize the chance for a positive efficacy signal and treatment benefit for difficult to treat PPP patients, it is proposed given the current excellent safety profile of this compound to study as high i.v. dose the dose of 900 mg (via every 4 weeks dosing) in this proof of concept study. The lower i.v. dose of 300 mg has been selected as it would allow, if positive, to proceed with sub-cutaneous (s.c.) dose regimen for treatment of PPP in further development.

A fixed-dose regimen has been selected as a fixed dose is standard for most current biologic treatments due to major advantages for healthcare professionals and patients include dosing simplicity which reduces the risk of dosing errors. Bodyweight (and other covariates impacting exposure) are likely to have a diminished impact assuming dosing at the higher end of the exposure-efficacy response. Furthermore, monoclonal antibodies are highly targetspecific and offer a relatively large therapeutic window as compared to new chemical entities (NCEs). Therefore, most monoclonal antibodies are approved at fixed doses in antibody/target excess in order to cover target turnover and maximize efficacy.

Body weight has been included in the current PK model as a covariate indicating decreased exposure with increasing body weight. The current model indicates that body weight explains less than 15% of between-subject variability in PK of BI 655130 when comparing a model with and without body-weight as a covariate of exposure. A fixed dose regimen will minimize the potential for dosing errors due to less complex dose calculation, study drug preparation and administration as compared with weight based dosing. It will also facilitate dose finding and PK-PD analyses due to covering a wider weight/exposure range.

Based on PK modelling informed by 1368.1, the exposures of the anti-IL-36R antibody of the present invention predicted in this trial are expected to only slightly exceed exposures tested and found safe in healthy volunteers (HV). For the 900 mg administered every 4 weeks at time 0, weeks 4, 8 and 12, the highest maximum measured concentration of the analyte in plasma (Cmax) and average concentrations within inter-dosing period of the 900 mg regimen will not exceed the Cmax with the 10 mg/kg regimen tested in 1368.2. The total (cumulative) Area under the Curve (AUC) assessed over 35 weeks is predicted to be 25% above the exposure levels expected with 10 mg/kg once a week (in 1368.2) over the same period. Of note, the current PK model based on data from the SRD study (1368.1) appears in agreement with preliminary, overlaid data for the 3 and the 6 mg/kg cohorts of 1368.2 supporting its use for SRD to MRD extrapolations (see current version of the Investigator's Brochure, c03320877). Importantly, these predictions are made based upon comparable exposures between HV and PPP patients, and using a 75 kg reference individual. For a 90 kg individual the projected total exposure (cumulative AUC) will decrease by 46%. The weight expectation for PPP patients is approximately 80 kg [R17-0364]. However, any safety risk of dosing such patients will likely be limited by expected lower systemic exposures in PPP patients compared to healthy subjects, presumably due to higher expression of the target molecule in diseased tissues as compared to peripheral blood of healthy subjects. Higher target expression may increase the target-mediated drug disposition component, contributing to increased clearance of the anti-IL-36R antibody of the present invention.

### 4.2 Other Treatments, Emergency Procedures, Restrictions

### 4.2.1 Other Treatments and Emergency Procedures

### 4.2.1.1 Rescue Medication

The use of a rescue medication will be left at the discretion of the investigator and should be based on the severity and progression of the disease. It is recommended to wait until at least four weeks after the study drug administration (week 16) before prescribing a rescue medication in case no improvement or no change in disease condition is observed (stable disease). In case a rescue medication is prescribed, the patient will stay in the trial and will be followed-up as initially planned until week 32 (End of Trial Visit). The sponsor will not supply the sites with the rescue medication.

### 4.2.1.2 Emergency Procedures

In case of infusion reactions emerging during or after infusion of study drug, the investigator should consider in accordance with severity of the reaction and local standard of care to
Immediately interrupt the infusion
Treat with systemic anti-histamines and intravenous steroids

Based on patient's clinical course and medical judgment, the infusion may be re-initiated in case of mild or moderate reactions (according to RCTC grading in ISF) at lower speed with gradual increase to complete the infusion as detailed in the Instructions for Preparation and Handling of the anti-IL-36R antibody of the present invention/placebo in the Investigator Site File.

### 4.2.1.3 Additional Treatments

No additional treatment is planned. However, in case of adverse events in need of treatment, the investigator can authorize symptomatic therapy. In those cases, patients will be treated as necessary and, if required, kept under supervision at the trial site or transferred to a hospital until all medical evaluation results have returned to an acceptable level.

Background therapy is not allowed throughout the trial.

### 4.2.2 Restrictions

### 4.2.2.1 Restrictions Regarding Concomitant Treatment

The medications (or classes of medications) listed in Table 4.2.2.1:1 must not be taken for the time periods as specified.

TABLE 42.2.1:1
Restricted Medications

| | Restriction duration (through Primary |
|---|---|
| Medication or class of medications | Endpoint Visit at Week 16)¹ |
| IL36R inhibitors other than the study drug | not allowed neither before nor during |
| | trial participation |
| Secukinumab (Cosentyx ®), ustekinumab | 12 weeks or 5 half-lives, whichever is |
| (Stelara ®), guselkumab, ixekizumab, | greater. prior to randomisation |
| tildrakzumab, brodahmab | |
| Adalimumab. infliximab | |
| Natalizumab or agents that deplete B or T | |
| cells (e.g, rituximab, alemtuzumab or | |
| visilizumab) | |
| Investigational products for psoriasis | |
| | Restriction duration (through Primary |
| Medication or class of medications | Endpaint Visit at Week 16)² |
| Etanercept | 6 weeks prior to randomisation |
| Live virus vaccinations⁴ | |
| Other systemic immunomodulating treatments | 4 weeks prior to randomisation |
| (e.g. corticosteroids², methotrexate, fumaric | |
| acid esters, acitretin, ciclosporin, apremilast | |
| Any investigational device or product (excludes | |
| psoriasis products) | |
| Phototherapy (e.g., UVA, UVB), topical | 14 days prior to randomisation. |
| treatment for psoriasis or any other skin | |
| condition (e.g. corticosteroids³, vitamin D | |
| analogues, salicylic acid, tar, anthralin) | |
| Anakinra | 7 days prior to randomization |

¹ In case of worsening of the PPP and/or psoriasis. the use of a rescue medication is left at the discretion of the investigator (refer to Section (4.2.142); In case of any other acute indication after the Primary Endpoint Visit at Week 16, the use of restricted medication is permitted.
²There is no restriction on corticosteroids with only a topical effect (e.g. inhaled corticosteroids to treat asthma or corticosteroids drops administered in the eye or ear).
3Exception: topical steroids of US class 6 (mild, such as desonide) or US class 7 (least potent. such as hydrocortisone) for use on the face, axilla, and or genitalia with a restriction of use within 24 hours prior to trial visit in which ppPASI is assessed.
⁴Live virus vaccination should be restricted until the end of the trial.

In the event a patient with prior use of systemic steroids, TNFa inhibitors, IL17/IL12/23 inhibitors, or anakinra is enrolled, past medical records are required to document when these treatments were stopped. All concomitant or rescue therapies will be recorded (including time of intake and dose on study days) on the appropriate pages of the CRF.

### 5.1 Trial Endpoints

### 5.1.1 Primary Endpoint(s)

Efficacy: PPP ASI50 at week 16
Safety: Number of patients with drug-related AEs

### 5.1.2 Secondary Endpoint(s)

Treatment success defined as achieving a clinical response of 0 or 1=clear/almost clear via PPP Physicians Global Assessment (PPP PGA) at week 16
PPP ASI75 at week 16
Percent change from baseline in the PPP ASI at week 16

### 5.1.3 Further Endpoint(s)

Change from baseline in Pain Visual Analog Scale (VAS) score at Week 16 and all other visits collected
Clinical Improvement assessed via Dermatology Life Quality Index (DLQI) at week 16 and all other visits collected compared to baseline.
PPP ASI50 at all other visits collected
Modified (precise) PPP ASI scores at week 16 and all other visits collected
Treatment success defined as achieving a clinical response of 0 or 1=clear/almost clear via PPP Physicians Global Assessment (PPP PGA) at all other visits collected
PPP ASI75 at all other visits collected
Percent change from baseline in the PPP ASI at all other visits collected
Time (days) to achieving PPP ASI50
Time (days) to loss of PPP ASI50
Change in plaque psoriasis BSA involvement at week 16 in patients with concurrent plaque psoriasis at baseline
Adverse reactions (including drug-related AEs)

### 5.2 Assessment of Efficacy

### Palmoplantar Pustulosis Physician Global Assessment (PPP PGA)

PPP PGA relies on clinical assessment of the patient's skin presentation on the palms and soles and will be measured at the timepoints scheduled in the Flow Chart. The investigator (or qualified site personnel) scores the lesions on the most severely affected palmoplantar surface from 0-4 as clear, almost clear, mild, moderate or severe (cf Table 5.2:1). Further practical guidance will be available in the ISF.

TABLE 52:1
PPP Physician Global Assessment (pppPGA)

| Score | Wording | Detailed description |
|---|---|---|
| 0 | Clear | No signs of PPP: no scaling or crusts or pustule remains |
| 1 | Almost clear | Slight scaling and/or erythema and/or slight crusts; very few new |
| | | (yellow) and/or old (brown) pustules |
| 2 | Mild | Scaling and/or erythema and/or crusts; visible new (yellow) and'or |
| | | old (brown) pustules of limited number and extent |
| 5 | Moderate | Prominent scaling and/or erythema and/or crusting; prominent |
| | | new (yellow) and/or old (brown) pustules covering |
| | | most of the area involved |
| 4 | Severe | Severe scaling and/or erythema and/or crusting; numerous new |
| | | (yellow) or old (brown) pustules with and/or without major conflence |
| | | covering the entire area of at least 2 palmoplantar surfaces |

### Palmoplantar Pustulosis Psoriasis Area and Severity Index (PPP ASI)

The PPP ASI is an investigator assessment of the extent and severity of pustular and plaque lesions on the palms and soles presenting in PPP patients. The adaptation from PASI, an established measure of severity and area of psoriatic lesions in patients with psoriasis, by Bhushan et. al will be used in this trial (cf Table 5.2:2).

This tool provides a numeric scoring for patients overall PPP disease state, ranging from 0 to 72. It is a linear combination of the percent of surface area of skin that is affected on the palms and soles of the body and the severity of erythema, pustules, and scaling (desquamation).

The PPP ASI will be measured at the timepoints scheduled in the Flow Chart.

TABLE 5.2:2
Palmoplantar Pustulosis Psoriasis Area and Seventy Index

| Score | 0 | 1 | 2 | 3 | 4 | 5 | 6 |
|---|---|---|---|---|---|---|---|
| Erythema (E) | None | Slight | Moderate | Severe | Very | | |
| | | | | | severe | | |
| Pustules (P) | None | Slight | Moderate | Severe | Very | | |
| (total) | | | | | severe | | |
| Desquamation | None | Slight | Moderate | Severe | Very | | |
| (D) (scaling) | | | | | severe | | |
| Area affected | 0 | <10 | 10 < 30 | 30 < 50 | 50 < 70 | 70 < 90 | 90-100 |
| (%)* | | | | | | | |

*where area assessed is glabrous skin on the palms/soles

(right sole)]+[(E+P+D)Area×0.3(left sole)]

Additionally, a modified PPP ASI score (precise PPP ASI) will be calculated based on the absolute number/percent affected area in addition to the ranges used in the original scale.

### Plaque Psoriasis Body Surface Area (BSA) Involvement

For the patients with concurrent plaque psoriasis, the percent body surface area (BSA) involved with plaque-type psoriasis lesions will be captured at time points indicated in the Flow Chart.

### Pain VAS

The pain VAS is a unidimensional measure of pain intensity. It is a continuous scale comprised of a horizontal line, anchored by word descriptors at the end (“no pain”, “severe pain”). It is divided into 10 equidistant segments by vertical marks labelled “0”, “1”, . . . “10”. The pain VAS is self-completed by the patient at visits indicated in the Flow Chart. The patient is asked to place an (X) at the point on the horizontal line that represents their pain intensity. Using a ruler, the score is determined by measuring the distance (mm) on the line between the “no pain” anchor and the patient's mark, divided by the overall length of the scale (mm), and multiplied by 100, providing a range of scores from 0-100. A higher score indicates greater pain intensity.

### 7.3 Planned Analyses

The efficacy analyses will be performed for the FAS which is based on the intent-to-treat principle, and comprises all participants who were randomised, received at least one dose during the trial, and had a baseline measurement for the primary endpoint. Efficacy analyses will be based on the planned treatment (i.e., the treatment assigned at randomisation). Safety analyses on patients who were randomised and received at least one dose during the trial will be based on the actual treatment received at the randomisation visit; this set of patients is called the Safety Analysis Set (SAF). All efficacy analyses will be conducted on the FAS. All safety analyses will be conducted on the SAF.

Important violations of the protocol will include key inclusion and exclusion violations, incorrect medications taken, compliance with study medication, concomitant use of restricted medications, and any other violations of the protocol deemed important by the study team. All decisions concerning important protocol violations will be made prior to un-blinding of the database for the final week 16 analysis. A per-protocol set (PPS) will be defined as a subset of the FAS which excludes all patients with a violation that potentially affects the Week 16 efficacy assessment.

Standard statistical parameters (number of non-missing values, mean, standard deviation (SD), median, quartiles, minimum and maximum) or frequency tables (including patient frequencies and percentages) will be calculated where appropriate.

For continuous secondary or further endpoints, mean changes from baseline will be analysed using a restricted maximum likelihood (REML)-based repeated measures approach. Analyses will include the fixed, categorical effects of treatment and visit, presence or absence of plaque psoriasis (yes/no), as well as the treatment-by-visit interaction, and continuous, fixed covariates of baseline “endpoint” and baseline-by-visit interaction. An unstructured covariance structure will be used to model the within-patient measurements. Exploratory confidence intervals will be based on least-squares mean differences to Placebo using a two sided α=0.05.

This is an exploratory trial and formal confirmatory statistical testing will not be performed.

### 7.3.1 Primary Endpoint Analyses

The achievement of PPP ASI50 at Week 16 is the primary endpoint in this trial and represents a binary variable with values of 0 (=non-response) or 1 (=response). Prior to treatment unblinding for the optional week 16 interim analysis, it may be decided to use the PPP ASI75 as the primary endpoint (instead of PPP ASI50 which will then be considered as a secondary endpoint); if applicable, such decision will be documented in the Trial Statistical Analysis Plan (TSAP).

The primary analysis of the unadjusted absolute risk difference versus Placebo will be calculated simply as the difference in the observed proportion of patients with PPP ASI50 at week 16 for each treatment scenario, for the FAS. A 95% Wilson confidence interval around this difference will also be provided. In addition, a parametric bootstrap 95% confidence interval will be generated by sampling from the binomial distribution on each treatment with number of patients and observed proportion of responders per treatment representing the sampling parameters. A hierarchical approach to the testing of both scenarios for the anti-IL-36R antibody of the present invention versus Placebo will, however, be performed for the primary analysis in order to control for multiplicity arising as a result of the multiple treatment comparisons.

Exploratory analyses of the primary endpoint will include, in the absence of model convergence issues due to occurrence of low cell frequencies, the difference in the proportion of patients with a PPP ASI50 between the anti-IL-36R antibody of the present invention and placebo being analysed, for the FAS, using a logistic regression approach with a logit link via PROC LOGISTIC in SAS®. Fixed classification effects will include treatment and presence or absence of plaque psoriasis (yes/no). A test for difference between treatments will be performed using the likelihood ratio test. The fitted logistic regression model will be used to predict the response rate, under the anti-IL-36R antibody of the present invention and placebo, for each patient in the trial [R16-5360] and the resulting difference in the average probability of response between treatments will give the risk difference for the anti-IL-36R antibody of the present invention versus placebo. The delta method will then be used to calculate the standard error and associated 95% confidence intervals around the adjusted risk difference estimates. If, however, model convergence issues do occur due to occurrence of low cell frequencies, then an exact approach may be used instead.

Other analyses of the primary endpoint will include:
Sensitivity analyses utilizing different patients sets (such as the PPS), as well as alternative methods for the handling of missing data as described in Section 7.5;
Exploration of the relationship between various demographic or baseline characteristics data and the primary endpoint will be performed via graphical methods as well as using a logit link with PROC LOGISTIC in SAS®

Further details will be provided in the TSAP.

### 7.3.2 Secondary Endpoint Analyses

For the secondary binary endpoints, for the FAS, the unadjusted absolute risk difference versus Placebo will be calculated and a 95% Wilson confidence interval around this difference will also be provided. In addition, a parametric bootstrap 95% confidence interval will also be generated.

For secondary continuous endpoints, mean changes from baseline will be analysed using a restricted maximum likelihood (REML)-based repeated measures approach (see Section 7.3).

### 7.3.3 Further Endpoint Analyses

Further endpoints will be analysed using the methods described above for the primary and secondary endpoint analyses. For time to event endpoints, such as the time to achieving PPP ASI50, KM-estimates of the survival/failure probabilities at monthly intervals, as well as the median time-to-event will be provided. Confidence intervals will be based on two-sided α=0.05.

Questionnaires such as the DLQI will be descriptively summarized by visit.

### 7.3.4 Safety Analyses

The safety set, described in Section 7.3, will be used to perform all safety analysis. In general, safety analyses will be descriptive in nature and will be based on BI standards. No hypothesis testing is planned.

Statistical analysis and reporting of adverse events will concentrate on treatment-emergent adverse events. To this end, all adverse events occurring between start of treatment and end of the residual effect period will be considered ‘treatment-emergent’. The REP is defined as 20 weeks after the last dose of trial medication. Adverse events that start before first drug intake and deteriorate under treatment will also be considered as ‘treatment-emergent’. Drug related AEs will be tabulated by system organ class and preferred term after coding according to the current version of the Medical Dictionary for Drug Regulatory Activities (MedDRA).

In addition, the frequency, severity, and causal relationship of adverse events will be tabulated by system organ class and preferred term after coding according to the current version of MedDRA.

Laboratory data will be analysed both quantitatively as well as qualitatively. The latter will be done via comparison of laboratory data to their reference ranges. Values outside the reference range as well as values defined as clinically relevant will be highlighted in the listings. Treatment groups will be compared descriptively with regard to distribution parameters as well as with regard to frequency and percentage of patients with abnormal values or clinically relevant abnormal values.

Vital signs, physical examinations, or other safety-relevant data observed at screening, baseline, during the course of the trial and at the end-of-trial evaluation will be assessed with regard to possible changes compared to findings before start of treatment.

Following the administration of the anti-IL-36R antibody (e.g., an anti-IL-36R antibody of the present invention), safety and efficacy assessments reveal the followings: At least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the patients achieve clinical remission as defined by (a) Psoriasis Area and Severity Index for PPP (PPP ASI) 75 at Week 16; (b) the proportion of patients with an adverse event (AE) in response to the administration is statistically the same or lower as compared to patients on placebo for one or more of end points; (c) PPP Physicians Global Assessment (PPP PGA) score of 0 or 1 (clear/almost clear) at Week 16; (d) Psoriasis Area and Severity Index for PPP (PPP ASI) 75 at Week 16; (e) Change from baseline in the PPP ASI at week 16; (f) Change from baseline in Pain Visual Analog Scale (VAS) score at Week 16; (g) Clinical Improvement assessed via Dermatology Life Quality Index (DLQI) at week 16; (h) PPP ASI50 at all other visits collected; (i) Modified (precise) PPP ASI scores at week 16 and all other visits collected; (j) PPP Physicians Global Assessment (PPP PGA) score of 0 or 1 (clear/almost clear) at all other visits collected; (k) PPP ASI75 at all other visits collected; (l) Percent change from baseline in the PPP ASI at all other visits collected; (m) Time (days) to achieving PPP ASI50; (n) Time (days) to loss of PPP ASI50; (o) Change in plaque psoriasis BSA involvement at week 16 in patients with concurrent plaque psoriasis at baseline. The proportion of patients with a response to the administration is statistically higher as compared to patients on placebo for one or more of end points (a)-(o).

### Example 2: Treating Patients with Acute PPP Flares (Including New Appearance or Worsening of Pustules)

In this example, an anti-IL36R antibody (e.g., an anti-IL-36R antibody of the present invention) is used to treat patients with PPP. The outcome measured, mode of administration and inclusion/exclusion criteria in this example are as follows:

### Primary Outcome (Endpoint) Measures:

PPP ASI50 at week 16;
Percent reduction in pustule severity as compared to baseline;
Number of patients with drug-related AEs;
Superior efficacy over guselkumab; and/or the primary endpoints listed in Example 1.

### Secondary Outcome (Endpoint) Measures:

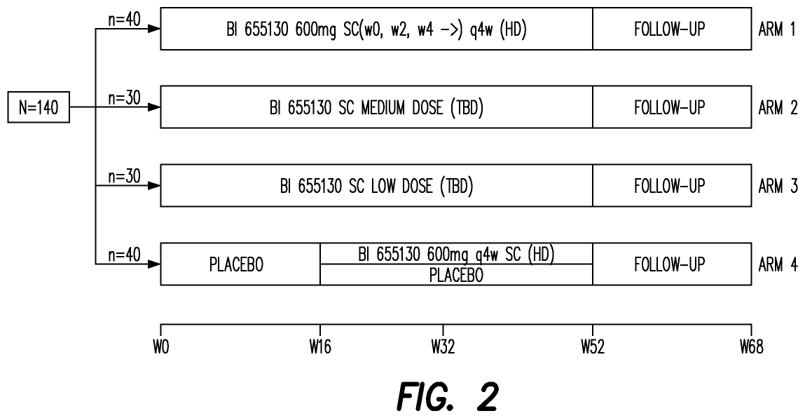

Treatment success defined as achieving a clinical response of 0 or 1=clear/almost clear via PPP Physicians Global Assessment (PPP PGA) at week 16;
PPP ASI75 at week 16;
Percent change from baseline in the PPP ASI at week 16;
At least 40% superior to placebo in achievement of PPP ASI50 at week 16; and/or secondadry endpoints listed in Example 1.
   Mode of Administration: SC, see also Tables 1-4; see also FIG. 2 for the study design.

### Inclusion Criteria:

Similar the inclusion criteria provided Example 1 but with the following modification/refinement:
Diagnosis of palmoplantar pustulosis=primary, persistent (>3 months duration), sterile, macroscopically visible pustules on the palms and/or soles, with or without plaque psoriasis
Pustular score 2 at screening and baseline
PPP ASI score of 12 at screening and baseline
PPP PGA of at least 3 at screening and baseline
Presence of active pustulation (white or yellow pustules) on palms and/or soles

### Exclusion Criteria:

Similar the exclusion criteria provided Example 1 but with the following modification/refinement:
Patients with known history of anti-TNF inhibitor-induced PPP-like disease
Improvement during screening (5 PPP ASI total score improvement during the screening period)

Following the administration of the anti-IL-36R antibody (e.g., an anti-IL-36R antibody of the present invention), data assessment reveals the followings: At least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the patients achieve clinical remission as defined by (a) Psoriasis Area and Severity Index for PPP (PPP ASI) 50 at Week 16; (b) Pustule severity compared to baseline; For example, on average, patients experience at least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more improvement in their pustule severity as compared to baseline; (c) Superior efficacy over guselkumab; e.g., historical data indicating that a compound or product of the present invention has superior efficacy over guselkumab by at least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more (d) PPP Physicians Global Assessment (PPP PGA) score of 0 or 1 (clear/almost clear) at Week 16; (d) Psoriasis Area and Severity Index for PPP (PPP ASI) 75 at Week 16; (e) Change from baseline in the PPP ASI at week 16; (f) Change from baseline in Pain Visual Analog Scale (VAS) score at Week 16; (g) Clinical Improvement assessed via Dermatology Life Quality Index (DLQI) at week 16; (h) PPP ASI50 at all other visits collected; (i) Modified (precise) PPP ASI scores at week 16 and all other visits collected; (j) PPP Physicians Global Assessment (PPP PGA) score of 0 or 1 (clear/almost clear) at all other visits collected; (k) PPP ASI75 at all other visits collected; (l) Percent change from baseline in the PPP ASI at all other visits collected; (m) Time (days) to achieving PPP ASI50; (n) Time (days) to loss of PPP ASI50; (o) Change in plaque psoriasis BSA involvement at week 16 in patients with concurrent plaque psoriasis at baseline; (P) Being at least about 40% superior to placebo in achiving PPP ASI50 at week 16. The proportion of patients with a response to the administration is statistically higher as compared to patients on placebo for one or more of end points (a)-(p). In addition, the proportion of patients with an adverse event (AE) in response to the administration (of a compound or product of the present invention) is statistically the same or lower as compared to patients on placebo for one or more of end points (a)-(p).

### Example 3: Treating Patients with Acute PPP Flares (Including New Appearance or Worsening of Pustules)

In this example, an anti-IL36R antibody (e.g., an anti-IL-36R antibody of the present invention) is used to treat patients with acute PPP flares (including new appearance or worsening of pustules).

Initially, each patient has one or more inclusion criteria listed in Example 1. A dose regimen according to those listed in Tables 1-4 is administered to each patient.

Following the administration of the anti-IL-36R antibody (e.g., an anti-IL-36R antibody of the present invention), safety and efficacy assessments reveal the followings: At least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the patients achieve clinical remission as defined by (a) Psoriasis Area and Severity Index for PPP (PPP ASI) 75 at Week 16; (b) the proportion of patients with an adverse event (AE) in response to the administration is statistically the same or lower as compared to patients on placebo for one or more of end points; (c) PPP Physicians Global Assessment (PPP PGA) score of 0 or 1 (clear/almost clear) at Week 16; (d) Psoriasis Area and Severity Index for PPP (PPP ASI) 75 at Week 16; (e) Change from baseline in the PPP ASI at week 16; (f) Change from baseline in Pain Visual Analog Scale (VAS) score at Week 16; (g) Clinical Improvement assessed via Dermatology Life Quality Index (DLQI) at week 16; (h) PPP ASI50 at all other visits collected; (i) Modified (precise) PPP ASI scores at week 16 and all other visits collected; (j) PPP Physicians Global Assessment (PPP PGA) score of 0 or 1 (clear/almost clear) at all other visits collected; (k) PPP ASI75 at all other visits collected; (l) Percent change from baseline in the PPP ASI at all other visits collected; (m) Time (days) to achieving PPP ASI50; (n) Time (days) to loss of PPP ASI50; (o) Change in plaque psoriasis BSA involvement at week 16 in patients with concurrent plaque psoriasis at baseline. The proportion of patients with a response to the administration is statistically higher as compared to patients on placebo for one or more of end points (a)-(o).

In an embodiment related to this example, the administration includes administering or having administered to the patient a therapeutically effective amount of the anti-IL-36R antibody subcutaneously. In a related embodiment, the subcutaneous administration comprises administration of 300 mg or 600 mg dose of the anti-IL-36R antibody. In a related embodiment, the subcutaneous administration is conducted at qw (once every week), q2w (once every 2 weeks), q4w (once every 4 weeks), q6w (once every 6 weeks) or q8w (once every 8 weeks) interval, or a combination thereof.

In an embodiment related to this example, the administration includes administering or having administered to the patient a therapeutically effective amount of the anti-IL-36R antibody subcutaneously or intravenously. In a related embodiment, the initial intravenous administration comprises administration of 600 mg, 750 mg or 900 mg dose of the anti-IL-36R antibody. In a related embodiment, the initial intravenous administration is conducted once at week 0 or twice at weeks 0 and 2. In a related embodiment, the initial subcutaneous administration comprises administration of 750 mg or 900 mg dose of the anti-IL-36R antibody. In a related embodiment, the initial subcutaneous administration is conducted once at week 0 or twice at weeks 0 and 2. In a related embodiment, the initial intravenous or subcutaneous administration is followed by a subsequent subcutaneous administration. In a related embodiment, the subsequent subcutaneous administration comprises administration of 300 mg or 600 mg dose of the anti-IL-36R antibody. In a related embodiment, the subsequent subcutaneous administration is conducted at q4w or q8w interval, or a combination thereof. In a related embodiment, a first dose of the subsequent subcutaneous administration is administered in 2 to 4 weeks after a last dose the initial intravenous or subcutaneous administration.

### Example 4: Preventing Flares from Recurring in PPP Patients

In this example, A dose regimen (according to Tables 1-4) of an anti-IL36R antibody of the present invention is used to present PPP flares from recurring. Subsequent to the administration, as shown in Tables 1-4, one or more doses of the anti-IL36R antibody are administered to prevent the PPP flares from recurring.

Following the administration of the anti-IL-36R antibody (e.g., an anti-IL-36R antibody of the present invention), at least 10%, 20%, 30%, 40%, 50%, 60%, 70% or 80% of the patients remain in clinical remission as measured by a change in PPP ASI from baseline at Week 12, 16, 24, 36, 48, 60 or 72. The improved effects are maintained at higher percentage with an anti-IL-36R antibody of the present invention than with placebo. At least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the mammals or patients maintain improved effects at Week 12, 16, 24, 36, 48, 60 or 72 after the last dose of the anti-IL-36R is administered, as compared to placebo.

Following the administration of the anti-IL-36R antibody (e.g., an anti-IL-36R antibody of the present invention), at least 10%, 20%, 30%, 40%, 50%, 60%, 70% or 80% of the patients remain in clinical remission as measured by a PPP PGA score of 0 or 1 at Week 12, 16, 24, 36, 48, 60 or 72. The improved effects are maintained at higher percentage with an anti-IL-36R antibody of the present invention than with placebo. At least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the mammals or patients maintain improved effects at Week 12, 16, 24, 36, 48, 60 or 72 after the last dose of the anti-IL-36R is administered, as compared to placebo.

Following the administration of the anti-IL-36R antibody (e.g., an anti-IL-36R antibody of the present invention), at least 10%, 20%, 30%, 40%, 50%, 60%, 70% or 80% of the patients remain in clinical remission as measured by a change in PPP ASI pustule, erythema or scaling severity subscore from baseline at Week 12, 16, 24, 36, 48, 60 or 72. The improved effects are maintained at higher percentage with an anti-IL-36R antibody of the present invention than with placebo. At least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the mammals or patients maintain improved effects at Week 12, 16, 24, 36, 48, 60 or 72 after the last dose of the anti-IL-36R is administered, as compared to placebo.

In an embodiment related to this example, the administration includes administering or having administered to the patient a therapeutically effective amount of the anti-IL-36R antibody subcutaneously. In a related embodiment, the subcutaneous administration comprises administration of 300 mg or 600 mg dose of the anti-IL-36R antibody. In a related embodiment, the 300 mg or 600 mg dose is administered qw (once every week), q2w (once every 2 weeks), q4w (once every 4 weeks), q6w (once every 6 weeks) or q8w (once every 8 weeks), or a combination thereof. In a related embodiment, the subcutaneous administration comprises an initial dose. In a related embodiment, the subcutaneous administration further comprises a subsequent dose. In a related embodiment, the initial dose is 150 mg, 300 mg or 600 mg. In a related embodiment, the initial dose of 150 mg or 300 mg is administered per day (in consecutive days) for two weeks. In a related embodiment, the initial dose of 600 mg is administered once per week for two weeks including weeks 0 and 1; weeks 0 and 2; weeks 0 and 3; or weeks 0 and 4. In a related embodiment, the initial dose of 600 mg is administered once per week for three weeks including weeks 0, 1 and 2; weeks 0, 1 and 3; weeks 0, 1 and 4; weeks 0, 2 and 3; weeks 0, 2 and 4; or weeks 0, 3 and 4. In a related embodiment, the initial dose of 600 mg is administered once per week for four weeks including weeks 0, 1, 2 and 3; weeks 0, 1, 2 and 4; weeks 0, 1, 3 and 4; or weeks 0, 2, 3 and 4. In a related embodiment, the initial dose of 600 mg is administered twice per week for 2 weeks. In a related embodiment, the initial dose of 600 mg is administered twice per week for 3 weeks. In a related embodiment, the initial dose of 600 mg is administered twice per week for 4 weeks. In a related embodiment, the subsequent dose is 300 mg or 600 mg. In a related embodiment, the subsequent dose administration begins two to four weeks after the initial dose administration ends. In a related embodiment, the subsequent dose of 300 mg or 600 mg is administered q2w (once every 2 weeks), q4w (once every 4 weeks), q6w (once every 6 weeks) or q8w (once every 8 weeks).

### Example 5. IL-36 Receptor Inhibition for Treatment of Palmoplantar Pustulosis (Results of the Trial Described in Example 1)

An antibody of the present invention, i.e. an anti-IL-36 receptor (IL-36R) antibody (spesolimab [BI 655130]), is a humanized antagonistic monoclonal IgG1 antibody that blocks human IL-36R signaling. Binding of an antibody of the present invention to IL-36R is anticipated to prevent the subsequent activation of IL-36R by cognate ligands (IL36 α, β and γ) and downstream activation of proinflammatory pathways with the aim to reduce epithelial cell/fibroblast/immune cell-mediated inflammation and interrupt the inflammatory response that drives pathogenic cytokine production in palmoplantar pustulosis (PPP).

Preclinical profiles of an antibody of the present invention and clinical data from trials with healthy volunteers and patients with generalized pustular psoriasis (GPP) suggest that an antibody of the present invention is safe, tolerable and may address an unmet medical need in patients with PPP.

### Background

PPP is a chronic, inflammatory, relapsing disease characterised by neutrophil-filled sterile pustules involving the palms and soles. PPP is a debilitating disorder that significantly affects patients' quality of life and can result in functional disability; pustulation severity is a major contributing factor to this. Dysregulated proinflammatory pathways involving IL-36 are thought to be involved in the pathogenesis of PPP. This multicentre, double-blind, randomised, placebo-controlled, Phase IIa study (NCT03135548) investigated the efficacy and safety of an antibody of the present invention in patients with PPP.

### Methods

Adults with PPP with a minimum PPP Area and Severity Index (PPP ASI) score of 12, PPP Physician Global Assessment (PPP PGA) of 3, and visible pustules on the palms/or soles at baseline (N=59) were randomised to one of two treatment arms of an antibody of the present invention (900 or 300 mg intravenously Q4W, up to Week 12) or placebo. The primary endpoints were 50% improvement in PPP ASI (PPP ASI50) at Week 16 and occurrence of drug-related adverse events (AEs). All patients were followed up to Week 32.

### Results

At baseline (prior to initiation of treatment), PPP disease characteristics were generally comparable across treatment arms. Overall, the mean (standard deviation [SD]) time since first diagnosis was 9.1 (11.3) years; in the placebo arm, it was slightly shorter (6.7 years) than in treatment arms of the antibody of the present invention (10.4 years). The mean (SD) baseline PPP ASI was 18.6 (6.3) overall and 16.9 (4.3), 20.3 (6.4), and 18.5 (7.6) in the 900 mg, 300 mg dose arms of the antibody of the present invention and placebo arms, respectively. In the overall population, the proportion of patients achieving PPP ASI50 at Week 16 was not statistically different for the treatment arms of an antibody of the present invention (900 mg or 300 mg) and placebo arms (31.6%, 31.6% vs 23.8%). A post-hoc subgroup analysis comparing patients with baseline PPP ASI above versus below the median (16.7) revealed a rapid improvement with an antibody of the present invention over placebo for total PPP ASI and specifically pustular severity (part of the PPP ASI score) in patients with moderate-to-severe disease above the median. In these patients, the mean (90% confidence interval [CI]) percent change from baseline at Week 16 in PPP ASI was −39.7% (−58.2%, −21.2%) and −23.7% (−42.1%, −5.2%) in the 900 mg (n=8) and 300 mg (n=10) arms for an antibody of the present invention, respectively, versus −8.0% (−35.0%, 19.0%) in the placebo (n=10) arm; the mean (90% CI) pustulation score percent change from baseline at Week 16 was −56.9% (−81.6%, −32.1%) and −29.9% (−42.4%, −17.5%) in the 900 mg and 300 mg arms for an antibody of the present invention, respectively, versus −5.4% (−35.6%, 24.9%) in the placebo arm, with improvement observed within two weeks of initiation with an antibody of the present invention. Overall, the antibody of the present invention was well tolerated with an adverse event (AE) profile comparable with placebo. Through 32 weeks, 16 patients (42.1%) receiving the antibody of the present invention had a drug-related AE; majority were graded as mild or moderate. No new or dose-dependent AEs were observed. Moreover, gene expression levels in skin biopsies from the worst affected areas (by PPP ASI of the region where the biopsy was taken; n=23) revealed a distinct molecular profile characterised by stronger expression of markers of the IL-36 pathway (IL36A/B/G), Th17 pathway (IL17A/F, DEFB4), neutrophil trafficking (CXCL1, CXCL2, CXCL6) and inflammation (TNF, S100A8/9/12) in patients with more severe lesions.

### Conclusions

Although this study failed to meet its primary endpoint, treatment with an antibody of the present invention was associated with decreases in PPP ASI and pustular severity in patients with higher disease severity. These interventional data, together with the differential upregulation of IL-36 pathway genes in more severe PPP lesions, suggests that IL-36 plays a functional role in PPP. Additional studies are needed to confirm the efficacy of IL-36R inhibition in patients with PPP.

### Introduction

Pustular psoriasis consists of a spectrum of rare inflammatory skin conditions that are characterised by neutrophilic infiltrations of the epidermis resulting in clinically visible sterile pustules. Generalized pustular psoriasis (GPP) and palmoplantar pustulosis (PPP) are the most prominent subphenotypes with GPP the most severe form of pustular psoriasis and PPP the most common. GPP is multisystemic and life-threatening, consisting of intermittent acute flares of a disseminated erythematous and pustular skin rash on non-acral skin, associated with general symptoms such as fever, malaise with asthenia, myalgia and arthralgia. The severity of general symptoms varies greatly from one case to another and often between flares within the same individual. Epidemiological studies report prevalence as low as 1.76/million, highlighting the rarity of the disease. In contrast to GPP, PPP is not considered to be life-threatening and is localised to the palms of the hands and/or soles of the feet. The disease tends to predominate on the thenar, hypothenar, and central areas of the palms, as well as the corresponding areas of the soles, and it can extend proximally to the patient's wrists and heels. Overall estimates of PPP prevalence in Western populations range from 0.01% to 0.05%; in Japan a higher prevalence of 0.12% has been reported.

Therapeutic intervention is a major challenge for both GPP and PPP with no biologic treatments currently approved in the US or Europe. Commonly used treatment are associated with undesirable side effects, limiting their long-term use. A wide range of anti-psoriatic strategies have been proposed for GPP and PPP based on the plaque psoriasis model, with the efficacy of apheresis, and inhibitors of tumor necrosis factor, interleukin-17 and interleukin-23 reported in open-label trials and case reports forming the basis of approval for GPP in Japan; recently, an interleukin-23 inhibitor has been approved for PPP in Japan. PPP may be exacerbated by acute tonsillitis and the use of tonsillectomy is reported to be highly effective in some Japanese patients; however, a study in a small cohort of patients with GPP found tonsillectomy to be largely ineffective.

The immunopathogenesis of each disease is yet to be fully elucidated, however, major advances have been gained from genetic studies that identified loss-of-function homozygous or compound heterozygous IL36RN gene mutations as a major pathogenic factor in GPP. These mutations severely alter the function of the IL36RN product, the interleukin-36 receptor antagonist (interleukin-36Ra), resulting in the dysregulation of the proinflammatory interleukin-36 (IL-36α, IL-36β and IL-36γ) pathway, and lead to GPP according to a monogenic model. While these mutations have been found in other pustular psoriasis subtypes, they have not been detected in patients with plaque psoriasis alone, unveiling the autoinflammatory nature of pustular psoriasis and establishing GPP as a distinct entity from plaque psoriasis. Mutations in other genes, such as CARD14 and AP1S3 have also been detected in subtypes of pustular psoriasis and almost 50% patients with GPP carry a variant in one or more genes that are associated with GPP disease (e.g. IL36RN, CARD14 or AP1S3). Further, IL-36 cytokines are highly expressed in GPP lesions and are involved in the recruitment and activation of inflammatory cells. Conversely, in PPP, a genetic association with the IL-36 pathway is less evident, with only ˜10% of patients with PPP being reported to having a loss-of-function mutation in IL36RN or AP1S3 genes. However, IL-36 cytokines have been reported to be highly expressed in PPP lesions and the IL-36 pathway is thought to be integral to the pathogenesis of PPP.

An antibody of the present invention, i.e. an anti-IL-36 receptor (IL-36R) antibody (spesolimab [BI 655130]), is a humanized antagonistic monoclonal IgG1 antibody that blocks human IL-36R signaling. This invention was investigated in a 20-week, multicenter, single-arm, open-label, phase I, proof-of-concept trial in seven patients who presented with a GPP flare (ClinicalTrials.gov number, NCT02978690). Eligible patients received a single intravenous (IV) dose of 10 mg/kg an anti-IL-36R antibody of the present invention and were monitored for 20 weeks. A Generalized Pustular Psoriasis Physician Global Assessment (GPPGA) score of 0 or 1 (clear or almost clear skin) was achieved in five patients by Week 1 and in all patients (with or without the IL36RN mutation) by Week 4. The patients were also evaluated with the use of the GPP Area and Severity Index (GPPASI), an adaptation of the PASI score in which the induration component is replaced by a pustule component, with a total score ranging from 0 (least severe) to 72 (most severe). Among the study patients, the mean percent improvement in the GPPASI score from baseline was 59.0% at Week 1, 73.2% at Week 2, and 79.8% at Week 4. Pustules were completely cleared in three patients within 48 hours after treatment, in five patients by Week 1, and in six patients by Week 2. GPPGA, GPPASI, and pustule subscores were maintained up to Week 20. A reduction in the mean (±SD) level of C-reactive protein that approached normalisation was observed from baseline to Week 2 (from 69.4±57.0 mg per deciliter to 4.5±7.5 mg per deciliter) and was sustained until the last measurement was obtained at Week 4. Treatment with the antibody of the present invention resulted in strong and rapid downregulation of lesional versus non-lesional biomarkers and serum biomarkers linked to inflammatory, neutrophilic, innate and Th1/Th17 pathways; these reductions correlated with decreases in clinical disease severity, highlighting the importance of inhibiting the IL-36 pathway in the skin and blood of patients with GPP (Baum P, et al. presented at SID 2019: Abstract LB1140). After the infusion of the study drug, all the patients had adverse events that were graded as mild or moderate, and no serious adverse events were reported.

Preclinical profiles of an antibody of the present invention and clinical data from trials with healthy volunteers and patients with GPP suggest that an antibody of the present invention is safe, tolerable and may address an unmet medical need in patients with PPP, as the IL-36 pathway is thought to be integral to the pathogenesis of this disease. The results of this first study assessing the safety and efficacy of an anti-IL-36R antibody of the present invention in patients with PPP are reported. To our knowledge, this is the first study to assess treatment in patients with PPP.

### Methods

Study Design

This 32-week, multinational, randomised, double-blind, placebo-controlled, parallel-design trial to investigate the safety and efficacy of an antibody of the present invention in patients with PPP was conducted in 18 sites across Canada, Denmark, Germany, Italy, Spain, and Sweden. The trial consisted of three consecutive study periods: screening (7-28 days), treatment (16 weeks), and follow-up (16 weeks). Eligible patients identified during screening were randomised to treatment with one of two dose arms of an antibody of the present invention (900 or 300 mg intravenously Q4W) or placebo. Patients were randomised 1:1:1 in a blinded fashion using an interactive response technology. Treatment was administered on visits 2, 6, 8, and 10, corresponding to Day 1 and Weeks 4, 8, and 12.

Patients

Patients aged 18-65 were eligible if they had PPP defined as the presence of primary, persistent (>3 months duration), sterile, macroscopically visible pustules on the palms and/or soles, with or without plaque psoriasis on less than 10% of the body surface area. Pustulation was required to be active (yellow pustules) on palms and/or soles and patients were required to have a minimum Palmoplantar Pustular Psoriasis Area and Severity Index (PPP ASI) score of 12 and a Palmoplantar Pustulosis Physicians Global Assessment (PPP PGA) of at least moderate severity at baseline.

Patients were excluded if they had: a severe, progressive, or uncontrolled renal, hepatic, haematological, endocrine, pulmonary, cardiac, neurologic, cerebral, or psychiatric disease, or signs and symptoms thereof; presence or known history of anti-TNF-induced PPP-like disease or SAPHO (Synovitis-acne-pustulosis-hyperostosis-osteitis) syndrome; received a transplanted organ (with exception of a corneal transplant >12 weeks prior to screening) or who have ever received stem cell therapy; a known history of lymphoproliferative disease or any documented active or suspected malignancy or history of malignancy within 5 years prior to screening. (See Table 6 for full inclusion/exclusion criteria). For patients satisfying the inclusion/exclusion criteria, randomisation and treatment was initiated at visit 2.

TABLE 6
Inclusion/Exclusion Criteria
Inclusion criteria
Patients will only be included into the trial if they meet the following criteria:

| | |
|---|---|
| 1. | Signed and dated written informed consent in accordance with Good |
| | Clinical Practice (GCP) and local legislation prior to the start of any |
| | screening procedures. |
| 2. | Male or female patients, 18 to 65 years of age at screening. |
| 3. | PPP defined as presence of primary, persistent (> 3months duration), |
| | sterile, macroscopically visible pustules on the palms and/or soles, without |
| | or with plaque psoriasis on less than 10% of the body surface area. |
| 4. | Presence of active pustulation (yellow pustules) on palms and /or soles. |
| 5. | A minimum PPP ASI score of 12 and PPP PGA of at least moderate |
| | severity at baseline. |
| 6. | Women of childbearing potential (WOCBP) and men able to father a child |
| | must use highly effective methods of birth control per ICH M3 (R2) that |
| | result in a low failure rate of less than 1% per year when used consistently |
| | and correctly. A list of contraception methods meeting these criteria is |
| | provided in the patient information. |

Exclusion criteria
Patients will be excluded from the trial if they have the following criteria:

| | |
|---|---|
| 1. | Patients with associated plaque psoriasis ≥10% of the body surface area. |
| 2. | Women who are pregnant, nursing, or who plan to become pregnant while |
| | in the trial. |
| 3. | Severe, progressive, or uncontrolled renal, hepatic, haematological, |
| | endocrine, pulmonary, cardiac, neurologic, cerebral, or psychiatric disease, |
| | or signs and symptoms thereof. |
| 4. | Presence or known history of anti-TNF-induced PPP-like disease. |
| 5. | Patients with SAPHO (Synovitis-acne-pustulosis-hyperostosis-osteitis) |
| | syndrome. |
| 6. | Patient with a transplanted organ (with exception of a corneal transplant > |
| | 12 weeks prior to screening) or who have ever received stem cell therapy |
| | (e.g., Prochymal). |
| 7. | Known history of lymphoproliferative disease, including lymphoma, or signs |
| | and symptoms suggestive of possible lymphoproliferative disease, such as |
| | lymphadenopathy and/or splenomegaly. |
| 8. | Any documented active or suspected malignancy or history of malignancy |
| | within 5 years prior to the screening visit, except appropriately treated basal |
| | or squamous cell carcinoma of the skin or in situ carcinoma of uterine |
| | cervix. |
| 9. | Patients who have previously undergone allergy immunotherapy for |
| | prevention of anaphylactic reactions. |
| 10. | Use of any restricted medication (see Table 7) or any drug considered likely |
| | to interfere with the safe conduct of the study, as assessed by the |
| | investigator. |
| 11. | Plans for administration of live vaccines during the study period or within 6 |
| | weeks prior to randomisation. |
| 12. | History of allergy/hypersensitivity to a systemically administered biologic |
| | agent or its excipients. |
| 13. | Active systemic infections during the last 2 weeks (exception: common cold) |
| | prior to randomisation, as assessed by the investigator. |
| 14. | Chronic or relevant acute infections including human immunodeficiency |
| | virus (HIV), viral hepatitis and (or) active or latent tuberculosis (patients with |
| | a positive QuantiFERON TB test are excluded. Patients with suspected |
| | false positive or undeterminable QuantiFERON TB result may be re-tested). |
| 15. | Major surgery performed within 12 weeks prior to randomisation or planned |
| | within 32 weeks after randomisation (e.g. hip replacement, aneurysm |
| | removal, stomach ligation), as assessed by the investigator. |
| 16. | Total white blood count (WBC) <3,000/uL, or platelets <100,000/μL or |
| | neutrophils <1,500/μL, or hemoglobin <8.5 g/dL at screening. |
| 17. | Aspartate aminotransferase (AST) or alanine aminotransferase (ALT) >2× |
| | the upper limit of normal, or total bilirubin >1.5× the upper limit of normal |
| | (patients with Gilbert’s syndrome are not excluded) at screening. |
| 18. | Currently enrolled in another investigational device or drug study, or less |
| | than 30 days since ending another investigational device or drug study(s), |
| | or receiving other investigational treatment(s). |
| 19. | Chronic alcohol or drug abuse or any condition that, in the investigator’s |
| | opinion, makes them an unreliable study subject or unlikely to complete the |
| | trial. |
| 20. | Previous randomisation in this trial. |

TABLE 7
Restricted Medications

| | Restriction from |
|---|---|
| Medication or class of medications | (through to Week 16)¹ |
| IL36R inhibitors other than the study | Not allowed neither before nor |
| drug | during trial participation |
| Secukinumab (Cosentyx ®), | 12 weeks or 5 half-lives, |
| ustekinumab (Stelara ®), | whichever is greater, prior to |
| guselkumab, ixekizumab, | randomisation |
| tildrakizumab,brodalumab | |
| Adalimumab, infliximab | |
| Natalizumab or agents that deplete B or | |
| T cells | |
| (e.g. rituximab, alemtuzumab or | |
| visilizumab) | |
| Investigational products for psoriasis | |
| Etanercept | 6 weeks prior to randomisation |
| Live virus vaccinations⁴ | |
| Other systemic immunomodulating | 4 weeks prior to randomisation |
| treatments | |
| (e.g. corticosteroids², methotrexate, | |
| fumaric acid esters, acitretin, | |
| ciclosporin, apremilast | |
| Any investigational device or product | |
| (excludes psoriasis products) | |
| Phototherapy (e.g. UVA, UVB), topical | 14 days prior to randomisation. |
| treatment for psoriasis or any other skin | |
| condition (e.g. corticosteroids³, vitamin | |
| D analogues, salicylic acid, tar, | |
| anthralin) | |
| Anakinra | 7 days prior to randomization |

¹In case of worsening of the PPP and/or psoriasis, the use of a rescue medication was left at the discretion of the investigator (refer to Section 9.4.2.1); In case of any other acute indication after the primary endpoint Visit at Week 16, the use of restricted medication was permitted.
²There was no restriction on corticosteroids with only a topical effect (e.g. inhaled corticosteroids to treat asthma or corticosteroids drops administered in the eye or ear).
³Exception: the use of topical steroids of US class 6 (mild, such as desonide) or US class 7 (least potent, such as hydrocortisone) on the face, axilla, and/or genitalia was only restricted within 24 h prior to trial visits in which PPP ASI was assessed.
⁴Live virus vaccination should be restricted until the end of the trial.

Efficacy and Safety Assessments

The primary endpoints were safety (number of patients with drug-related adverse events [AEs] over 32-weeks) and the proportion of patients achieving a PPP ASI50 at Week 16 following treatment with an anti-IL-36R antibody of the present invention. Safety assessments included AEs (coded with the use of the Medical Dictionary for Drug Regulatory Activities [MedDRA] version 21.1; intensity of AEs assessed by the Rheumatology Common Toxicity Criteria [RCTC] version 2.0), serious adverse events, laboratory assessments, physical examination, vital signs, and 12-lead electrocardiograms over the duration of the trial (32-weeks).

Secondary endpoints included: proportion of patients achieving PPP ASI75, percent change from baseline in PPP ASI, and proportion of patients achieving PPP PGA 0 or 1 at Week 16. Further exploratory endpoints included: proportion of patients achieving PPP ASI50 or 75, percent change from baseline in PPP ASI, and proportion of patients achieving PPP PGA 0 or 1 at all other visits; time (days) to achieving and loss of PPP ASI50 response; biomarker to evaluate the treatment response in PPP (e.g. granulocytes, SAA, IL8, CRP, IL1β).

Because the primary analysis did not show a significant difference between the anti-IL-36R antibody of the present invention and placebo treatment in terms of efficacy, exploratory analyses were conducted based on the database snapshot taken for the primary analysis to dissect the results. Post hoc analyses included: percent change from baseline in PPP ASI at Week 16 versus percent change from baseline in PPP ASI at screening; percent change in PPP ASI from baseline, pustule severity, and change from baseline in pain-VAS in those patients with improvement and those without improvement in PPP ASI from screening to baseline; and percent change in PPP ASI from baseline, pustule severity and change from baseline in pain-VAS in patients with baseline PPP ASI above and below the median baseline PPP ASI score.

PPP ASI and Related Assessments

The PPP ASI is an investigator assessment of the extent and severity of pustular and plaque lesions on the palms and soles presenting in PPP patients. The adaptation from PASI, an established measure of severity and area of psoriatic lesions in patients with psoriasis, was used in this trial. This tool provides a numeric scoring for patients overall PPP disease state, ranging from 0 to 72. It is a linear combination of the percent of surface area of skin that is affected on the palms and soles and the severity of erythema, pustules, and scaling (desquamation). The PPP ASI is calculated as follows as a weighted sum of the scores obtained for erythema, pustules, desquamation and percent area affected (Table 8):

PPP ASI=[(E+P+D)×A×0.2(right palm)]+[(E+P+D)×A×0.2(left palm)]+[(E+P+D)×A×0.3(right sole)]+[(E+P+D)×A×0.3(left sole)]

TABLE 8
PPP ASI

| | Score | | | | | | |
|---|---|---|---|---|---|---|---|
| Symptom | 0 | 1 | 2 | 3 | 4 | 5 | 6 |
| Erythema (E) | None | Slight | Moderate | Severe | Very | | |
| | | | | | severe | | |
| Pustules (P) | None | Slight | Moderate | Severe | Very | | |
| | | | | | severe | | |
| Desquamation | None | Slight | Moderate | Severe | Very | | |
| (D) | | | | | severe | | |
| Area affected (%) | 0 | <10 | 10 < 30 | 30 < 50 | 50 < 70 | 70 < 90 | 90-100 |
| (A) | | | | | | | |

Achievement of a response in PPP ASI from baseline was assessed and is typically reported as a XX % change in PPP ASI and is denoted as PPP ASIXX, whereby XX is most commonly reported as 50, 75 or 90% improvement in PPP ASI. Proportion of patients achieving a PPP ASIXX response is reported.

PPP ASI severity was assessed by each component and by palms or soles. For the assessment by component, the mean severity within each component (E, P, or D) across all body areas (both palms and both soles) was calculated and presented for each component separately. Within a component, a missing value in one body area led to a missing value for the component. For the assessment by palms or soles, the PPP ASI score was calculated for either palms or soles via the components E, P, and D as well as the area (A) but replacing the region factor with a factor of 0.5 to achieve a total score range of 0 to 72. If either of the two palms or soles had a missing value, then the PPP ASI score was missing.

PPP PGA

The PPP PGA relies on clinical assessment of the patient's skin presentation on the palms and soles. The investigator (or qualified site personnel) scores the lesions on the most severely affected palmoplantar surface from 0-4 as clear, almost clear, mild, moderate, or severe (Table 9).

TABLE 9
PPP PGA

| Score | Description | Detailed description |
|---|---|---|
| 0 | Clear | No signs of PPP; no scaling or crusts or pustule remains |
| 1 | Almost | Slight scaling and/or erythema and/or slight crusts; very few |
| | clear | new (yellow) and/or old (brown) pustules |
| 2 | Mild | Scaling and/or erythema and/or crusts; visible new (yellow) |
| | | and/or old (brown) pustules of limited number and extent |
| 3 | Moderate | Prominent scaling and/or erythema and/or crusting; |
| | | prominent new (yellow) and/or old (brown) pustules covering |
| | | most of the area involved |
| 4 | Severe | Severe scaling and/or erythema and/or crusting; numerous |
| | | new (yellow) or old (brown) pustules with and/or without major |
| | | confluence covering the entire area of at least 2 palmoplantar |
| | | surfaces |

Photographic documentation of skin lesions was performed at baseline, and post-treatment.

Biochemical, cellular, and pharmacogenomic biomarkers were evaluated in skin and whole blood (see below for biomarker and pharmacogenomics methodologies). Skin biopsies were performed at baseline (Day 1) and Week 6 (Day 29±3).

Biomarker Assessments

Assessment of CRP levels (non-high sensitive) and absolute neutrophil count were conducted using standard methodologies at a central laboratory service. Samples for assessments were collected at baseline before treatment initiation (day 1) and on days 8 (week 1), 29 (Week 2), 43 (Week 6), 57 (week 8), 85 (Week 12), 113 (Week 16) and 225 (Week 32).

Pharmacogenomic Biomarker Assessments

Global transcriptome-wide sequencing of RNA from lesion and non-lesional skin biopsy samples and whole blood from all patients was achieved using the Illumina Hi-Seq 3000 (IIlumina Inc., San Diego, Calif.). Data were normalized by trimmed mean of M values (TMM) using the edgeR package; log 2 fold changes and corresponding FDR-adjusted p-values were calculated using the limma-voom package (Bioconductor, US). Briefly, the data were voom-transformed and correlations between paired measurements per patient were estimated by the duplicate Correlation function. A linear model was fitted using the ImFit-function and moderated t-statistics were computed for lesional versus non-lesional and pre-versus post-treatment with an anti-IL36R antibody of the present invention. Adjusted P-values of <0.05 were considered significant.

In order to confirm the results obtained by RNA sequencing the gene expression of selected genes was confirmed via quantitative real-time PCR (TaqMan). Genes analysed with PCR include ATP12A, C15orf48, CCL20, CCL4, CHI3L2, CXCL1, CXCL2, CXCL5, CXCL6, CXCL8, CXCR2, CXCR4, DEFB4B; DEFB4A, IGH, IGHA1, IL17A, IL17F, IL19, IL1A, IL1B, IL1F10, IL23A, IL36A, IL36B, IL36G, IL36RN, KLK6, LCN2, MIR155HG, MMP12, PI3, RHCG, S100A12, S100A7, S100A8, S100A9, SERPINB4, SPRR2D, TCN1, TMPRSS11D, TNF, VNN1, VNN3, WNTSA.

Gene expression analysis was performed on total RNA extracted from skin biopsies samples from all patients at baseline (Visit 2) and 6 weeks after drug administration (V6). Gene expression was analysed by TaqMan qRT-PCR for all available samples according to the manufacture's protocol.

Prior to gene expression analysis totalRNA was extracted from skin biopsies. The process flow of TaqMan-based gene expression analysis consisted of cDNA synthesis from extracted totalRNA and quantitative real-time PCR (TaqMan) to amplify the specific genetic target sites and the record and analysis of received data.

### Immunogenicity Assessments

Plasma samples from all patients for anti-drug antibody assessment were taken at pre-dose (Day 1) and on days 15±3, 29±3, 57±3, 85±3, 113±3, 169±7 and 225±7. The samples were analysed for anti-an anti-IL-36R antibody of the present invention antibodies using a validated Meso Scale Discovery® (MSD) drug bridging electrochemiluminescent (ECL) method with acid dissociation at QPS, LLC, Newark, Del., USA. Anti-drug antibody plasma samples and controls were first diluted in 0.3M acetic acid before neutralization with 1.5M tris base and master mix, which included biotin-labeled drug and sulfo-tag-labeled drug, prior to transfer and incubation on a blocked MSD streptavidin plate. In the presence of tripropylaminecontaining read buffer, sulfo-tag produces an ECL signal that is triggered when voltage is applied using the MSD Sector Imager 600s. The resulting chemiluminescence is measured in relative light units which is proportional to the amount of anti-drug antibody present in the plasma samples. The immunogenicity of an anti-IL-36R antibody of the present invention was assessed using a three-tiered approach. All anti-drug antibody samples were first analyzed in the anti-drug antibody screening assay. A sample was considered positive for anti-an anti-IL-36R antibody of the present invention antibodies if its response in the screening assay was greater than or equal to the screening plate-specific cut point, and if it was confirmed positive in the confirmatory assay (ECL response inhibited by addition of excess an anti-IL-36R antibody of the present invention above the confirmatory cutpoint). Samples that were confirmed positive for anti-an anti-IL-36R antibody of the present invention antibodies were further characterized in the titration assay. Titers were determined by analysis of 2-fold serial dilutions of a sample. The reported titer was the highest dilution that produced a mean ECL value greater than or equal to the plate specific titration cutpoint. The anti-drug antibody assay validation demonstrated that the sensitivity of the screening assay in PPP plasma was 2.5 ng/mL using an anti-an anti-IL-36R antibody of the present invention rabbit polyclonal antibody positive control. In addition, 100 and 250 ng/mL levels of the positive control were detected in the presence of at least 2000 μg/mL an anti-IL-36R antibody of the present invention. None of the ADA samples had an anti-IL-36R antibody of the present invention levels greater than 2000 μg/mL. The assay performance data indicated that the method was reliable for screening, confirmation, and determination of titers of anti-an anti-IL-36R antibody of the present invention antibodies in plasma samples from patients in this study.

Statistical Analyses

This was an exploratory trial and formal confirmatory statistical testing was not performed. The efficacy analyses will be performed for the full analysis set (FAS) which is based on the intent-to-treat principle, and comprises all participants who were randomised, received at least one dose during the trial, and had a baseline measurement for the primary endpoint. Safety analyses on patients who were randomised and received at least one dose during the trial will be based on the actual treatment received at the randomisation visit (safety analysis set [SAF]).

The achievement of PPP ASI50 at Week 16 is the primary endpoint in this trial and represents a binary variable with values of 0 (=non-response) or 1 (=response). The primary analysis of the unadjusted absolute risk difference versus Placebo was calculated simply as the difference in the observed proportion of patients with PPP ASI50 at Week 16 for each treatment scenario, for the FAS. A 95% Wilson confidence interval around this difference was provided. In addition, a parametric bootstrap 95% confidence interval was generated by sampling from the binomial distribution on each treatment with number of patients and observed proportion of responders per treatment representing the sampling parameters. Sensitivity analyses utilizing different patients sets (such as the per-protocol set [PPS]), as well as alternative methods for the handling of missing data were conducted, in addition to exploration of the relationship between various demographic or baseline characteristics data and the primary endpoint using graphical methods as well as using a logit link with PROC LOGISTIC in SAS®. Secondary and exploratory endpoints were analysed using the same methodology described for the primary endpoint. For continuous endpoints, mean changes from baseline were analysed using a restricted maximum likelihood (REML)-based measures approach. Analysis of safety was conducted descriptively and focused on treatment-emergent events.

### Results

Patients

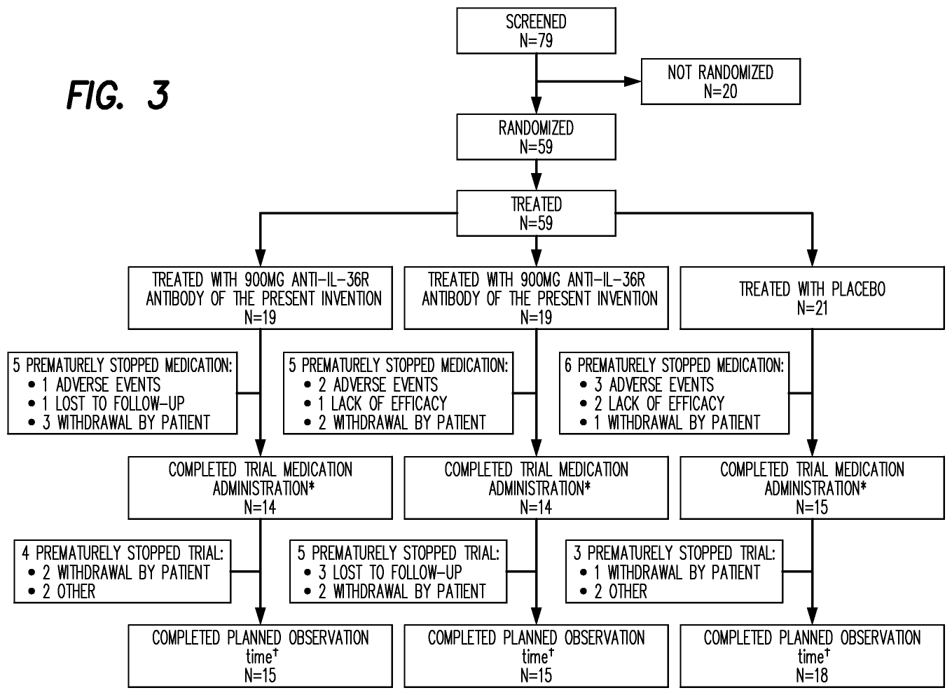

Of 79 patients screened, a total of 59 patients at 18 study sites were randomly assigned to either 900 mg (19 patients) or 300 mg (19 patients) of an anti-IL-36R antibody of the present invention or to placebo (40 patients; FIG. 3). Baseline demographics and disease characteristics were generally well balanced between treatment arms; the mean (standard deviation [SD]) time since first diagnosis was 9.1 years (11.3 years) in the overall trial population (Table 10). It was slightly shorter in the placebo group (mean 6.7 years) than in the investigational treatment groups (mean 10.4 years), possibly due to the slightly younger patients in this treatment group. The vast majority of patients (91.5%) had chronic or persistent signs of active disease within the last year. The mean (SD) PPP ASI total score at baseline was 18.56 (6.34) and the median PPP ASI total score was 16.7 (range 12 to 37)(Table 10). A total of 43 patients (72.9%) completed trial medication administration; all patients, regardless whether they completed the administration of trial medication as planned or whether they discontinued treatment prematurely, were to be followed until the end-of-trial visit at Week 32. Fifty-three patients (89.8%) completed the primary endpoint visit at Week 16 and 47 patients (79.7%) completing the trial observation period. The frequency of patients who discontinued treatment prematurely was similar in all treatment groups. The most frequent reasons for discontinuing were for AEs or withdrawal by the patient. For three patients discontinuing due to AEs, the AE was worsening of PPP; three patients also discontinued due to lack of efficacy, thus a total of 6 patients discontinued treatment due to worsening of disease or lack of improvement (two of these patients were in the 900 mg dose group of the anti-IL-36R antibody of the present invention and four were in the placebo group).

TABLE 10
Baseline Demographics and Disease Characteristics

| | | Anti-IL-36R antibody of the | | | |
|---|---|---|---|---|---|
| | | present invention | | | Total |
| | Placebo | 300 mg | 900 mg | Total | overall |
| | (N=21) | (N = 19) | (N = 19) | (N = 38) | (N = 59) |
| Sex, n (%) | | | | | |
| Female | 17 (81.0) | 16 (84.2) | 16(84.2) | 32 (84.2) | 49 (83.1) |
| Male | 4 (19.0) | 3 (15.8) | 3 (15.8) | 6 (15.8) | 10 (16.9) |
| Mean age ± SD, y | 46.3 ± 11.7 | 54.6 ± 7.7 | 49.4 ± 11.3 | 52.0 ± 9.9 | 50.0 ± 10.9 |
| Race, n (%) | | | | | |
| White | 19 (90.5) | 18 (94.5) | 19 (100) | 37 (97.4) | 56 (94.9) |
| Asian | 1 (4.8) | 0 | 0 | 0 | 1 (1.7) |
| Black | 1 (4.8) | 0 | 0 | 0 | 1 (1.7) |
| Multiple | 0 | 1 (5.3) | 0 | 1 (2.6) | 1 (1.7) |
| Weight, mean ± SD, kg | 79.0 ± 15.8 | 81.3 ± 12.7 | 76.8 ± 19.2 | 79.1 ± 16.3 | 79.0 ± 15.9 |
| BMI, mean ± SD, kg/m² | 29.0 ± 5.5 | 29.5 ± 5.2 | 27.2 ± 5.9 | 28.4 ± 5.6 | 28.3 ± 5.5 |
| Smoking status, n (%) | | | | | |
| Current | 1 (4-8) | 0 | 0 | 0 | 1 (1-7) |
| Former | 4 (19.0) | 6 (31.6) | 8 (42.1) | 14 (36.8) | 18 (30.5) |
| Never | 16 (76.2) | 13 (68.4) | 11 (57.9) | 24 (63.2) | 40 (67.8) |
| Alcohol status, n (%) | 4 (19.0) | 6 (31.6) | 5 (26.3) | 11 (28.9) | 15 (25.4) |
| Current | 1 (4.8) | 2 (10.5) | 1 (5.3) | 3 (7.9) | 4 (6.8) |
| Former | 16 (76.2) | 11 (57.9) | 13 (68.4) | 24 (63.2) | 40 (67.8) |
| Never | | | | | |
| Mean PPP ASI ± SD at | 18.5 ± 7.6 | 20.3 ± 6.4 | 16.9 ± 4.3 | 18.6 ± 5.7 | 18.6 ± 6.3 |
| baseline | | | | | |
| Mean PPP ASI ± SD at | 18.5 ± 7.4 | 16.5 ± 5.3 | 16.2 ± 3.9 | 16.4 ± 4.6 | 17.1 ± 5.8 |
| screening | | | | | |
| Time since first diagnosis | 6.7 ± 7.0 | 9.5 ± 12.1 | 11.2 ± 14.0 | 10.4 ± 13.0 | 9.1 ± 11.3 |
| of PPP, mean ± SD, y | | | | | |
| PPP PGA score, n (%) | | | | | |
| 4 | 6 (28.6) | 7 (36.8) | 4 (21.1) | 11 (28.9) | 17 (28.8) |
| 3 | 15 (71.4) | 12 (63.2) | 15(78.9) | 27 (71.1) | 42 (71.2) |
| Mean pain-VAS ± SD | 58.8 ± 28.2 | 58.4 ± 25.4 | 67.9 ± 23.6 | 63.2 ± 24.7 | 61.6 ± 25.8 |
| C-reactive protein, | 4.8 ± 5.5 | 4.4 ± 2.7 | 4.3 ± 3.9 | 4.4 ± 3.3 | 4.5 ± 4.2 |
| mean ± SD, mg/L | | | | | |

Safety

Overall, the anti-IL-36R antibody of the present invention was well tolerated with an AE profile comparable with placebo (Table 11). Through 32 weeks, 16 patients receiving the anti-IL-36R antibody of the present invention (42.1%) had a drug-related AE; majority were graded as mild or moderate. the most frequently reported AEs were nasopharyngitis, headache, PPP, arthralgia, and cough. No new or dose-dependent AEs were observed.

TABLE 11
Summary of Adverse Events

| | | | Anti-IL-36R antibody of the | | | | | |
|---|---|---|---|---|---|---|---|---|
| | | | present invention | | | | | |
| | Placebo | | 300 mg | | 900 mg | | Total | |
| Patients, n (%) | (N = 21) | | (N = 19) | | (N = 19) | | (N = 38) | |
| Any AE | 18 | (85.7) | 17 | (89.5) | 17 | (89.5) | 34 | (89.5) |
| Severe AEs (RCTC | 2 | (9.5) | 2 | (10.5) | 2 | (10.5) | 4 | (10.5) |
| Grade 3 or 4) | | | | | | | | |
| Investigator defined | 9 | (42.9) | 8 | (42.1) | 8 | (42.1) | 16 | (42.1) |
| drug-related AEs | | | | | | | | |
| AEs leading to drug | 3 | (14.3) | 1 | (5.3) | 3 | (15.8) | 4 | (10.5) |
| discontinuation | | | | | | | | |
| AEs of special interest | 0 | | 0 | | 0 | | 0 | |
| Serious AEs | 1 | (4.8) | 1 | (5.3) | 0 | | 1 | (2.6) |
| Common AEs* | | | | | | | | |
| Nasopharyngitis | 8 | (38.1) | 5 | (26.3) | 8 | (42.1) | 13 | (34.2) |
| Headache | 7 | (33.1) | 4 | (21.1) | 6 | (31.6) | 10 | (26.3) |
| PPP | 4 | (19.0) | 2 | (10.5) | 3 | (15.8) | 5 | (13.2) |
| Acne | 0 | | 1 | (5.3) | 2 | (10.5) | 3 | (7.9) |
| Arthralgia | 1 | (4.8) | 3 | (15.8) | 2 | (10.5) | 5 | (13.2) |
| Cough | 1 | (4.8) | 3 | (15.8) | 2 | (10.5) | 5 | (13.2) |
| Alopecia | 0 | | 0 | | 2 | (10.5) | 2 | (5.3) |
| Back pain | 1 | (4.8) | 1 | (5.3) | 2 | (10.5) | 3 | (7.9) |

| | | | | | | | |
|---|---|---|---|---|---|---|---|
| Lipase increase | 1 | (4.8) | 0 | 2 | (10.5) | 2 | (5.3) |

| | | | | | | |
|---|---|---|---|---|---|---|
| Myalgia | 0 | 2 | (10.5) | 0 | 2 | (5.3) |

| Pruritus | 0 | | 0 | 2 | (10.5) | 2 | (5.3) |
|---|---|---|---|---|---|---|---|
| Psoriasis | 1 | (4.8) | 0 | 2 | (10.5) | 2 | (5.3) |

*Common AEs were reported in ≥10% of patients in any treatment group

Adverse events were coded using MedDRA v21.1. The severity of AEs was graded according to RCTC v2.0.

Treatment-emergent anti-drug antibodies were detected in 44.7% of patients receiving the anti-IL-36R antibody of the present invention (17 of 38 patients). In most patients these were transient and/or low titer. The anti-drug antibody level in one patient in the 300 mg dose group of the anti-IL-36R antibody of the present invention, may have contributed to a lack of efficacy of treatment observed in that patient during the trial.

Efficacy

Clinical Endpoints

At Week 16, 6 of 19 patients (31.6%) each in the 900 mg and the 300 mg dose groups of an anti-IL-36R antibody of the present invention had achieved PPP ASI50 (i.e. they had achieved a ≥50% decrease from baseline in their PPP ASI score). In the placebo group, 5 of 21 patients (23.8%) had achieved PPP ASI50. The risk difference vs. placebo was 0.078 (95% CI −0.190, 0.338) for both investigational treatment groups (Table 7). Thus, there was no relevant difference between investigational treatments and placebo in the proportion of patients achieving PPP ASI50 at Week 16.

At Week 16, 4 of 19 patients (21.1%) in the 900 mg and 0 of 19 patients in the 300 mg dose groups of an anti-IL-36R antibody of the present invention had achieved PPP ASI75 (i.e. they had achieved a ≥75% decrease from baseline in their PPP ASI score). In the placebo group, 2 of 21 patients (9.5%) had achieved PPP ASI75. The risk difference vs. placebo was 0.115 (95% CI −0.116, 0.348) for the 900 mg dose group and −0.095 (95% CI −0.289, 0.086) for the 300 mg dose group of an anti-IL-36R antibody of the present invention (Table 12).

The mean percent change in PPP ASI at Week 16 was highest in the 900 mg dose group of an anti-IL-36R antibody of the present invention (−45.80% [95% CI −60.75%, −30.85%]) followed by the placebo group (−39.97% [95% CI −58.22%, −21.73%]); it was lowest in the 300 mg dose group of an anti-IL-36R antibody of the present invention (−32.74% [95% CI −54.98%, —10.50%]) (Table 12).

The proportion of patients who achieved PPP PGA clear/almost clear (i.e. PPP PGA≤1) at Week 16 was comparable in the 900 mg dose group of an anti-IL-36R antibody of the present invention (3 of 19 patients, 15.8%) and the placebo group (3 of 21 patients, 14.3%). In the 300 mg dose group of an anti-IL-36R antibody of the present invention, no patient had PPP PGA at Week 16 (Table 12).

The mean (SD) absolute change in pain VAS at Week 16 was highest in the 900 mg dose group of an anti-IL-36R antibody of the present invention (−25.5 [28.4]) followed by the placebo group (−16.3 [30.2]); it was lowest in the 300 mg dose group of an anti-IL-36R antibody of the present invention 2.3 [26.9]) (Table 12).

TABLE 12
Efficacy Endpoints at Week 16

| | | Anti-IL-36R antibody of the | |
|---|---|---|---|
| | | present invention | |
| | Placebo | 300 mg | 900 mg |
| Endpoint | (N = 21) | (N = 19) | (N = 19) |
| Primary | | | |
| PPP ASI50 responders, % | 23.8 | 31.6 | 31.6 |
| (95% CI) | (10.6, 45.1) | (15.4, 54.0) | (15.4, 54.0) |
| risk difference vs placebo | | 0.078 | 0.078 |
| (95% CI) | | (−0.190, 0.338) | (−0.190, 0.338) |
| Secondary | | | |
| PPP ASI75 responders, % | 9.5 | 0 | 21.1 |
| (95% CI) | (2.7, 28.9) | −32.7 (38.5) | (8.5, 43.3) |
| PPP ASI, mean % change | −40.0 (33.0) | 0 | −45.8 (27.0) |
| from baseline (SD) | 14.3 | | 15.8 |
| PPP PGA 0 or 1, % | (5.0, 34.6) | | (5.5, 37.6) |
| (95% CI) | | | |
| Further endpoints | | | |
| Pain-VAS, mean change from | −16.3 (30.2) | 2.3 (26.9) | −25.5 (28.4) |
| baseline(SD) | | | |

95% confidence intervals (CI) are calculated using the method of Wison/Newcombe. Full analysis set, last observation carried forward.

Biomarker Analyses

A substudy comparing gene expression levels for patients (n=23) with a PPP

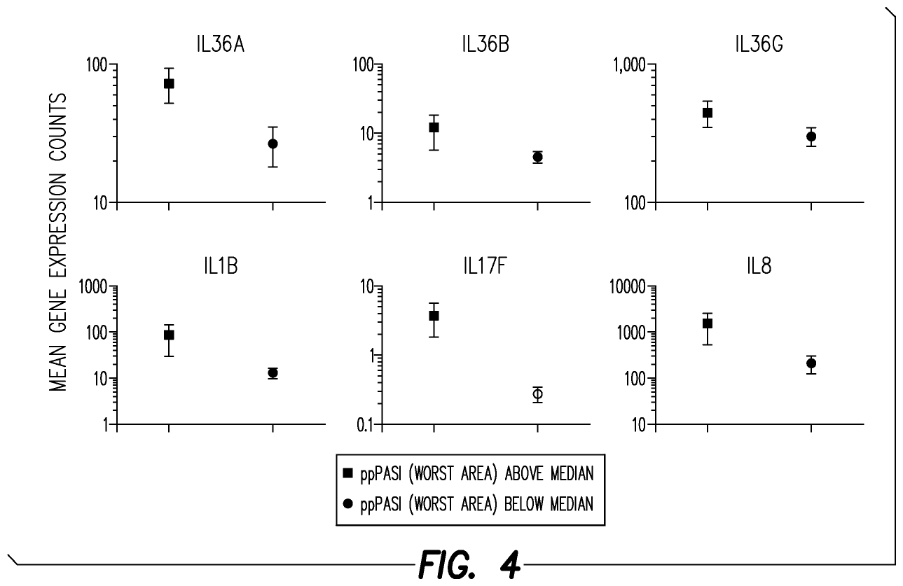

ASI above/below the median at baseline revealed a distinct molecular profile in patients with more severe lesions including a stronger expression of markers of the IL-36 pathway (IL36A/B/G), Th17 pathway (IL17A/F, DEFB4), neutrophil trafficking (CXCL1, CXCL2, CXCL6[IL8]) and inflammation (TNF, S100A8/9/12) (FIG. 4).

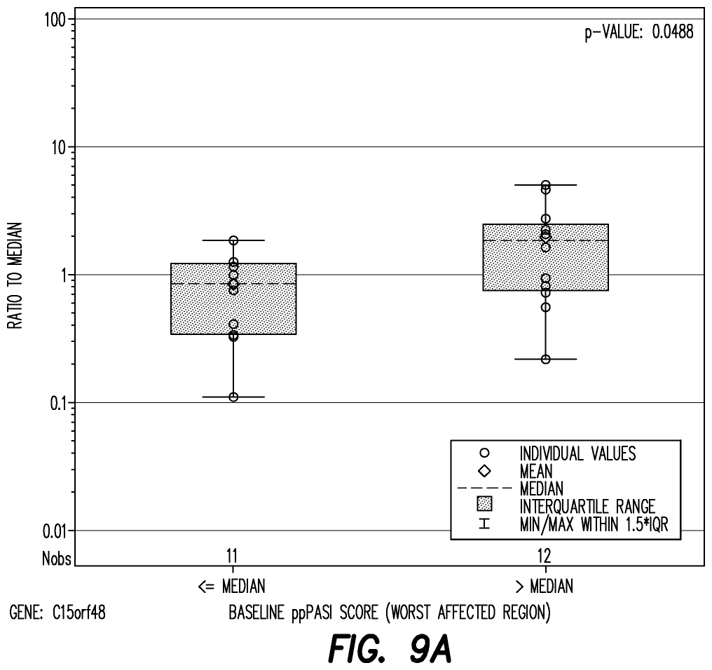

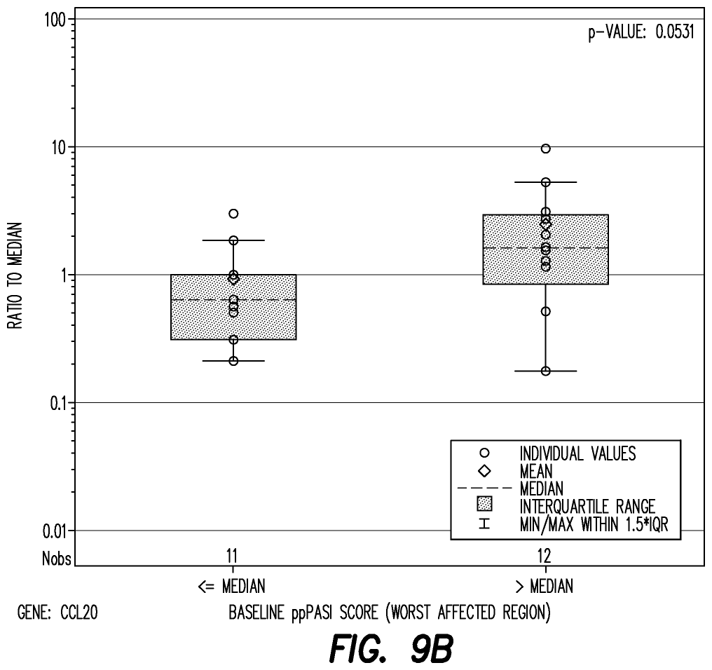

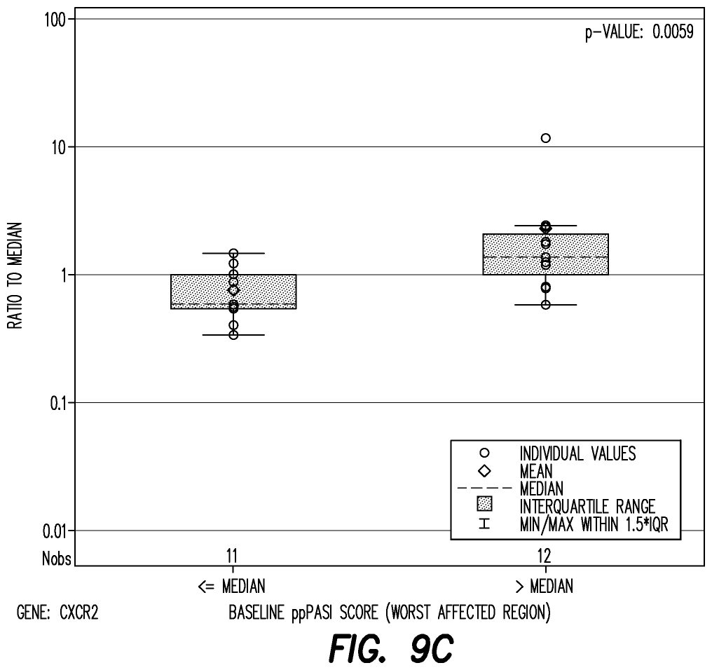

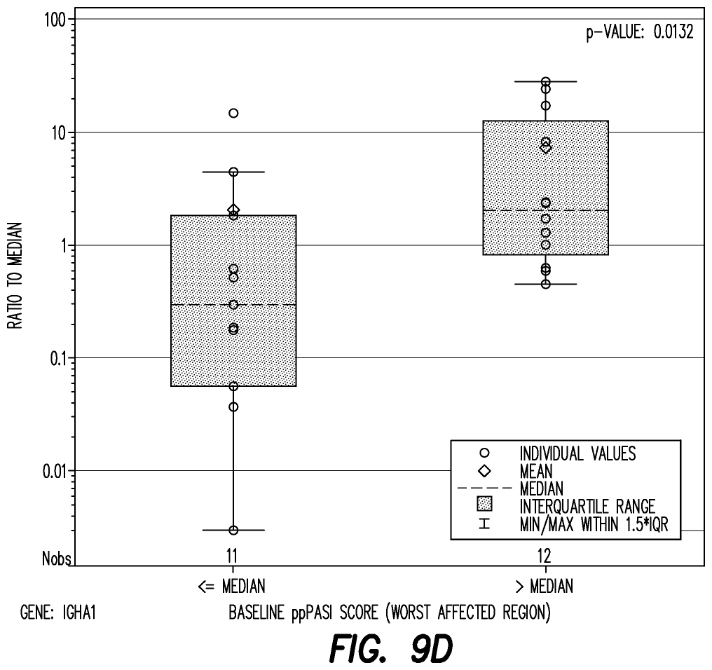

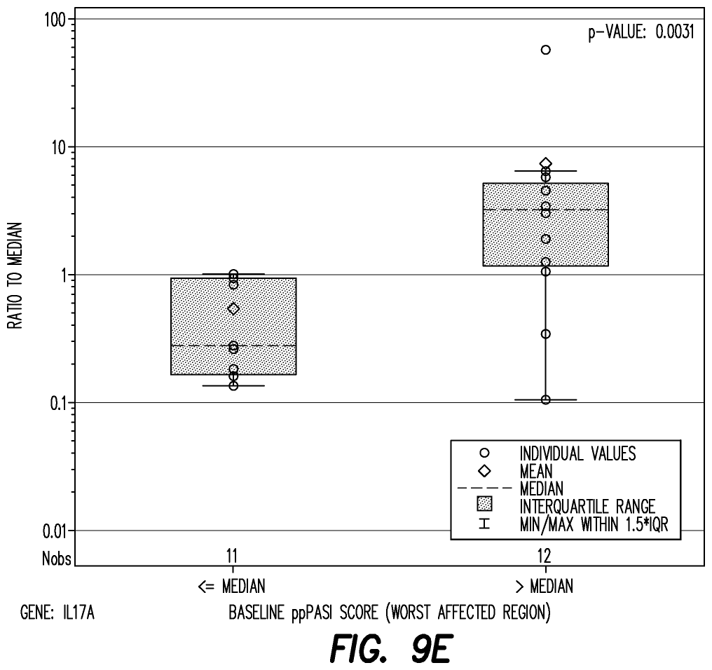

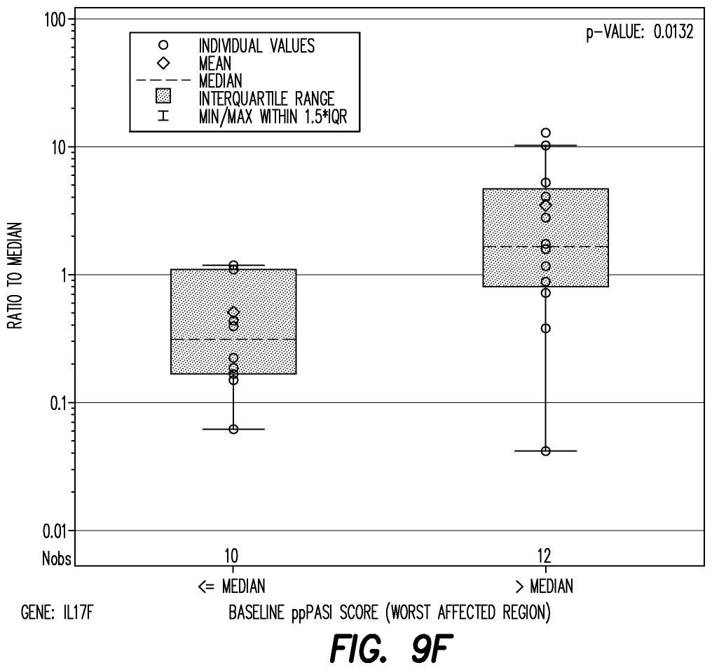

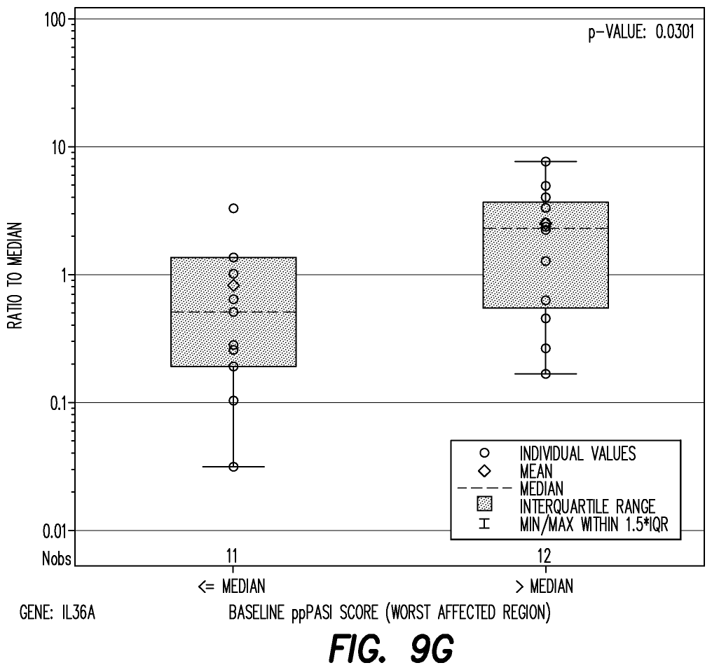

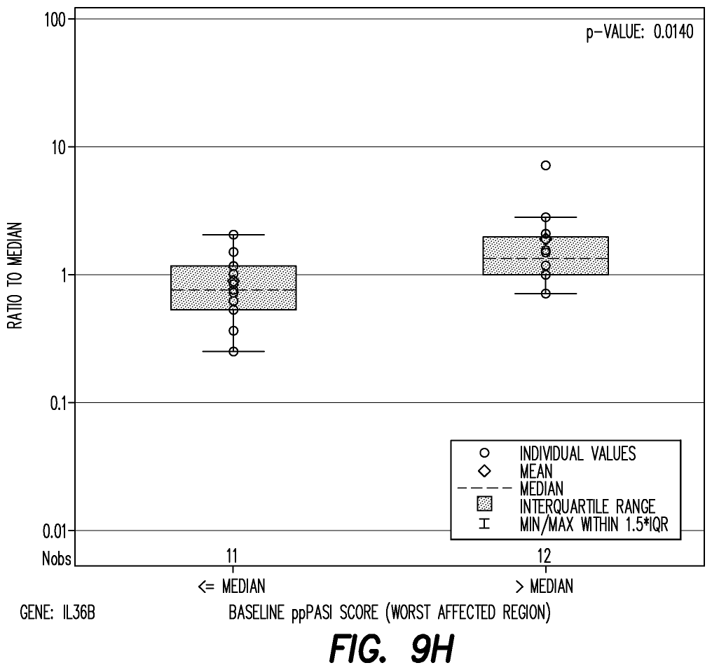

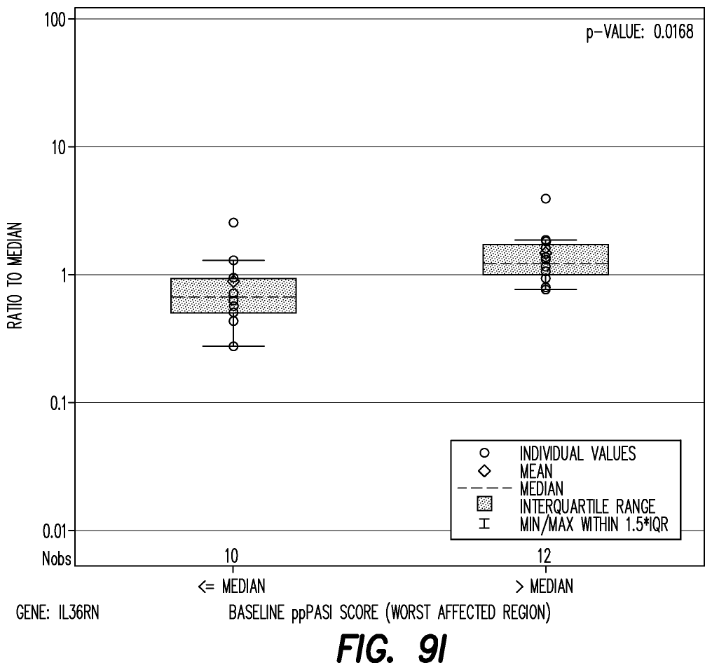

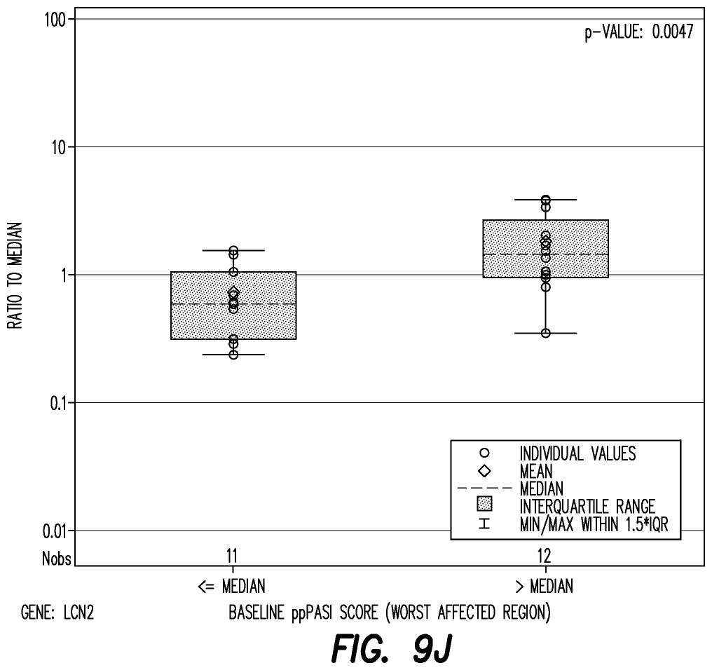

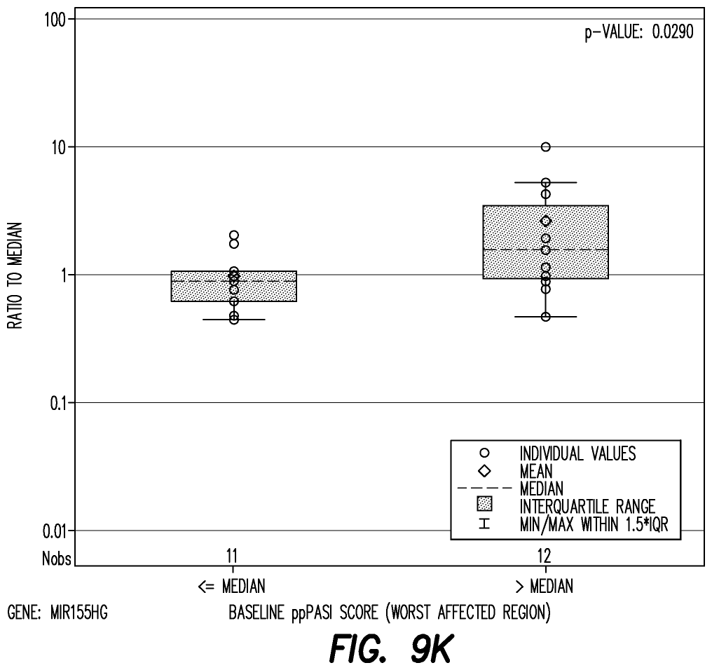

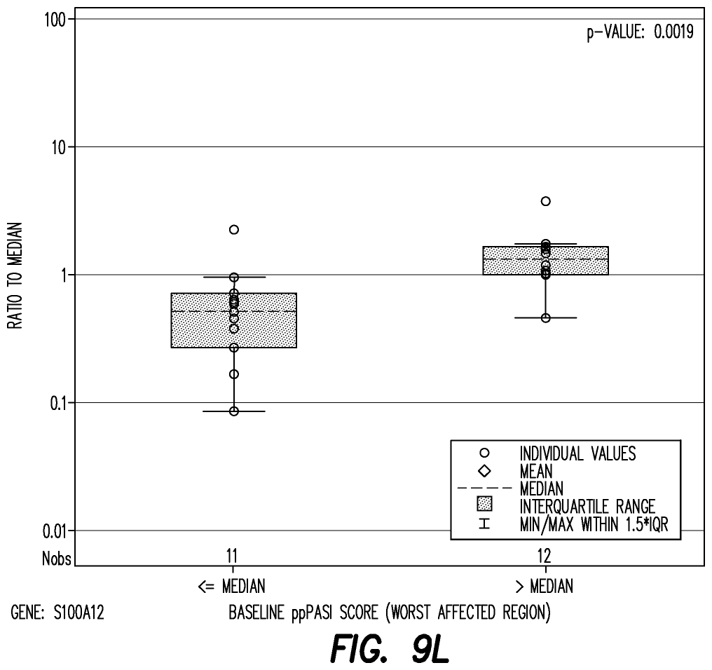

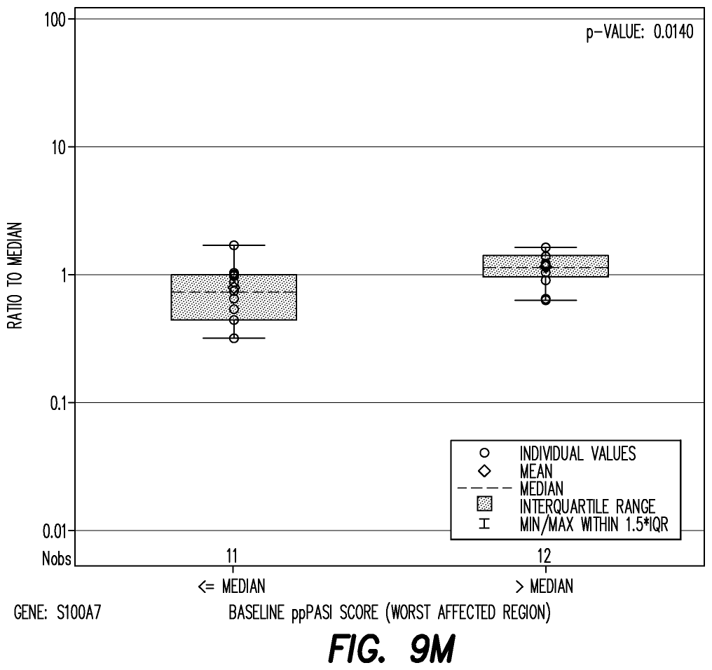

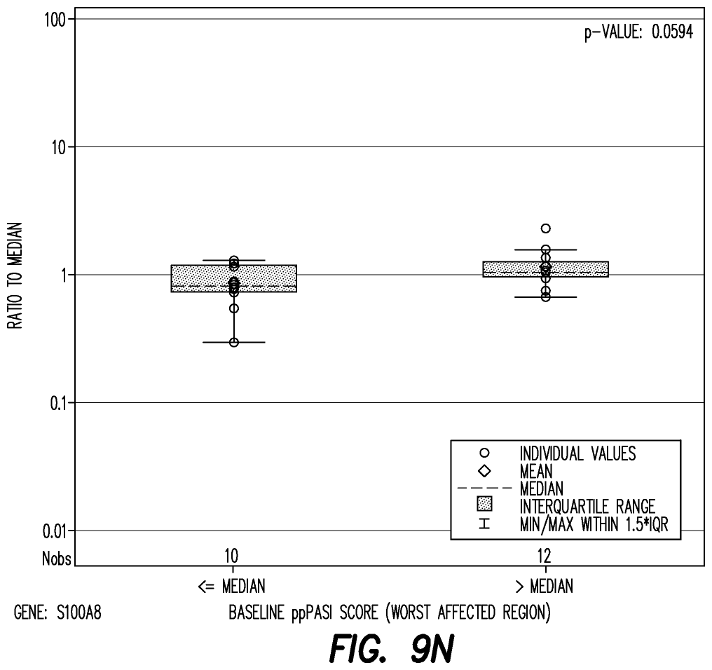

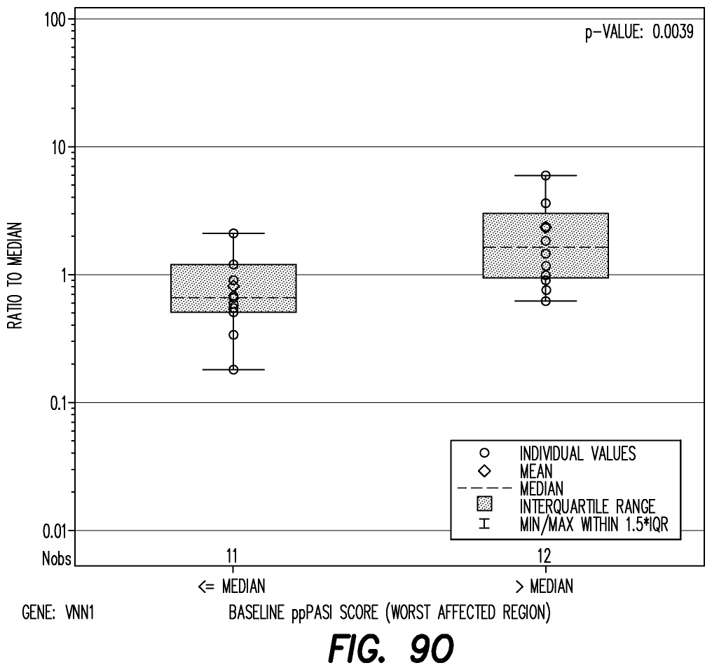

Based on this TaqMan-based gene expression analysis the following genes also show a statistical significant (p-value <0.05) higher expression at baseline patients above the median (worst area): C15orf48, CCL20, CXCR2, IGHA1, IL17A, IL17F, IL36A, IL36B, IL36RN, LCN2, MIR155HG, S100A12, S100A7, S100A8, and VNN1. (See FIG. 9 In addition we have also confirmed the following genes to be statistically significant (p-value <0.05) higher expressed in patients with an overall PPP ASI above the median: CXCR2, IL36G, IL36RN, PI3, S100A12, VNN3.

Post Hoc Analyses

Because the primary analysis did not show a significant difference between the anti-IL-36R antibody of the present invention and placebo treatment in terms of efficacy, exploratory analyses were conducted to dissect the results.

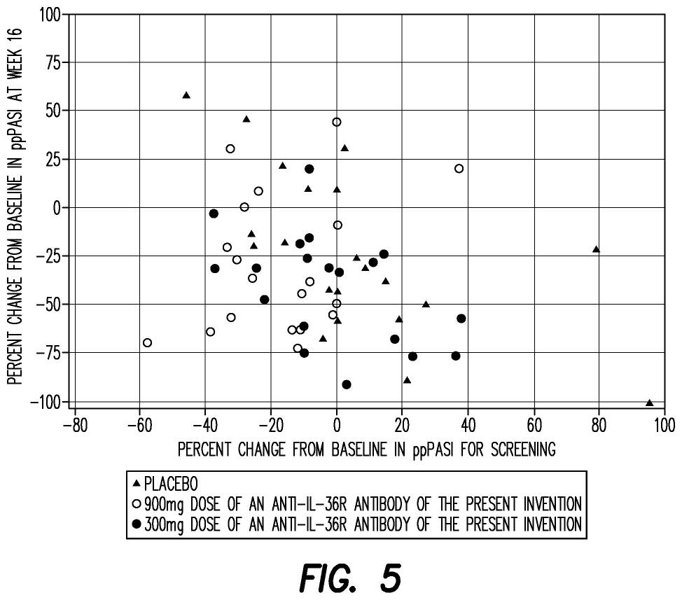

Percent change in PPP ASI was considered the most sensitive endpoint to assess changes in the PPP ASI score; it was therefore examined more closely. Furthermore, the PPP ASI score changed considerably from screening to baseline in several patients. Therefore, percent change in PPP ASI at Week 16 was plotted against the percent change in the PPP ASI score from screening to baseline for each patient (FIG. 5). This plot suggested a positive correlation between the change in the PPP ASI score from screening to baseline and the change in the PPP ASI score from baseline to Week 16: the more favourable the change from screening to baseline, the larger the decline (i.e. improvement) from baseline to Week 16. Furthermore, it showed that the PPP ASI score change from screening to baseline was reasonably well distributed around 0 in the placebo group and the 900 mg dose group of the anti-IL-36R antibody of the present invention while it was skewed towards a worsening in the 300 mg dose group of the anti-IL-36R antibody of the present invention (FIG. 5). Thus, the course of disease was less favourable in the 300 mg dose group of the anti-IL-36R antibody of the present invention than in the other two groups at baseline.

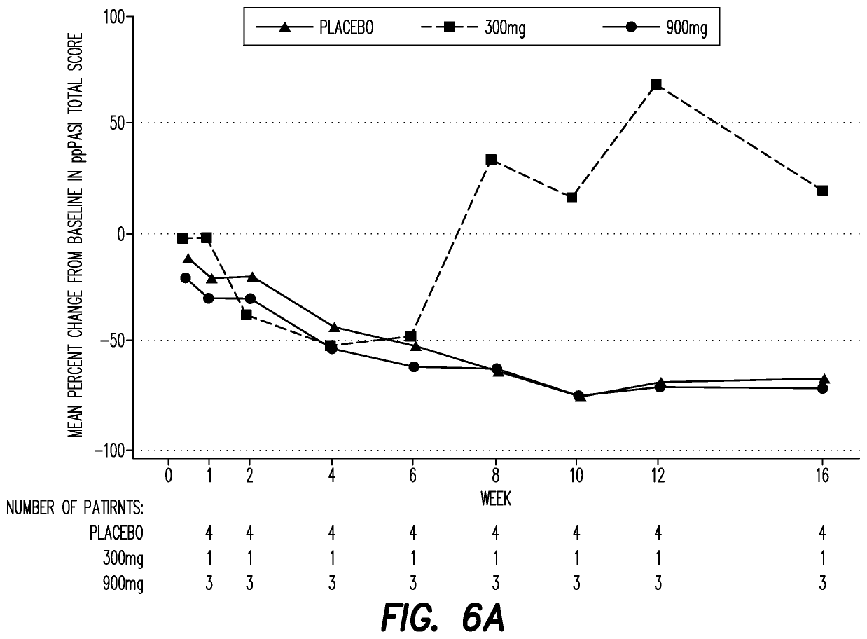

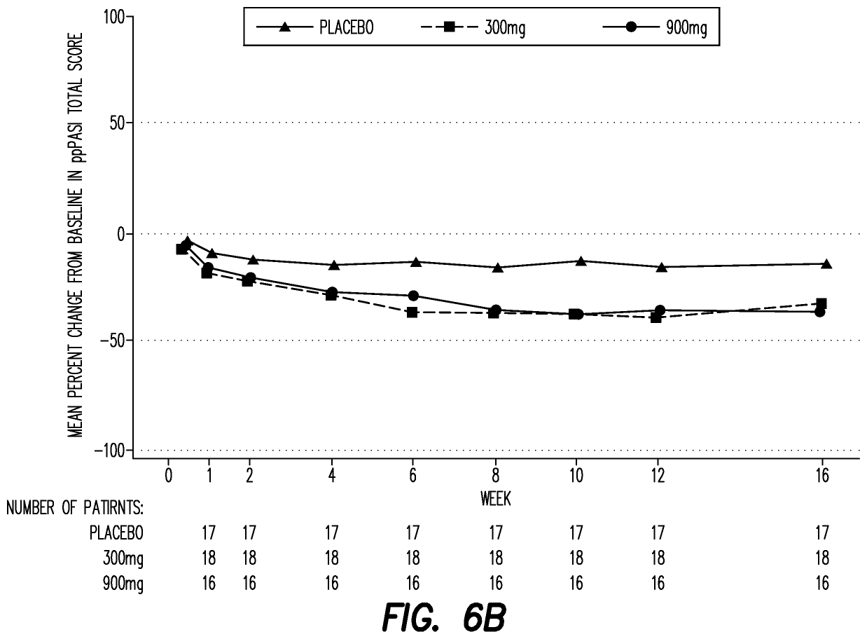

Thus, there was reason to assume that patients with considerable decline in the PPP ASI score from screening to baseline were at a point in their individual course of disease with clearance of skin symptoms. To differentiate between patients who were naturally improving (spontaneous remission) at baseline and those who were not, post-hoc analyses were performed dividing the population (i.e. all patients, as per inclusion criteria patients with PPP with a minimum PPP ASI score of 12, PPP PGA and visible pustules on the palms and/or soles) at baseline into those with improvement in the PPP ASI score from screening to baseline (screening 1.2× baseline) and those with no improvement (screening <1.2× baseline). In the group of patients with improvement in the PPP ASI score from screening to baseline, the mean PPP ASI score declined in all treatment groups until Week 6 and further declined at a comparable level in the 900 mg dose group of the anti-IL-36R antibody of the present invention and the placebo group. In the 300 mg dose group of the anti-IL-36R antibody of the present invention it increased after Week 6; this group, however, consisted of a single patient only (FIG. 6A).

In the group of patients without improvement in the PPP ASI score from screening to baseline, the decline in the mean PPP ASI totals score was larger in the treatment groups for the anti-IL-36R antibody of the present invention than in the placebo group at each time point up to Week 16 (FIG. 6B). The extent of the decline was similar in the two dose groups of the anti-IL-36R antibody of the present invention.

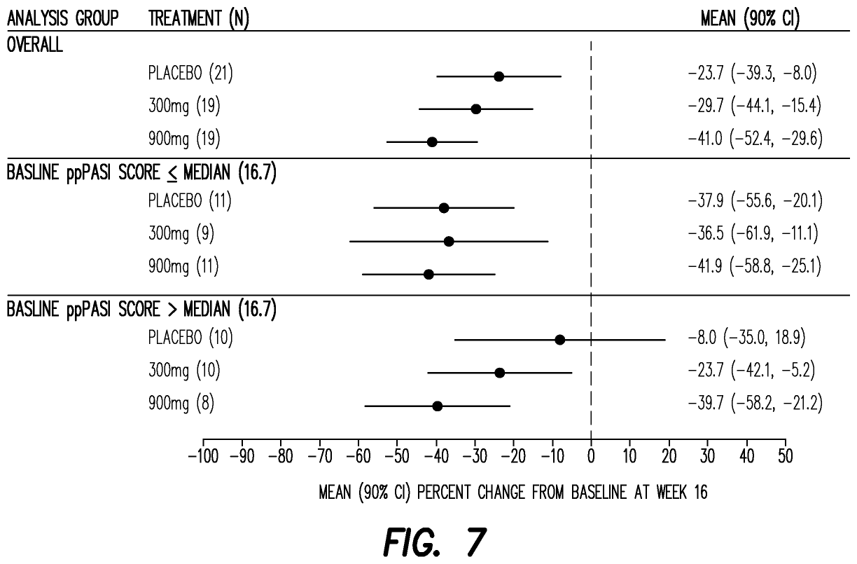

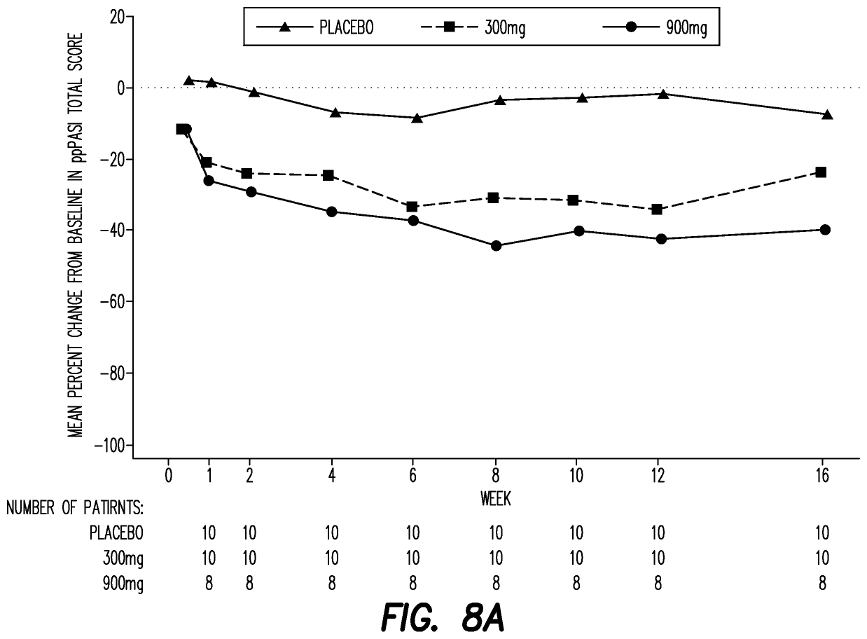

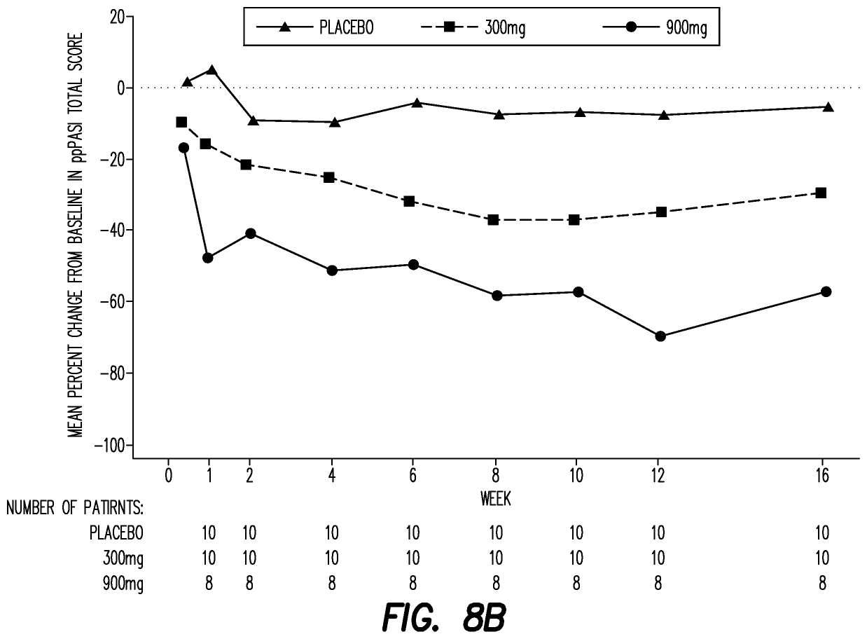

Because the disease severity in the overall trial population was relatively low, the trial population was divided into a subgroup with lower disease severity and a subgroup with higher disease severity, using the median baseline PPP ASI value (i.e. 16.7) as a cut-off. In patients with baseline PPP ASI above the median, the mean (90% confidence interval [CI]) percent change from baseline at Week 16 in PPP ASI was −39.7% (−58.2%, −21.2%) and −23.7% (−42.1%, −5.2%) in the 900 mg (n=8) and 300 mg (n=10) arms for an anti-IL-36R antibody of the present invention, respectively, versus −8.0% (−35.0%, 19.0%) in the placebo (n=10) arm (FIGS. 7 and 8A); the mean (90% CI) pustulation score percent change from baseline at Week 16 was −56.9% (−81.6%, −32.1%) and −29.9% (−42.4%, −17.5%) in the 900 mg and 300 mg arms for an antibody of the present invention, respectively, versus −5.4% (−35.6%, 24.9%) in the placebo arm, with improvement observed within two weeks of initiation with an anti-IL-36R antibody of the present invention (FIG. 8B). The results suggest a treatment effect of the anti-IL-36R antibody of the present invention on both endpoints in patients with a PPP ASI score>median at baseline which was especially pronounced for pustule severity.

### Discussion

This randomised, double-blind, placebo-controlled, parallel-design trial is the first study to investigate the safety and efficacy of an anti-IL-36R antibody of the present invention in patients with PPP. The trial consisted of a screening period (7-28 days), a 16-week treatment period (including 12 weeks of treatment and the primary endpoint assessment at Week 16), and a 16-week follow-up period. For the safety analyses, patients were considered to be ‘on-treatment’ until the end of the follow-up period.

As there are currently no established or validated endpoints available to specifically assess clinician- or patient reported outcomes in PPP, several endpoints were explored in this proof-of-concept trial. The endpoints included a PPP-specific PASI (PPP ASI) where induration was replaced with pustulation, as the sterile pustule is the primary component of PPP, while the scaling and erythema components remained unchanged. The primary efficacy endpoint was PPP ASI50 at Week 16.

In this study in patients with PPP, no clear treatment-emergent safety signal with the anti-IL-36R antibody of the present invention was identified, adding to previous safety data in 124 healthy volunteers (unpublished data), 7 patients with GPP, and are consistent with the recent characterisation of individuals with IL36R knockout mutations, resulting in the complete absence of the interleukin-36R but without any evidence of an increased risk of superinfection, nor of a significant impact on the innate and adaptive immune responses.

The assessment of efficacy did not show a significant difference between the doses of the anti-IL-36R antibody of the present invention and placebo in the proportion of patients who achieved PPP ASI50 at Week 16. Similarly, no significant difference between the anti-IL-36R antibody of the present invention and placebo was observed for any of the secondary endpoints (PPP ASI75 at Week 16, percent change from baseline in the PPP ASI score at Week 16, clinical response of 0 or 1=clear/almost clear via PPP PGA at Week 16). Looking at the PPP ASI score over time, the score declined in all treatment groups after start of treatment, with a more rapid decline in the treatment groups for the anti-IL-36R antibody of the present invention than in the placebo group. This pointed towards a treatment effect of the anti-IL-36R antibody of the present invention that was further explored in post hoc analyses.

There may be a number of factors which contributed to the lack of a statistically significant treatment effect of the anti-IL-36R antibody of the present invention in this trial. Without doubt, the relative small study size can mean that effects with a small number of patients (e.g. those patients with spontaneous improvement between screening and baseline) can have major effects on overall proportional results. Additionally, it may be that the primary endpoint assessment was performed at a point in time when the treatment effect of the anti-IL-36R antibody of the present invention coincided with the natural improvement of chronic PPP, which is characterised by a course of exacerbations and partial remissions. Also, the distinction between fluctuations of disease intensity and treatment effects might have been hindered by the relatively low severity of PPP in the trial population.

Post hoc analyses suggested a positive correlation between the change in the PPP ASI score from screening to baseline and the change in the PPP ASI score from baseline to Week 16: the more favourable the change from screening to baseline, the larger the decline (i.e. improvement) from baseline to Week 16. Thus, patients with a considerable decline in the PPP ASI score from screening to baseline showed high responses at Week 16, irrespective of the treatment group including placebo. It is probable that these patients were already on the way to remission in the course of disease when entering the trial and that this course continued at least until Week 16. To better assess a possible treatment effect of the anti-IL-36R antibody of the present invention, further post hoc analyses were conducted, excluding these patients with a presumably self-resolving state of disease. The analyses focussed on the mean change in the PPP ASI score, which was considered the most sensitive endpoint to assess changes in the PPP ASI score, as well as pustule severity, which is an important symptom of PPP. In patients with no improvement in the PPP ASI score from screening to baseline (i.e. patients, as per inclusion criteria patients with PPP with a minimum PPP ASI score of 12, PPP PGA and visible pustules on the palms and/or soles), the decline in the mean PPP ASI score was larger in the investigational treatment groups than in the placebo group at each time point up to Week 16. The extent of the decline was similar in the two dose groups of the anti-IL-36R antibody of the present invention. Furthermore, a pronounced effect was observed on pustule severity in both dose groups of the anti-IL-36R antibody of the present invention. Similar results were observed in patients with a PPP ASI score>median at baseline.

Of note, while PPP ASI change from screening to baseline was reasonably well distributed around 0 in the placebo group and the 900 mg group of the anti-IL-36R antibody of the present invention, it was skewed towards a worsening in the 300 mg group of the anti-IL-36R antibody of the present invention. Thus, a treatment effect was observable despite an unfavourable disease status at baseline in this treatment group.

Moreover, gene expression levels in skin biopsies from the worst affected areas (by PPP ASI of the region where the biopsy was taken; n=23) revealed a distinct molecular profile characterised by stronger expression of markers of the IL-36 pathway (IL36A/B/G), Th17 pathway (IL17A/F, DEFB4), neutrophil trafficking (CXCL1, CXCL2, CXCL6) and inflammation (TNF, S100A8/9/12) in patients with more severe lesions.

The positive effect on PPP ASI and pustule severity in the post hoc analyses supports concept that IL-36 upregulation is causally involved in PPP and that inhibition by the anti-IL-36R antibody of the present invention may become a safe and effective new treatment. However, it is noted that these subgroup analyses include only a small number of patients and no formal statistical analyses were performed, thus further studies with a larger patient population are planned to confirm the efficacy of IL-36R inhibition in patients with PPP.

### Example 6: Multi-Center, Double-Blind, Randomised, Placebo-Controlled, Phase 11b Dose-Finding Study to Evaluate Efficacy and Safety of Different Subcutaneous Doses of BI 655130 in Patients with Moderate to Severe Palmoplantar Pustulosis (PPP)

The purpose of this study is to test how effective and safe different doses of BI 655130 are in patients with a moderate to severe form of the skin disease Palmoplantar Pustulosis.

Trial rationale: The trial rationale is to demonstrate proof-of-concept with respect to a non-flat dose response curve and to define a suitable dose range for BI 655130 regarding efficacy and safety for further pivotal testing in Phase III in patients with PPP.

Trial objective(s): The primary objective is to provide dose-ranging data for 4 dose regimens of BI 655130 (with each regimen consisting of a loading and a separate maintenance subcutaneous dose) compared to placebo. The target dose(s) will be estimated from the model by incorporating information on the minimum clinically relevant effect and accounting for safety. The additional objectives are to explore long-term efficacy, safety and tolerability of multiple dose regimens of BI 655130 in patients with PPP.

Trial endpoints: The primary endpoint to assess efficacy of BI 655130 is % change in PPP ASI (Palmoplantar Pustulosis Area and Severity Index) from baseline at Week 16.

Secondary endpoints:
a. Change from baseline in Pain Visual Analogue Scale (VAS) score at Week 4 and 16.
b. PPP SI change from baseline at Week 16.
c. PPP ASI50 at Week 16.
d. PPP ASI75 at Week 16.
e. PPP PGA clear/almost clear at Week 16.
f. PPP PGA pustules clear/almost clear at Week 16.
g. Percent change in PPP ASI from baseline at Week 52.
h. Trial design: Placebo-controlled, double-blind, randomised, parallel-design comparison of 5 arms over 52 weeks.

Inclusion Criteria
i. 18 to 75 years of legal age (according to local legislation) at screening.
j. Diagnosis of Palmoplantar Pustulosis defined as presence of primary, persistent (>3 months duration), sterile, macroscopically visible pustules on the palms and/or soles, without or with plaque psoriasis elsewhere on the body.
k. Presence of white or yellow pustules on palms and/or soles at screening and baseline.
l. Pustular severity score in at least one region and 0 well-demarcated pustules (white or yellow pustules) across all regions at screening and baseline.
m. PPP PGA of at least moderate severity (3) at screening and baseline.
n. A minimum PPP ASI score of 12 at screening and baseline.
o. Male or female patients. Women of childbearing potential (WOCBP) must be ready and able to use highly effective methods of birth control per ICH M3 (R2) that result in a low failure rate of less than 1% per year when used consistently and correctly. A list of contraception methods meeting these criteria is provided in the patient information and in Section 4.2.2.3.
p. Signed and dated written informed consent in accordance with ICH-GCP and local legislation prior to admission to the trial.

Exclusion Criteria
a. Reduction in PPP ASI total score 5 from screening visit (Visit 1) to baseline (randomisation visit, Visit 2).
b. Patients with plaque psoriasis with worsening of plaque psoriasis within the last 3 months prior to screening.
c. Skin conditions that affect ability to score area and severity of PPP components (such as dyshidrotic eczema, calluses, tinea, xerotic scaling on heels, or maceration of interdigital areas).
d. Women who are pregnant, nursing, or who plan to become pregnant while in the trial.
e. Severe, progressive, or uncontrolled condition such as renal, hepatic, haematological, endocrine, pulmonary, cardiac, neurologic, cerebral, or psychiatric disease, or signs and symptoms thereof.
f. Presence or known history of anti-TNF-induced PPP-like disease.
g. Patient with a transplanted organ (with exception of a corneal transplant >12 weeks prior to screening) or who have ever received stem cell therapy (e.g., Prochymal).
h. Known history of lymphoproliferative disease, including lymphoma, or signs and symptoms suggestive of possible lymphoproliferative disease, such as lymphadenopathy and/or splenomegaly.
i. Any documented active or suspected malignancy or history of malignancy within 5 years prior to screening, except appropriately treated basal cell carcinoma of the skin, squamous cell carcinoma of the skin or in situ carcinoma of uterine cervix.
j. Use of any restricted medication as specified in Table 4.2.2.1:1 or any drug considered likely to interfere with the safe conduct of the study, as assessed by the investigator.
k. Plans for administration of live vaccines during the study period or within 6 weeks prior to randomisation.
l. History of allergy/hypersensitivity to the systemically administered trial medication agent or its excipients.
m. Active systemic infections during the last 2 weeks (exception: common cold) prior to randomisation, as assessed by the investigator.
n. Chronic or relevant acute infections including human immunodeficiency virus (HIV), viral hepatitis and (or) active or latent tuberculosis (TB):
o. QuantiFERON® TB test will be performed at screening. If the result is positive, the patient may participate in the study if further work up (according to local practice/guidelines) establishes conclusively that the patient has no evidence of active tuberculosis. Active TB patients must be excluded. If presence of latent tuberculosis is established, then treatment should have been initiated and maintained according to local country guidelines.
p. Major surgery (major according to the investigator's assessment) performed within 12 weeks prior to randomisation or planned within 52 weeks after randomisation (e.g. hip replacement, aneurysm removal, stomach ligation), as assessed by the investigator.
q. Patient has received surgical treatment of focal infection (e.g. tonsillectomy or dental therapy) within 6 months of randomisation.
r. Total white blood count (WBC)<3,000/μL, or platelets <100,000/μL or neutrophils <1,500/μL, or hemoglobin <8.5 g/dL at screening.
s. Aspartate aminotransferase (AST) or alanine aminotransferase (ALT)>2× the upper limit of normal, or total bilirubin >1.5× the upper limit of normal (patients with Gilbert's syndrome are not excluded) at screening.
t. Currently enrolled in another investigational device or drug study, or less than 30 days since ending another investigational device or drug study(s), or receiving other investigational treatment(s).
u. Chronic alcohol or drug abuse or any condition that, in the investigator's opinion, makes the patient an unreliable trial participant or unlikely to complete the trial Section 4.2.2.1.
v. Patients not expected to comply with the protocol requirements or not expected to complete the trial as scheduled.
w. Previous randomisation in this trial.

Dose:
q. Arm 1: 3000 mg total loading dose (600 mg weekly at Day 1, Week 1, Week 2, Week 3, and Week 4) followed by maintenance treatment 600 mg every 4 weeks (q4w) from Week 8
r. Arm 2: 3000 mg total loading dose (600 mg weekly at Day 1, Week 1, Week 2, Week 3, and Week 4) followed by maintenance treatment 300 mg every 4 weeks (q4w) from Week 8
s. Arm 3: 1500 mg total loading dose (300 mg weekly at Day 1, Week 1, Week 2, Week 3, and Week 4) followed by maintenance treatment 600 mg every 4 weeks (q4w) from Week 8
t. Arm 4: 1500 mg total loading dose (300 mg weekly at Day 1, Week 1, Week 2, Week 3, and Week 4), followed by maintenance treatment 300 mg every 4 weeks (q4w) until W16 (Week 8, Week 12, and Week 16) and 300 mg every 8 weeks (q8w) from Week 16

Mode of administration of the test product BI 655130: subcutaneous (SC)

Comparator product: Placebo

Dose: Arm 5: Loading dose Placebo; maintenance treatment placebo until Week 16 followed by maintenance treatment 600 mg q4w starting at Week 16

Mode of administration of placebo: subcutaneous (SC)

Statistical methods: The primary analysis consists of a combination of MCPMod-based testing (with respect to a non-flat dose response curve) and an evaluation of the dose-wise benefit at Week 16. As a basis for the MCPMod analysis a mixed effect model for repeated measurements (MMRM) is used. The MCPMod procedure allows for simultaneous evaluation of different potential dose response patterns, whilst protecting the overall probability of Type I error.

### Trial Objectives and Endpoints

### Main Objectives, Primary and Secondary Endpoints

### Main Objectives

The present trial will be performed to demonstrate proof of concept with respect to a non-flat dose response curve, and to define a suitable dose range for BI 655130 regarding efficacy and safety for further pivotal testing in Phase III in patients with PPP. For this purpose, a multiple comparison procedure with modelling techniques (MCPMod) approach is considered.

The primary objective is to provide dose-ranging data for 4 dose regimens of BI 655130 (with each regimen consisting of a loading and a separate maintenance subcutaneous dose) compared to placebo on the primary endpoint of percentage change from baseline in PPP ASI at Week 16. The target dose(s) will be estimated from the model by incorporating information on the minimum clinically relevant effect and accounting for safety. Supportive dose-ranging assessments will also be done on pre-specified secondary endpoints.

The primary endpoint comparison will be performed for all randomised and treated patients who have a baseline value for the primary endpoint. The primary treatment comparison will be performed as if all patients took randomised treatment for the duration of the trial that is excluding the effects of either treatment discontinuation or use of rescue therapy.

The additional objectives are to explore long-term efficacy, safety and tolerability of multiple dose regimens of BI 655130 in patients with PPP.

### Primary Endpoint(s)

The primary endpoint to assess efficacy of BI 655130 is % change in PPP ASI from baseline at Week 16. Any data collected after use of any rescue therapy or after 6 weeks following discontinuation of treatment (to allow for incorporation of the continuing maximum treatment effect period) are censored for the purpose of the primary estimand.

### Secondary Endpoint(s)

Secondary endpoints are defined as described below. Note that for the secondary endpoints, any data collected after use of any rescue therapy or after 6 weeks following discontinuation of treatment (to allow for incorporation of the continuing maximum treatment effect period) are censored for the purpose of the primary estimand.
a. Change from baseline in PPP Pain Visual Analog Scale (VAS) score at Week 4 and 16
b. PPP SI change from baseline at Week 16
c. PPP ASI50 at Week 16
d. PPP ASI75 at Week 16
e. PPP PGA clear/almost clear at Week 16
f. PPP PGA pustules clear/almost clear at Week 16
g. Percent change in PPP ASI from baseline at Week 52
h. FURTHER OBJECTIVES AND FURTHER ENDPOINTS
i. Further objectives
j. Further objectives are the assessment of long-term efficacy, safety and tolerability, pharmacokinetics and pharmacodynamics of different dose regimens of BI 655130 compared to placebo in patients with PPP.
k. Further endpoints
l. The occurrence of Treatment Emergent Adverse Events (TEAEs)
m. Percent change in pustule count from baseline over time
n. Percent change in pustular severity (as component of PPP ASI) from baseline over time
o. PPP PGA clear/almost clear over time
p. PPP PGA pustule clear/almost clear over time
q. Change from baseline in total score of PPQLI, DLQI, PSS, and BASDAI over time
r. PPP ASI50 over time
s. PPP ASI75 over time
t. Percent change from baseline in the PPP ASI over time
u. PPSI change from baseline over time (derived from PPP ASI severity scores)
v. Change from baseline in both Pain Visual Analog Scale (VAS) scores over time
w. Time (weeks) to achieving PPP ASI75
x. Time (weeks) to achieving PPP ASI50
y. Time (weeks) to loss of PPP ASI75
z. Time (weeks) to loss of PPP ASI50
aa. Percent change in PASI (for the patients with concurrent psoriasis) over time
bb. sPGA (for the patients with concurrent psoriasis) over time
cc. Percent change in TPSS (for the patients with concurrent psoriasis) from baseline over time
dd. PK concentration
ee. Biomarkers to evaluate treatment response in PPP

### Description of Design and Trial Population

### Overall Trial Design and Plan

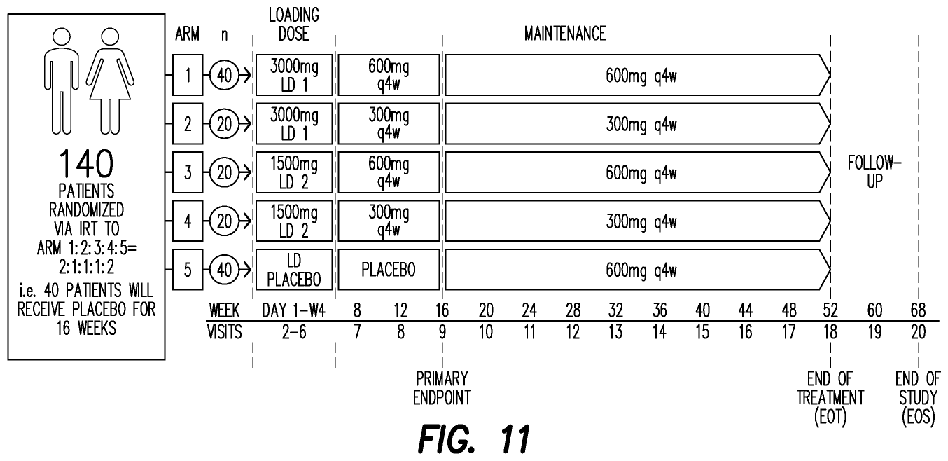

This is a randomised, double-blind, placebo-controlled, parallel-design dose-finding trial comprising of 4 active doses compared to placebo. Active treatment arms consist of active loading dose and active maintenance treatment. Two different loading doses and two different maintenance treatment doses are to be tested up to Week 16 (to give four different BI 655130 dose regimens). From Week 16 onwards, three different maintenance treatment doses are to be tested. The trial design is illustrated in FIG. 11.

### Example 7: Treating Patients Suffering from PPP (Palmoplantar Pustulosis) with an Anti-IL-36R Antibody

In this example, an anti-IL36R antibody (e.g., an anti-IL-36R antibody of the present invention) is used to treat a patient with PPP, or to treat a patient with moderate to severe PPP, or to treat a patient with chronic conditions associated with PPP (including periodic appearance or worsening of pustules), or to reduce or alleviate signs or symptoms of an acute (including new appearance or worsening of pustules) or chronic PPP in a patient, or to reduce the severity and/or duration of PPP flares (including new appearance or worsening of pustules) in patients, or to treat a skin disorder associated with acute PPP (including new appearance or worsening of pustules) in a patient, or to prevent recurrence of PPP flares (including new appearance or worsening of pustules) in a patient.

Initially, a patient is diagnosed to have PPP, or moderate to severe PPP, or a chronic condition associated with PPP (including periodic appearance or worsening of pustules), or a sign or symptom of an acute (including new appearance or worsening of pustules) or chronic PPP, or PPP flares (including new appearance or worsening of pustules), or a skin disorder associated with acute PPP (including new appearance or worsening of pustules), or recurring PPP flares (including new appearance or worsening of pustules). A dose regimen of the anti-IL-36R antibody according any of those listed in Tables 1-4 is administered to the patient.

Following the administration of the anti-IL-36R antibody, data assessment reveals one or more of the followings:
(a) the patient achieves a 50% reduction in PPP ASI (PPP ASI50) at or after about week 4, week 6, week 8, week 12, week 16 or week 24; or
(b) the patient experience at about 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% reduction in the number of drug-related Adverse Events (AEs) as compared to other treatments (e.g., guselkumab);
(c) the patient experiences at least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more improvement in his or her pustule severity (as compared to baseline) at or after about week 4, week 6, week 8, week 12, week 16 or week 24; or
(d) the anti-IL-36R antibody treatment shows a superior efficacy over guselkumab by at least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, about 60%, about 70%, about 80%, about 90%, about 100%, about 150%, about 200% or more at or after about week 4, week 6, week 8, week 12, week 16 or week 24 or over time; or
(e) the patient achieves a PPP Physicians Global Assessment (PPP PGA) score of 0 or 1 (clear/almost clear) at or after about week 4, week 6, week 8, week 12, week 16 or week 24; or
(f) the patient achieves a Psoriasis Area and Severity Index for PPP (PPP ASI) 75 at or after about week 4, week 6, week 8, week 12, week 16 or week 24; or
(g) the patient experiences 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, about 60%, about 70%, about 80%, about 90%, about 100%, about 150%, about 200% or more improvement from baseline in the PPP ASI at or after about week 4, week 6, week 8, week 12, week 16 or week 24; or
(h) the patient achieves an improved change of 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, about 60%, about 70%, about 80%, about 90%, about 100%, about 150%, about 200% or more from baseline in Pain Visual Analog Scale (VAS) score at or after about week 4, week 6, week 8, week 12, week 16 or week 24; or
(i) the patient achieves a clinical improvement of 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, about 60%, about 70%, about 80%, about 90%, about 100%, about 150%, about 200% or more from baseline as assessed via Dermatology Life Quality Index (DLQI) at or after about week 4, week 6, week 8, week 12, week 16 or week 24; or
(j) the patient achieves a PPP ASI50 at visit 1, 2, 3, 4, 5, 6, 7, 8, 9, 10 or all other visits; or
(k) the patient achieves 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, about 60%, about 70%, about 80%, about 90%, or about 100% reduction in PPP ASI scores at week 16 and all other visits; or
(l) the patient achieves PPP Physicians Global Assessment (PPP PGA) score of 0 or 1 (clear/almost clear) at visit 1, 2, 3, 4, 5, 6, 7, 8, 9, 10 or all other visits;
(m) the patient achieves a PPP ASI75 at visit 1, 2, 3, 4, 5, 6, 7, 8, 9, 10 or all other visits;
(n) the patient experiences 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, about 60%, about 70%, about 80%, about 90%, or about 100% percent change from baseline in the PPP ASI at visit 1, 2, 3, 4, 5, 6, 7, 8, 9, 10 or all other visits; or
(o) the patient experiences a lesser time (in days, e.g., about 5, about 10, about 15, about 20, about 25, about 30, about 40, about 50, about 60, about 70, about 80, about 90, about 100, about 120, about 140, about 160, about 180, about 200, about 250, about 300 or more days) to achieving PPP ASI50 as compared to other treatments (e.g., guselkumab); or
(p) the patient experiences a longer time (in days, e.g., about 5, about 10, about 15, about 20, about 25, about 30, about 40, about 50, about 60, about 70, about 80, about 90, about 100, about 120, about 140, about 160, about 180, about 200, about 250, about 300 or more days) to loss of PPP ASI50 as compared to other treatments (e.g., guselkumab);
(q) the patient experiences 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, about 60%, about 70%, about 80%, about 90%, or about 100% of improved change in plaque psoriasis BSA involvement at or after about week 4, week 6, week 8, week 12, week 16 or week 24 in patients with concurrent plaque psoriasis at baseline; or
(r) the patient experiences at least about 10%, about 20%, about 30%, about 40%, about 50%, about 60%, about 70%, about 80%, about 90%, about 100%, about 150%, about 200%, or about 300% superiority over placebo in achieving PPP ASI50 at or after about week 4, week 6, week 8, week 12, week 16 or week 24; or
(s) the patient achieves about 5%, about 10%, about 15%, about 20%, about 30%, about 40%, about 50%, about 60%, about 70%, about 80%, about 90%, about 100% or more of change in PPP ASI from baseline at or after about week 4, week 6, week 8, week 12, week 16 or week 24; or
(t) the patient achieves about 5%, about 10%, about 15%, about 20%, about 30%, about 40%, about 50%, about 60%, about 70%, about 80%, about 90%, about 100% or more of positive or improved change in Pain VAS score from baseline at or after about week 4, week 6, week 8, week 12, week 16 or week 24; or
(u) the patient achieves about 5%, about 10%, about 15%, about 20%, about 30%, about 40%, about 50%, about 60%, about 70%, about 80%, about 90%, about 100% or more of positive or improved PPP SI change from baseline at or after about week 4, week 6, week 8, week 12, week 16 or week 24; or
(v) the patient achieves about 5%, about 10%, about 15%, about 20%, about 30%, about 40%, about 50%, about 60%, about 70%, about 80%, about 90%, about 100% or more of positive or improved PPP ASI change from baseline at week 52; or
(w) the patient achieves about 5%, about 10%, about 15%, about 20%, about 30%, about 40%, about 50%, about 60%, about 70%, about 80%, about 90%, about 100% or more of reduction in occurrence of Treatment Emergent Adverse Events (TEAEs) from baseline overtime or at or after about week 4, week 6, week 8, week 12, week 16 or week 24; or
(x) the patient achieves about 5%, about 10%, about 15%, about 20%, about 30%, about 40%, about 50%, about 60%, about 70%, about 80%, about 90%, about 100% or more of a positive or improved change in pustule count from baseline over time or at or after about week 4, week 6, week 8, week 12, week 16 or week 24; or
(y) the patient achieves about 5%, about 10%, about 15%, about 20%, about 30%, about 40%, about 50%, about 60%, about 70%, about 80%, about 90%, about 100% or more of a positive or improved change in pustular severity from baseline over time or at or after about week 4, week 6, week 8, week 12, week 16 or week 24; or
(z) the patient achieves a PPP PGA clear/almost clear as compared to baseline or placebo over time or at or after about week 4, week 6, week 8, week 12, week 16 or week 24; or
(aa) the patient achieves a PPP PGA pustule clear/almost clear as compared to baseline or placebo over time or at or after about week 4, week 6, week 8, week 12, week 16 or week 24; or
(bb) the patient achieves about 5%, about 10%, about 15%, about 20%, about 30%, about 40%, about 50%, about 60%, about 70%, about 80%, about 90%, about 100% or more of a positive change from baseline in total score of PPQLI (Palmoplantar Quality of Life Instrument), DLQI (Dermatology Life Quality Index), PSS (Psoriasis Symptom Scale), and BASDAI (Bath Ankylosing Spondylitis Disease Activity Index) over time or at or after about week 4, week 6, week 8, week 12, week 16 or week 24; or
(cc) the patient achieves a PPP ASI50 over time or at or after about week 4, week 6, week 8, week 12, week 16 or week 24 or week 52; or
(dd) the patient achieves a PPP ASI75 over time or at or after about week 4, week 6, week 8, week 12, week 16 or week 24 or week 52; or
(ee) the patient achieves about 5%, about 10%, about 15%, about 20%, about 30%, about 40%, about 50%, about 60%, about 70%, about 80%, about 90%, about 100% or more of a positive or improved percent change from baseline in the PPP ASI over time or at or after about week 4, week 6, week 8, week 12, week 16 or week 24 or week 52; or
(ff) the patient achieves about 5%, about 10%, about 15%, about 20%, about 30%, about 40%, about 50%, about 60%, about 70%, about 80%, about 90%, about 100% or more of a positive or improved PPSI change as compared to baseline over time or at or after about week 4, week 6, week 8, week 12, week 16 or week 24 or week 52; or
(gg) the patient achieves about 5%, about 10%, about 15%, about 20%, about 30%, about 40%, about 50%, about 60%, about 70%, about 80%, about 90%, about 100% or more of a positive or improved change in Pain VAS score for pain on palm and/or soles (PPP Pain VAS) and/or one for muscular and joint pain as compared to baseline or placebo over time or at or after about week 4, week 6, week 8, week 12, week 16 or week 24 or week 52; or
(hh) the patient achieves a shorter time to PPP ASI75 (in days, e.g., about 5, about 10, about 15, about 20, about 25, about 30, about 40, about 50, about 60, about 70, about 80, about 90, about 100, about 120, about 140, about 160, about 180, about 200, about 250, about 300 or more days; or in weeks, e.g., 4 weeks, 8 weeks, 12 weeks, 16 weeks, 20 weeks, 24 weeks, or more) as compared to baseline or placebo over time or at or after about week 4, week 6, week 8, week 12, week 16 or week 24 or week 52; or
(ii) the patient achieves a shorter time to PPP ASI50 (in days, e.g., about 5, about 10, about 15, about 20, about 25, about 30, about 40, about 50, about 60, about 70, about 80, about 90, about 100, about 120, about 140, about 160, about 180, about 200, about 250, about 300 or more days; or in weeks, e.g., 4 weeks, 8 weeks, 12 weeks, 16 weeks, 20 weeks, 24 weeks, or more) as compared to baseline or placebo over time or at or after about week 4, week 6, week 8, week 12, week 16 or week 24 or week 52; or
(jj) the patient achieves a longer time to loss of PPP ASI75 (in days, e.g., about 5, about 10, about 15, about 20, about 25, about 30, about 40, about 50, about 60, about 70, about 80, about 90, about 100, about 120, about 140, about 160, about 180, about 200, about 250, about 300 or more days; or in weeks, e.g., 4 weeks, 8 weeks, 12 weeks, 16 weeks, 20 weeks, 24 weeks, or more) as compared to baseline or placebo over time or at or after about week 4, week 6, week 8, week 12, week 16 or week 24 or week 52; or
(kk) the patient achieves a longer time to loss of PPP ASI50 (in days, e.g., about 5, about 10, about 15, about 20, about 25, about 30, about 40, about 50, about 60, about 70, about 80, about 90, about 100, about 120, about 140, about 160, about 180, about 200, about 250, about 300 or more days) as compared to baseline or placebo over time or at or after about week 4, week 6, week 8, week 12, week 16 or week 24 or week 52; or
(ll) the patient achieves about 5%, about 10%, about 15%, about 20%, about 30%, about 40%, about 50%, about 60%, about 70%, about 80%, about 90%, about 100% or more of a positive or improved change in PASI as compared to baseline or placebo over time or at or after about week 4, week 6, week 8, week 12, week 16 or week 24 or week 52; or
(mm) the patient achieves about 5%, about 10%, about 15%, about 20%, about 30%, about 40%, about 50%, about 60%, about 70%, about 80%, about 90%, about 100% or more of a positive or improved change in sPGA as compared to baseline or placebo over time or at or after about week 4, week 6, week 8, week 12, week 16 or week 24 or week 52; or
(nn) the patient achieves about 5%, about 10%, about 15%, about 20%, about 30%, about 40%, about 50%, about 60%, about 70%, about 80%, about 90%, about 100% or more of a positive or improved percent change in TPSS as compared with baseline or placebo over time or at or after about week 4, week 6, week 8, week 12, week 16 or week 24 or week 52; or
(oo) the patient achieves about 5%, about 10%, about 15%, about 20%, about 30%, about 40%, about 50%, about 60%, about 70%, about 80%, about 90%, about 100% or more of a positive or improved pharmacokinetic as compared to baseline or placebo over time or at or after about week 4, week 6, week 8, week 12, week 16 or week 24 or week 52; or
(pp) the patient achieves about 1.2 fold, about 1.5 fold, about 2 fold, about 2.5 fold, about 3 fold, about 3.5 fold, about 4 fold or more of an improved gene expression change for the genes disclosed herein as an indication that the treatment is efficacious as compared with baseline or placebo over time at or after about week 4, week 6, week 8, week 12, week 16 or week 24 or week 52; or
(qq) the patient achieves a PPP PGA of 0 or 1 at a reduced time as compared with baseline or placebo over time or at or after about week 4, week 6, week 8, week 12, week 16 or week 24 or week 52.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

All patents and/or publications including journal articles cited in this disclosure are expressly incorporated herein by reference.

1. A method of treating palmoplantar pustulosis (PPP) in a patient, said method comprising administering or having administered to the patient a therapeutically effective amount of an anti-IL-36R antibody.

2. A method of treating moderate to severe PPP in a patient, comprising administering or having administered to the patient a therapeutically effective amount of an anti-IL-36R antibody.

3. A method of reducing or alleviating signs and symptoms of an acute phase flare-up of PPP in a patient, said method comprising administering or having administered to the patient a therapeutically effective amount of an anti-IL-36R antibody.

4. A method of reducing the severity and duration of PPP flares, said method comprising administering or having administered to the patient a therapeutically effective amount of an anti-IL-36R antibody.

5. A method of treating a skin disorder associated with acute PPP, said method comprising administering or having administered to the patient a therapeutically effective amount of an anti-IL-36R antibody.

6. The method of claim 1, wherein the anti-IL-36R antibody comprises: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

7. The method of claim 1, wherein the anti-IL-36R antibody comprises:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

8. The method of claim 1, wherein the anti-IL-36R antibody comprises:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

9. The method of claim 1, wherein the anti-IL-36R antibody comprises:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

10. The method of claim 1, wherein the anti-IL-36R antibody is administered or having been administered subcutaneously.

11. The method of claim 10, wherein the subcutaneous administration comprises administration of 300 mg or 600 mg dose of the anti-IL-36R antibody.

12. The method of claim 11, wherein the subcutaneous administration is conducted qw (once every week), q2w (once every 2 weeks), q4w (once every 4 weeks), q6w (once every 6 weeks) or q8w (once every 8 weeks), or a combination thereof.

13. The method of claim 10, wherein the subcutaneous administration comprises an initial dose.

14. The method of claim 13, wherein the subcutaneous administration further comprises a subsequent dose.

15. The method of claim 13, wherein the initial dose is 150 mg, 300 mg or 600 mg.

16. The method of claim 15, wherein the initial dose of 150 mg or 300 mg is administered per day (in consecutive days) for two weeks.

17. The method of claim 15, wherein the initial the initial dose of 600 mg is administered once per week for two weeks comprising weeks 0 and 1; weeks 0 and 2; weeks 0 and 3; or weeks 0 and 4.

18. The method of claim 15, wherein the initial dose of 600 mg is administered once per week for three weeks comprising weeks 0, 1 and 2; weeks 0, 1 and 3; weeks 0, 1 and 4; weeks 0, 2 and 3; weeks 0, 2 and 4; or weeks 0, 3 and 4.

19. The method of claim 15, wherein the initial dose of 600 mg is administered once per week for four weeks comprising weeks 0, 1, 2 and 3; weeks 0, 1, 2 and 4; weeks 0, 1, 3 and 4; or weeks 0, 2, 3 and 4.

20. The method of claim 15, wherein the initial dose of 600 mg is administered twice per week for 2 weeks, twice per week for 3 weeks or twice per week for 4 weeks.

21. The method of claim 14, wherein the subsequent dose is 300 mg or 600 mg.

22. The method of claim 21, wherein the subsequent dose administration begins two to four weeks after the initial dose administration ends.

23. The method of claim 21, wherein the subsequent dose of 300 mg or 600 mg is administered q2w (once every 2 weeks), q4w (once every 4 weeks), q6w (once every 6 weeks) or q8w (once every 8 weeks).

24. The method of claim 1, wherein the administration results in one or more of the following outcomes in the patient:
(a) Palmoplantar Pustular Psoriasis Area and Severity Index 50 (PPP ASI50) at week 16;
(b) reduction in the number of patients with drug-related Adverse Events (AEs);
(c) PPP Physicians Global Assessment (PPP PGA) score of 0 or 1=clear/almost clear at week 16;
(d) PPP ASI75 at week 16;
(e) Percent change from baseline in the PPP ASI at week 16;
(f) Change from baseline in Pain Visual Analog Scale (VAS) score at Week 16 and all other visits collected;
(g) Clinical Improvement assessed via Dermatology Life Quality Index (DLQI) at week 16 and all other visits collected compared to baseline;
(h) PPP ASI50 at all other visits collected;
(i) Modified (precise) PPP ASI scores at week 16 and all other visits collected;
(j) Treatment success defined as achieving a clinical response of 0 or 1=clear/almost clear via PPP Physicians Global Assessment (PPP PGA) at all other visits collected;
(k) PPP ASI75 at all other visits collected;
(l) Percent change from baseline in the PPP ASI at all other visits collected;
(m) Time (days) to achieving PPP ASI50;
(n) Time (days) to loss of PPP ASI50;
(o) Change in plaque psoriasis BSA involvement at week 16 in patients with concurrent plaque psoriasis at baseline;
(p) Superior efficacy over guselkumab; and/or
(q) At least about 40% superiority to placebo in achieving PPP ASI50 at week 16.
